(19) United States
(12) Patent Application Publication
(10) Pub. No.: US 2025/0319131 A1
(43) Pub. Date: Oct. 16, 2025

(54) TUNABLE CYTOKINE RECEPTOR SIGNALING DOMAINS

(71) Applicant: The Board of Trustees of the Leland Stanford Junior University, Stanford, CA (US)

(72) Inventors: Kenan Christopher Garcia, Menlo Park, CA (US); Leon Lih-Ren Su, Mountain View, CA (US); Grayson Rodriguez, Fayetteville, NC (US)

(21) Appl. No.: 18/881,601

(22) PCT Filed: Jul. 10, 2023
(86) PCT No.: PCT/US2023/069856

§ 371 Date: Jan. 6, 2025

Related U.S. Application Data
(60) Provisional application No. 63/388,128, filed on Jul. 11, 2022.

(51) Int. Cl. A61K 35/17; A61K 40/11; A61K 40/30; C07K 14/715; C12N 15/86

(52) U.S. Cl. CPC A61K 35/17; A61K 40/11; A61K 40/30; C07K 14/7153; C07K 14/7155; C07K 14/7156; C12N 15/86; C07K 2319/03; C12N 2740/13043

(57) ABSTRACT

Engineered tunable chimeric receptor/ligand pairs, and methods of use thereof, are provided.

### CROSS REFERENCE TO OTHER APPLICATIONS

This application claims the benefit of PCT Application No. PCT/US2023/069856, filed Jul. 10, 2023, which claims benefit of U.S. Provisional Application No. 63/388,128, filed Jul. 11, 2022, the contents of which applications are hereby incorporated by reference in their entirety.

### INCORPORATION BY REFERENCE OF SEQUENCE LISTING

A Sequence Listing is provided herewith as a Sequence Listing XML, STAN-1987_Sequence_listing created on Jan. 3, 2025 and having a size of 172,028 bytes. The contents of the Sequence Listing XML are incorporated herein by reference in their entirety.

### BACKGROUND

The ability to manipulate receptors to provide novel intracellular signal combinations is a significant challenge in protein engineering. Of particular interest is the ability to “tune” cytokine receptor signaling in way that can provide for a desired activation of specific intracellular pathways.

Cytokines are secreted glycoproteins that act as intercellular messengers to control hematopoietic and immune systems. A number of cytokines signal through binding to cell surface receptors that transduce signaling through the JAK/STAT cascade. The JAK/STAT cascade utilizes a receptor, kinase, and transcription factor to elicit a response. A general rule of cytokine signaling is that each cytokine binds to a specific receptor, this binding induces activation of specific JAK(s) and STAT(s).

A cytokine binds to a specific receptor on the surface of its target cell. These receptors contain intracellular domains that are constitutively associated with members of the JAK (Janus Kinase) family of tyrosine kinases. JAKs are inactive, but specific binding of a cognate cytokine to the extracellular domain (ECD) receptor induces their auto-activation by transphosphorylation. Once activated, JAKs phosphorylate the intracellular tails of the receptors on specific tyrosines which in turn act as docking sites for members of the Signal Transducers and Activators of Transcription (STAT) family of transcription factors. Receptor-localized STATs are then phosphorylated by JAK which leads to their disassociation from the receptor and translocation to the nucleus, where they drive the expression of cytokine-responsive genes.

The human JAK family contains four JAKs: JAK1, JAK2, JAK3 and TYK2. These proteins are tyrosine kinases. Two regions on the cytoplasmic tail of receptors, termed Box 1 and Box 2, are critical for the association of JAKs with receptor. Box 1 is proline rich and is located approximately 10 residues from the C-terminus of the transmembrane region of the receptor; Box 2 is about 10-50 residues further downstream and is rich in hydrophobic residues. Sequence differences within the Box 1 and Box 2 motifs of different receptors determine which JAK is bound by the receptor.

JAK phosphorylation of distal tyrosines on the receptor intracellular domains enables those sequences to act as docking sites for STAT proteins, as well as are other non-STAT family proteins that also can be recruited to the receptor and activate important signaling pathways. The human STAT family contains seven STATs: STAT1, STAT2, STAT3, STAT4, STAT5A, STAT5B and STAT6. Just as different receptors bind different JAKs, so they also bind different STATs. The ability of a certain cytokine to induce activation of a particular set of STATs is driven purely by the STAT-binding sites contained within the receptor ICD. STAT-binding sites from one receptor can be replaced with binding sites for different STATs from other receptors and thereby activate nonphysiological STATs (see, for example, Stahl et al. (1995) Science 267:1349-1353). It has been proposed that the affinity for one STAT over another is a function of the sequence immediately surrounding the phosphotyrosine, for example pYxxP, pYxxQ, pYxxL, and pYxxF sequences are associated with recruitment of STAT1, STAT3, STAT5, and STAT6, respectively.

The activation of transcription by STAT proteins drives the phenotype of immune cells during the processes of activation, effector function, memory, and the like. Activated STAT proteins translocate to the cell nucleus, bind to specific regulatory elements, and in coordination with other transcription factors, regulate transcription of particular genes to provide a specific transcriptional profile. It is this gene profile, is in the context of a network of many other signaling pathways and combination of transcription factors, that yield specific but diverse transcriptional profiles that results in the different phenotypes and functions of immune cells. Tools to tune these responses are of great interest, for example for cell mediated therapies. The present disclosure provides such tools.

### SUMMARY

Compositions and methods are provided for tunable cytokine receptor polypeptides, which polypeptides comprise: (a) an extracellular domain (ECD) that, when bound to its cognate ligand, multimerizes, and through an intracellular domain (ICD) activates intracellular signaling pathways; (b) a transmembrane domain (TM); and (c) an intracellular domain (ICD), which has a sequence other than the naturally occurring ICD of the extracellular domain, where the ICD may comprise targeted amino acid modifications that alter the STAT protein profile activated by the receptor. The phenotype of a cell, e.g. an immune cell, can be altered by transducing the cell with a tunable cytokine receptor, which delivers customized STAT activation patterns upon binding to its cognate ligand. The set of STAT proteins activated by a tunable cytokine receptor can influence the transduced cell phenotype, including without limitation by altering the balance of proliferation or stemness.

Binding of its cognate ligand to the ECD activates signaling via the ICD of the receptor. Selection of the ICD, and targeted amino acid modification of the ICD, e.g. in STAT binding sequences, allows for tuning of the receptor to provide a desired intracellular signal. In some specific embodiments, the STAT activation profile of a receptor is tuned to provide for a desired balance of activation and stemness in a cell expressing the tunable receptor.

Suitable ICD domains comprise a functional fragment derived from a receptor subunit, for example derived from a cytokine receptor. In some such embodiments the ICD is a functional fragment derived from a receptor and is substantially or entirely the ICD and transmembrane domain (TM) of the receptor. In some embodiments the ICD of the tunable cytokine receptor comprises phosphosites for one or more STAT signaling proteins, e.g. STAT1, STAT2, STAT3, STAT4, STAT5A, STAT5B, STAT6, etc.

In some embodiments the ICD comprises the TM and ICD sequence of a receptor subunit selected from IL3Rα; IL4Rα; IL5Rα; IL6Rα; IL7Rα; IL9Rα; IL10Rα; IL10Rβ; IL12Rβ1; IL12Rβ2; IL12p40; IL13RA1; IL15Rα; IL20RB; IL21Rα; IL22R; IL23R; IL28R; IL31Rα; GMCSFRα; LIFRB; CNTFR; CLF1; OSMR; GCSFR; EPOR; TPOR; GHR; PRLR; LEPR; IFNAR2; IFNAR1; IFNGR1; IFNGR2. In some embodiments the ICD comprises an ICD selected from the ICD present in the constructs of SEQ ID NO:2; SEQ ID NO: 4; SEQ ID NO:6; SEQ ID NO:8; SEQ ID NO:10; SEQ ID NO: 12; SEQ ID NO: 14; SEQ ID NO: 16; SEQ ID NO: 18; SEQ ID NO:22; SEQ ID NO:24; SEQ ID NO: 26; SEQ ID NO: 28; SEQ ID NO: 30; SEQ ID NO:32; SEQ ID NO:34; SEQ ID NO:36; SEQ ID NO: 38; SEQ ID NO: 42; SEQ ID NO: 44; SEQ ID NO: 46; SEQ ID NO:48; SEQ ID NO:50; SEQ ID NO: 52; SEQ ID NO:54; SEQ ID NO:56; SEQ ID NO:58; SEQ ID NO:60; SEQ ID NO:64; SEQ ID NO: 66; SEQ ID NO: 68; SEQ ID NO: 70; SEQ ID NO:72; SEQ ID NO:74; SEQ ID NO:76; SEQ ID NO: 78; SEQ ID NO: 80; SEQ ID NO:82; SEQ ID NO:86, SEQ ID NO:88; SEQ ID NO:90; SEQ ID NO: 92 or a derivative thereof. In some embodiments an ICD comprises an interferon receptor subunit ICD, e.g. IFNAR2; IFNAR1; IFNGR1; and IFNGR2. In some embodiments an ICD comprises an IL-9R, IL-10R, IL-21R, IL-22R, or GCSFR ICD.

In some embodiments the TM domain is the TM domain sequence of a receptor subunit selected from IL3Rα; IL4Rα; IL5Rα; IL6Rα; IL7Rα; IL9Rα; IL10Rα; IL10Rβ; IL12Rβ1; IL12Rβ2; IL12p40; IL13RA1; IL15Rα; IL20Rβ; IL21Rα; IL22R; IL23R; IL28R; IL31Rα; GMCSFRα; LIFRβ; CNTFR; CLF1; OSMR; GCSFR; EPOR; TPOR; GHR; PRLR; LEPR; IFNAR2; IFNAR1; IFNGR1; IFNGR2 as disclosed above. In some other embodiments the TM is the TM sequence of the ECD, e.g. and without limitation, IL-2RB, ortho-IL-2RB, etc. Alternative TM domains may also find use.

In some embodiments the ICD comprises one or more amino acid modifications relative to the ICD of the wild-type receptor, particularly modifications at a phosphosite that is phosphorylated by an intracellular kinase. In some such embodiments the phosphosite is a site for JAK phosphorylation. Examples of such modifications are set forth in any of SEQ ID NO:18, 20, 22, 24, 26, 28, 30, 32, 34, 36, 38, 42, 44, 46, 48, 64, 66, 68, 70, 72, 74, 78, 80 and 82.

In referring to such phosphosite sites, the phosphorylated tyrosine (pY) is arbitrarily set to zero (0), and the residues before are referred to as (−1), (−2) etc; and residues after are referred to as (+1), (+2) etc. Reference may be made herein to variants in which the −2, −1, +1 and +2 sites are varied. In some specific examples, the Y(+2) site is mutagenized to generate a library of ICD sequences comprising each possible amino acid, e.g. having the sequence YLXQ where X is any amino acid. In some such embodiments the −2 and −1 residues are also modified, e.g. to comprise the motif SAYLXQ, or DAYLXQ, where X is any amino acid, or any amino acid other than the wild-type residue. The selection of a particular amino acid at X is chosen to tune the STAT activation profile to modulate activation of, for example, STAT1, STAT3, STAT5, etc.

In some embodiments, alone or in combination with the phosphosite modification disclosed above, an additional phosphosite is added to the ICD sequence. The additional site may comprise the sequence SEQ ID NO:93 (LNTDAYLSLQE)ₓ, where X is from 1, 2, 3, or more, which site can enhance STAT5 activation. The phosphosite may be placed close to the terminus of the ICD, for example at the C-terminus or flanked by the wild-type C-terminal sequence, or within 1, 2, 3, 4, 5, amino acids from the terminus.

Suitable ECD domains for use in tunable cytokine receptors are ECDs from receptor subunits that dimerize or multimerize in response to ligand binding to transmit signals across the cell membrane, typically resulting in modification, e.g. phosphorylation, of the ICD. The ECD can be an ECD of a receptor subunit that forms a heteromultimer or a homomultimer upon binding. Various multimerization modalities find use, e.g. zipper motifs, FK506 or rapamycin binding, etc. In some embodiments the multimerization results in recruitment of a cellular kinase, including without limitation, recruitment of a JAK kinase. In some embodiments, the ECD is the ECD of a receptor subunit that multimerizes with a “common” chain, e.g. γc (CD132); gp130; βc; IL10Rβ; etc.

In some specific embodiments, the ECD is the extracellular domain of an IL-2 receptor, e.g. IL2Rβ, including a wild-type receptor or a modified receptor. In some specific embodiments, the IL2Rβ ECD is modified to comprise sequence modifications that alter its binding specificity, such that the ECD binds to an orthogonal ligand counterpart of its naturally-occurring ligand (OECD). An orthogonal ligand specifically binds to its counterpart OECD. The OECD exhibits significantly reduced binding to its endogenous ligand, including to the naturally-occurring counterpart of the orthogonal ligand. The orthogonal ligand exhibits significantly reduced binding to its endogenous receptors, including to the naturally-occurring counterpart of the orthogonal receptor.

In some embodiments, a tunable cytokine receptor subunit polypeptide or polynucleotide encoding such as receptor subunit polypeptide is provided, which polypeptides comprise: (a) an extracellular domain (ECD) that, when bound to its cognate ligand, multimerizes and activates intracellular signaling pathways; (b) a transmembrane domain; and (c) an intracellular domain (ICD) having a sequence other than the naturally occurring ICD of the extracellular domain, where the ICD may comprise targeted amino acid modifications that alter the STAT protein profile activated by the receptor. In some embodiments, a library of such tunable cytokine receptor subunit polypeptides are provided, e.g. comprising a library of polypeptides comprising the phosphosite YLXQ where X is any amino acid, or any amino acid other than the wild-type residue. A library may comprise polypeptides having the phosphosite SAYLXQ, or DAYLXQ, where X is any amino acid, or any amino acid other than the wild-type residue. Polypeptides in the library may be alternatively or in addition modified to comprise the sequence SEQ ID NO:93 (LNTDAYLSLQE)ₓ, where X is from 1, 2, 3, or more, which site can enhance STAT5 activation.

Libraries of polypeptides or polynucleotide coding sequences thereof find use in tuning a STAT activation profile, wherein each of the polypeptides are expressed in a cell, including without limitation a T cell, and contacted with the appropriate ligand for the ECD. The STAT activation profile can be determined by any convenient method. The STAT activation profile provides a basis for selection of a tunable cytokine receptor subunit for use in stimulating a cell population to a desired STAT activation profile.

In some embodiments a vector comprising a polynucleotide coding sequence that encodes a tunable cytokine receptor subunit of the invention is provided, where the coding sequence is operably linked to a promoter active in the desired cell for expression, where an active promoter may be constitutively active or may be regulated. Various vectors are known in the art and can be used for this purpose, e.g. replication competent, replication deficient or conditionally replicating viral vectors, plasmid vectors, minicircle vectors, which vectors can be integrated into the target cell genome or can be episomally maintained.

The vectors provided herein may be provided in a kit, optionally combined with a vector encoding a ligand, including without limitation an orthogonal ligand, that binds to and activates the ECD of the tunable cytokine receptor subunit. In some embodiments the coding sequence for the orthogonal ligand is operably linked to a high expression promoter. In other embodiments, a kit is provided in which the vector encoding the tunable cytokine receptor subunit is provided with a purified composition of the ligand, e.g. in a unit dose, packaged for administration to a patient (e.g. a prefilled syringe). In still some other embodiments, a kit is provided in which the vector encoding the tunable cytokine receptor subunit is provided with a vector encoding the ligand to enable expression of the tunable cytokine receptor subunit in a cell and also expression of the ligand intended for secretion by the same cell (or other cell) to enable autocrine, endocrine, or paracrine ligand/receptor signaling.

In some embodiments, an engineered cell is provided, in which the cell has been modified by introduction of a tunable cytokine receptor subunit of the disclosure. Any cell can be used for this purpose. In some embodiments the cell is a T cell, including without limitation naïve CD8⁺ T cells, cytotoxic CD8⁺ T cells, naïve CD4⁺ T cells, helper T cells, e.g. T_{H}1, T_{H}2, T_{H}9, T_{H}11, T_{H}22, T_{FH}; regulatory T cells, e.g. T_{R}1, natural T_{Reg}, inducible T_{Reg}; memory T cells, e.g. central memory T cells, effector memory T cells, NKT cells, αβ T cells, γδ T cells and engineered variants of such T cells including CAR T cells; etc., and T cell populations such as TILs (tumor infiltrating lymphocytes). In other embodiments the engineered cell is a stem cell, including but not limited to a hematopoietic stem cell, an NK cell, a macrophage, or a dendritic cell. In some embodiments the cell is genetically modified in an ex vivo procedure, prior to transfer into a subject, to introduce a coding sequence for the tunable cytokine receptor subunit. In some embodiments the cell is genetically engineered to express an engineered T cell receptor; a chimeric antigen receptor; and the like. The engineered cell can be provided in a unit dose for therapy, and can be allogeneic, autologous, etc. with respect to an intended recipient.

In some embodiments a therapeutic method is provided, the method comprising introducing into a subject in need thereof a therapeutically effective quantity of an engineered cell population, wherein all or a part of the cell population has been modified by introduction of a nucleic acid sequence encoding a tunable cytokine receptor subunit of the invention. The cell population may be engineered ex vivo, and may be autologous or allogeneic with respect to the subject. In some embodiments, the introduced cell population is contacted with the cognate ligand in vivo following administration of the engineered cells. In some embodiments the engineered cell is a T cell. In some embodiments the engineered cell is a CAR T cell.

In some embodiments a tunable cytokine receptor comprises or consists essentially of a protein as set forth in any of SEQ ID NO:2; SEQ ID NO: 4; SEQ ID NO:6; SEQ ID NO:8; SEQ ID NO: 10; SEQ ID NO: 12; SEQ ID NO: 14; SEQ ID NO: 16; SEQ ID NO:18; SEQ ID NO:22; SEQ ID NO: 24; SEQ ID NO: 26; SEQ ID NO: 28; SEQ ID NO: 30; SEQ ID NO:32; SEQ ID NO:34; SEQ ID NO: 36; SEQ ID NO: 38; SEQ ID NO: 42; SEQ ID NO:44; SEQ ID NO:46; SEQ ID NO:48; SEQ ID NO: 50; SEQ ID NO:52; SEQ ID NO:54; SEQ ID NO:56; SEQ ID NO:58; SEQ ID NO:60; SEQ ID NO: 64; SEQ ID NO: 66; SEQ ID NO: 68; SEQ ID NO: 70; SEQ ID NO:72; SEQ ID NO:74; SEQ ID NO: 76; SEQ ID NO: 78; SEQ ID NO:80; SEQ ID NO:82; SEQ ID NO:86, SEQ ID NO:88; SEQ ID NO: 90; SEQ ID NO:92, or a mature form thereof.

### BRIEF DESCRIPTION OF THE DRAWINGS

The invention may be understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1A-1B. Summary of cytokine receptor engineering. (A) Non-natural chimeric receptors: signaling competent non-natural oIL2Rb-ICD receptor w/γc common chain. (B) site-specific phosphosite replacement or additions to modulate STAT signaling and effector functions.

FIG. 2. Mouse ortho-IL2Rβ ECD with Interferon receptor pairing. STAT signaling of moRb (mouse orthoIL2Rb)-Interferon Receptor (IFNR) family chimeric receptors. Mouse T cell blasts were virally transduced with either moRb/IFNAR1, /IFNAR2 (Type I IFN receptors, left); /IFNGR1, /IFNGR2 (Type II IFN receptors, middle); or/IFNLR1 (Type III IFN receptor, right).

Transduced cells were stimulated with dose titration concentrations of orthoIL2 for 20′, then fixed in paraformaldehyde (PFA), methanol (MeOH) permeabilized and stained with anti-pSTAT5-A647 or anti-pSTAT1-A647. Samples were analyzed on a Cytoflex (Beckman Coulter), gating on YFP+ cells; data were plotted in Prism (GraphPad) n=3, SEM. MoRb/2Rb receptor was included as positive control for orthoIL2 signaling.

FIG. 3. OrthoRb/2Rb versus IFNAR2 CTV T cell proliferation assay. moRb/IFNAR2 chimera phenocopies native IFN anti-proliferative effect. moRb/IFNAR2 expressing cells proliferate in presence of IL2, but are anti-proliferative in presence of orthoIL2. Mouse T cell blasts were transduced with moRb/IFNAR2-IRES-YFP retrovirus. Cells were labeled with CellTracer Violet (CTV) on day 0, and cultured with indicated concentration of mIL2, molL2, or m1FNb. On day 4, samples were analyzed on Cytoflex, gating on live, YFP(+) or (−) cells.

FIG. 4. STAT signaling of moRb (mouse orthoIL2Rb)-IL10 Receptor (IL10R) family chimeric receptors. Mouse T cell blasts were virally transduced with either moRb/IL10Rα, /IL20Rα, /IL22R. Transduced cells were stimulated with dose titration concentrations of orthoIL2 for 20′, then fixed in paraformaldehyde (PFA), methanol (MeOH) permeabilized and stained with anti-pSTAT5-A647, anti-pSTAT3-A647 or anti-pSTAT1-A647. Samples were analyzed on a Cytoflex (Beckman Coulter), gating on YFP+ cells; data were plotted in Prism (GraphPad). MoRb/2Rb receptor was included as positive control for orthoIL2 signaling.

FIG. 5. moRb/IL10R family chimera have T effector cell properties. Mouse T cell blasts were virally transduced with either moRb/IL10Rα, /IL20Rα, /IL22R, then cultured in indicated concentration of oIL2 for 3 days, then recall stimulated with anti-CD3 for 4H. Supernatant was harvested and level of IFNγ was determined by ELISA (Biolegend).

FIG. 6. Direct comparison of moRb chimeric receptor on pSTAT signaling. Mouse T cell blasts were virally transduced with either moRb/2Rb, /9R, /IFNLR1, or/IL22R. Transduced cells were stimulated with orthoIL2 for 20′, then fixed in paraformaldehyde (PFA), methanol (MeOH) permeabilized and stained with anti-pSTAT5-A647, anti-pSTAT3-A647 or anti-pSTAT1-A647. Samples were analyzed on a Cytoflex (Beckman Coulter), gating on YFP+ cells; data were plotted in Prism (GraphPad).

FIG. 7. Direct comparison of moRb chimeric receptors on proliferation. Mouse T cell blasts were virally transduced with either moRb/2Rb, /9R, /IL22R, /IFNAR2, /IFNGR1, /IFNLR1. Transduced cells were stimulated with dose titration concentration of IL2 or orthoIL2. On day 4, samples were acquired on a Cytoflex (Beckman Coulter), gating on YFP+ cells; data were plotted in Prism (GraphPad).

FIG. 8. Direct comparison of moRb chimeric receptors on cell expression markers. Mouse T cell blasts were virally transduced with either moRb/2Rb, /9R, /IL22R, /IFNAR2, /IFNGR1, /IFNLR1. Transduced cells were cultured in dose titration concentrations of IL2 or orthoIL2. On day 4, samples were stained with antibodies to indicated cell surface markers and acquired on a Cytoflex (Beckman Coulter), gating on YFP+ cells; data were plotted in Prism (GraphPad).

FIG. 9. STAT signaling of mouse orthoIL2Rb/IL9Receptor (mo9R) phosphosite variants. Mouse T cell blasts were virally transduced with mo9R containing either a single point mutation in the STAT binding phosphosite or addition of the IL2Rb pSTAT5 phosphosite, in single, double, or triple tandem repeats, to the C-terminus of the o9R receptor. Transduced cells were stimulated with orthoIL2 for 20′, then fixed in paraformaldehyde (PFA), methanol (MeOH) permeabilized and stained with anti-pSTAT5-A647, anti-pSTAT3-A647 or anti-pSTAT1-A647. Samples were analyzed on a Cytoflex (Beckman Coulter), gating on YFP+ cells; data were plotted in Prism (GraphPad). Mo2Rb receptor was included as positive control for orthoIL2 signaling.

FIG. 10. T cells transduced with mo9R phosphosite variants respond similarly to IL2. T cells from FIG. 9 were stimulated with wild-type IL2 for 20′, then fixed in paraformaldehyde (PFA), methanol (MeOH) permeabilized and stained with anti-pSTAT5-A647, anti-pSTAT3-A647 or anti-pSTAT1-A647. Samples were analyzed on a Cytoflex (Beckman Coulter), gating on YFP+ cells; data were plotted in Prism (GraphPad).

FIG. 11. T cells transduced with mo9R phosphosite variants exhibit varying levels of proliferation and surface expression of stem-ness markers. Chimeric receptor transduced T cells were stimulated with orthoIL2 (5 uM, left) or wild-type IL2 (50 nM, right). On day 4, samples were stained for Sca1 and Fas (CD95) and acquired on a Cytoflex (Beckman Coulter), gating on YFP+ cells; data were plotted in Prism (GraphPad).

FIG. 12A-12B. STAT signaling of mouse orthoIL2Rb/IL9Receptor (mo9R) phosphosite variants (amino acid scan of Y+2 position). Mouse T cell blasts were virally transduced with mo9R containing a single point mutation in the STAT binding phosphosite. Transduced cells were stimulated with dose titration concentration of orthoIL2 for 20′, then fixed in paraformaldehyde (PFA), methanol (MeOH) permeabilized and stained with anti-pSTAT5-A647, anti-pSTAT3-A647 or anti-pSTAT1-A647. Samples were analyzed on a Cytoflex (Beckman Coulter), gating on YFP+ cells; data were plotted in Prism (GraphPad). Mo2Rb receptor was included as positive control for orthoIL2 signaling.

FIGS. 13A-13B. STAT signaling of mouse orthoIL2Rb/IL9Receptor (mo9R) phosphosite variants. oIL2, 5 uM data from prior figure plotted as bar graph. Solid and dotted lines represent baseline pSTAT3 and pSTAT1 of mo9R (WT) and mo9R (YLKQ) variant, respectively.

FIG. 14. Sequence summary of mo9R phosphosite variants.

FIG. 15. STAT signaling and proliferation of mouse orthoIL2Rb/IL21Receptor (mo21R) variants. Mouse T cell blasts were virally transduced with mo21R containing addition of the IL2Rb pSTAT5 phosphosite, in single, tandem or triple repeat, to the C-terminus of the 021R receptor. Transduced cells were stimulated with dose titration concentrations of orthoIL2 for 20′, then fixed in paraformaldehyde (PFA), methanol (MeOH) permeabilized and stained with anti-pSTAT5-A647, anti-pSTAT3-A647 or anti-pSTAT1-A647. Samples were analyzed on a Cytoflex (Beckman Coulter), gating on YFP+ cells; data were plotted in Prism (GraphPad). Mo2Rb receptor was included as positive control for orthoIL2 signaling.

FIG. 16. T cells transduced with mo21R phosphosite variants show slight increase in proliferation while retaining stem-ness markers. Chimeric receptor transduced T cells were stimulated with orthoIL2 (5 uM, left) or wild-type IL2 (50 nM, right). On day 4, samples were stained for Sca1, Fas (CD95), and CD62L and acquired on a Cytoflex (Beckman Coulter), gating on YFP+ cells; data were plotted in Prism (GraphPad).

FIG. 17. STAT signaling of human orthoIL2Rb/IL9Receptor (ho9R). Human PBMCs were virally transduced with ho2R or ho9R. Transduced cells were stimulated with human orthoIL2 or human IL2 for 20′, then fixed in paraformaldehyde (PFA), methanol (MeOH) permeabilized and stained with anti-pSTAT5-A647, anti-pSTAT3-A647 or anti-pSTAT1-A647. Samples were analyzed on a Cytoflex (Beckman Coulter), gating on YFP+ cells; data were plotted in Prism (GraphPad). UTD: untransduced control.

FIG. 18. In vitro profiling of human orthoIL2Rb/IL9Receptor (ho9R). Human PBMCs were virally transduced with ho2Rb or ho9R. Transduced cells were stimulated with human orthoIL2 or human IL2 for 2 days, and then stained for CD27 and CD45RA. Samples were analyzed on a Cytoflex (Beckman Coulter), gating on YFP+ cells; data were plotted in Prism (GraphPad). UTD: untransduced control.

FIG. 19A-19C. A. mouse orthoIL2Rb chimeric receptors (mo2R, mo9R, or moGCSFR (SEQ ID NO:87) were transduced into mouse T cells. Cells were stimulated with dose titration of MSA-orthoIL2 for 20′, then fixed, permeabilized, stained for pSTAT and analyzed by flow cytometry. B. T cells were transduced with orthoIL2Rb/GCSFR chimeric receptor (SEQ ID NO: 85). Cells were labeled w/CTV and cultured in either dose titration of MSA-orthoIL2 or MSA-IL2. 96h later, cells were analyzed by flow cytometry. C. T cells were transduced with orthoIL2Rb/GCSFR chimeric receptor (SEQ ID NO:85), then cultured in either MSA-orthoIL2 or MSA-IL2. 96h later, cells were stained for indicated surface markers, and analyzed by flow cytometry.

FIGS. 20A-20C. YT1 (CD25+) cell line was transduced w/hoR chimeric receptors, then sorted and expanded. The receptors correspond to (A) hu-orthoIL 10R (SEQ ID 55/56); (B) hu-orthoIL22R (SEQ ID 59/60); and (C) hu-orthoGCSFR (SEQ ID 91/91). Cells were stimulated with MSA-hoIL2 (SQVLKA) for 20′, starting at 1 uM, 10× dilution. Cells were then fixed, permeabilized and stained for indicated phosphoSTAT. Samples were analyzed by flow cytometry. Data plotted in Prism, gated on YFP (+), unless otherwise noted. n=2, SEM. Results: human orthoIL2R chimeric receptor signal on YT1 cells, and pSTAT profile trends similarly to mouse profile.

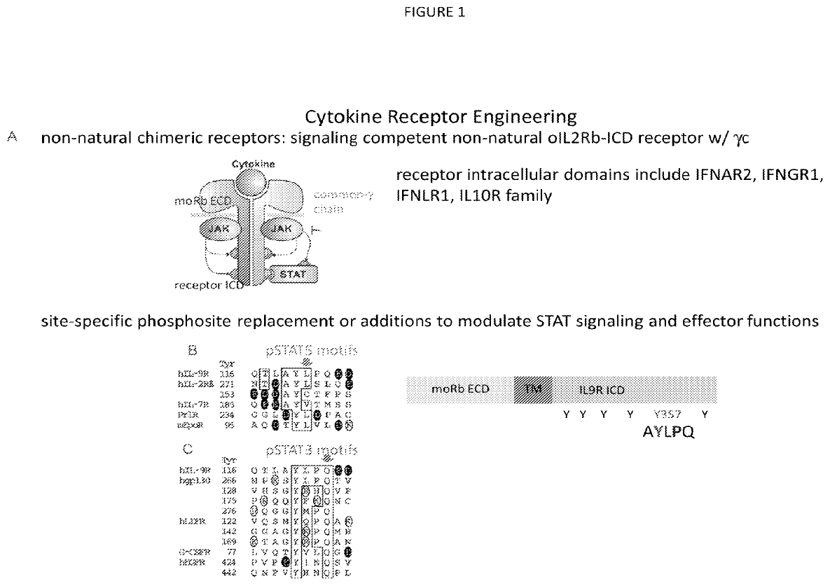

### DETAILED DESCRIPTION OF THE EMBODIMENTS

In order for the present disclosure to be more readily understood, certain terms and phrases are defined below as well as throughout the specification. The definitions provided herein are non-limiting and should be read in view of what one of skill in the art would know at the time of invention.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It should be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “polypeptide,” “protein” or “peptide” refer to any chain of amino acid residues, regardless of its length or post-translational modification (e.g., glycosylation or phosphorylation).

The term “identity,” as used herein in reference to polypeptide or DNA sequences, refers to the relative sequence identity between two molecules. The similarity between two amino acid or two nucleotide sequences is a direct function of the number of identical positions and is frequently expressed as a percentage (“percent identity”). In general, when determining identity of two sequences, the sequences are aligned so that the highest order match is obtained (greatest percent identity). Identity can be evaluated using published techniques and may be assessed using widely available computer programs, such as the GCS program package (Devereux et al., Nucleic Acids Res. 12:387, 1984), BLASTP, BLASTN, FASTA (Atschul et al., J. Molecular Biol. 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

As used herein, the terms “protein variant” or “variant protein” or “variant polypeptide” and the like refer to a protein that differs from a reference polypeptide by virtue of at least one amino acid modification. The reference polypeptide may be a naturally occurring or wild-type (WT) polypeptide or may be a modified version of a WT polypeptide. In some embodiments, the variant polypeptide comprises at least one amino acid modification relative to a reference parent polypeptide. In some embodiments, the variant polypeptide comprises from about one to about ten amino acid modifications relative to a reference parent polypeptide. In some embodiments, the variant polypeptide comprises from about one to about five amino acid modifications, relative to a reference parent polypeptide. In some embodiments, the variant polypeptide is at least about 99% identical to the reference protein, alternatively at least about 98% identical, alternatively at least about 97% identical, alternatively at least about 95% identical, alternatively at least about 90% identical. A variant protein may, for example, be at least about 99% identical to the reference protein, at least about 98% identical, at least about 97% identical, at least about 95% identical, at least about 90% identical to any one or more of the ICD provided in the constructs of any of SEQ ID NO: 2; SEQ ID NO:4; SEQ ID NO:6; SEQ ID NO:8; SEQ ID NO: 10; SEQ ID NO:12; SEQ ID NO: 14; SEQ ID NO: 16; SEQ ID NO:18; SEQ ID NO:22; SEQ ID NO:24; SEQ ID NO:26; SEQ ID NO: 28; SEQ ID NO:30; SEQ ID NO:32; SEQ ID NO:34; SEQ ID NO:36; SEQ ID NO:38; SEQ ID NO: 42; SEQ ID NO: 44; SEQ ID NO:46; SEQ ID NO:48; SEQ ID NO:50; SEQ ID NO:52; SEQ ID NO: 54; SEQ ID NO:56; SEQ ID NO:58; SEQ ID NO:60; SEQ ID NO:64; SEQ ID NO:66; SEQ ID NO: 68; SEQ ID NO: 70; SEQ ID NO:72; SEQ ID NO:74; SEQ ID NO:76; SEQ ID NO:78; SEQ ID NO: 80; SEQ ID NO:82; SEQ ID NO:86, SEQ ID NO:88; SEQ ID NO:90; SEQ ID NO:92 fused to a non-naturally-occurring ECD.

In some embodiments a variant protein polypeptide comprises a sequence that is identical or at least about 99% identical to the ICD sequence provided in the constructs of any one of SEQ ID NO: 18; SEQ ID NO:30; SEQ ID NO:32; SEQ ID NO:34; SEQ ID NO:36; SEQ ID NO:38; SEQ ID NO: 42; SEQ ID NO: 44; SEQ ID NO:46; SEQ ID NO:48; SEQ ID NO:64; SEQ ID NO:66; SEQ ID NO: 68; SEQ ID NO: 70; SEQ ID NO:72; SEQ ID NO:74; SEQ ID NO:78; SEQ ID NO:80; and SEQ ID NO:82; SEQ ID NO:86, SEQ ID NO:88; SEQ ID NO:90; SEQ ID NO:92, alternatively at least about 98% identical, alternatively at least about 97% identical, alternatively at least about 95% identical, alternatively at least about 90% identical.

As used herein, the terms “wild type” or “WT” or “native” or “naturally occurring” refer to an amino acid sequence or a nucleotide sequence that is found in nature, including allelic variations. A wild-type polypeptide (e.g. protein, antibody, receptor, immunoglobulin, IgG, etc.) has an amino acid sequence or a nucleotide sequence that has not been modified by intervention of the hand of man.

The terms “recipient”, “individual”, “subject”, “host”, and “patient”, are used interchangeably herein and refer to any mammalian subject suffering from a disease, disorder or condition for whom diagnosis, treatment, or therapy is desired. “Mammal” for purposes of treatment refers to any animal classified as a mammal, including humans, domestic and farm animals, and zoo, sports, or pet animals, such as dogs, horses, cats, cows, sheep, goats, pigs, etc. In some embodiments the mammal is human.

As used herein, the term a “therapeutically effective amount” refers to that amount of the therapeutic agent sufficient to prevent, treat or manage the symptoms of a condition, disease or disorder. A therapeutically effective amount may refer to the amount of therapeutic agent sufficient to delay or minimize the onset of disease, e.g., delay or minimize the spread of cancer, or the amount effect to decrease or increase signaling from a receptor of interest. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the terms “prevent”, “preventing” and “prevention” refer to the prevention of the recurrence or onset of one or more symptoms of a disorder in a subject as result of the administration of a prophylactic or therapeutic agent.

As used herein, the term “in combination” refers to the use of more than one prophylactic and/or therapeutic agents. The use of the term “in combination” does not restrict the order in which prophylactic and/or therapeutic agents are administered to a subject with a disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with (e.g. simultaneously, in separate preparations or in a co-formulation, or in separate preparations the first provided agent administered to the subject within about 5 minutes of the administration of a second agent in the multiagent protocol), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject with a disorder.

Cytokines that signal through the JAK/STAT pathway. A number of cytokines utilize the JAK/STAT pathway to activate transcription programs and produce changes in phenotype of responding cells. Included are the cytokines and receptors listed below in Table 1.

In some embodiments, a tunable cytokine receptor of the disclosure comprises an ECD, selected from the receptors listed in Table 1, where the ECD is other than the naturally-occurring ECD associated with the ICD. In certain embodiments the ECD is the ECD of a subunit other than a common subunit, i.e. other than γc, gp130, IL-10RB. In some embodiments the ECD is of a subunit associated with γc, including without limitation: IL2Rβ; IL4Rα; IL7Rα; IL9Rα; IL15Rα; IL21Rα. In certain embodiments the ECD is an orthogonal variant of such an ECD, e.g. as disclosed in U.S. Pat. No. 10,869,887, herein specifically incorporated by reference.

TABLE 1

Cognate Receptors

Class I cytokines
IL-2 family

| | |
|---|---|
| IL-2 Interleukin-2 | γc, IL2Rβ, IL2Rα |
| IL-4 Interleukin-4 | γc, IL4Rα |
| IL-7 Interleukin-7 | γc, IL7Rα |
| IL-9 Interleukin-9 | γc, IL9Rα |
| IL-15 Interleukin-15 | γc, IL2Rβ, IL15Rα |
| IL-21 Interleukin-21 | γc, IL21Rα |

IL-3 family

| | |
|---|---|
| IL-3 Interleukin-3 | βc, IL3Rα |
| IL-5 Interleukin-5 | βc, IL5Rα |
| GM-CSF Granulocye Macrophage | βc, GMCSFRα |
| Colony Stimulating Factor | |

IL-6 family

| | |
|---|---|
| IL-6 Interleukin-6 | gp130, IL6Rα |
| LIF Leukemia Inhibitory Factor | gp130, LIFRβ |
| CNTF Ciliary Neurotrophic | gp130, LIFRβ, CNTFR |
| growth Factor | |
| CT1 Cardiotrophin 1 | gp130, LIFRβ, CNTFR |
| CLC Cardiotrophin-like cytokine | gp130, LIFRβ, CNTFR, CLF1 |
| OSM Oncostatin M Pleiotropic, | gp130, OSMR |
| bone formation | |
| IL-31 Interleukin-31 | IL31Rα, OSMR |
| NP Neuropoietin | Gp130, CNTFR, LIFRβ |

Homodimeric

| | |
|---|---|
| G-CSF Granulocyte Colony | GCSFR |
| Stimulating Factor | |
| EPO Erythropoietin | EPOR |
| TPO Thrombopoietin | TPOR |
| GH Growth Hormone | GHR |
| PRL Prolactin | PRLR |
| LEP Leptin | LEPR |

Others

| | |
|---|---|
| IL-12 Interleukin-12 | IL12Rβ1, IL12Rβ2, IL12p40 |
| IL-13 Interleukin-13 | IL13RA1, IL4Rα, |
| IL-23 Interleukin-23 | IL12Rβ1, IL23R, IL12p40 |
| TSLP Thymic Stromal | |
| Lymphopoietin | |

Class II cytokines
Type I interferon

| | |
|---|---|
| IFNα Interferon alpha | IFNAR2, IFNAR1 |
| IFNβ Interferon beta | IFNAR2, IFNAR1 |
| IFNε Interferon epsilon | IFNAR2, IFNAR1 |
| IFNκ Interferon kappa | IFNAR2, IFNAR1 |
| IFNω Interferon omega | IFNAR2, IFNAR1 |

Type II interferon

| | |
|---|---|
| IFNγ Interferon gamma | IFNGR1, IFNGR2 |

Type III interferon

| | |
|---|---|
| IFNλ1 Interferon lambda1 | IL10Rβ, IL28R |
| IFNλ2 Interferon lambda2 | IL10Rβ, IL28R |
| IFNλ3 Interferon lambda3 | IL10Rβ, IL28R |

IL-10 family

| | |
|---|---|
| IL-10 Interleukin-10 | IL10Rβ, IL10Rα |
| IL-19 Interleukin-19 | IL20Rβ, IL20Rα |
| IL-20 Interleukin-20 | IL20Rβ, IL22R |
| IL-22 Interleukin-22 | IL10Rβ, IL22R |
| IL-24 Interleukin-24 | IL20Rβ, IL22R |
| IL-26 Interleukin-26 | IL10Rβ, IL10Rα |

STAT activation profile. JAK phosphorylation of distal tyrosines on the receptor intracellular domains enables those sequences to act as docking sites for STAT proteins, where a specific set of STAT proteins is associated with signaling from a specific ICD. The ability of a ligand to induce activation of a particular set of STATs is driven by the STAT-binding sites contained within the receptor ICD.

The human STAT family contains seven STATs: STAT1, STAT2, STAT3, STAT4, STAT5A, STAT5B and STAT6. STAT-binding sites from one receptor can be replaced with binding sites for different STATs from other receptors and thereby activate nonphysiological STATs (see, for example, Stahl et al. (1995) Science 267:1349-1353). It has been proposed that the affinity for one STAT over another is a function of the sequence immediately surrounding the phosphotyrosine, for example pYxxP, pYxxQ, pYxxL, and pYxxF sequences are associated with recruitment of STAT1, STAT3, STAT5, and STAT6, respectively.

The set of STAT proteins activated from a specific wild-type or engineered ICD may be referred to herein as the STAT activation profile. An ICD may activate one or more STAT proteins, and may further be characterized by degree of activation, in addition to the characterization of specific STAT proteins that are activated. The level of activation of STAT1, STAT3 and STAT5 are of particular interest.

The level of activation of STAT proteins can be determined by any convenient method, including without limitation assays set forth in the Examples. For example, cells expressing a tunable cytokine receptor can be stimulated with an appropriate ligand in vitro for a period of time sufficient to activate the JAK/STAT pathway. The level of phosphorylated STAT proteins in the cell is then determined, e.g. by binding with specific, detectably labeled antibodies. The level of binding provides quantitation of the level of a phosphorylated STAT protein of interest. The STAT activation profile is used to determine the signaling properties of an ICD of interest.

As exemplary controls for the level of signaling, IL-2RB provides a strong (+++) signal for activation of STAT5, and very low (−) for STAT1 and STAT3. Interferon receptors, e.g. IFNAR1 provide a strong (+++) signal for STAT1. IL-22R provides a strong (+++) signal for STAT3.

Stemness. A clinical correlate of effective T cell therapy relates to the T cell properties of stemness, which may be defined by reduced effector cell differentiation, enhanced expression of co-stimulatory receptors, retention of key stemness-related transcription factors such as TCF1 (TCF7) and enhanced self-renewal capacity, relative to, for example, bulk peripheral blood T cells. The presence of stem-like T cells can play a role in mediating responses to immune checkpoint inhibitor therapy. Cell surface markers for stemness include, for example, CD39⁻CD69⁻ T cells. These cells also exhibited increased persistence after infusion. Retaining a population of cells with self-renewal characteristics is an important goal to deliver effective T cell mediated therapy.

Manipulation of signaling pathways with tunable cytokine receptors can enhance the stemness of a T cell population. For example, activation of STAT3 is associated with stem cell properties in a number of cell types, including T cells. Tunable receptors that provide for increased STAT3 activation in the STAT activation profile can be useful in increasing stemness in a T cell population. The level of stemness can be determined by, for example, quantitating the retention of TCF1 in the cell population, determining the number of CD39⁻CD69⁻ T cells in the population, determining persistence after infusion of the T cells, and the like.

It will be understood by one of skill in the art that desirable cell populations may be balanced between the level of activation, e.g. STAT5 activation, and the level of stemness, e.g. STAT3 activation.

Proliferation. Another clinical correlate of effective T cell therapy is the proliferation of T cells, which can be associated with activation. The level of proliferation can readily be measured by means known in the art, e.g. uptake of 3H-thymidine, dilution of dyes, and the like.

### Polypeptides

Compositions and methods are provided for tunable cytokine receptor polypeptides, which polypeptides comprise: (a) an extracellular domain (ECD) that, when bound to its cognate ligand, multimerizes and activates intracellular signaling pathways; (b) a transmembrane domain, and (c) an intracellular domain (ICD) having a sequence other than the naturally-occurring ICD of the extracellular domain, where the ICD may comprise targeted amino acid modifications that alter the STAT protein profile activated by the receptor. The phenotype of a cell, e.g. an immune cell, can be altered by transducing the cells with a tunable cytokine receptor, which can deliver customized STAT activation patterns upon binding to its cognate ligand. The set of STAT proteins activated by a tunable cytokine receptor can influence the transduced cell phenotype, including without limitation by altering the balance of proliferation or stemness.

Binding of the cognate ligand to the ECD activates signaling via the ICD of the receptor. Selection of the ICD, and targeted amino acid modification of ICD, e.g. in STAT binding sequences, allows for tuning of the receptor to provide a desired intracellular signal. In some specific embodiments, the STAT activation profile of a receptor is tuned to provide for a desired balance of activation and stemness in a cell expressing the tunable receptor.

Suitable ICD domains comprise a functional fragment derived from a receptor subunit, for example derived from a cytokine receptor. In some such embodiments the ICD is a functional fragment derived from a receptor and is substantially or entirely the ICD and transmembrane domain (TM) of the receptor. In some embodiments the ICD of the tunable cytokine receptor comprises phosphosites for one or more STAT signaling proteins, e.g. STAT1, STAT2, STAT3, STAT4, STAT5A, STAT5B, STAT6, etc.

In some embodiments the ICD comprises the TM and ICD sequence of a receptor subunit selected from IL3Rα; IL4Rα; IL5Rα; IL6Rα; IL7Rα; IL9Rα; IL10Rα; IL10Rβ; IL12Rβ1; IL12Rβ2; IL12p40; IL13RA1; IL15Rα; IL20Rβ; IL21Rα; IL22R; IL23R; IL28R; IL31Rα; GMCSFRα; LIFRβ; CNTFR; CLF1; OSMR; GCSFR; EPOR; TPOR; GHR; PRLR; LEPR; IFNAR2; IFNAR1; IFNGR1; IFNGR2. In some embodiments the ICD comprises an ICD provided in a construct sequence selected from SEQ ID NO:2; SEQ ID NO:4; SEQ ID NO:6; SEQ ID NO:8; SEQ ID NO: 10; SEQ ID NO: 12; SEQ ID NO:14; SEQ ID NO: 16; SEQ ID NO: 18; SEQ ID NO:22; SEQ ID NO:24; SEQ ID NO: 26; SEQ ID NO:28; SEQ ID NO:30; SEQ ID NO:32; SEQ ID NO:34; SEQ ID NO:36; SEQ ID NO: 38; SEQ ID NO: 42; SEQ ID NO:44; SEQ ID NO:46; SEQ ID NO:48; SEQ ID NO:50; SEQ ID NO: 52; SEQ ID NO:54; SEQ ID NO:56; SEQ ID NO:58; SEQ ID NO:60; SEQ ID NO:64; SEQ ID NO: 66; SEQ ID NO: 68; SEQ ID NO: 70; SEQ ID NO:72; SEQ ID NO:74; SEQ ID NO:76; SEQ ID NO: 78; SEQ ID NO:80; SEQ ID NO:82; SEQ ID NO:86, SEQ ID NO:88; SEQ ID NO:90; SEQ ID NO: 92 or a derivative thereof. In some embodiments an ICD comprises an interferon receptor subunit ICD, e.g. IFNAR2; IFNAR1; IFNGR1; and IFNGR2. In some embodiments an ICD comprises an IL-9R, IL-10R, IL-21R, IL-22R, GCSFR ICD.

A human counterpart may be provided for any mouse ICD sequence, e.g. SEQ ID NO:2; SEQ ID NO: 4; SEQ ID NO:6; SEQ ID NO:8; SEQ ID NO: 10; SEQ ID NO: 12; SEQ ID NO:14; SEQ ID NO: 16; SEQ ID NO:18; SEQ ID NO:22; SEQ ID NO:24; SEQ ID NO:26; SEQ ID NO:28; SEQ ID NO: 30; SEQ ID NO:32; SEQ ID NO:34; SEQ ID NO:36; SEQ ID NO:38; SEQ ID NO:42; SEQ ID NO: 44; SEQ ID NO:46; SEQ ID NO:48.

In some embodiments the ICD comprises one or more amino acid modifications relative to the ICD of the wild-type receptor, particularly modifications at a phosphosite that is phosphorylated by an intracellular kinase. In some such embodiments the phosphosite is a site for JAK phosphorylation. Examples of such modifications are set forth in any of SEQ ID NO:18, 20, 22, 24, 26, 28, 30, 32, 34, 36, 38, 42, 44, 46, 48, 64, 66, 68, 70, 72, 74, 78, 80 and 82.

In referring to such phosphosite sites, the pY is set to zero (0), and the residues before are referred to (−1), (−2) etc; and residues after are referred to (+1), (+2) etc. Reference may be made herein to variants in which the −2, −1, +1 and +2 sites are varied. In some specific examples, the Y(+2) site is mutagenized to generate a library of ICD sequences comprising each possible amino acid, e.g. having the sequence YLXQ where X is any amino acid. In some such embodiments the −2 and −1 residues are also modified, e.g. to comprise the motif SAYLXQ, or DAYLXQ, where X is any amino acid, or any amino acid other than the wild-type residue. The selection of a particular amino acid at X is chosen to tune the STAT activation profile to modulate activation of, for example, STAT1, STAT3, STAT5, etc.

In some embodiments, alone or in combination with the phosphosite modification disclosed above, an additional phosphosite is added to the ICD sequence. The additional site may comprise the sequence SEQ ID NO:93 (LNTDAYLSLQE)ₓ, where X is from 1, 2, 3, or more, which site can enhance STAT5 activation. The phosphosite may be placed close to the terminus of the ICD, for example at the C-terminus or flanked by the wild-type C-terminal sequence.

The intracellular signaling pathways activated by binding the cognate ligand to the ECD of the tunable cytokine receptor can reflect the signal characteristic pattern of activation of the ICD of the chimeric receptor. For example, the STAT activation pattern may be substantially similar to the pattern of activation that results from activation of the naturally-occurring receptor from which the ICD is derived with its naturally-occurring ligand. The STAT activation pattern may be modified by the alteration and/or addition of STAT phosphosites as described above.

The transmembrane domain (TM) of the tunable cytokine receptor may be the TM sequence of the same receptor protein from which the ICD is derived, in which instance the referenced ICD may be understood to include the TM domain, or the TM may be the TM sequence of the ECD. Alternatively the transmembrane domain may comprise a polypeptide sequence which is thermodynamically stable in a eukaryotic cell membrane, long enough to span the membrane and typically composed of non-polar amino acids. The transmembrane spanning domain may be derived from the transmembrane domain of a naturally occurring membrane spanning protein or may be synthetic. In designing synthetic transmembrane domains, amino acids favoring alpha-helical structures are preferred. Transmembrane domains are typically comprised of approximately 10, 11, 12, 13, 14, 15, 16, 17, 18, 19, 20, 21, 22, 22, 23, or 24 amino acids favoring the formation having an alpha-helical secondary structure. Amino acids having a to favor alpha-helical conformations are well known in the art. See, e.g., Pace, et al. (1998) Biophysical Journal 75:422-427. Amino acids that are particularly favored in alpha helical conformations include methionine, alanine, leucine, glutamate, and lysine.

Suitable ECD domains for use in tunable cytokine receptors are ECDs from receptor subunits that dimerize or multimerize in response to ligand binding to transmit signals across the cell membrane, typically resulting in modification, e.g. phosphorylation, of the ICD. The ECD can be an ECD of a receptor subunit that forms a heteromultimer or a homomultimer upon binding. In some embodiments the multimerization results in recruitment of a cellular kinase, including without limitation, recruitment of a JAK kinase. In some embodiments, the ECD is the ECD of a receptor subunit that multimerizes with a “common” chain, e.g. γc (CD132); gp130; βc; IL10Rβ; etc. In some embodiments the ECD is an ECD of a receptor set forth in Table 1.

Also included as suitable ECD, without limitation, are engineered receptor domains, e.g. an FK506 (see Blau et al. (1997) PNAS 94 (7) 3076-3081) or a rapamycin-inducible dimerization (RiD) receptor system that triggers receptor-specific signaling (see, for example, Kim et al. (2921) Molecular Plant 14 (8): 1379-1390; or light induced dimerization (see. Karginov et al. Am. Chem. Soc. 2011, 133, 3, 420-423). Other dimerization strategies utilize leucine zippers and coiled coils, for example see Nakase et al. (2012) Angew Chem Int Ed Engl 51 (30): 7464-7, describing signal transduction using an artificial receptor system that undergoes dimerization upon addition of a bivalent leucine-zipper ligand; DiMarco et al. (1996) Protein Chemistry and Structure 271 (48): 30386-30391 describing functional replacement of cytokine receptor extracellular domains with leucine zippers; Mossner et al. (2007) Nucleic Acids Research 35 (2): 517-528 review multimerization strategies for efficient production and purification of highly active synthetic cytokine receptor ligands. Synthetic cytokine receptors (SyCyRs) consisting of green fluorescent protein (GFP)- and mCherry-nanobodies as extracellular domains are described in PLOS ONE 15 (9): e0238925.

In some specific embodiments, the ECD is the extracellular domain of an IL-2 receptor, e.g. IL2Rβ, including a wild-type receptor or a modified receptor. In some specific embodiments, the IL2Rβ ECD is modified to comprise sequence modifications that alter its binding specificity, such that the ECD binds to an orthogonal ligand counterpart of its naturally-occurring ligand (i.e. an ortho ECD). An orthogonal ligand specifically binds to its counterpart ortho ECD. The ortho ECD exhibits significantly reduced binding to its endogenous ligand, including to the naturally-occurring counterpart of the orthogonal ligand. The orthogonal ligand exhibits significantly reduced binding to its endogenous receptors, including to the naturally-occurring counterpart of the orthogonal receptor (see, for example, U.S. Pat. No. 10,869,887).

In some embodiments, the receptor that contributes the ECD to the chimeric receptor is a chain of the IL-2 receptor, including but not limited to a polypeptide selected from interleukin 2 receptor beta (IL-2RB; also referred to as CD122), and interleukin 2 receptor gamma (IL-2RY; also referred to as CD132; also referred to as the “common gamma chain”). In some specific embodiments, the orthogonal receptor comprises a CD122 ECD. Exemplary orthogonal ECDs are provided in, for example, any of the orthogonal ECD sequences included in the constructs of SEQ ID NO:1-SEQ ID NO:82, which provide both human and mouse orthogonal ECDs.

An exemplary sequence for an orthogonal human IL-2 protein is provided as SEQ ID NO: 85. An exemplary sequence for orthogonal mouse IL-2 protein is provided as SEQ ID NO:86. Alternatively an orthogonal ligand may be designed based on the naturally-occurring human protein (refseq NP_000577.2) or the naturally-occurring mouse protein (NP_032392). In some embodiments, the amino acid substitutions for mIL-2 comprise one or more of: [H27W], [L28M, L28W], [E29D, E29T, E29A], [Q30N], [M33V, M331, M33A], [D34L, D34M], [Q36S, Q36T, Q36E, Q36K, Q36E], [E37A, E37W, E37H, E37Y, E37F, E37A, E37Y], [R41K, R41S], [N103E, N103Q]; and for hIL-2 is one or more of: [Q13W], [L14M, L14W], [E15D, E15T, E15A, E15S], [H16N, H16Q], [L19V, L191, L19A], [D20L, D20M], [Q22S, Q22T, Q22E, Q22K, Q22E], [M23A, M23W, M23H, M23Y, M23F, M23Q, M23Y], [G27K, G27S], [R81D, R81Y], [N88E, N88Q], [T51]]. In some embodiments the set of amino acid substitutions comprises one of the following sets of substitutions for mIL-2: [Q30N, M33V, D34N, Q36T, E37H, R41K]; [E29D, Q30N, M33V, D34L, Q36T, E37H]; [E29D, Q30N, M33V, D34L, Q36T, E37A], and [E29D, Q30N, M33V, D34L, Q36K, E37A] and for hIL-2: [H16N, L19V, D20N, Q22T, M23H, G27K]; [E15D, H16N, L19V, D20L, Q22T, M23H]; [E15D, H16N, L19V, D20L, Q22T, M23A], and [E15D, H16N, L19V, D20L, Q22K, M23A]; or a conservative variant thereof.

In some embodiments amino acid substitutions for orthogonal hIL-2 comprises one or more of: [E15S, E15T, E15Q, E15H]; [H16Q]; [L19V, L19I]; [D20T, D20S, D20M, D20L]; [Q22K, Q22N]; [M23L, M23S, M23V, M23T]. In some embodiments a consensus set of mutations for orthogonal hIL-2 is [E15S, H16Q, L19V, D20T/S/M; Q22K; M23L/S]. In some embodiments a consensus set of mutations for orthogonal hIL-2 is [E15S, H16Q, L19V, D20L, M23 Q/A] and optionally Q22K. In some embodiments the set of amino acid substitutions comprises one of the following sets of substitutions for orthogonal hIL-2: [E15S; H16Q; L19V, D20T/S; Q22K, M23L/S]; [E15S; H16Q; L191; D20S; Q22K; M23L]; [E15S; L19V; D20M; Q22K; M23S]; [E15T; H16Q; L19V; D20S; M23S]; [E15Q; L19V; D20M; Q22K; M23S]; [E15Q; H16Q; L19V; D20T; Q22K; M23V]; [E15H; H16Q; L191; D20S; Q22K; M23L]; [E15H; H16Q; L191; D20L; Q22K; M23T]; [L19V; D20M; Q22N; M23S]; [E15S, H16Q, L19V, D20L, M23Q, R81D, T511], [E15S, H16Q, L19V, D20L, M23Q, R81Y], [E15S, H16Q, L19V, D20L, Q22K, M23A], [E15S, H16Q, L19V, D20L,M23A].

In some embodiments, a ligand, including without limitation an orthogonal ligand, is conjugated to additional molecules to provide desired pharmacological properties, such as extended half-life. In one embodiment, a ligand is fused to the Fc domain of IgG, albumin (including human serum albumin), or other molecules to extend its half-life, e.g. by pegylation, glycosylation, and the like as known in the art. In some embodiments the ligand is conjugated to polyethylene glycol molecules or “PEGylated.” The molecular weight of the PEG conjugated to the ligand includes but is not limited to PEGs having molecular weights between 5 kDa and 80 kDa, in some embodiments the PEG has a molecular weight of approximately 5 kDa, in some embodiments the PEG has a molecular weight of approximately 10 kDa, in some embodiments the PEG has a molecular weight of approximately 20 kDa, in some embodiments the PEG has a molecular weight of approximately 30 kDa, in some embodiments the PEG has a molecular weight of approximately 40 kDa, in some embodiments the PEG has a molecular weight of approximately 50 kDa, in some embodiments the PEG has a molecular weight of approximately 60 kDa, in some embodiments the PEG has a molecular weight of approximately 70 kDa, in some embodiments the PEG has a molecular weight of approximately 80 kDa. In some embodiments, the PEG has an average molecular mass from about 5 kDa to about 80 kDa, from about 5 kDa to about 60 kDa, from about 5 kDa to about 40 kDa, from about 5 kDa to about 20 kDa. The PEG conjugated to the polypeptide sequence may be linear or branched. The PEG may be fused directly to the ligand or via a linker molecule. The processes and chemical reactions necessary to achieve PEGylation of biological compounds are well known in the art.

In addition to extending the serum half-life, Fc-fusion can also endow the fusion partner with alternative Fc receptor mediated properties in vivo. An “Fc region” can be a naturally occurring or synthetic polypeptide that is homologous to an IgG C-terminal domain produced by digestion of IgG with papain. The ligand can be fused to the entire Fc region, or a smaller portion that retains the ability to extend the circulating half-life of a polypeptide of which it is a part. In addition, full-length or fragmented Fc regions can be variants of the wild-type molecule. That is, they can contain mutations that may or may not affect the function of the polypeptides. For example, see Wang X, Mathieu M, Brezski RJ. IgG Fc engineering to modulate antibody effector functions. Protein Cell. 2018; 9 (1): 63-73. doi: 10.1007/s13238-017-0473-8.

In other embodiments, a ligand comprises a polypeptide that functions as an antigenic tag, such as a FLAG sequence. FLAG sequences are recognized by biotinylated, highly specific, anti-FLAG antibodies, as described herein (see also Blanar et al., Science 256:1014, 1992; LeClair et al., Proc. Natl. Acad. Sci. USA 89:8145, 1992). In some embodiments, the chimeric polypeptide further comprises a C-terminal c-myc epitope tag. Ligands can also be synthesized with a HIS-tag, as known in the art, for ease in purification.

Ligands can be acetylated. In some embodiments, the acetylation may occur at the N-terminus, using methods known in the art, e.g. by enzymatic reaction with N-terminal acetyltransferase and, for example, acetyl COA. In some embodiments, the ligand may be acetylated at one or more lysine residues, e.g. by enzymatic reaction with a lysine acetyltransferase. See, for example Choudhary et al. (2009). Science. 325 (5942): 834-840.

### Nucleic Acids and Expression

In the present methods, a tunable cytokine receptor subunit polypeptide may be produced by recombinant methods. A nucleic acid sequence encoding the tunable cytokine receptor subunit polypeptide may be incorporated into an expression vector in operable association with one or more expression control sequences (e.g. promoters, enhancers) into the cell to be engineered. The nucleic acid sequence encoding tunable cytokine receptor subunit polypeptide may be obtained from various sources as designed during the engineering process. Exemplary nucleic coding sequences are provided as SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59 61, 63, 65, 67, 69, 71, 73, 75, 77, 79, 81, 85, 87, 89 or 90 which may be provided as a ssDNA, dsDNA, DNA: RNA hybrid, ssRNA, dsRNA, or analogs thereof.

The tunable cytokine receptor subunit polypeptide and variants thereof may be prepared by introducing appropriate nucleotide changes into the coding sequences, as described herein. Such variants comprise insertions, substitutions, and/or deletions of residues as noted. Any combination of insertion, substitution, and/or specified deletion is made to arrive at the final construct, provided that the final construct possesses the desired biological activity as defined herein.

To achieve expression of the recombinant protein, a nucleic acid encoding a tunable cytokine receptor subunit polypeptide is inserted into a replicable vector for expression. Many such vectors are available. The vector components generally include, but are not limited to, one or more of the following: an origin of replication, an internal ribosome entry site (IRES), one or more marker genes, an enhancer element, a promoter, and a transcription termination sequence. Vectors include viral vectors, plasmid vectors, integrating vectors, and the like.

Expression vectors for expression of the tunable cytokine receptor subunit polypeptide may be viral vectors or non-viral vectors. Plasmids are examples of non-viral vectors. In order to facilitate transfection of the target cells, the target cell may be exposed directly to the non-viral vector under conditions that facilitate uptake of the non-viral vector. Examples of conditions which facilitate uptake of foreign nucleic acid by mammalian cells are well known in the art and include but are not limited to chemical means (such as Lipofectamine®, Thermo-Fisher Scientific), high salt, electroporation, and magnetic fields (electroporation). For example, see Novickij et al. (2016) Scientific Reports volume 6, Article number: 33537, “Pulsed Electromagnetic Field Assisted in vitro Electroporation”. In one embodiment, a non-viral vector may be provided in a non-viral delivery system. Non-viral delivery systems are typically complexes to facilitate transduction of the target cell with a nucleic acid cargo wherein the nucleic acid is complexed with agents such as cationic lipids (DOTAP, DOTMA), surfactants, biologicals (gelatin, chitosan), metals (gold, magnetic iron) and synthetic polymers (PLG, PEI, PAMAM). Numerous embodiments of non-viral delivery systems are well known in the art including lipidic vector systems (Lee et al. (1997) Crit Rev Ther Drug Carrier Syst. 14:173-206); polymer coated liposomes (Marin et al., U.S. Pat. No. 5,213,804, issued May 25, 1993; Woodle, et al., U.S. Pat. No. 5,013,556, issued May 7, 1991); cationic liposomes (Epand et al., U.S. Pat. No. 5,283,185, issued Feb. 1, 1994; Jessee, J. A., U.S. Pat. No. 5,578,475, issued Nov. 26, 1996; Rose et al, U.S. Pat. No. 5,279,833, issued Jan. 18, 1994; Gebeyehu et al., U.S. Pat. No. 5,334,761, issued Aug. 2, 1994).

In another embodiment, the expression vector may be a viral vector. When a viral vector system is to be employed, retroviral, e.g. lentiviral expression vectors, are preferred. In particular, the viral vector is a gamma retrovirus (. (Pule, et al. (2008) Nature Medicine 14 (11): 1264-1270), self-inactivating lentiviral vectors (June, et al. (2009) Nat Rev Immunol 9 (10): 704-716) and retroviral vectors as described in Naldini, et al. (1996) Science 272:263-267; Naldini, et al. (1996) Proc. Natl. Acad. Sci. USA Vol. 93, pp. 11382-11388; Dull, et al. (1998) J. Virology 72 (11): 8463-8471; Milone, et al. (2009) 17 (8): 1453-1464; Kingsman, et al. U.S. Pat. No. 6,096,538 issued Aug. 1, 2000 and Kingsman, et al. U.S. Pat. No. 6,924,123 issued Aug. 2, 2005.

Viral vectors of interest also include retroviral vectors (e.g. derived from MoMLV, MSCV, SFFV, MPSV, SNV etc), adeno-associated virus (AAV) vectors, adenoviral vectors (e.g. derived from Ad5 virus), SV40-based vectors, Herpes Simplex Virus (HSV)-based vectors etc.

Transduction of cells with an expression vector may be accomplished using techniques well known in the art including but not limited co-incubation with host T cells with viral vectors, electroporation, and/or chemically enhanced delivery.

A tunable cytokine receptor subunit polypeptide may also be produced with the naturally occurring signal sequence of the ECD, as provided in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59 61, 63, 65, 67, 69, 71, 73, 75, 77, 79, 81, 85, 87, 89 or 90, or the signal sequence may be a heterologous polypeptide, e.g. a signal sequence or other polypeptide having a specific cleavage site at the N-terminus of the mature protein or polypeptide. In general, the signal sequence may be a component of the vector, or it may be a part of the coding sequence that is inserted into the vector. The heterologous signal sequence selected preferably is one that is recognized and processed (i.e., cleaved by a signal peptidase) by the host cell. In mammalian cell expression the naturally-occurring signal sequence may be used, or other mammalian signal sequences may be suitable, such as signal sequences from secreted polypeptides of the same or related species, as well as viral secretory leaders, for example, the herpes simplex gD signal.

Expression vectors may contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

Expression vectors will contain a promoter that is recognized by the host organism and is operably linked to a tunable cytokine receptor subunit polypeptide coding sequence. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

Transcription from vectors in mammalian host cells may be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus (such as murine stem cell virus), hepatitis-B virus and most preferably Simian Virus 40 (SV40), from heterologous mammalian promoters, e.g., the actin promoter, PGK (phosphoglycerate kinase), or an immunoglobulin promoter, from heat-shock promoters, provided such promoters are compatible with the host cell systems. The early and late promoters of the SV40 virus are conveniently obtained as an SV40 restriction fragment that also contains the SV40 viral origin of replication.

Transcription by higher eukaryotes is often increased by inserting an enhancer sequence into the vector. Enhancers are cis-acting elements of DNA, usually about from 10 to 300 bp, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, alpha-fetoprotein, and insulin). Typically, however, one will use an enhancer from a eukaryotic cell virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence but is preferably located at a site 5′ from the promoter.

Expression vectors used in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. Construction of suitable vectors containing one or more of the above-listed components employs standard techniques.

Suitable host cells for cloning or expressing the DNA in the vectors herein are the prokaryote, yeast, or higher eukaryote cells described above. Examples of useful mammalian host cell lines are mouse L cells (L-M [TK-], ATCC #CRL-2648), monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); Chinese hamster ovary cells/-DHFR (CHO); mouse sertoli cells (TM4); monkey kidney cells (CV1 ATCC CCL 70); African green monkey kidney cells (VERO-76, ATCC CRL-1 587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2).

Host cells, including engineered T cells, can be transfected with the above-described expression vectors. Cells may be cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression and will be apparent to the ordinarily skilled artisan.

Nucleic acids are “operably linked” when placed into a functional relationship with another nucleic acid sequence. For example, DNA for a signal sequence is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in the same protein-coding open reading frame. However, enhancers do not have to be contiguous or in frame.

### Engineered Cells

In some embodiments, an engineered cell is provided, in which the cell has been modified by introduction of an expression vector comprising a nucleic acid sequence encoding a tunable cytokine receptor subunit polypeptide. Any cell can be used for this purpose. In some embodiments the cell is a T cell, including without limitation naïve CD8⁺ T cells, cytotoxic CD8⁺ T cells, naïve CD4⁺ T cells, helper T cells, e.g. T_{H}1, T_{H}2, T_{H}9, T_{H}11, T_{H}22, T_{FH}; regulatory T cells, e.g. T_{R}1, natural T_{Reg}, inducible T_{Reg}; memory T cells, e.g. central memory T cells, effector memory T cells, NKT cells, γδ T cells and engineered variants of such T cells including CAR T cells; etc., or a cell population such as TILs. In other embodiments the engineered cell is a stem cell, e.g. a hematopoietic stem cell, an NK cell, a macrophage, or a dendritic cell. In some embodiments the cell is genetically modified in an ex vivo procedure, prior to transfer into a subject, to introduce a coding sequence for the chimeric receptor. The engineered cell can be provided in a unit dose for therapy, and can be allogeneic, autologous, or xenogeneic with respect to an intended recipient.

T cells useful for engineering by introduction of the constructs described herein include TILs, naïve T cells, central memory T cells, effector memory T cells, regulatory T cells, or combination thereof. T cells for engineering as described above can be collected from a subject or a donor, and may be separated from a mixture of cells by techniques that enrich for desired cells or may be engineered and cultured without separation. An appropriate solution may be used for dispersion or suspension of the cells. Such solution will generally be a sterile balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. Techniques for affinity separation may include magnetic separation, using antibody-coated magnetic beads, affinity chromatography, cytotoxic agents linked to a monoclonal antibody or used in conjunction with a monoclonal antibody, e.g., complement and cytotoxins, and “panning” with antibody attached to a solid matrix, e.g., a plate, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g., propidium iodide). Any technique may be employed which is not unduly detrimental to the viability of the selected cells. The affinity reagents may be specific receptors or ligands for the cell surface molecules indicated above. In addition to antibody reagents, peptide-MHC antigen and T cell receptor pairs may be used; peptide ligands and receptor; effector and receptor molecules, and the like.

The separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum (FCS). The collected and optionally enriched cell population may be used immediately for genetic modification, or may be frozen at liquid nitrogen temperatures and stored, being thawed and capable of being reused. The cells will usually be stored in 10% DMSO, 50% FCS, 40% RPMI 1640 medium.

In some embodiments, the engineered cells comprise a complex mixture of immune cells, e.g., tumor infiltrating lymphocytes (TILs) isolated from an individual in need of treatment. See, for example, Yang and Rosenberg (2016) Adv Immunol. 130:279-94, “Adoptive T Cell Therapy for Cancer; Feldman et al (2015) Semin Oncol. 42 (4): 626-39 “Adoptive Cell Therapy-Tumor-Infiltrating Lymphocytes, T cell Receptors, and Chimeric Antigen Receptors”; Clinical Trial NCT01174121, “Immunotherapy Using Tumor Infiltrating Lymphocytes for Patients With Metastatic Cancer”; Tran et al. (2014) Science 344 (6184) 641-645, “Cancer immunotherapy based on mutation-specific CD4+ T cells in a patient with epithelial cancer”.

In some embodiments, an engineered T cell is allogeneic with respect to the individual that is treated, e.g. see clinical trials NCT03121625; NCT03016377; NCT02476734; NCT02746952; NCT02808442. See for review Graham et al. (2018) Cells. 7 (10) E155. In some embodiments an allogeneic engineered T cell is fully HLA matched.

Allogeneic T cells may be genetically modified to reduce graft versus host disease. For example the engineered cells may be TCRαβ receptor knock-outs achieved by gene editing techniques. TCRαβ is a heterodimer and both alpha and beta chains need to be present for it to be expressed. A single gene codes for the alpha chain (TRAC), whereas there are 2 genes coding for the beta chain, therefore the TRAC locus has been deleted for this purpose. A number of different approaches have been used to accomplish this deletion, e.g. CRISPR/Cas9; meganuclease; engineered I-Crel homing endonuclease, etc. See, for example, Eyquem et al. (2017) Nature 543:113-117, in which the TRAC coding sequence is replaced by a CAR coding sequence; and Georgiadis et al. (2018) Mol. Ther. 26:1215-1227, which linked CAR expression with TRAC disruption by clustered regularly interspaced short palindromic repeats (CRISPR)/Cas9) without directly incorporating the CAR into the TRAC loci. An alternative strategy to prevent GVHD modifies T cells to express an inhibitor of TCRαβ signaling, for example using a truncated form of CD3ζ as a TCR inhibitory molecule.

The preparation of T cells useful in the practice of the present invention is achieved by transforming isolated T cells with an expression vector comprising a nucleic acid sequence encoding a tunable cytokine receptor polypeptide; optionally in combination with a nucleic acid sequence encoding a CAR polypeptide. The nucleic acid sequences encoding a CAR and a tunable cytokine receptor polypeptide may each be provided on separate expression vectors, each nucleic acid sequence being operably linked to one or more expression control elements to achieve expression of the CAR and tunable cytokine receptor subunit polypeptide in the target cell, the vectors being co-transfected into the target cell. Alternatively, the nucleic acid sequences encoding the CAR and a tunable cytokine receptor polypeptide may each be provided on a single vector each nucleic acid sequence under the control of one or more expression control elements to achieve expression of the associated nucleic acid sequence. Alternatively, both nucleic acid sequences may be under the control of a single promoter with intervening or downstream control elements that facilitate co-expression of the two sequences from the vector.

Ex vivo T cell activation may be achieved by procedures well-established in the art including cell-based T cell activation, antibody-based activation or activation using a variety of bead-based activation reagents. Cell-based T cell activation may be achieved by exposure of the T cells to antigen presenting cells, such as dendritic cells or artificial antigen presenting cells such as irradiated K562 cells. Antibody based activation of T cell surface CD3 molecules with soluble anti-CD3 monoclonal antibodies and soluble anti-CD28 antibodies also supports T cell activation.

T cells are optionally expanded by culturing the cells in contact with a surface providing an agent that stimulates a CD3 TCR complex associated signal (e.g., an anti-CD3 antibody) and an agent that stimulates a co-stimulatory molecule on the surface of the T cells (e.g an agonistic anti-CD28 antibody). Bead-based T cell activation has gained acceptance in the art for the preparation of T cells for clinical use. Bead-based activation of T cells may be achieved using commercially available T cell activation reagents including but not limited to the Invitrogen® CTS Dynabeads® CD3/28 (Life Technologies, Inc. Carlsbad CA) or Miltenyi MACS® GMP ExpAct Treg beads or Miltenyi MACS GMP TransAct™ CD3/28 beads (Miltenyi Biotec, Inc.). Conditions appropriate for T cell culture are well known in the art. Lin, et al. (2009) Cytotherapy 11 (7): 912-922; Smith, et al. (2015) Clinical & Translational Immunology 4: e31 published online 16 Jan. 2015. The target cells are maintained under conditions necessary to support growth, for example, an appropriate temperature (e.g., 37° C.) and atmosphere (e.g., air plus 5% CO2).

The engineered cells may be infused to the subject in any physiologically acceptable medium by any convenient route of administration, normally intravascularly, although they may also be introduced by other routes, where the cells may find an appropriate site for growth. Usually, at least 1×10⁶ cells/kg will be administered, at least 1×10⁷ cells/kg, at least 1×10⁸ cells/kg, at least 1×10⁹ cells/kg, at least 1×10¹⁰ cells/kg, or more, usually being limited by the number of T cells that are obtained during collection.

In one embodiment, a T cell expressing a tunable cytokine receptor subunit polypeptide is a T cell which has been modified to surface express a chimeric antigen receptor (a ‘CAR T’ cell). As used herein, the terms “chimeric antigen receptor T cell” and “CAR T cell” are used interchangeably to refer to a T cell that has been recombinantly modified to express a chimeric antigen receptor. As used herein, the terms “chimeric antigen receptor” and “CAR” are used interchangeably to refer to a polypeptide comprising multiple functional domains arranged from amino to carboxy terminus in the sequence: (a) an antigen binding domain (ABD), (b) a transmembrane domain (TM); and (c) one or more cytoplasmic signaling domains (CSDs) wherein the foregoing domains may optionally be linked by one or more spacer domains. The CAR may also further comprise a signal peptide sequence which is conventionally removed during post-translational processing and presentation of the CAR on the cell surface. CARs useful in the practice of the present invention are prepared in accordance with principles well known in the art. See e.g., Eshhaar et al. U.S. Pat. No. 7,741,465 B1 issued Jun. 22, 2010; Sadelain, et al (2013) Cancer Discovery 3 (4): 388-398; Jensen and Riddell (2015) Current Opinions in Immunology 33:9-15; Gross, et al. (1989) PNAS (USA) 86 (24): 10024-10028; Curran, et al. (2012) J Gene Med 14 (6): 405-15. Examples of commercially available CAR T cell products that may be modified to incorporate an tunable cytokine receptor of the present invention include axicabtagene ciloleucel (marketed as Yescarta® commercially available from Gilead Pharmaceuticals) and tisagenlecleucel (marketed as Kymriah® commercially available from Novartis).

As used herein, the term antigen binding domain (ABD) refers to a polypeptide that specifically binds to an antigen expressed on the surface of a target cell. The ABD may be any polypeptide that specifically binds to one or more antigens expressed on the surface of a target cell. In certain embodiments, the target cell antigen is a tumor antigen. Non-limiting examples of tumor antigens that may be targeted by a CAR include one or more antigens selected from the group including, but not limited to, the CD19, CD20, HER2, NY-ESO-1, MUC1, CD123, FLT3, B7-H3, CD33, IL1RAP, CLL1 (CLEC12A) PSA, CEA, VEGF, VEGF-R2, CD22, ROR1, mesothelin, c-Met, Glycolipid F77, FAP, EGFRvIII, MAGE A3, 5T4, WT1, KG2D ligand, a folate receptor (FRa), and Wnt1 antigens.

In one embodiment, the ABD is a single chain Fv (ScFv). An ScFv is a polypeptide comprised of the variable regions of the immunoglobulin heavy and light chain of an antibody covalently connected by a peptide linker (Bird, et al. (1988) Science 242:423-426; Huston, et al. (1988) PNAS (USA) 85:5879-5883; S-z Hu, et al. (1996) Cancer Research, 56, 3055-3061. The generation of ScFvs based on monoclonal antibody sequences is well known in the art. See, e.g. The Protein Protocols Handbook, John M. Walker, Ed. (2002) Humana Press Section 150 “Bacterial Expression, Purification and Characterization of Single-Chain Antibodies” Kipriyanov, S. Antibodies used in the preparation of scFvs may be optimized to select for those molecules which possess particular desirable characteristics (e.g. enhanced affinity) through techniques well known in the art such as phage display and directed evolution. In some embodiments, the ABD comprises an anti-CD19 scFv, an anti-PSA scFv, an anti-HER2 scFv, an anti-CEA scFv, an anti-EGFR scFv, an anti-EGFRvIII scFv, an anti-NY-ESO-1 scFv, an anti-MAGE scFv, an anti-5T4 scFv, or an anti-Wnt1 scFv. In another embodiment, the ABD is a single domain antibody obtained through immunization of a camel or llama with a target cell derived antigen, in particular a tumor antigen. See, e.g. Muyldermans, S. (2001) Reviews in Molecular Biotechnology 74:277-302. Alternatively, the ABD may be generated wholly synthetically through the generation of peptide libraries and isolating compounds having the desired target cell antigen binding properties in substantial accordance with the teachings or Wigler, et al. U.S. Pat. No. 6,303,313 B1 issued Nov. 12, 1999; Knappik, et al., U.S. Pat. No. 6,696,248 B1 issued Feb. 24, 2004, Binz, et al. (2005) Nature Biotechnology 23:1257-1268, and Bradbury, et al. (2011) Nature Biotechnology 29:245-254.

The ABD may have affinity for more than one target antigen. For example, an ABD of the present invention may comprise chimeric bispecific binding members, i.e. have capable of providing for specific binding to a first target cell expressed antigen and a second target cell expressed antigen. Non-limiting examples of chimeric bispecific binding members include bispecific antibodies, bispecific conjugated monoclonal antibodies (mab) 2, bispecific antibody fragments (e.g., F (ab) 2, bispecific scFv, bispecific diabodies, single chain bispecific diabodies, etc.), bispecific T cell engagers (BiTE), bispecific conjugated single domain antibodies, micabodies and mutants thereof, and the like. Non-limiting examples of chimeric bispecific binding members also include those chimeric bispecific agents described in Kontermann (2012) MAbs. 4 (2): 182-197; Stamova et al. (2012) Antibodies, 1 (2), 172-198; Farhadfar et al. (2016) Leuk Res. 49:13-21; Benjamin et al. Ther Adv Hematol. (2016) 7 (3): 142-56; Kiefer et al. Immunol Rev. (2016) 270 (1): 178-92; Fan et al. (2015) J Hematol Oncol. 8:130; May et al. (2016) Am J Health Syst Pharm. 73 (1): e6-e13. In some embodiments, the chimeric bispecific binding member is a bivalent single chain polypeptides. See, e.g. Thirion, et al. (1996) European J. of Cancer Prevention 5 (6): 507-511; DeKruif and Logenberg (1996) J. Biol. Chem 271 (13) 7630-7634; and Kay, et al. United States Patent Application Publication Number 2015/0315566 published Nov. 5, 2015. In some instances, a chimeric bispecific binding member may be a bispecific T cell engager (BiTE). A BiTE is generally made by fusing a specific binding member (e.g., a scFv) that binds an antigen to a specific binding member (e.g., a scFv) with a second binding domain specific for a T cell molecule such as CD3. In some instances, a chimeric bispecific binding member may be a CAR T cell adapter. As used herein, by “CAR T cell adapter” is meant an expressed bispecific polypeptide that binds the antigen recognition domain of a CAR and redirects the CAR to a second antigen. Generally, a CAR T cell adapter will have to binding regions, one specific for an epitope on the CAR to which it is directed and a second epitope directed to a binding partner which, when bound, transduces the binding signal activating the CAR. Useful CAR T cell adapters include but are not limited to e.g., those described in Kim et al. (2015) J Am Chem Soc. 137 (8): 2832-5; Ma et al. (2016) Proc Natl Acad Sci USA. 113 (4): E450-8 and Cao et al. (2016) Angew Chem Int Ed Engl. 55 (26): 7520-4.

In some embodiments, a linker polypeptide molecule is optionally incorporated into the CAR between the antigen binding domain and the transmembrane domain to facilitate antigen binding. Moritz and Groner (1995) Gene Therapy 2 (8) 539-546. In one embodiment, the linker is the hinge region from an immunoglobulin, e.g. the hinge from any one of IgG1, IgG2a, IgG2b, IgG3, IgG4, particularly the human protein sequences. Alternatives include the CH2CH3 region of immunoglobulin and portions of CD3. In those instances where the ABD is an scFv, an IgG hinge may be employed. In some embodiments the linker comprises the amino acid sequence (G₄S)ₙ where n is 1, 2, 3, 4, 5, etc., and in some embodiments n is 3.

CARs useful in the practice of the present invention further comprise a transmembrane (TM) domain joining the ABD (or linker, if employed) to the intracellular cytoplasmic domain of the CAR. The transmembrane domain is comprised of any polypeptide sequence which is thermodynamically stable in a eukaryotic cell membrane. The transmembrane spanning domain may be derived from the transmembrane domain of a naturally occurring membrane spanning protein or may be synthetic. In designing synthetic transmembrane domains, amino acids favoring alpha-helical structures are preferred. Transmembrane domains useful in construction of CARs are comprised of approximately 10, 11, 12, 13, 14, 15, 16, 17, 18, 19, 20, 21, 22, 22, 23, or 24 amino acids favoring the formation having an alpha-helical secondary structure. Amino acids having a to favor alpha-helical conformations are well known in the art. See, e.g. Pace, et. al. (1998) Biophysical Journal 75:422-427. Amino acids that are particularly favored in alpha helical conformations include methionine, alanine, leucine, glutamate, and lysine. In some embodiments, the CAR transmembrane domain may be derived from the transmembrane domain from type I membrane spanning proteins, such as CD33, CD4, CD8, CD28, etc.

The cytoplasmic domain of the CAR polypeptide comprises one or more intracellular signal domains. In one embodiment, the intracellular signal domains comprise the cytoplasmic sequences of the T cell receptor (TCR) and co-receptors that initiate signal transduction following antigen receptor engagement and functional derivatives and sub-fragments thereof. A cytoplasmic signaling domain, such as those derived from the T cell receptor ζ-chain, is employed as part of the CAR in order to produce stimulatory signals for T lymphocyte proliferation and effector function following engagement of the chimeric receptor with the target antigen. Examples of cytoplasmic signaling domains include but are not limited to the cytoplasmic domain of CD27, the cytoplasmic domain S of CD28, the cytoplasmic domain of CD137 (also referred to as 4-1BB and TNFRSF9), the cytoplasmic domain of CD278 (also referred to as ICOS), p110a, β, or δ catalytic subunit of PI3 kinase, the human CD3ζ− chain, cytoplasmic domain of CD134 (also referred to as OX40 and TNFRSF4), FcεR1γ and β chains, MB1 (Igα) chain, B29 (Igβ) chain, etc.), CD3 polypeptides (δ, Δ and ε), syk family tyrosine kinases (Syk, ZAP 70, etc.), src family tyrosine kinases (Lck, Fyn, Lyn, etc.) and other molecules involved in T cell transduction, such as CD2, CD5 and CD28.

In some embodiments, the CAR may also provide a co-stimulatory domain. The term “co-stimulatory domain”, refers to a signaling endodomain of a CAR that provides a secondary non-specific activation mechanism through which a primary specific stimulation is propagated. The co-stimulatory domain refers to the portion of the CAR which enhances the proliferation, survival or development of memory cells. Examples of co-stimulation include antigen nonspecific T cell co-stimulation following antigen specific signaling through the T cell receptor and antigen nonspecific B cell co-stimulation following signaling through the antigen-specific B cell receptor. Co-stimulation, e.g., T cell co-stimulation, and the factors involved have been described in Chen & Flies. (2013) Nat Rev Immunol 13 (4): 227-42. In some embodiments of the present disclosure, the CSD comprises one or more of members of the TNFR superfamily, CD28, CD137 (4-1BB), CD134 (OX40), Dap10, CD27, CD2, CD5, ICAM-1, LFA-1 (CD11a/CD18), Lck, TNFR-I, TNFR-II, Fas, CD30, CD40 or combinations thereof.

CARs are often referred to as first, second, third or fourth generation. The term first-generation CAR refers to a CAR wherein the cytoplasmic domain transmits the signal from antigen binding through only a single signaling domain, for example a signaling domain derived from the high-affinity receptor for IgE FcεRIγ, or the CD3ζ chain. The single signaling domain contains one or three immunoreceptor tyrosine-based activating motif(s) [ITAM(s)] for antigen-dependent T cell activation. The ITAM-based activating signal endows T cells with the ability to lyse the target tumor cells and secret cytokines in response to antigen binding. Second-generation CARs include a co-stimulatory signal in addition to the CD3-domain. Coincidental delivery of the delivered co-stimulatory signal enhances persistence, cytokine secretion and antitumor activity induced by CAR Transduced T cells. The co-stimulatory domain is usually located membrane proximal relative to the CD3 domain. Third-generation CARs include a tripartite signaling domain, comprising for example a CD28, a CD33, and a OX40 or 4-1BB signaling region. Fourth generation CARs, or “armored car” CAR T cells are further gene modified to express or block molecules and/or receptors to enhance immune activity.

Exemplary intracellular signaling domains that may be incorporated into the CAR disclosed herein comprise (amino to carboxy): CD33; CD28-41BB-CD33; CD28-OX40-CD3ζ; CD28-41BB-CD3ζ; 41BB-CD-28-CD3ζ and 41BB-CD3ζ.

The term CAR includes CAR variants including but not limited split CARs, ON-switch CARS, bispecific or tandem CARs, inhibitory CARs (iCARs) and induced pluripotent stem (IPS) CAR T cells.

The term “Split CARs” refers to CARs wherein the extracellular portion, the ABD and the cytoplasmic signaling domain of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled. CAR molecules and derivatives thereof (i.e., CAR variants) are described, e.g., in PCT Application Nos. US2014/016527, US1996/017060, US2013/063083; Fedorov et al. Sci Transl Med (2013) 5 (215): 215ra172; Glienke et al. Front Pharmacol (2015) 6:21; Kakarla & Gottschalk 52 Cancer J (2014) 20 (2): 151-5; Riddell et al. Cancer J (2014) 20 (2): 141-4; Pegram et al. Cancer J (2014) 20 (2): 127-33; Cheadle et al. Immunol Rev (2014) 257 (1): 91-106; Barrett et al. Annu Rev Med (2014) 65:333-47; Sadelain et al. Cancer Discov (2013) 3 (4): 388-98; Cartellieri et al., J Biomed Biotechnol (2010) 956304; the disclosures of which are incorporated herein by reference in their entirety.

The terms “bispecific or tandem CARs” refer to CARs which include a secondary CAR binding domain that can either amplify or inhibit the activity of a primary CAR.

The terms “inhibitory chimeric antigen receptors” or “iCARs” are used interchangeably herein to refer to a CAR where binding iCARs use the dual antigen targeting to shut down the activation of an active CAR through the engagement of a second suppressive receptor equipped with inhibitory signaling domains of a secondary CAR binding domain results in inhibition of primary CAR activation. Inhibitory CARs (iCARs) are designed to regulate CAR T cell activity through inhibitory receptors signaling modules activation. This approach combines the activity of two CARs, one of which generates dominant negative signals limiting the responses of CAR T cells activated by the activating receptor. iCARs can switch off the response of the counteracting activator CAR when bound to a specific antigen expressed only by normal tissues. In this way, iCARs-T cells can distinguish cancer cells from healthy ones, and reversibly block functionalities of transduced T cells in an antigen-selective fashion. CTLA-4 or PD-1 intracellular domains in iCARs trigger inhibitory signals on T lymphocytes, leading to less cytokine production, less efficient target cell lysis, and altered lymphocyte motility.

The terms “tandem CAR” or “TanCAR” refer to CARs which mediate bispecific activation of T cells through the engagement of two chimeric receptors designed to deliver stimulatory or costimulatory signals in response to an independent engagement of two different tumor associated antigens.

### Therapeutic Cell Formulations and Uses

Methods and compositions are provided for enhancing cellular responses, by engineering cells from a recipient or donor by introduction of a tunable cytokine receptor subunit, and stimulating the tunable cytokine receptor subunit by contacting the engineered cell with the cognate ligand that specifically binds to and activates the ECD of the tunable cytokine receptor subunit. As discussed above, the subject methods include a step of obtaining the targeted cells, e.g. T cells, hematopoietic stem cells, etc., which may be isolated from a biological sample, or may be derived in vitro from a source of progenitor cells. The cells are transduced or transfected with an expression vector comprising a sequence encoding the tunable cytokine receptor subunit, which step may be performed in any suitable culture medium.

In some embodiments a therapeutic method is provided, the method comprising introducing into a recipient in need thereof of an engineered cell population, wherein the cell population has been modified by introduction of a vector comprising a sequence encoding an tunable cytokine receptor subunit. The cell population may be engineered ex vivo, and is usually autologous or allogeneic with respect to the recipient. In some embodiments, the introduced cell population is contacted with the cognate cytokine in vivo, following administration of the engineered cells.

Without being bound by theory, cells expressing a tunable cytokine receptor subunit are selectively activated by the ligand for the ECD. The STAT activation profile is determined by the TM and the ICD. In some embodiments the signaling pathways that are being activated are substantially similar to the signaling pathways activated by the receptor from which the ICD is derived, for example in the activity of specific JAK/STAT proteins, including, for example, STAT1, STAT3 and STAT5.

Where the engineered cells are T cells, an enhanced immune response may manifest as an increase in the cytolytic response of T cells towards the target cells present in the recipient, e.g. towards elimination of tumor cells and infected cells; a decrease in symptoms of autoimmune disease; and the like. The stemness of the engineered T cells can also be modified, to achieve a desired balance between activation and stemness in a population.

Where the cells are contacted with the ligand in vitro, the cytokine is added to the engineered cells in a dose and for a period of time sufficient to activate signaling from the receptor, which may utilize the native cellular machinery, e.g. accessory proteins, co-receptors, and the like. Any suitable culture medium may be used. The cells thus activated may be used for any desired purpose, including experimental purposes relating to determination of antigen specificity, cytokine profiling, and the like, and for delivery in vivo.

Where the contacting is performed in vivo, an effective dose of engineered cells, including without limitation CAR T cells modified to express a tunable cytokine receptor subunit, are infused to the recipient, in combination with or prior to administration of the cognate ligand, e.g., IL-2 and allowed to contact cells in their native environment, e.g. in lymph nodes, etc. Dosage and frequency may vary depending on the agent; mode of administration; nature of the cytokine; and the like. It will be understood by one of skill in the art that such guidelines will be adjusted for the individual circumstances. The dosage may also be varied for localized administration, e.g. intranasal, inhalation, etc., or for systemic administration. Parenteral infusions include intramuscular, intravenous (bolus or slow infusion), intraarterial, intraperitoneal, intrathecal, intratumoral, subcutaneous administration; etc.

Engineered T cells can be provided in pharmaceutical compositions suitable for therapeutic use, e.g. for human treatment. Therapeutic formulations comprising such cells can be frozen, or prepared for administration with physiologically acceptable carriers, excipients or stabilizers (Remington's Pharmaceutical Sciences 16th edition, Osol, A. Ed. (1980)), in the form of aqueous solutions. The cells will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

Generally at least about 10⁴ engineered cells/kg are administered, at least about 10⁵ engineered cells/kg; at least about 10⁶ engineered cells/kg, at least about 10⁷ engineered cells/kg, at least about 10⁸ engineered cells/kg, or more. For example, typical ranges for the administration of cells for use in the practice of the present invention range from about 1×10⁵ to 5×10⁸ viable cells per kg of subject body weight per course of therapy. Consequently, adjusted for body weight, typical ranges for the administration of viable cells in human subjects ranges from approximately 1×10⁶ to approximately 1×10¹³ viable cells, alternatively from approximately 5×10⁶ to approximately 5×10¹² viable cells, alternatively from approximately 1×10⁷ to approximately 1×10¹² viable cells, alternatively from approximately 5×10⁷ to approximately 1×10¹² viable cells, alternatively from approximately 1×10⁸ to approximately 1×10¹² viable cells, alternatively from approximately 5×10⁸ to approximately 1×10¹² viable cells, alternatively from approximately 1×10⁹ to approximately 1×10¹² viable cells per course of therapy. In one embodiment, the dose of the cells is in the range of 2.5-5×10⁹ viable cells per course of therapy.

A course of therapy may be a single dose or in multiple doses over a period of time. In some embodiments, the cells are administered in a single dose. In some embodiments, the cells are administered in two or more split doses administered over a period of 1, 2, 3, 4, 5, 6, 7, 8, 9, 10, 11, 12, 13, 14, 21, 28, 30, 60, 90, 120 or 180 days. The quantity of engineered cells administered in such split dosing protocols may be the same in each administration or may be provided at different levels. Multi-day dosing protocols over time periods may be provided by the skilled artisan (e.g. physician) monitoring the administration of the cells taking into account the response of the subject to the treatment including adverse effects of the treatment and their modulation as discussed above.

The preferred formulation depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In still some other embodiments, pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Also provided are kits for use in the methods. The subject kits include an expression vector encoding a tunable cytokine receptor subunit, or a cell comprising the expression vector. Kits may further comprise the cognate ligand. In some embodiments, the components are provided in a dosage form (e.g., a therapeutically effective dosage form), in liquid or solid form in any convenient packaging (e.g., stick pack, dose pack, etc.). Reagents for the selection or in vitro derivation of cells may also be provided, e.g. growth factors, differentiation agents, tissue culture reagents; and the like.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

### Therapeutic Methods

In some embodiments the subject compositions, methods and kits are used to enhance a T cell mediated immune response. In some embodiments the immune response is directed towards a condition where it is desirable to deplete or regulate target cells, e.g., cancer cells, infected cells, autoimmune cells, regulation of immune cells, including without limitation immune cells involved in autoimmune disease, immune cells involved in transplantation, undesirable inflammatory responses, enhancing erythropoiesis, enhancing thrombopoiesis, etc. Immune conditions may include, without limitation, autoimmune diseases, graft v host disease, hematopoietic bone marrow transplantation, adoptive cell therapy, tumor infiltrating cell (TIL) therapy, inflammation, graft rejection, and the like.

In some embodiments the condition is cancer. As used herein, the terms “cancer” (or “cancerous”), “hyperproliferative,” and “neoplastic” to refer to cells having the capacity for autonomous or unregulated growth (e.g., an abnormal state or condition characterized by rapidly proliferating cell growth). Hyperproliferative and neoplastic disease states may be categorized as pathologic (e.g., characterizing or constituting a disease state), or they may be categorized as non-pathologic (e.g., as a deviation from normal but not associated with a disease state). The terms are meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair. The terms “cancer” or “neoplasm” are used to refer to malignancies of the various organ systems, including those affecting the lung, breast, thyroid, lymph glands and lymphoid tissue, gastrointestinal organs, and the genitourinary tract, as well as to adenocarcinomas which are generally considered to include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. An “adenocarcinoma” refers to a carcinoma derived from glandular tissue or in which the tumor cells form recognizable glandular structures.

Examples of tumor cells include but are not limited to AML, ALL, CML, adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, Hodgkin's lymphoma, Kaposi's sarcoma, kidney cancer, laryngeal and hypopharyngeal cancer, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, or Waldenstrom's macroglobulinemia. Any cancer is a suitable cancer to be treated by the subject methods and compositions.

The compositions and method of the present invention may be combined with additional therapeutic agents. For example, when the disease, disorder or condition to be treated is a neoplastic disease (e.g. cancer) the methods may be combined with conventional chemotherapeutic agents or other biological anti-cancer drugs such as checkpoint inhibitors (e.g. PD1 or PDL1 inhibitors) or therapeutic monoclonal antibodies (e.g., Avastin®, Herceptin®).

Examples of chemical agents identified in the art as useful in the treatment of neoplastic disease, include without limitation, abitrexate, adriamycin, adrucil, amsacrine, asparaginase, anthracyclines, azacitidine, azathioprine, bicnu, blenoxane, busulfan, bleomycin, camptosar, camptothecins, carboplatin, carmustine, cerubidine, chlorambucil, cisplatin, cladribine, cosmegen, cytarabine, cytosar, cyclophosphamide, cytoxan, dactinomycin, docetaxel, doxorubicin, daunorubicin, ellence, elspar, epirubicin, etoposide, fludarabine, fluorouracil, fludara, gemcitabine, gemzar, hycamtin, hydroxyurea, hydrea, idamycin, idarubicin, ifosfamide, ifex, irinotecan, lanvis, leukeran, leustatin, matulane, mechlorethamine, mercaptopurine, methotrexate, mitomycin, mitoxantrone, mithramycin, mutamycin, myleran, mylosar, navelbine, nipent, novantrone, oncovin, oxaliplatin, paclitaxel, paraplatin, pentostatin, platinol, plicamycin, procarbazine, purinethol, ralitrexed, taxotere, taxol, teniposide, thioguanine, tomudex, topotecan, valrubicin, velban, vepesid, vinblastine, vindesine, vincristine, vinorelbine, VP-16, and vumon.

Targeted therapeutics that can be administered in combination may include, without limitation, tyrosine-kinase inhibitors, such as Imatinib mesylate (Gleevec, also known as STI-571), Gefitinib (Iressa, also known as ZD1839), Erlotinib (marketed as Tarceva), Sorafenib (Nexavar), Sunitinib (Sutent), Dasatinib (Sprycel), Lapatinib (Tykerb), Nilotinib (Tasigna), and Bortezomib (Velcade), Jakafi (ruxolitinib); Janus kinase inhibitors, such as tofacitinib; ALK inhibitors, such as crizotinib; Bcl-2 inhibitors, such as obatoclax, venclexta, and gossypol; FLT3 inhibitors, such as midostaurin (Rydapt), IDH inhibitors, such as AG-221, PARP inhibitors, such as Iniparib and Olaparib; PI3K inhibitors, such as perifosine; VEGF Receptor 2 inhibitors, such as Apatinib; AN-152 (AEZS-108) doxorubicin linked to [D-Lys (6)]-LHRH; Braf inhibitors, such as vemurafenib, dabrafenib, and LGX818; MEK inhibitors, such as trametinib; CDK inhibitors, such as PD-0332991 and LEE011; Hsp90 inhibitors, such as salinomycin; and/or small molecule drug conjugates, such as Vintafolide; serine/threonine kinase inhibitors, such as Temsirolimus (Torisel), Everolimus (Afinitor), Vemurafenib (Zelboraf), Trametinib (Mekinist), and Dabrafenib (Tafinlar).

Examples of biological agents identified in the art as useful in the treatment of neoplastic disease, include without limitation, cytokines or cytokine antagonists such as IL-12, INFα, or anti-epidermal growth factor receptor, radiotherapy, irinotecan; tetrahydrofolate antimetabolites such as pemetrexed; antibodies against tumor antigens, a complex of a monoclonal antibody and toxin, a T cell adjuvant, bone marrow transplant, or antigen presenting cells (e.g., dendritic cell therapy), anti-tumor vaccines, replication competent viruses, signal transduction inhibitors (e.g., Gleevec® or Herceptin®) or an immunomodulator to achieve additive or synergistic suppression of tumor growth, cyclooxygenase-2 (COX-2) inhibitors, steroids, TNF antagonists (e.g., Remicade® and Enbrel®), interferon-β1a (Avonex®), and interferon-β1b (Betaseron®) as well as combinations of one or more of the foregoing as practiced in known chemotherapeutic treatment regimens readily appreciated by the skilled clinician in the art.

Tumor specific monoclonal antibodies that can be administered in combination with an engineered cell may include, without limitation, Rituximab (marketed as MabThera® or Rituxan®), Alemtuzumab, Panitumumab, Ipilimumab (Yervoy®), etc.

In some embodiments the compositions and methods of the present invention may be combined with immune checkpoint therapy. Examples of immune checkpoint therapies include inhibitors of the binding of PD1 to PDL1 and/or PDL2. PD1 to PDL1 and/or PDL2 inhibitors are well known in the art. Examples of commercially available monoclonal antibodies that interfere with the binding of PD1 to PDL1 and/or PDL2 include nivolumab (Opdivo®, BMS-936558, MDX1106, commercially available from BristolMyers Squibb, Princeton NJ), pembrolizumab (Keytruda®MK-3475, lambrolizumab, commercially available from Merck and Company, Kenilworth NJ), and atezolizumab (Tecentriq®, Genentech/Roche, South San Francisco CA). Additional examples of PD1 inhibitory antibodies include but are not limited to durvalumab (MEDI4736, Medimmune/AstraZeneca), pidilizumab (CT-011, CureTech), PDR001 (Novartis), BMS-936559 (MDX1105, Bristol Myers Squibb), and avelumab (MSB0010718C, Merck Serono/Pfizer) and SHR-1210 (Incyte). Additional antibody PD1 pathway inhibitors are described in U.S. Pat. No. 8,217,149 (Genentech, Inc) issued Jul. 10, 2012; U.S. Pat. No. 8,168,757 (Merck Sharp and Dohme Corp.) issued May 1, 2012, U.S. Pat. No. 8,008,449 (Medarex) issued Aug. 30, 2011, U.S. Pat. No. 7,943,743 (Medarex, Inc) issued May 17, 2011. Additionally, small molecule PD1 to PDL1 and/or PDL2 inhibitors are known in the art. See, e.g. Sasikumar, et al as WO2016142833A1 and Sasikumar, et al. WO2016142886A2, BMS-1166 and BMS-1001 (Skalniak, et al (2017) Oncotarget 8 (42): 72167-72181).

In other embodiments the methods of the invention are used in the treatment of infection. As used herein, the term “infection” refers to any state in at least one cell of an organism (i.e., a subject) is infected by an infectious agent (e.g., a subject has an intracellular pathogen infection, e.g., a chronic intracellular pathogen infection). For example, infectious agents include, but are not limited to bacteria, viruses, protozoans, and fungi. Intracellular pathogens are of particular interest. Infectious diseases are disorders caused by infectious agents. Some infectious agents cause no recognizable symptoms or disease under certain conditions, but have the potential to cause symptoms or disease under changed conditions. The subject methods can be used in the treatment of chronic pathogen infections, for example including but not limited to viral infections, e.g. retrovirus, lentivirus, hepadna virus, herpes viruses, pox viruses, human papilloma viruses, etc.; intracellular bacterial infections, e.g. Mycobacterium, Chlamydophila, Ehrlichia, Rickettsia, Brucella, Legionella, Francisella, Listeria, Coxiella, Neisseria, Salmonella, Yersinia sp, Helicobacter pylori etc.; and intracellular protozoan pathogens, e.g. Plasmodium sp, Trypanosoma sp., Giardia sp., Toxoplasma sp., Leishmania sp., etc.

Treatment may be combined with other active agents. Classes of antibiotics include penicillins, e.g. penicillin G, penicillin V, methicillin, oxacillin, carbenicillin, nafcillin, ampicillin, etc.; penicillins in combination with β-lactamase inhibitors, cephalosporins, e.g. cefaclor, cefazolin, cefuroxime, moxalactam, etc.; carbapenems; monobactams; aminoglycosides; tetracyclines; macrolides; lincomycins; polymyxins; sulfonamides; quinolones; cloramphenical; metronidazole; spectinomycin; trimethoprim; vancomycin; etc. Cytokines may also be included, e.g. interferon Y, tumor necrosis factor a, interleukin 12, etc. Antiviral agents, e.g. acyclovir, gancyclovir, etc., may also be used in treatment.

In yet other embodiments, regulatory T cells are engineered for the treatment of autoimmune disease. The spectrum of inflammatory diseases and diseases associated with inflammation is broad and includes autoimmune diseases such rheumatoid arthritis (RA), systemic lupus erythematosus (SLE), multiple sclerosis (MS), and autoimmune hepatitis; insulin dependent diabetes mellitus, degenerative diseases such as osteoarthritis (OA), Alzheimer's disease (AD), and macular degeneration.

Many, if not most, autoimmune and inflammatory diseases involve multiple types of T cells, e.g. TH1, TH2, TH17, and the like. Autoimmune diseases are characterized by T and B lymphocytes that aberrantly target self-proteins, -polypeptides, -peptides, and/or other self-molecules causing injury and or malfunction of an organ, tissue, or cell-type within the body (for example, pancreas, brain, thyroid or gastrointestinal tract) to cause the clinical manifestations of the disease. Autoimmune diseases include diseases that affect specific tissues as well as diseases that can affect multiple tissues, which can depend, in part on whether the responses are directed to an antigen confined to a particular tissue or to an antigen that is widely distributed in the body.

The invention now being fully described, it will be apparent to one of ordinary skill in the art that various changes and modifications can be made without departing from the spirit or scope of the invention.

### Example 1

### Materials and Methods

Mammalian expression vectors. cDNA encoding chimeric receptors were PCR cloned into pMSCV-IRES-YFP retroviral vector.

Cell culture and Retrovirus production. HEK293T cells were maintained in DMEM supplemented with 10% Fetal Bovine Serum (FBS), 1% L-glutamine (L-glu), and 1% penicillin/streptomycin (P/S). To produce retrovirus, HEK293T cells were transfected with pMSCV retroviral vector and pCL-Eco packaging vector at ratio of 1.5:1 using X-tremeGene™ HP (Roche). 24h post transfection, media was removed and replenished with DMEM containing 5% FBS and cultured for an additional 24h. Media was collected (RV supe), clarified using a 0.45 μm filter, and flash frozen in liquid nitrogen for storage at −80 C. Media was replenished (DMEM/5% FBS) and cells were cultured for an additional 24 h and virus was collected and stored as above.

Isolation and retroviral transduction of primary mouse T cells. Cells from the spleen and lymph nodes of C57BL/6J mice were harvested, processed to a single cell suspension, and activated on plate-bound anti-CD3 (145-2C11, 2.5 μg/ml) and soluble anti-CD28 (37.51, 5 ug/ml) in T cell media (RPMI-1640, 10% FBS, HEPES, 1% Pen/Strep, Glutamax, β-mercaptoethanol, Sodium pyruvate, and NEAA) supplemented with 100 IU/ml mIL2. 24h post activation, cells were resuspended in viral supernatant (RV supe) containing polybrene and 100 IU/ml mIL2, and spinfected at 2700 rpm, 32 C for 90′. RV supe was then removed, and cells replenished with T cell media containing mIL2. 24h post transduction, cells were harvested and expanded in T cell media containing mIL2 for 24h. Media was then exchanged, and cells allowed to rest in T cell media lacking mIL2 for an additional 24h before being used for in vitro signaling or proliferation assays.

In Vitro phospho-signaling assay. RV transduced activated/rested primary mouse T cells were plated at 1×10⁵ cells per well in ultra-low binding 96-well round bottom plate (Cat. 7007; Costar) in 100 μL warm media. Cells were stimulated by addition of 100 μL solution of serial dilutions of ligand, e.g. ortho-IL-2 for 20′ at 37° C. and the reaction was terminated by 1.5% paraformaldehyde (PFA) fixation for 10′ at RT with agitation. Cells were then permeabilized with 100% ice-cold methanol for at least 45′ on ice or stored at −80° C. overnight. Fixed, permeabilized cells were washed 3× with FACS buffer and intracellular phosphorylated STAT proteins were detected with anti-STAT5 pY694-Alexa647, anti-STAT3 pY705-Alexa647 (BD Biosciences), or anti-STAT1 pY701-Alexa647 (Cell Signaling) diluted 1:100 in FACS buffer and incubated for 1 h at 4 C. Cells were washed and analyzed on a CytoFLEX equipped with a high-throughput autosampler (Beckman Coulter). Data represent the mean fluorescence intensity (MFI) and points were fit to a sigmoidal dose response curve using Prism 8 (GraphPad). All data are presented as mean (n=2/3)±SEM.

In vitro primary mouse T cell proliferation assay. RV transduced activated/rested primary mouse T cells were labeled with CellTracer Violet according to manufacturer protocol (Molecular Probes), and cultured at 1×10⁵ cells per well in ultra-low binding 96-well round bottom with serial dilutions of ligand. 72h later, cells were analyzed on the CytoFLEX (Beckman Coulter).

In Vitro cell surface marker profiling. RV transduced activated/rested primary mouse T cells were cultured at 1×10⁵ cells per well in ultra-low binding 96-well round bottom plate in presence of stimulating ligand, i.e. ortho-IL-2 (5 uM_{(f)}) or wild-type-IL-2 (50 nM_{(f)}) for 2-4 days at 37° C. Cells were harvested and stained with fluorophore-conjugated antibody to cell surface markers of interest (i.e. CD44, CD62L, Sca1, CD95, CD27, or CCR7) for 30′ on ice. Cells were washed, resuspend in propidium iodine (PI) containing buffer for live-dead discrimination, and analyzed on a CytoFLEX equipped with a high-throughput autosampler (Beckman Coulter). Data represent the mean fluorescence intensity (MFI). All data are presented as mean (n=2/3)±SEM.

Animals. C57BL/6J (Cat. 000664) mice were purchased from Jackson Labs, and housed at Stanford University Animal Facility according to approved protocols.

TABLE 2

| | |
|---|---|
| SEQ ID NO: 1 | DNA sequence mouse ortho IL-2 receptor β protein ECD; mouse Interferon alpha R1 |
| | receptor TM and ICD |
| SEQ ID NO: 2 | AA sequence mouse ortho IL-2 receptor β protein ECD, mouse Interferon alpha R1 |
| | receptor protein TM and ICD |
| SEQ ID NO: 3 | DNA sequence mouse ortho IL-2 receptor β protein ECD, mouse Interferon alpha R2 |
| | receptor TM and ICD |
| SEQ ID NO: 4 | AA sequence mouse ortho IL-2 receptor β protein ECD, mouse Interferon alpha R2 |
| | receptor TM and ICD |
| SEQ ID NO: 5 | DNA sequence mouse ortho IL-2 receptor β protein ECD, mouse Interferon gamma |
| | R1 receptor TM and ICD |
| SEQ ID NO: 6 | AA sequence mouse ortho IL-2 receptor β protein ECD, mouse Interferon gamma R1 |
| | receptor TM and ICD |
| SEQ ID NO: 7 | DNA sequence mouse ortho IL-2 receptor β protein ECD; mouse Interferon gamma |
| | R2 receptor, TM and ICD |
| SEQ ID NO: 8 | AA sequence mouse ortho IL-2 receptor β protein ECD; mouse Interferon gamma R2 |
| | receptor, TM and ICD |
| SEQ ID NO: 9 | DNA sequence mouse ortho IL-2 receptor β protein ECD; Interferon lambda R1 |
| | receptor, TM and ICD |
| SEQ ID NO: 10 | AA sequence mouse ortho IL-2 receptor β protein ECD; Interferon lambda R1 |
| | receptor TM and ICD |
| SEQ ID NO: 11 | DNA sequence mouse ortho IL-2 receptor β protein ECD, mouse IL-10 receptor alpha |
| | TM and ICD |
| SEQ ID NO: 12 | AA sequence mouse ortho IL-2 receptor β protein ECD, mouse IL-10 receptor alpha |
| | TM and ICD |
| SEQ ID NO: 13 | DNA sequence mouse ortho IL-2 receptor β protein ECD, mouse IL20Ra TM and ICD |
| SEQ ID NO: 14 | AA sequence mouse ortho IL-2 receptor β protein ECD, mouse IL20Ra TM and ICD |
| SEQ ID NO: 15 | DNA sequence mouse ortho IL-2 receptor β protein ECD, mouse IL-22 receptor α TM |
| | and ICD |
| SEQ ID NO: 16 | AA sequence mouse ortho IL-2 receptor β protein ECD, mouse IL-22 receptor α TM |
| | and ICD |
| SEQ ID NO: 17 | DNA sequence mouse ortho IL-2 receptor β protein ECD, mouse IL22 receptor a |
| | mutated (Y437F) |
| SEQ ID NO: 18 | AA sequence mouse ortho IL-2 receptor β protein ECD, mouse IL22 receptor a |
| | mutated (Y437F) |
| SEQ ID NO: 19 | DNA sequence mouse ortho IL-2 receptor β protein ECD, IL-9 receptor WT (YLPQ) |
| | (Kalbasi et al. Nature (2022) TM and ICD |
| SEQ ID NO: 20 | AA sequence mouse ortho IL-2 receptor β protein ECD, IL-9 receptor WT (YLPQ) |
| | (Kalbasi et al. Nature (2022) TM and ICD |
| SEQ ID NO: 21 | DNA sequence mouse ortho IL-2 receptor β protein ECD, IL-9 receptor mut1 |
| | Phosphosite Y384 changed to FLPQ TM and ICD |
| SEQ ID NO: 22 | AA sequence mouse ortho IL-2 receptor β protein ECD, IL-9 receptor mut1 |
| | Phosphosite Y384 changed to FLPQ TM and ICD |
| SEQ ID NO: 23 | DNA sequence mouse ortho IL-2 receptor β protein ECD, IL-9 receptor mut 6 |
| | Phosphosite Y384 changed to YRPQ TM and ICD |
| SEQ ID NO: 24 | AA sequence mouse ortho IL-2 receptor β protein ECD, IL-9 receptor mut 6 |
| | Phosphosite Y384 changed to YRPQ TM and ICD |
| SEQ ID NO: 25 | DNA sequence mouse ortho IL-2 receptor β protein ECD, IL-9 receptor mut 7 |
| | Phosphosite Y384 changed to YLPL TM and ICD |
| SEQ ID NO: 26 | AA sequence mouse ortho IL-2 receptor β protein ECD, IL-9 receptor mut 7 |
| | Phosphosite Y384 changed to YLPL TM and ICD |
| SEQ ID NO: 27 | DNA sequence mouse ortho IL-2 receptor β protein ECD, IL-9 receptor mut9 |
| | Phosphosite Y384 changed to YLKQ DNA TM and ICD of sequence |
| SEQ ID NO: 28 | AA sequence mouse ortho IL-2 receptor β protein ECD, IL-9 receptor mut9 |
| | Phosphosite Y384 changed to YLKQ TM and ICD |
| SEQ ID NO: 29 | DNA sequence mouse ortho IL-2 receptor β protein ECD, IL-9 receptor mutX |
| | Phosphosite Y384 changed to YLXQ, X = any 20 AA TM and ICD |
| SEQ ID NO: 30 | AA sequence mouse ortho IL-2 receptor β protein ECD, IL-9 receptor mutX |
| | Phosphosite Y384 changed to YLXQ, X = any 20 AA TM and ICD |
| SEQ ID NO: 31 | DNA sequence mouse ortho IL-2 receptor β protein ECD, IL-9 receptor 1x pY with |
| | single copy of IL2Rb pSTAT5 phosphosite TM and ICD |
| SEQ ID NO: 32 | AA sequence mouse ortho IL-2 receptor β protein ECD, IL-9 receptor 1x pY with |
| | single copy of IL2Rb pSTAT5 phosphosite TM and ICD |
| SEQ ID NO: 33 | DNA sequence mouse ortho IL-2 receptor β protein ECD, IL-9 receptor 2x pY with |
| | two IL2Rb pSTAT5 phosphosites in tandem TM and ICD |
| SEQ ID NO: 34 | AA sequence mouse ortho IL-2 receptor β protein ECD, IL-9 receptor 2x pY with two |
| | IL2Rb pSTAT5 phosphosites in tandem TM and ICD |
| SEQ ID NO: 35 | DNA sequence mouse ortho IL-2 receptor β protein ECD, IL-9 receptor 3x pY with |
| | three IL2Rb pSTAT5 phosphosites in tandem TM and ICD |
| SEQ ID NO: 36 | AA sequence mouse ortho IL-2 receptor β protein ECD, IL-9 receptor 3x pY with three |
| | IL2Rb pSTAT5 phosphosites in tandem TM and ICD |
| SEQ ID NO: 37 | DNA sequence mouse ortho IL-2 receptor β protein ECD, IL-9 receptor mutX (YLXQ) |
| | 1x pY YLXQ, x = any 20 AA, with 1-3 IL2Rb pSTAT5 phosphosite TM and ICD of IL- |
| | 9 receptor mutX (YLXQ) 1x pY YLXQ, x = any 20 AA, with 1-3 IL2Rb pSTAT5 |
| | phosphosite |
| SEQ ID NO: 38 | AA sequence mouse ortho IL-2 receptor β protein ECD, IL-9 receptor mutX (YLXQ) |
| | 1x pY YLXQ, x = any 20 AA, with 1-3 IL2Rb pSTAT5 phosphosite TM and ICD of IL- |
| | 9 receptor mutX (YLXQ) 1x pY YLXQ, x = any 20 AA, with 1-3 IL2Rb pSTAT5 phosphosite |
| SEQ ID NO: 39 | DNA sequence mouse ortho IL-2 receptor β protein ECD, mouse IL-21 receptor DNA |
| | sequence (Kalbasi et al. Nature (2022) TM and ICD |
| SEQ ID NO: 40 | AA sequence mouse ortho IL-2 receptor β protein ECD, mouse IL-21 receptor DNA |
| | sequence (Kalbasi et al. Nature (2022) TM and ICD |
| SEQ ID NO: 41 | DNA sequence mouse ortho IL-2 receptor β protein ECD, mouse IL-21 receptor 1x |
| | pY addition of 1-321 AA of IL2Rb C-terminus TM and ICD |
| SEQ ID NO: 42 | AA sequence mouse mouse ortho IL-2 receptor β protein ECD, mouse IL-21 receptor |
| | 1x pY addition of 1-321 AA of IL2Rb C-terminus TM and ICD |
| SEQ ID NO: 43 | DNA sequence mouse ortho IL-2 receptor β protein ECD, mouse IL-21 receptor with |
| | phosphosite changed to SAY TM and ICD |
| SEQ ID NO: 44 | AA sequence mouse ortho IL-2 receptor β protein ECD, mouse IL-21 receptor with |
| | phosphosite changed to SAY TM and ICD |
| SEQ ID NO: 45 | DNA sequence mouse ortho IL-2 receptor β protein ECD, of mouse IL-21 receptor |
| | with phosphosite changed to DAY TM and ICD |
| SEQ ID NO: 46 | AA sequence mouse ortho IL-2 receptor β protein ECD, of mouse IL-21 receptor with |
| | phosphosite changed to DAY TM and ICD |
| SEQ ID NO: 47 | DNA sequence mouse ortho IL-2 receptor β protein ECD, mouse IL-21 receptor with |
| | phosphosite changed to DAYLXQ, X = any 20 AA TM and ICD |
| SEQ ID NO: 48 | AA sequence mouse ortho IL-2 receptor β protein ECD, mouse IL-21 receptor with |
| | phosphosite changed to DAYLXQ, X = any 20 AA TM and ICD |
| SEQ ID NO: 49 | DNA sequence human ortho IL-2 receptor β ECD, human Interferon alpha R2 |
| | receptor TM and ICD of DNA sequence |
| SEQ ID NO: 50 | AA sequence human ortho IL-2 receptor β ECD, human Interferon alpha R2 receptor |
| | TM and ICD of DNA sequence |
| SEQ ID NO: 51 | DNA sequence human ortho IL-2 receptor β ECD, of human Interferon gamma R1 |
| | receptor TM and ICD |
| SEQ ID NO: 52 | AA sequence human ortho IL-2 receptor β ECD, of human Interferon gamma R1 |
| | receptor TM and ICD |
| SEQ ID NO: 53 | DNA sequence human ortho IL-2 receptor β ECD, human Interferon lambda R1 |
| | receptor TM and ICD |
| SEQ ID NO: 54 | AA sequence human ortho IL-2 receptor β ECD, human Interferon lambda R1 |
| | receptor TM and ICD |
| SEQ ID NO: 55 | DNA sequence human ortho IL-2 receptor β ECD, human IL-10 receptor alpha TM |
| | and ICD |
| SEQ ID NO: 56 | AA sequence human ortho IL-2 receptor β ECD, human IL-10 receptor alpha TM and |
| | ICD |
| SEQ ID NO: 57 | DNA sequence human ortho IL-2 receptor β ECD, human IL20Ra TM and ICD |
| SEQ ID NO: 58 | AA sequence human ortho IL-2 receptor β ECD, human IL20Ra TM and ICD |
| SEQ ID NO: 59 | DNA sequence human ortho IL-2 receptor β ECD, human IL22 receptor α TM and |
| | CD |
| SEQ ID NO: 60 | AA sequence human ortho IL-2 receptor β ECD, human IL22 receptor α TM and ICD |
| SEQ ID NO: 61 | DNA sequence human ortho IL-2 receptor β ECD, of human IL-9 receptor WT (YLPQ) |
| | (Kalbasi et al. Nature (2022) TM and ICD |
| SEQ ID NO: 62 | AA sequence human ortho IL-2 receptor β ECD, of human IL-9 receptor WT (YLPQ) |
| | (Kalbasi et al. Nature (2022) TM and ICD |
| SEQ ID NO: 63 | DNA sequence human ortho IL-2 receptor β ECD, human IL-9 receptor mut1 |
| | Phosphosite Y384 changed to FLPQ TM and ICD |
| SEQ ID NO: 64 | AA sequence human ortho IL-2 receptor β ECD, human IL-9 receptor mut1 |
| | Phosphosite Y384 changed to FLPQ TM and ICD |
| SEQ ID NO: 65 | DNA sequence human ortho IL-2 receptor β ECD, TM and ICD of human IL-9 receptor |
| | mut6 Phosphosite Y384 changed to YRPQ DNA sequence |
| SEQ ID NO: 66 | AA sequence human ortho IL-2 receptor β ECD, human IL-9 receptor mut6 |
| | Phosphosite Y384 changed to YRPQ TM and ICD |
| SEQ ID NO: 67 | DNA sequence human ortho IL-2 receptor β ECD, human IL-9 receptor mut7 |
| | Phosphosite Y384 changed to YLPL TM and ICD |
| SEQ ID NO: 68 | AA sequence human ortho IL-2 receptor β ECD, human IL-9 receptor mut7 |
| | Phosphosite Y384 changed to YLPL TM and ICD |
| SEQ ID NO: 69 | DNA sequence human ortho IL-2 receptor β ECD, human IL-9 receptor mutX |
| | Phosphosite Y384 changed to YLXQ, X = any 20 AA TM and ICD |
| SEQ ID NO: 70 | AA sequence human ortho IL-2 receptor B ECD, human IL-9 receptor mutX |
| | Phosphosite Y384 changed to YLXQ, X = any 20 AA TM and ICD |
| SEQ ID NO: 71 | DNA sequence human ortho IL-2 receptor β ECD, human IL-9 receptor 1x pY with 1- |
| | 3 copies of IL2Rb pSTAT5 phosphosite TM and ICD |
| SEQ ID NO: 72 | AA sequence human ortho IL-2 receptor β ECD, human IL-9 receptor 1x pY with 1-3 |
| | copies of IL2Rb pSTAT5 phosphosite TM and ICD |
| SEQ ID NO: 73 | DNA sequence human ortho IL-2 receptor β ECD, human IL-9 receptor mutX (YLXQ) |
| | 1x pY YLXQ, x = any 20 AA, with 1-3 copies IL2Rb pSTAT5 phosphosite TM and ICD |
| SEQ ID NO: 74 | AA sequence human ortho IL-2 receptor β ECD, human IL-9 receptor mutX (YLXQ) |
| | 1x pY YLXQ, x = any 20 AA, with 1-3 copies IL2Rb pSTAT5 phosphosite TM and ICD |
| SEQ ID NO: 75 | DNA sequence human ortho IL-2 receptor β ECD, human IL-21 receptor WT TM and CD |
| SEQ ID NO: 76 | AA sequence human ortho IL-2 receptor β ECD, human IL-21 receptor WT TM and ICD |
| SEQ ID NO: 77 | DNA sequence human ortho IL-2 receptor β ECD; human IL-21 receptor 1x pY with |
| | addition of 1-3 copies 21 AA of IL2Rb C-terminus TM and ICD |
| SEQ ID NO: 78 | AA sequence human ortho IL-2 receptor β ECD; human IL-21 receptor 1x pY with |
| | addition of 1-3 copies 21 AA of IL2Rb C-terminus TM and ICD |
| SEQ ID NO: 79 | DNA sequence human ortho IL-2 receptor β ECD; human IL-21 receptor with |
| | phophosite changed to SAY TM and ICD |
| SEQ ID NO: 80 | AA sequence human ortho IL-2 receptor β ECD; human IL-21 receptor with |
| | phophosite changed to SAY TM and ICD |
| SEQ ID NO: 81 | DNA sequence human ortho IL-2 receptor β ECD; human IL-21 receptor with |
| | phophosite changed to DAYLXQ, X = any 20 AA TM and ICD |
| SEQ ID NO: 82 | AA sequence human ortho IL-2 receptor β ECD; human IL-21 receptor with |
| | phophosite changed to DAYLXQ, X = any 20 AA TM and ICD |
| SEQ ID NO: 83 | DNA sequence of IL2Rb carboxy-terminus containing 3x pSTAT5 binding sites |
| SEQ ID NO: 84 | AA sequence IL2Rb carboxy-terminus containing 3x pSTAT5 binding sites |
| SEQ ID NO: 85 | DNA sequence mouse ortho IL-2 receptor β ECD and TM; mouse GCSFR ICD |
| SEQ ID NO: 86 | AA sequence mouse ortho IL-2 receptor β ECD and TM; mouse GCSFR ICD |
| SEQ ID NO: 87 | DNA sequence mouse ortho IL-2 receptor β ECD; mouse GCSFR TM and ICD |
| SEQ ID NO: 88 | AA sequence mouse ortho IL-2 receptor β ECD; mouse GCSFR TM and ICD |
| SEQ ID NO: 89 | DNA sequence human ortho IL-2 receptor β ECD and TM; human GCSFR ICD |
| SEQ ID NO: 90 | AA sequence human ortho IL-2 receptor β ECD and TM; human GCSFR ICD |
| SEQ ID NO: 91 | DNA sequence human ortho IL-2 receptor β ECD; human GCSFR ICD and TM |
| SEQ ID NO: 92 | AA sequence human ortho IL-2 receptor β ECD; human GCSFR ICD and TM |

The extracellular domain sequences are single underlined, and may include the signal sequence, which for human and mouse IL-2RB is normally cleaved at residue 26 in the mature form. The transmembrane and intracellular domain sequences are double underlined. The transmembrane domains are wavy underlined. Variant sequences are as noted below. Where reference is made to the ICD sequence, unless specifically indicated, the sequence encompasses all residues after the ECD, as indicated below.

As known in the art, in referring to phosphosite sites, the pY is set to zero (0), and the residues before are referred to (−1), (−2) etc; and residues after are referred to (+1), (+2) etc. Reference may be made herein to variants in which the −2, −1, +1 and +2 sites are varied. In some specific examples, the Y(+2) site is mutagenized to generate a library with all amino acids.

SEQ ID NO:1-50 provide DNA and protein sequences for mouse chimeric constructs. SEQ ID NO:1, 3, 5, 7, 9, 11, 13, 15, 17,19, 21, 23, 25, 27, 29, 31, 33, 35, 37, 39, 41, 43, 45, 47 and 49 provide exemplary coding sequences. SEQ ID NO:2, 4, 6, 8, 10, 12, 14, 16, 18, 20, 22, 24, 26, 28, 30, 32, 34, 36, 38, 40, 42, 44, 46, 48 and 50 provide protein sequences, each of which comprises an ECD from residues 1-235; and TM/ICD sequences from residues 236 to the carboxy terminus. Specific amino acid modifications include:

SEQ ID NO:18 comprises an amino acid substitution Y437F in the mIL-22R STAT3 binding site.

SEQ ID NO:20 comprises the wild-type mIL-9R STAT binding site (YLPQ) at residues 384-387.

SEQ ID NO:22 comprises a mutated mIL-9R STAT binding site FLPQ at residues 384-387.

SEQ ID NO:24 comprises a mutated mIL-9R STAT binding site YRPQ at residues 384-387.

SEQ ID NO:26 comprises a mutated mIL-9R STAT binding site YLPL at residues 384-387.

SEQ ID NO:28 comprises a mutated mIL-9R STAT binding site YLKQ at residues 384-387.

SEQ ID NO:30 comprises a mutated mIL-9R STAT binding site YLXQ at residues 384-387, where X may be any amino acid other than the wild-type P.

SEQ ID NO:30 comprises a carboxy terminal addition of an IL-2RB STAT5 phosphosite sequence, provided herein as SEQ ID NO:83. SEQ ID NO:34 comprises two STAT5 phosphosite sequences; and SEQ ID NO:36 comprises three STAT5 phosphosite sequences.

SEQ ID NO:38 comprises a mutated mIL-9R STAT binding site YLXQ at residues 384-387, where X may be any amino acid other than the wild-type P, in combination with a carboxy terminal addition of from 1-3 IL-2RB STAT5 phosphosite sequence(s), provided herein as SEQ ID NO: 83.

SEQ ID NO:42 comprises a mIL-21R ICD, in combination with a carboxy terminal addition of from 1-3 IL-2RB STAT5 phosphosite sequence(s), provided herein as SEQ ID NO:83.

SEQ ID NO:44 comprises a modification of the mIL-21R ICD in the STAT3 binding sequence, where wild-type residues 516-518 (RSY) are substituted with SAY.

SEQ ID NO:46 comprises a modification of the mIL-21R ICD in the STAT3 binding sequence, where wild-type residues 516-518 (RSY) are substituted with DAY.

SEQ ID NO:48 comprises a modification of the mIL-21R ICD in the STAT3 binding sequence, where wild-type residues 516-521 (RSYLRQ) are substituted with DAYLXQ, wherein X is any amino acid.

SEQ ID NO:51-82 provide DNA and protein sequences for human chimeric constructs.

SEQ ID NO:51, 53, 55, 57, 59, 61, 63, 65, 67, 69, 71, 73, 75, 77, 79, 81 provide exemplary coding sequences. SEQ ID NO:52, 54, 56, 58, 60, 62, 64, 66, 68, 70, 72, 74, 76, 78 80 and 82 provide protein sequences, each of which comprises an ECD from residues 1-234; and TM/ICD sequences from residues 235 to the carboxy terminus. Specific amino acid modifications include:

SEQ ID NO: 62 comprises the wild-type hIL-9R STAT binding site (YLPQ) at residues 384-387.

SEQ ID NO:64 comprises a mutated hIL-9R STAT binding site FLPQ at residues 384-387.

SEQ ID NO:66 comprises a mutated hIL-9R STAT binding site YRPQ at residues 384-387.

SEQ ID NO:68 comprises a mutated hIL-9R STAT binding site YLPL at residues 384-387.

SEQ ID NO:70 comprises a mutated hIL-9R STAT binding site YLKQ at residues 384-387.

SEQ ID NO:72 comprises a mutated hIL-9R STAT binding site YLXQ at residues 384-387, where X may be any amino acid other than the wild-type P.

SEQ ID NO:74 comprises a mutated hIL-9R STAT binding site YLXQ at residues 384-387, where X may be any amino acid other than the wild-type P, in combination with a carboxy terminal addition of from 1-3 IL-2RB STAT5 phosphosite sequence(s), provided herein as SEQ ID NO: 84.

SEQ ID NO: 78 comprises a hIL-21R ICD, in combination with a carboxy terminal addition of from 1-3 IL-2RB STAT5 phosphosite sequence(s), provided herein as SEQ ID NO:84.

SEQ ID NO:80 comprises a modification of the hIL-21R ICD in the STAT3 binding sequence, where wild-type residues 524-526 (RSY) are substituted with SAY.

SEQ ID NO:82 comprises a modification of the hIL-21R ICD in the STAT3 binding sequence, where wild-type residues 524-529 (RSYLRQ) are substituted with DAYLXQ, wherein X is any amino acid.

In the sequences disclosed herein, an additional STAT binding site can be added to the ICD sequence, usually of the form SEQ ID NO:93 (LNTDAYLSLQE)ₓ, where X is from 1, 2, 3, or more, e.g. as shown in SEQ ID NO:83 and 84. The phosphosite may be placed close to the terminus, for example at the C-terminus or flanked by the normal C-terminal sequence.

| | |
|---|---|
| | 1. mouse orthoIL2Rb-IFNAR1 (molFNAR1) |
| SEQ ID | ATGGCTACCATAGCTCTTCCCTGGAGCCTGTCCCTCTACGTCTTCCTCCTGCTCCTGGCTACACCT |
| NO: 1 | TGGGCATCTGCAGCAGTGAAAAACTGTTCCCATCTTGAATGCTTCTACAACTCAAGAGCCAATG |
| | TCTCTTGCATGTGGAGCCATGAAGAGGCTCTGAATGTCACAACCTGCCACGTCCATGCCAAGTC |
| | GAACCTGCGACACTGGAACAAAACCTGTGAGCTAACTCTTGTGAGGCAGGCATCCTGGGCCTG |
| | CAACCTGATCCTCGGGTCGTTCCCAGAGTCCCAGTCACTGACCTCCGTGGACCTCCTTGACATAA |
| | ATGTGGTGTGCTGGGAAGAGAAGGGTTGGCGTAGGGTAAAGACCTGCGACTTCCATCCCTTTG |
| | ACAACCTTCGCCTGGTGGCCCCTCATTCCCTCCAAGTTCTGCACATTGATACCCAGAGATGTAAC |
| | ATAAGCTGGAAGGTCTCCCAGGTCTCTGACTTCATTGAACCATACTTGGAATTTGAGGCCCGTA |
| | GACGTCTTCTGGGCCACAGCTGGGAGGATGCATCCGTATTAAGCCTCAAGCAGAGACAGCAGT |
| | GGCTCTTCTTGGAGATGCTGATCCCTAGTACCTCATATGAGGTCCAGGTGAGGGTCAAAGCTCA |
| | ACGAAACAATACCGGGACCTGGAGTCCCTGGAGCCAGCCCCTGACCTTTCGGACAAGGCCAGC |
| | 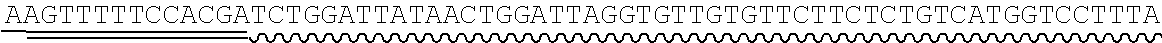 |
| | 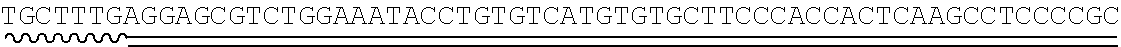 |
| | AGTATTGATGAGTTTTTCTCTGAGCCGCCTTCAAAAAACCTTGTACTTCTGACGGCTGAGGAGC |
| | ACACGGAAAGATGCTTCATCATTGAGAATACAGACACGGTCGCTGTAGAAGTAAAGCACGCGC |
| | CTGAGGAGGACCTCAGGAAGTACAGCTCACAGACCAGCCAGGACTCGGGCAACTATTCCAACG |
| | AAGAGGAGGAGAGTGTGGGCACCGAGAGCGGCCAAGCTGTGCTCTCCAAAGCTCCCTGCGGG |
| | GGGCCATGCAGCGTGCCTAGCCCTCCTGGGACCTTGGAAGACGGGACCTGCTTCCTGGGAAAT |
| | GAAAAATATCTTCAGAGCCCAGCCCTGAGGACAGAGCCAGCTCTTCTCTGCTGA |
| SEQ ID | MATIALPWSLSLYVFLLLLATPWASAAVKNCSHLECFYNSRANVSCMWSHEEALNVTTCHVHAKS |
| NO: 2 | NLRHWNKTCELTLVRQASWACNLILGSFPESQSLTSVDLLDINVVCWEEKGWRRVKTCDFHPFDN |
| | LRLVAPHSLQVLHIDTQRCNISWKVSQVSDFIEPYLEFEARRRLLGHSWEDASVLSLKQRQQWLFLE |
| | 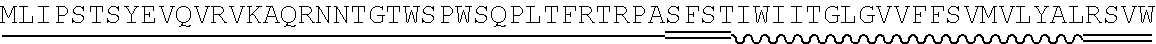 |
| | KYLCHVCFPPLKPPRSIDEFFSEPPSKNLVLLTAEEHTERCFIIENTDTVAVEVKHAPEEDLRKYSSQTS |
| | QDSGNYSNEEEESVGTESGQAVLSKAPCGGPCSVPSPPGTLEDGTCFLGNEKYLQSPALRTEPALLC* |
| | 2. mouse orthoIL2Rb-IFNAR2 (molFNAR2) |
| SEQ ID | ATGGCTACCATAGCTCTTCCCTGGAGCCTGTCCCTCTACGTCTTCCTCCTGCTCCTGGCTACACCT |
| NO: 3 | TGGGCATCTGCAGCAGTGAAAAACTGTTCCCATCTTGAATGCTTCTACAACTCAAGAGCCAATG |
| | TCTCTTGCATGTGGAGCCATGAAGAGGCTCTGAATGTCACAACCTGCCACGTCCATGCCAAGTC |
| | GAACCTGCGACACTGGAACAAAACCTGTGAGCTAACTCTTGTGAGGCAGGCATCCTGGGCCTG |
| | CAACCTGATCCTCGGGTCGTTCCCAGAGTCCCAGTCACTGACCTCCGTGGACCTCCTTGACATAA |
| | ATGTGGTGTGCTGGGAAGAGAAGGGTTGGCGTAGGGTAAAGACCTGCGACTTCCATCCCTTTG |
| | ACAACCTTCGCCTGGTGGCCCCTCATTCCCTCCAAGTTCTGCACATTGATACCCAGAGATGTAAC |
| | ATAAGCTGGAAGGTCTCCCAGGTCTCTGACTTCATTGAACCATACTTGGAATTTGAGGCCCGTA |
| | GACGTCTTCTGGGCCACAGCTGGGAGGATGCATCCGTATTAAGCCTCAAGCAGAGACAGCAGT |
| | GGCTCTTCTTGGAGATGCTGATCCCTAGTACCTCATATGAGGTCCAGGTGAGGGTCAAAGCTCA |
| | ACGAAACAATACCGGGACCTGGAGTCCCTGGAGCCAGCCCCTGACCTTTCGGACAAGGCCAGC |
| | 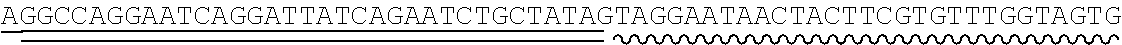 |
| | 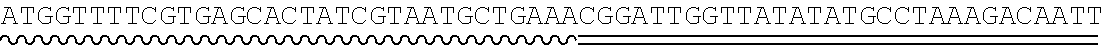 |
| | TGCCCAATGTCTTGAACTTCCGCCACTTTTTAACCTGGATAATCCCTGAACGGTCACCTTCAGAA |
| | GCCATCGATCGGCTGGAAATCATCCCCACAAACAAGAAGAAGAGACTGTGGAATTACGATTAT |
| | GAGGATGGCAGTGACAGTGACGAAGAGGTCCCCACAGCAAGTGTCACTGGCTACACCATGCAT |
| | GGACTGACGGGCAAGCCTCTGCAACAAACCTCTGACACCTCAGCCAGCCCCGAGGATCCCCTG |
| | CATGAAGAAGATTCAGGCGCTGAGGAATCTGATGAAGCTGGAGCAGGGGCTGGAGCTGAGCC |
| | AGAACTCCCCACAGAGGCGGGGGGGGGCCTTCAGAAGACCCCACTGGCCCCTATGAGAGAA |
| | GAAAGAGTGTGCTCGAGGACTCATTCCCCAGGGAGGACAACAGCTCCATGGATGAGCCTGGG |
| | GACAACATTATCTTCAACGTGAACTTAAACTCTGTGTTTCTGAGGGTTCTCCATGATGAAGATGC |
| | CTCAGAGACATTATCTCTCGAAGAAGACACCATCCTCCTAGACGAAGGTCCCCAGAGGACAGA |
| | GTCAGACCTTCGGATAGCTGGTGGGGACAGGACACAGCCGCCCCTCCCCAGCCTTCCTTCCCAG |
| | GATCTATGGACTGAAGATGGGTCATCTGAGAAATCAGACACCTCAGATTCCGATGCTGATGTG |
| | GGGGACGGCTACATCATGAGATGA |
| SEQ ID | MATIALPWSLSLYVFLLLLATPWASAAVKNCSHLECFYNSRANVSCMWSHEEALNVTTCHVHAKS |
| NO: 4 | NLRHWNKTCELTLVRQASWACNLILGSFPESQSLTSVDLLDINVVCWEEKGWRRVKTCDFHPFDN |
| | LRLVAPHSLQVLHIDTQRCNISWKVSQVSDFIEPYLEFEARRRLLGHSWEDASVLSLKQRQQWLFLE |
| | 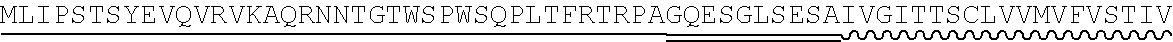 |
| | 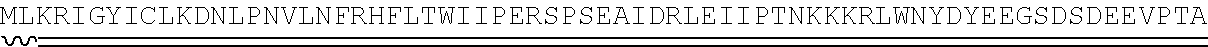 |
| | SVTGYTMHGLTGKPLQQTSDTSASPEDPLHEEDSGAEESDEAGAGAGAEPELPTEAGAGPSEDPTG |
| | PYERRKSVLEDSFPREDNSSMDEPGDNIIFNVNLNSVFLRVLHDEDASETLSLEEDTILLDEGPQRTES |
| | DLRIAGGDRTQPPLPSLPSQDLWTEDGSSEKSDTSDSDADVGDGYIMR* |
| | 3. mouse orthoIL2Rb-IFNGR1 (molFNGR1) |
| SEQ ID | ATGGCTACCATAGCTCTTCCCTGGAGCCTGTCCCTCTACGTCTTCCTCCTGCTCCTGGCTACACCT |
| NO: 5 | TGGGCATCTGCAGCAGTGAAAAACTGTTCCCATCTTGAATGCTTCTACAACTCAAGAGCCAATG |
| | TCTCTTGCATGTGGAGCCATGAAGAGGCTCTGAATGTCACAACCTGCCACGTCCATGCCAAGTC |
| | GAACCTGCGACACTGGAACAAAACCTGTGAGCTAACTCTTGTGAGGCAGGCATCCTGGGCCTG |
| | CAACCTGATCCTCGGGTCGTTCCCAGAGTCCCAGTCACTGACCTCCGTGGACCTCCTTGACATAA |
| | ATGTGGTGTGCTGGGAAGAGAAGGGTTGGCGTAGGGTAAAGACCTGCGACTTCCATCCCTTTG |
| | ACAACCTTCGCCTGGTGGCCCCTCATTCCCTCCAAGTTCTGCACATTGATACCCAGAGATGTAAC |
| | ATAAGCTGGAAGGTCTCCCAGGTCTCTGACTTCATTGAACCATACTTGGAATTTGAGGCCCGTA |
| | GACGTCTTCTGGGCCACAGCTGGGAGGATGCATCCGTATTAAGCCTCAAGCAGAGACAGCAGT |
| | GGCTCTTCTTGGAGATGCTGATCCCTAGTACCTCATATGAGGTCCAGGTGAGGGTCAAAGCTCA |
| | ACGAAACAATACCGGGACCTGGAGTCCCTGGAGCCAGCCCCTGACCTTTCGGACAAGGCCAGC |
| | 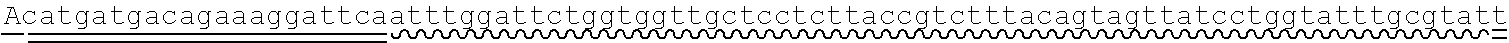 |
| | ggtatactaagaagaattcattcaagagaaaaagcataatgttacctaagtccttgctctctgtggtaaaaagtgccacgtta |
| | gagacaaaacctgaatcgaagtattcacttgtcacaccgcaccagccagctgtcctagagagtgagacggtgatctgtgaag |
| | agcccctgtccacagtgacagctccagacagccccgaagcagcagaacaggaagaactttcaaaagaaacaaaggctctg |
| | gaggctggaggaagcacgtctgccatgaccccagacagccctccaactccgacacaaagacgcagcttttccctgttaagta |
| | gtaaccagtcaggcccttgtagcctcaccgcctatcactcccgaaacggctctgacagtggcctcgtgggatcgggcagctcc |
| | atatcggacttggaatctctcccaaacaacaactcagaaacaaagatggcagagcacgaccctccacccgtgagaaaggcc |
| | cccatggcctccggttatgacaaaccgcacatgttggtggacgtgcttgtggatgttggggggaaggagtctctcatggggta |
| | tagactcacaggagaggcccaggagctgtcctaa |
| SEQ ID | MATIALPWSLSLYVFLLLLATPWASAAVKNCSHLECFYNSRANVSCMWSHEEALNVTTCHVHAKS |
| NO: 6 | NLRHWNKTCELTLVRQASWACNLILGSFPESQSLTSVDLLDINVVCWEEKGWRRVKTCDFHPFDN |
| | LRLVAPHSLQVLHIDTQRCNISWKVSQVSDFIEPYLEFEARRRLLGHSWEDASVLSLKQRQQWLFLE |
| | 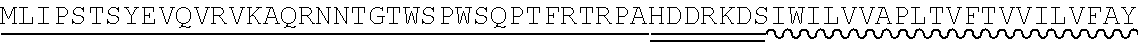 |
| | WYTKKNSFKRKSIMLPKSLLSVVKSATLETKPESKYSLVTPHQPAVLESETVICEEPLSTVTAPDSPEAA |
| | EQEELSKETKALEAGGSTSAMTPDSPPTPTQRRSFSLLSSNQSGPCSLTAYHSRNGSDSGLVGSGSSI |
| | SDLESLPNNNSETKMAEHDPPPVRKAPMASGYDKPHMLVDVLVDVGGKESLMGYRLTGEAQELS* |
| | 4. mouse orthoIL2Rb-IFNGR2 (molFNGR2) |
| SEQ ID | ATGGCTACCATAGCTCTTCCCTGGAGCCTGTCCCTCTACGTCTTCCTCCTGCTCCTGGCTACACCT |
| NO: 7 | TGGGCATCTGCAGCAGTGAAAAACTGTTCCCATCTTGAATGCTTCTACAACTCAAGAGCCAATG |
| | TCTCTTGCATGTGGAGCCATGAAGAGGCTCTGAATGTCACAACCTGCCACGTCCATGCCAAGTC |
| | GAACCTGCGACACTGGAACAAAACCTGTGAGCTAACTCTTGTGAGGCAGGCATCCTGGGCCTG |
| | CAACCTGATCCTCGGGTCGTTCCCAGAGTCCCAGTCACTGACCTCCGTGGACCTCCTTGACATAA |
| | ATGTGGTGTGCTGGGAAGAGAAGGGTTGGCGTAGGGTAAAGACCTGCGACTTCCATCCCTTTG |
| | ACAACCTTCGCCTGGTGGCCCCTCATTCCCTCCAAGTTCTGCACATTGATACCCAGAGATGTAAC |
| | ATAAGCTGGAAGGTCTCCCAGGTCTCTGACTTCATTGAACCATACTTGGAATTTGAGGCCCGTA |
| | GACGTCTTCTGGGCCACAGCTGGGAGGATGCATCCGTATTAAGCCTCAAGCAGAGACAGCAGT |
| | GGCTCTTCTTGGAGATGCTGATCCCTAGTACCTCATATGAGGTCCAGGTGAGGGTCAAAGCTCA |
| | ACGAAACAATACCGGGACCTGGAGTCCCTGGAGCCAGCCCCTGACCTTTCGGACAAGGCCAGC |
| | 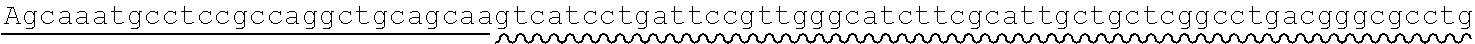 |
| | 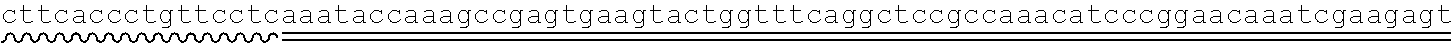 |
| | atctaaaggacccagaccaattcatcttagaggtcttggacaaggacggttcaccgaaggaggactcctgggactccgtgtc |
| | aattatttcttctccagaaaaggagcgagatgatgtgctccaaacaccgtga |
| SEQ ID | MATIALPWSLSLYVFLLLLATPWASAAVKNCSHLECFYNSRANVSCMWSHEEALNVTTCHVHAKS |
| NO: 8 | NLRHWNKTCELTLVRQASWACNLILGSFPESQSLTSVDLLDINVVCWEEKGWRRVKTCDFHPFDN |
| | LRLVAPHSLQVLHIDTQRCNISWKVSQVSDFIEPYLEFEARRRLLGHSWEDASVLSLKQRQQWLFLE |
| | 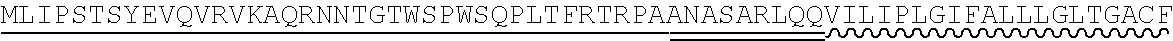 |
| | 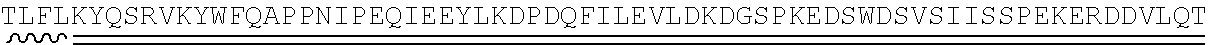 |
| | 5. mouse orthoIL2Rb-IFNLR1 (molFNLR1) |
| SEQ ID | ATGGCTACCATAGCTCTTCCCTGGAGCCTGTCCCTCTACGTCTTCCTCCTGCTCCTGGCTACACCT |
| NO: 9 | TGGGCATCTGCAGCAGTGAAAAACTGTTCCCATCTTGAATGCTTCTACAACTCAAGAGCCAATG |
| | TCTCTTGCATGTGGAGCCATGAAGAGGCTCTGAATGTCACAACCTGCCACGTCCATGCCAAGTC |
| | GAACCTGCGACACTGGAACAAAACCTGTGAGCTAACTCTTGTGAGGCAGGCATCCTGGGCCTG |
| | CAACCTGATCCTCGGGTCGTTCCCAGAGTCCCAGTCACTGACCTCCGTGGACCTCCTTGACATAA |
| | ATGTGGTGTGCTGGGAAGAGAAGGGTTGGCGTAGGGTAAAGACCTGCGACTTCCATCCCTTTG |
| | ACAACCTTCGCCTGGTGGCCCCTCATTCCCTCCAAGTTCTGCACATTGATACCCAGAGATGTAAC |
| | ATAAGCTGGAAGGTCTCCCAGGTCTCTGACTTCATTGAACCATACTTGGAATTTGAGGCCCGTA |
| | GACGTCTTCTGGGCCACAGCTGGGAGGATGCATCCGTATTAAGCCTCAAGCAGAGACAGCAGT |
| | GGCTCTTCTTGGAGATGCTGATCCCTAGTACCTCATATGAGGTCCAGGTGAGGGTCAAAGCTCA |
| | ACGAAACAATACCGGGACCTGGAGTCCCTGGAGCCAGCCCCTGACCTTTCGGACAAGGCCAGC |
| | 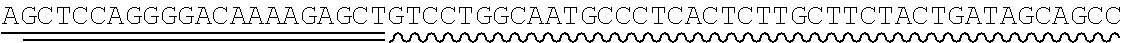 |
| | 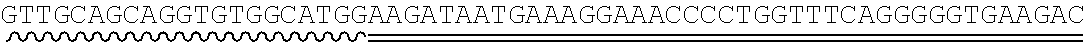 |
| | GCCCCGGGCACTGGACTTTTCTGAATACAGATACCCAGTGGCAACCTTTCAGCCCAGTGGACCT |
| | GAGTTCTCTGATGACTTGATCCTTTGTCCCCAGAAGGAACTGACCATAAGGAACAGGCCAGCCC |
| | CTCAGGTCAGAAACCCAGCCACGCTACAGGCAGGACCAGAAAGAGACAGTACTGAGGATGAA |
| | GACGAGGACACAGACTACGATGACGACGGTGACAGCGTCCAGCCCTACCTGGAACGGCCCCTC |
| | TTCATCAGCGAGAAGCCCCGGGTTATGGAACACTCGGAGACAGACGAGTCTGGGGTGGATTCA |
| | GGGGGGCCTTGGACATCCCCAGTTGGGAGTGACGGCTCCTCTGCATGGGACTCTTCAGACAGG |
| | AGCTGGTCCAGCACAGGGGACTCCTCATATAAGGATGAAGTTGGGTCTTCCAGCTGTTTGGAC |
| | CGAAAGGAGCCGGACCAGGCGCCCTGTGGGGATTGGCTCCAGGAGGCTCTCCCATGCCTGGA |
| | ATTTTCTGAGGACTTGGGCACCGTGGAAGAGCCTCTGAAGGATGGCCTCTCCGGGTGGAGGAT |
| | TTCTGGTTCCTTATCCTCAAAGAGAGATCTGGCTCCTGTGGAGCCCCCAGTTTCTCTTCAGACAC |
| | TGACTTTCTGCTGGGTCAACAATCCTGAGGGGGAGGAGGAACAGGAGGACGAGGAGGAAGA |
| | GGAGGAGGAGGAGGAGGAGGAAGACTGGGAATCAGAACCTAAGGGCAGCAATGCCGGCTGT |
| | TGGGGCACTTCAAGCGTGCAGAGGACGGAGGTCAGGGGCCGGATGCTGGGAGATTACTTGGT |
| | CAGGTGA |
| SEQ ID | MATIALPWSLSLYVFLLLLATPWASAAVKNCSHLECFYNSRANVSCMWSHEEALNVTTCHVHAKS |
| NO: 10 | NLRHWNKTCELTLVRQASWACNLILGSFPESQSLTSVDLLDINVVCWEEKGWRRVKTCDFHPFDN |
| | LRLVAPHSLQVLHIDTQRCNISWKVSQVSDFIEPYLEFEARRRLLGHSWEDASVLSLKQRQQWLFLE |
| | MLIPSTSYEVQVRVKAQRNNTGTWSPWSQPLTFRTRPAAPGDKRAVLAMPSLLLLLIAAVAAGVA |
| | WKIMKGNPWFQGVKTPRALDFSEYRYPVATFQPSGPEFSDDLILCPQKELTIRNRPAPQVRNPATL |
| | QAGPERDSTEDEDEDTDYDDDGDSVQPYLERPLFISEKPRVMEHSETDESGVDSGGPWTSPVGSD |
| | GSSAWDSSDRSWSSTGDSSYKDEVGSSSCLDRKEPDQAPCGDWLQEALPCLEFSEDLGTVEEPLKD |
| | GLSGWRISGSLSSKRDLAPVEPPVSLQTLTFCWVNNPEGEEEQEDEEEEEEEEEEEDWESEPKGSNA |
| | GCWGTSSVQRTEVRGRMLGDYLVR* |
| | 6. mouse orthoIL2Rb-IL10Ra (molL10Ra) |
| SEQ ID | aTGGCTACCATAGCTCTTCCCTGGAGCCTGTCCCTCTACGTCTTCCTCCTGCTCCTGGCTACACCT |
| NO: 11 | TGGGCATCTGCAGCAGTGAAAAACTGTTCCCATCTTGAATGCTTCTACAACTCAAGAGCCAATG |
| | TCTCTTGCATGTGGAGCCATGAAGAGGCTCTGAATGTCACAACCTGCCACGTCCATGCCAAGTC |
| | GAACCTGCGACACTGGAACAAAACCTGTGAGCTAACTCTTGTGAGGCAGGCATCCTGGGCCTG |
| | CAACCTGATCCTCGGGTCGTTCCCAGAGTCCCAGTCACTGACCTCCGTGGACCTCCTTGACATAA |
| | ATGTGGTGTGCTGGGAAGAGAAGGGTTGGCGTAGGGTAAAGACCTGCGACTTCCATCCCTTTG |
| | ACAACCTTCGCCTGGTGGCCCCTCATTCCCTCCAAGTTCTGCACATTGATACCCAGAGATGTAAC |
| | ATAAGCTGGAAGGTCTCCCAGGTCTCTGACTTCATTGAACCATACTTGGAATTTGAGGCCCGTA |
| | GACGTCTTCTGGGCCACAGCTGGGAGGATGCATCCGTATTAAGCCTCAAGCAGAGACAGCAGT |
| | GGCTCTTCTTGGAGATGCTGATCCCTAGTACCTCATATGAGGTCCAGGTGAGGGTCAAAGCTCA |
| | ACGAAACAATACCGGGACCTGGAGTCCCTGGAGCCAGCCCCTGACCTTTCGGACAAGGCCAGC |
| | 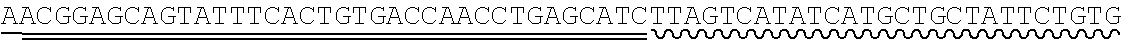 |
| | 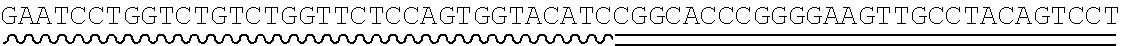 |
| | GGTCTTCAAGAAGCCTCACGACTTCTTCCCAGCCAACCCTCTCTGCCCAGAAACTCCCGATGCCA |
| | TTCACATCGTGGACCTGGAGGTTTTCCCAAAGGTGTCACTAGAGCTGAGAGACTCAGTCCTGCA |
| | TGGCAGCACCGACAGTGGCTTTGGCAGTGGTAAACCATCACTTCAGACTGAAGAGTCCCAATTC |
| | CTCCTCCCTGGCTCCCACCCCCAGATACAGGGGACTCTGGGAAAAGAAGAGTCTCCAGGGCTA |
| | CAGGCCACCTGTGGGGACAACACGGACAGTGGGATCTGCCTGCAGGAGCCCGGCTTACACTCC |
| | AGCATGGGGCCCGCCTGGAAGCAGCAGCTTGGATATACCCATCAGGACCAGGATGACAGTGA |
| | CGTTAACCTAGTCCAGAACTCTCCAGGGCAGCCTAAGTACACACAGGATGCATCTGCCTTGGGC |
| | CATGTCTGTCTCCTAGAACCTAAAGCCCCTGAGGAGAAAGACCAAGTCATGGTGACATTCCAG |
| | GGCTACCAGAAACAGACCAGATGGAAGGCAGAGGCAGCAGGCCCAGCAGAATGCTTGGACGA |
| | AGAGATTCCCTTGACAGATGCCTTTGATCCTGAACTTGGGGTACACCTGCAGGATGATTTGGCT |
| | TGGCCTCCACCAGCTCTGGCCGCAGGTTATTTGAAACAGGAGTCTCAAGGGATGGCTTCTGCTC |
| | CACCAGGGACACCAAGTAGACAGTGGAATCAACTGACCGAAGAGTGGTCACTCCTGGGTGTG |
| | GTTAGCTGTGAAGATCTAAGCATAGAAAGTTGGAGGTTTGCCCATAAACTTGACCCTCTGGACT |
| | GTGGGGCAGCCCCTGGTGGCCTCCTGGATAGCCTTGGCTCTAACCTGGTCACCCTGCCGTTGAT |
| | CTCCAGCCTGCAGGTAGAAGAATGA |
| SEQ ID | MATIALPWSLSLYVFLLLLATPWASAAVKNCSHLECFYNSRANVSCMWSHEEALNVTTCHVHAKS |
| NO: 12 | NLRHWNKTCELTLVRQASWACNLILGSFPESQSLTSVDLLDINVVCWEEKGWRRVKTCDFHPFDN |
| | LRLVAPHSLQVLHIDTQRCNISWKVSQVSDFIEPYLEFEARRRLLGHSWEDASVLSLKQRQQWLFLE |
| | 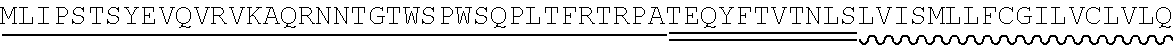 |
| | 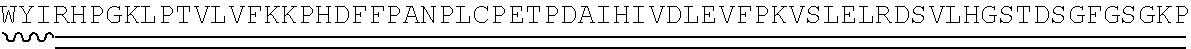 |
| | SLQTEESQFLLPGSHPQIQGTLGKEESPGLQATCGDNTDSGICLQEPGLHSSMGPAWKQQLGYTH |
| | QDQDDSDVNLVQNSPGQPKYTQDASALGHVCLLEPKAPEEKDQVMVTFQGYQKQTRWKAEAA |
| | GPAECLDEEIPLTDAFDPELGVHLQDDLAWPPPALAAGYLKQESQGMASAPPGTPSRQWNQLTEE |
| | WSLLGVVSCEDLSIESWRFAHKLDPLDCGAAPGGLLDSLGSNLVTLPLISSLQVEE* |
| | 7. mouse orthoIL2Rb-IL20R (moIL20R) |
| SEQ ID | aTGGCTACCATAGCTCTTCCCTGGAGCCTGTCCCTCTACGTCTTCCTCCTGCTCCTGGCTACACCT |
| NO: 13 | TGGGCATCTGCAGCAGTGAAAAACTGTTCCCATCTTGAATGCTTCTACAACTCAAGAGCCAATG |
| | TCTCTTGCATGTGGAGCCATGAAGAGGCTCTGAATGTCACAACCTGCCACGTCCATGCCAAGTC |
| | GAACCTGCGACACTGGAACAAAACCTGTGAGCTAACTCTTGTGAGGCAGGCATCCTGGGCCTG |
| | CAACCTGATCCTCGGGTCGTTCCCAGAGTCCCAGTCACTGACCTCCGTGGACCTCCTTGACATAA |
| | ATGTGGTGTGCTGGGAAGAGAAGGGTTGGCGTAGGGTAAAGACCTGCGACTTCCATCCCTTTG |
| | ACAACCTTCGCCTGGTGGCCCCTCATTCCCTCCAAGTTCTGCACATTGATACCCAGAGATGTAAC |
| | ATAAGCTGGAAGGTCTCCCAGGTCTCTGACTTCATTGAACCATACTTGGAATTTGAGGCCCGTA |
| | GACGTCTTCTGGGCCACAGCTGGGAGGATGCATCCGTATTAAGCCTCAAGCAGAGACAGCAGT |
| | GGCTCTTCTTGGAGATGCTGATCCCTAGTACCTCATATGAGGTCCAGGTGAGGGTCAAAGCTCA |
| | ACGAAACAATACCGGGACCTGGAGTCCCTGGAGCCAGCCCCTGACCTTTCGGACAAGGCCAGC |
| | 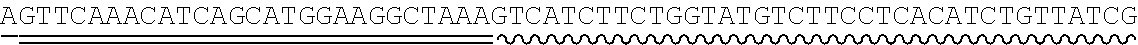 |
| | 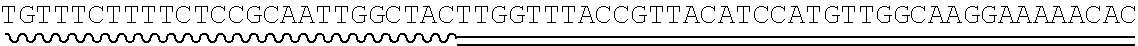 |
| | CCAGCAAATTTGGTACTGATTTATAGAAATGAAATTGGCACAAGAGTCTTTGAACCTACTGAAA |
| | CAATCACACTTAATTTTATCACCTTCAGTATGTTGGATGATACTAAAATTTCTCCAAAGGATATG |
| | AATTTACTGGACAAAAGCAGTGATGACATCAGTGTTAATGACCCTGAGCACAATGAGGCCTGG |
| | GAGCCGCACTGGGAGGAGGTGGAGGGGCAACATTTAGGATGCTCTTCGCATTTGATGGACGC |
| | TGTCTGTGGTGCTGAGCAAAGAGACGGAGACACCTCCCTAACCCAGCATGGGTGGCTTAACAG |
| | CACCATCCCCACAGGAGAGACAGACACTGAGCCTCAATACAAAGTCCTAAGTGACTTCTACGG |
| | GGAGGGTGAAATCCAACTGTCCTGTGAGCCGGAAGAGGCGGCCAGAACAGAGAAAATATCTG |
| | AGCCACTGGTGACTTCAGCAAACTTGGACCCACAGCTTGAAGACCTACATCACCTGGGTCAGG |
| | AGCATACTGTCTCCGAGGATGGGCCAGAGGAAGAGACATCTATAACAGTAGTGGATTGGGAC |
| | CCTCAAACTGGCAGGCTGTGTATCCCTTCCTTACCTATCTTTGGCCGTGATCCTGAGAACTATGG |
| | TCATTATGAGAGAGACCAGCTCTTAGAGGGTGGCCTTTTGTCTAGACTCTATGAGAACCAGGCA |
| | CCTGACAAGCCAGAGAAAGAAAATGAAAACTGTCTCACACGGTTTATGGAGGAATGGGGGTTA |
| | CATGTACAAATGGAAAGCTAG |
| SEQ ID | MATIALPWSLSLYVFLLLLATPWASAAVKNCSHLECFYNSRANVSCMWSHEEALNVTTCHVHAKS |
| NO: 14 | NLRHWNKTCELTLVRQASWACNLILGSFPESQSLTSVDLLDINVVCWEEKGWRRVKTCDFHPFDN |
| | LRLVAPHSLQVLHIDTQRCNISWKVSQVSDFIEPYLEFEARRRLLGHSWEDASVLSLKQRQQWLFLE |
| | 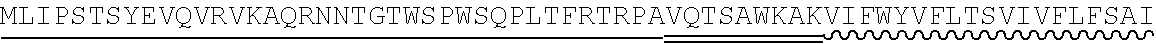 |
| | 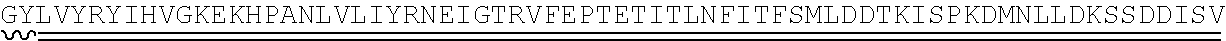 |
| | NDPEHNEAWEPHWEEVEGQHLGCSSHLMDAVCGAEQRDGDTSLTQHGWLNSTIPTGETDTEPQ |
| | YKVLSDFYGEGEIQLSCEPEEAARTEKISEPLVTSANLDPQLEDLHHLGQEHTVSEDGPEEETSITVVD |
| | WDPQTGRLCIPSLPIFGRDPENYGHYERDQLLEGGLLSRLYENQAPDKPEKENENCLTRFMEEWGL |
| | HVQMES* |
| | 8. mouse orthoIL2Rb-IL22R (moIL22R) |
| SEQ ID | aTGGCTACCATAGCTCTTCCCTGGAGCCTGTCCCTCTACGTCTTCCTCCTGCTCCTGGCTACACCT |
| NO: 15 | TGGGCATCTGCAGCAGTGAAAAACTGTTCCCATCTTGAATGCTTCTACAACTCAAGAGCCAATG |
| | TCTCTTGCATGTGGAGCCATGAAGAGGCTCTGAATGTCACAACCTGCCACGTCCATGCCAAGTC |
| | GAACCTGCGACACTGGAACAAAACCTGTGAGCTAACTCTTGTGAGGCAGGCATCCTGGGCCTG |
| | CAACCTGATCCTCGGGTCGTTCCCAGAGTCCCAGTCACTGACCTCCGTGGACCTCCTTGACATAA |
| | ATGTGGTGTGCTGGGAAGAGAAGGGTTGGCGTAGGGTAAAGACCTGCGACTTCCATCCCTTTG |
| | ACAACCTTCGCCTGGTGGCCCCTCATTCCCTCCAAGTTCTGCACATTGATACCCAGAGATGTAAC |
| | ATAAGCTGGAAGGTCTCCCAGGTCTCTGACTTCATTGAACCATACTTGGAATTTGAGGCCCGTA |
| | GACGTCTTCTGGGCCACAGCTGGGAGGATGCATCCGTATTAAGCCTCAAGCAGAGACAGCAGT |
| | GGCTCTTCTTGGAGATGCTGATCCCTAGTACCTCATATGAGGTCCAGGTGAGGGTCAAAGCTCA |
| | ACGAAACAATACCGGGACCTGGAGTCCCTGGAGCCAGCCCCTGACCTTTCGGACAAGGCCAGC |
| | 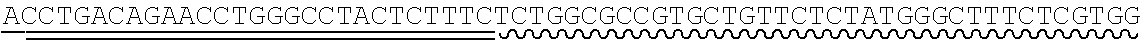 |
| | 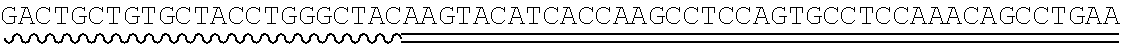 |
| | CGTGCAGAGAGTGCTGACCTTCCAGCCTCTGAGATTCATCCAAGAGCATGTGCTGATCCCCGTG |
| | CTGGACCTGTCTGGACCATCTTCTCTGCCCCAGCCTATCCAGTACAGCCAGGTGGTGGTTTCTG |
| | GCCCCAGAGAACCTCCTGGCGCAGTTTGGAGACAGAGCCTGAGCGACCTGACATACGTGGGCC |
| | AGAGCGACGTGTCAATCCTGCAGCCTACAAACGTGCCCGCTCAGCAGACACTGAGCCCTCCATC |
| | TTACGCCCCTAAGGCTGTGCCAGAAGTGCAGCCTCCTAGCTACGCTCCTCAGGTGGCCTCTGAT |
| | GCCAAGGCTCTGTTCTACAGCCCTCAGCAGGGCATGAAGACCAGACCTGCCACATACGACCCTC |
| | AGGACATCCTGGACTCTTGCCCTGCCAGCTACGCTGTGTGCGTGGAAGATAGCGGCAAGGACA |
| | GCACACCTGGCATCCTGAGCACCCCTAAGTACCTCAAGACCAAGGGCCAGCTGCAAGAGGACA |
| | CACTCGTGCGGTCTTGTCTGCCTGGCGATCTGAGCCTGCAGAAAGTGACATCTCTCGGCGAGG |
| | GCGAGACACAGAGGCCTAAGTCTCTGCCTTCTCCTCTGGGCTTCTGCACCGACAGAGGACCTGA |
| | TCTGCACACCCTGAGAAGCGAGGAACCCGAGACACCCAGATACCTGAAGGGCGCACTGTCCCT |
| | GCTGTCCAGCGTGCAGATTGAGGGACACCCTGTGTCTCTGCCACTGCATGTGCACAGCGTGTCC |
| | TGCTCTCCATCTGACGAGGGACCTTCTCCATGGGGCCTGCTGGATTCTCTCGTGTGCCCTAAGG |
| | ATGAGGGCCCTGCCGTGGAAACAGAGGCCATGTGTCCATCTGCTGCCGCCTCTGAGCTGGAAC |
| | AGTCTACCGAACTGGACAGCCTGTTCAAAGGCCTGGCTCTGACAGTGCAGTGGGAGAGCTGA |
| SEQ ID | MATIALPWSLSLYVFLLLLATPWASAAVKNCSHLECFYNSRANVSCMWSHEEALNVTTCHVHAKS |
| NO: 16 | NLRHWNKTCELTLVRQASWACNLILGSFPESQSLTSVDLLDINVVCWEEKGWRRVKTCDFHPFDN |
| | LRLVAPHSLQVLHIDTQRCNISWKVSQVSDFIEPYLEFEARRRLLGHSWEDASVLSLKQRQQWLFLE |
| | 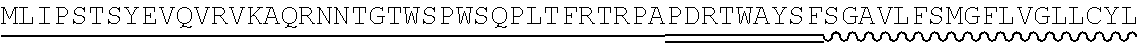 |
| | 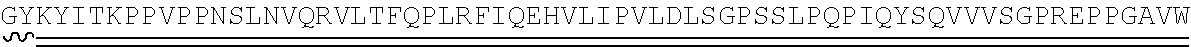 |
| | RQSLSDLTYVGQSDVSILQPTNVPAQQTLSPPSYAPKAVPEVQPPSYAPQVASDAKALFYSPQQGM |
| | KTRPATYDPQDILDSCPASYAVCVEDSGKDSTPGILSTPKYLKTKGQLQEDTLVRSCLPGDLSLQKVTS |
| | LGEGETQRPKSLPSPLGFCTDRGPDLHTLRSEEPETPRYLKGALSLLSSVQIEGHPVSLPLHVHSVSCS |
| | PSDEGPSPWGLLDSLVCPKDEGPAVETEAMCPSAAASELEQSTELDSLFKGLALTVQWES* |
| | 9. mouse orthoIL2Rb-IL22R (Y437F) (molL22R-Y437F) |
| SEQ ID | aTGGCTACCATAGCTCTTCCCTGGAGCCTGTCCCTCTACGTCTTCCTCCTGCTCCTGGCTACACCT |
| NO: 17 | TGGGCATCTGCAGCAGTGAAAAACTGTTCCCATCTTGAATGCTTCTACAACTCAAGAGCCAATG |
| | TCTCTTGCATGTGGAGCCATGAAGAGGCTCTGAATGTCACAACCTGCCACGTCCATGCCAAGTC |
| | GAACCTGCGACACTGGAACAAAACCTGTGAGCTAACTCTTGTGAGGCAGGCATCCTGGGCCTG |
| | CAACCTGATCCTCGGGTCGTTCCCAGAGTCCCAGTCACTGACCTCCGTGGACCTCCTTGACATAA |
| | ATGTGGTGTGCTGGGAAGAGAAGGGTTGGCGTAGGGTAAAGACCTGCGACTTCCATCCCTTTG |
| | ACAACCTTCGCCTGGTGGCCCCTCATTCCCTCCAAGTTCTGCACATTGATACCCAGAGATGTAAC |
| | ATAAGCTGGAAGGTCTCCCAGGTCTCTGACTTCATTGAACCATACTTGGAATTTGAGGCCCGTA |
| | GACGTCTTCTGGGCCACAGCTGGGAGGATGCATCCGTATTAAGCCTCAAGCAGAGACAGCAGT |
| | GGCTCTTCTTGGAGATGCTGATCCCTAGTACCTCATATGAGGTCCAGGTGAGGGTCAAAGCTCA |
| | ACGAAACAATACCGGGACCTGGAGTCCCTGGAGCCAGCCCCTGACCTTTCGGACAAGGCCAGC |
| | 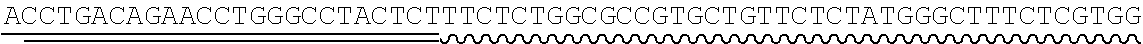 |
| | 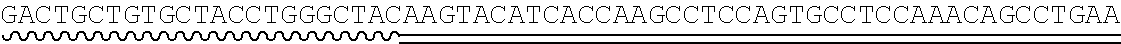 |
| | CGTGCAGAGAGTGCTGACCTTCCAGCCTCTGAGATTCATCCAAGAGCATGTGCTGATCCCCGTG |
| | CTGGACCTGTCTGGACCATCTTCTCTGCCCCAGCCTATCCAGTACAGCCAGGTGGTGGTTTCTG |
| | GCCCCAGAGAACCTCCTGGCGCAGTTTGGAGACAGAGCCTGAGCGACCTGACATACGTGGGCC |
| | AGAGCGACGTGTCAATCCTGCAGCCTACAAACGTGCCCGCTCAGCAGACACTGAGCCCTCCATC |
| | TTACGCCCCTAAGGCTGTGCCAGAAGTGCAGCCTCCTAGCTACGCTCCTCAGGTGGCCTCTGAT |
| | GCCAAGGCTCTGTTCTACAGCCCTCAGCAGGGCATGAAGACCAGACCTGCCACATACGACCCTC |
| | AGGACATCCTGGACTCTTGCCCTGCCAGCTACGCTGTGTGCGTGGAAGATAGCGGCAAGGACA |
| | GCACACCTGGCATCCTGAGCACCCCTAAGTTCCTCAAGACCAAGGGCCAGCTGCAAGAGGACA |
| | CACTCGTGCGGTCTTGTCTGCCTGGCGATCTGAGCCTGCAGAAAGTGACATCTCTCGGCGAGG |
| | GCGAGACACAGAGGCCTAAGTCTCTGCCTTCTCCTCTGGGCTTCTGCACCGACAGAGGACCTGA |
| | TCTGCACACCCTGAGAAGCGAGGAACCCGAGACACCCAGATACCTGAAGGGCGCACTGTCCCT |
| | GCTGTCCAGCGTGCAGATTGAGGGACACCCTGTGTCTCTGCCACTGCATGTGCACAGCGTGTCC |
| | TGCTCTCCATCTGACGAGGGACCTTCTCCATGGGGCCTGCTGGATTCTCTCGTGTGCCCTAAGG |
| | ATGAGGGCCCTGCCGTGGAAACAGAGGCCATGTGTCCATCTGCTGCCGCCTCTGAGCTGGAAC |
| | AGTCTACCGAACTGGACAGCCTGTTCAAAGGCCTGGCTCTGACAGTGCAGTGGGAGAGCTGA |
| SEQ ID | MATIALPWSLSLYVFLLLLATPWASAAVKNCSHLECFYNSRANVSCMWSHEEALNVTTCHVHAKS |
| NO: 18 | NLRHWNKTCELTLVRQASWACNLILGSFPESQSLTSVDLLDINVVCWEEKGWRRVKTCDFHPFDN |
| | LRLVAPHSLQVLHIDTQRCNISWKVSQVSDFIEPYLEFEARRRLLGHSWEDASVLSLKQRQQWLFLE |
| | 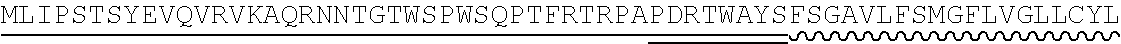 |
| | 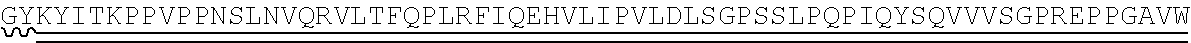 |
| | RQSLSDLTYVGQSDVSILQPTNVPAQQTLSPPSYAPKAVPEVQPPSYAPQVASDAKALFYSPQQGM |
| | KTRPATYDPQDILDSCPASYAVCVEDSGKDSTPGILSTPKFLKTKGQLQEDTLVRSCLPGDLSLQKVTS |
| | LGEGETQRPKSLPSPLGFCTDRGPDLHTLRSEEPETPRYLKGALSLLSSVQIEGHPVSLPLHVHSVSCS |
| | PSDEGPSPWGLLDSLVCPKDEGPAVETEAMCPSAAASELEQSTELDSLFKGLALTVQWES* |
| | 10. mouse orthoIL2Rb-IL9R WT (YLPQ) (moIL9R-YLPQ) |
| SEQ ID | ATGGCTACCATAGCTCTTCCCTGGAGCCTGTCCCTCTACGTCTTCCTCCTGCTCCTGGCTACACCT |
| NO: 19 | TGGGCATCTGCAGCAGTGAAAAACTGTTCCCATCTTGAATGCTTCTACAACTCAAGAGCCAATG |
| | TCTCTTGCATGTGGAGCCATGAAGAGGCTCTGAATGTCACAACCTGCCACGTCCATGCCAAGTC |
| | GAACCTGCGACACTGGAACAAAACCTGTGAGCTAACTCTTGTGAGGCAGGCATCCTGGGCCTG |
| | CAACCTGATCCTCGGGTCGTTCCCAGAGTCCCAGTCACTGACCTCCGTGGACCTCCTTGACATAA |
| | ATGTGGTGTGCTGGGAAGAGAAGGGTTGGCGTAGGGTAAAGACCTGCGACTTCCATCCCTTTG |
| | ACAACCTTCGCCTGGTGGCCCCTCATTCCCTCCAAGTTCTGCACATTGATACCCAGAGATGTAAC |
| | ATAAGCTGGAAGGTCTCCCAGGTCTCTGACTTCATTGAACCATACTTGGAATTTGAGGCCCGTA |
| | GACGTCTTCTGGGCCACAGCTGGGAGGATGCATCCGTATTAAGCCTCAAGCAGAGACAGCAGT |
| | GGCTCTTCTTGGAGATGCTGATCCCTAGTACCTCATATGAGGTCCAGGTGAGGGTCAAAGCTCA |
| | ACGAAACAATACCGGGACCTGGAGTCCCTGGAGCCAGCCCCTGACCTTTCGGACAAGGCCAGC |
| | 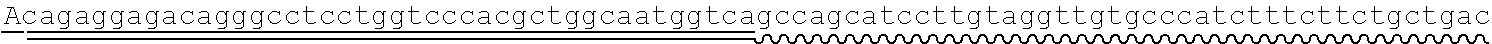 |
| | 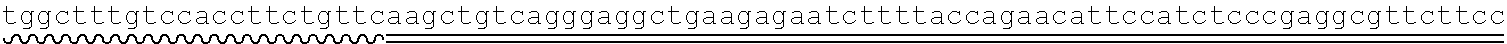 |
| | atcctctctacagtgtgtaccatggggacttccagagttggacaggggcccgcagagccggaccacaagcaagacagaatg |
| | gtgtcagtacttcatcagcaggctcagagtccagcatctgggaggccgtcgccacactcacctatagcccggcatgccctgtg |
| | cagtttgcctgcctgaagtgggaggccacagccccgggcttcccagggctcccaggctcagagcatgtgctgccggcagggt |
| | gtctggagttggaaggacagccatctgcctacctgccccaggaggactgggccccactgggctctgccaggccccctcctcca |
| | gactcagacagcggcagcagcgactattgcatgttggactgctgtgaggaatgccacctctcagccttcccaggacacaccg |
| | agagtcctgagctcacgctagctcagcctgtggcccttcctgtgtccagcagggcctga |
| SEQ ID | MATIALPWSLSLYVFLLLLATPWASAAVKNCSHLECFYNSRANVSCMWSHEEALNVTTCHVHAKS |
| NO: 20 | NLRHWNKTCELTLVRQASWACNLILGSFPESQSLTSVDLLDINVVCWEEKGWRRVKTCDFHPFDN |
| | LRLVAPHSLQVLHIDTQRCNISWKVSQVSDFIEPYLEFEARRRLLGHSWEDASVLSLKQRQQWLFLE |
| | 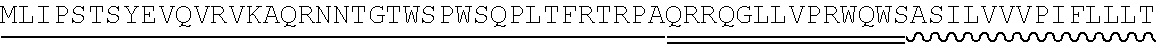 |
| | 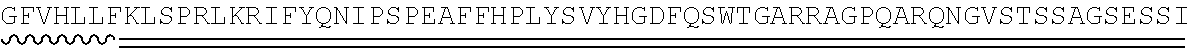 |
| | WEAVATLTYSPACPVQFACLKWEATAPGFPGLPGSEHVLPAGCLELEGQPSAYLPQEDWAPLGSA |
| | RPPPPDSDSGSSDYCMLDCCEECHLSAFPGHTESPELTLAQPVALPVSSRA* |
| | 11. mouse orthoIL2Rb-IL9R mut1 (FLPQ) (molL9R-FLPQ) |
| SEQ ID | ATGGCTACCATAGCTCTTCCCTGGAGCCTGTCCCTCTACGTCTTCCTCCTGCTCCTGGCTACACCT |
| NO: 21 | TGGGCATCTGCAGCAGTGAAAAACTGTTCCCATCTTGAATGCTTCTACAACTCAAGAGCCAATG |
| | TCTCTTGCATGTGGAGCCATGAAGAGGCTCTGAATGTCACAACCTGCCACGTCCATGCCAAGTC |
| | GAACCTGCGACACTGGAACAAAACCTGTGAGCTAACTCTTGTGAGGCAGGCATCCTGGGCCTG |
| | CAACCTGATCCTCGGGTCGTTCCCAGAGTCCCAGTCACTGACCTCCGTGGACCTCCTTGACATAA |
| | ATGTGGTGTGCTGGGAAGAGAAGGGTTGGCGTAGGGTAAAGACCTGCGACTTCCATCCCTTTG |
| | ACAACCTTCGCCTGGTGGCCCCTCATTCCCTCCAAGTTCTGCACATTGATACCCAGAGATGTAAC |
| | ATAAGCTGGAAGGTCTCCCAGGTCTCTGACTTCATTGAACCATACTTGGAATTTGAGGCCCGTA |
| | GACGTCTTCTGGGCCACAGCTGGGAGGATGCATCCGTATTAAGCCTCAAGCAGAGACAGCAGT |
| | GGCTCTTCTTGGAGATGCTGATCCCTAGTACCTCATATGAGGTCCAGGTGAGGGTCAAAGCTCA |
| | ACGAAACAATACCGGGACCTGGAGTCCCTGGAGCCAGCCCCTGACCTTTCGGACAAGGCCAGC |
| | 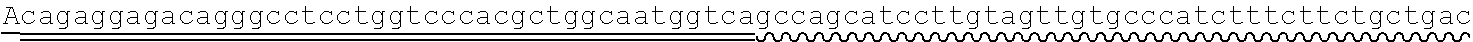 |
| | 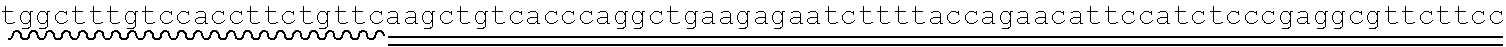 |
| | atcctctctacagtgtgtaccatggggacttccagagttggacaggggcccgcagagccggaccacaagcaagacagaatg |
| | gtgtcagtacttcatcagcaggctcagagtccagcatctgggaggccgtcgccacactcacctatagcccggcatgccctgtg |
| | cagtttgcctgcctgaagtgggaggccacagccccgggcttcccagggctcccaggctcagagcatgtgctgccggcagggt |
| | gtctggagttggaaggacagccatctgcctTcctgccccaggaggactgggccccactgggctctgccaggccccctcctcca |
| | gactcagacagcggcagcagcgactattgcatgttggactgctgtgaggaatgccacctctcagccttcccaggacacaccg |
| | agagtcctgagctcacgctagctcagcctgtggcccttcctgtgtccagcagggcctga |
| SEQ ID | MATIALPWSLSLYVFLLLLATPWASAAVKNCSHLECFYNSRANVSCMWSHEEALNVTTCHVHAKS |
| NO: 22 | NLRHWNKTCELTLVRQASWACNLILGSFPESQSLTSVDLLDINVVCWEEKGWRRVKTCDFHPFDN |
| | LRLVAPHSLQVLHIDTQRCNISWKVSQVSDFIEPYLEFEARRRLLGHSWEDASVLSLKQRQQWLFLE |
| | 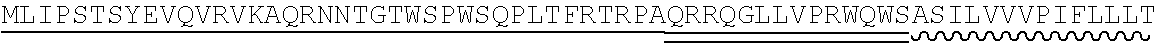 |
| | 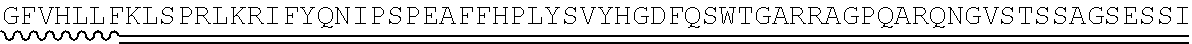 |
| | WEAVATLTYSPACPVQFACLKWEATAPGFPGLPGSEHVLPAGCLELEGQPSAFLPQEDWAPLGSA |
| | RPPPPDSDSGSSDYCMLDCCEECHLSAFPGHTESPELTLAQPVALPVSSRA* |
| | 12. mouse orthoIL2Rb-IL9R mut6 (YRPQ) (molL9R-YRPQ) |
| SEQ ID | ATGGCTACCATAGCTCTTCCCTGGAGCCTGTCCCTCTACGTCTTCCTCCTGCTCCTGGCTACACCT |
| NO: 23 | TGGGCATCTGCAGCAGTGAAAAACTGTTCCCATCTTGAATGCTTCTACAACTCAAGAGCCAATG |
| | TCTCTTGCATGTGGAGCCATGAAGAGGCTCTGAATGTCACAACCTGCCACGTCCATGCCAAGTC |
| | GAACCTGCGACACTGGAACAAAACCTGTGAGCTAACTCTTGTGAGGCAGGCATCCTGGGCCTG |
| | CAACCTGATCCTCGGGTCGTTCCCAGAGTCCCAGTCACTGACCTCCGTGGACCTCCTTGACATAA |
| | ATGTGGTGTGCTGGGAAGAGAAGGGTTGGCGTAGGGTAAAGACCTGCGACTTCCATCCCTTTG |
| | ACAACCTTCGCCTGGTGGCCCCTCATTCCCTCCAAGTTCTGCACATTGATACCCAGAGATGTAAC |
| | ATAAGCTGGAAGGTCTCCCAGGTCTCTGACTTCATTGAACCATACTTGGAATTTGAGGCCCGTA |
| | GACGTCTTCTGGGCCACAGCTGGGAGGATGCATCCGTATTAAGCCTCAAGCAGAGACAGCAGT |
| | GGCTCTTCTTGGAGATGCTGATCCCTAGTACCTCATATGAGGTCCAGGTGAGGGTCAAAGCTCA |
| | ACGAAACAATACCGGGACCTGGAGTCCCTGGAGCCAGCCCCTGACCTTTCGGACAAGGCCAGC |
| | 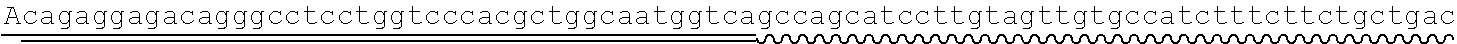 |
| | 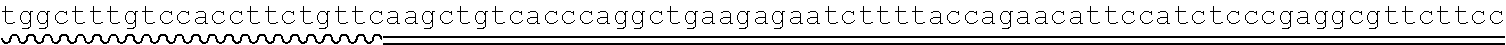 |
| | atcctctctacagtgtgtaccatggggacttccagagttggacaggggcccgcagagccggaccacaagcaagacagaatg |
| | gtgtcagtacttcatcagcaggctcagagtccagcatctgggaggccgtcgccacactcacctatagcccggcatgccctgtg |
| | cagtttgcctgcctgaagtgggaggccacagccccgggcttcccagggctcccaggctcagagcatgtgctgccggcagggt |
| | gtctggagttggaaggacagccatctgcctacAGgccccaggaggactgggccccactgggctctgccaggccccctcctcc |
| | agactcagacagcggcagcagcgactattgcatgttggactgctgtgaggaatgccacctctcagccttcccaggacacacc |
| | gagagtcctgagctcacgctagctcagcctgtggcccttcctgtgtccagcagggcctga |
| SEQ ID | MATIALPWSLSLYVFLLLLATPWASAAVKNCSHLECFYNSRANVSCMWSHEEALNVTTCHVHAKS |
| NO: 24 | NLRHWNKTCELTLVRQASWACNLILGSFPESQSLTSVDLLDINVVCWEEKGWRRVKTCDFHPFDN |
| | LRLVAPHSLQVLHIDTQRCNISWKVSQVSDFIEPYLEFEARRRLLGHSWEDASVLSLKQRQQWLFLE |
| | 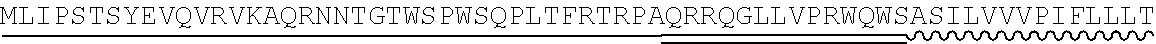 |
| | 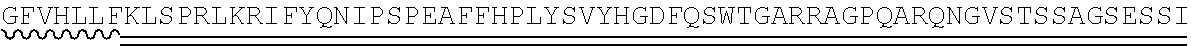 |
| | WEAVATLTYSPACPVQFACLKWEATAPGFPGLPGSEHVLPAGCLELEGQPSAYRPQEDWAPLGSA |
| | RPPPPDSDSGSSDYCMLDCCEECHLSAFPGHTESPELTLAQPVALPVSSRA* |
| | 13. mouse orthoIL2Rb-IL9R mut7 (YLPL) (molL9R-YLPL) |
| SEQ ID | ATGGCTACCATAGCTCTTCCCTGGAGCCTGTCCCTCTACGTCTTCCTCCTGCTCCTGGCTACACCT |
| NO: 25 | TGGGCATCTGCAGCAGTGAAAAACTGTTCCCATCTTGAATGCTTCTACAACTCAAGAGCCAATG |
| | TCTCTTGCATGTGGAGCCATGAAGAGGCTCTGAATGTCACAACCTGCCACGTCCATGCCAAGTC |
| | GAACCTGCGACACTGGAACAAAACCTGTGAGCTAACTCTTGTGAGGCAGGCATCCTGGGCCTG |
| | CAACCTGATCCTCGGGTCGTTCCCAGAGTCCCAGTCACTGACCTCCGTGGACCTCCTTGACATAA |
| | ATGTGGTGTGCTGGGAAGAGAAGGGTTGGCGTAGGGTAAAGACCTGCGACTTCCATCCCTTTG |
| | ACAACCTTCGCCTGGTGGCCCCTCATTCCCTCCAAGTTCTGCACATTGATACCCAGAGATGTAAC |
| | ATAAGCTGGAAGGTCTCCCAGGTCTCTGACTTCATTGAACCATACTTGGAATTTGAGGCCCGTA |
| | GACGTCTTCTGGGCCACAGCTGGGAGGATGCATCCGTATTAAGCCTCAAGCAGAGACAGCAGT |
| | GGCTCTTCTTGGAGATGCTGATCCCTAGTACCTCATATGAGGTCCAGGTGAGGGTCAAAGCTCA |
| | ACGAAACAATACCGGGACCTGGAGTCCCTGGAGCCAGCCCCTGACCTTTCGGACAAGGCCAGC |
| | 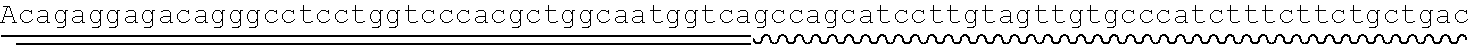 |
| | 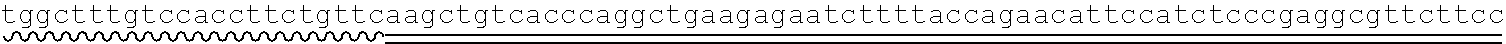 |
| | atcctctctacagtgtgtaccatggggacttccagagttggacaggggcccgcagagccggaccacaagcaagacagaatg |
| | gtgtcagtacttcatcagcaggctcagagtccagcatctgggaggccgtcgccacactcacctatagcccggcatgccctgtg |
| | cagtttgcctgcctgaagtgggaggccacagccccgggcttcccagggctcccaggctcagagcatgtgctgccggcagggt |
| | gtctggagttggaaggacagccatctgcctacctgccccTggaggactgggccccactgggctctgccaggccccctcctcca |
| | gactcagacagcggcagcagcgactattgcatgttggactgctgtgaggaatgccacctctcagccttcccaggacacaccg |
| | agagtcctgagctcacgctagctcagcctgtggcccttcctgtgtccagcagggcctga |
| SEQ ID | MATIALPWSLSLYVFLLLLATPWASAAVKNCSHLECFYNSRANVSCMWSHEEALNVTTCHVHAKS |
| NO: 26 | NLRHWNKTCELTLVRQASWACNLILGSFPESQSLTSVDLLDINVVCWEEKGWRRVKTCDFHPFDN |
| | LRLVAPHSLQVLHIDTQRCNISWKVSQVSDFIEPYLEFEARRRLLGHSWEDASVLSLKQRQQWLFLE |
| | 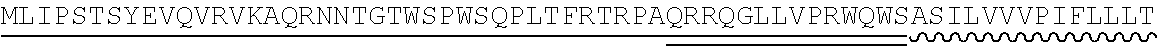 |
| | 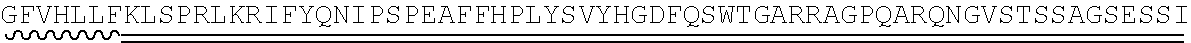 |
| | WEAVATLTYSPACPVQFACLKWEATAPGFPGLPGSEHVLPAGCLELEGQPSAYLPLEDWAPLGSAR |
| | PPPPDSDSGSSDYCMLDCCEECHLSAFPGHTESPELTLAQPVALPVSSRA* |
| | 14. mouse orthoIL2Rb-IL9R mut9 (YLKQ) (molL9R-YLKQ) |
| SEQ ID | ATGGCTACCATAGCTCTTCCCTGGAGCCTGTCCCTCTACGTCTTCCTCCTGCTCCTGGCTACACCT |
| NO: 27 | TGGGCATCTGCAGCAGTGAAAAACTGTTCCCATCTTGAATGCTTCTACAACTCAAGAGCCAATG |
| | TCTCTTGCATGTGGAGCCATGAAGAGGCTCTGAATGTCACAACCTGCCACGTCCATGCCAAGTC |
| | GAACCTGCGACACTGGAACAAAACCTGTGAGCTAACTCTTGTGAGGCAGGCATCCTGGGCCTG |
| | CAACCTGATCCTCGGGTCGTTCCCAGAGTCCCAGTCACTGACCTCCGTGGACCTCCTTGACATAA |
| | ATGTGGTGTGCTGGGAAGAGAAGGGTTGGCGTAGGGTAAAGACCTGCGACTTCCATCCCTTTG |
| | ACAACCTTCGCCTGGTGGCCCCTCATTCCCTCCAAGTTCTGCACATTGATACCCAGAGATGTAAC |
| | ATAAGCTGGAAGGTCTCCCAGGTCTCTGACTTCATTGAACCATACTTGGAATTTGAGGCCCGTA |
| | GACGTCTTCTGGGCCACAGCTGGGAGGATGCATCCGTATTAAGCCTCAAGCAGAGACAGCAGT |
| | GGCTCTTCTTGGAGATGCTGATCCCTAGTACCTCATATGAGGTCCAGGTGAGGGTCAAAGCTCA |
| | ACGAAACAATACCGGGACCTGGAGTCCCTGGAGCCAGCCCCTGACCTTTCGGACAAGGCCAGC |
| | 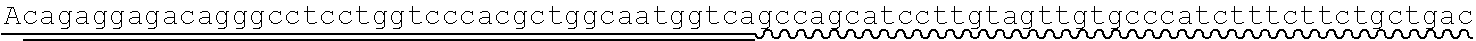 |
| | 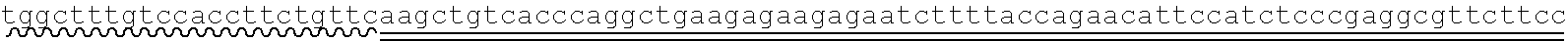 |
| | atcctctctacagtgtgtaccatggggacttccagagttggacaggggcccgcagagccggaccacaagcaagacagaatg |
| | gtgtcagtacttcatcagcaggctcagagtccagcatctgggaggccgtcgccacactcacctatagcccggcatgccctgtg |
| | cagtttgcctgcctgaagtgggaggccacagccccgggcttcccagggctcccaggctcagagcatgtgctgccggcagggt |
| | gtctggagttggaaggacagccatctgcctacctgAAGcaggaggactgggccccactgggctctgccaggccccctcctcc |
| | agactcagacagcggcagcagcgactattgcatgttggactgctgtgaggaatgccacctctcagccttcccaggacacacc |
| | gagagtcctgagctcacgctagctcagcctgtggcccttcctgtgtccagcagggcctga |
| SEQ ID | MATIALPWSLSLYVFLLLLATPWASAAVKNCSHLECFYNSRANVSCMWSHEEALNVTTCHVHAKS |
| NO: 28 | NLRHWNKTCELTLVRQASWACNLILGSFPESQSLTSVDLLDINVVCWEEKGWRRVKTCDFHPFDN |
| | LRLVAPHSLQVLHIDTQRCNISWKVSQVSDFIEPYLEFEARRRLLGHSWEDASVLSLKQRQQWLFLE |
| | 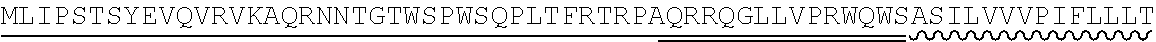 |
| | 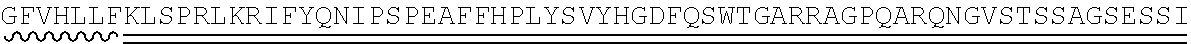 |
| | WEAVATLTYSPACPVQFACLKWEATAPGFPGLPGSEHVLPAGCLELEGQPSAYLKQEDWAPLGSA |
| | RPPPPDSDSGSSDYCMLDCCEECHLSAFPGHTESPELTLAQPVALPVSSRA* |
| | 15. mouse orthoIL2Rb-IL9R mutX (YLXQ) (moIL9R-YLXQ), X = any 20 AA |
| SEQ ID | ATGGCTACCATAGCTCTTCCCTGGAGCCTGTCCCTCTACGTCTTCCTCCTGCTCCTGGCTACACCT |
| NO: 29 | TGGGCATCTGCAGCAGTGAAAAACTGTTCCCATCTTGAATGCTTCTACAACTCAAGAGCCAATG |
| | TCTCTTGCATGTGGAGCCATGAAGAGGCTCTGAATGTCACAACCTGCCACGTCCATGCCAAGTC |
| | GAACCTGCGACACTGGAACAAAACCTGTGAGCTAACTCTTGTGAGGCAGGCATCCTGGGCCTG |
| | CAACCTGATCCTCGGGTCGTTCCCAGAGTCCCAGTCACTGACCTCCGTGGACCTCCTTGACATAA |
| | ATGTGGTGTGCTGGGAAGAGAAGGGTTGGCGTAGGGTAAAGACCTGCGACTTCCATCCCTTTG |
| | ACAACCTTCGCCTGGTGGCCCCTCATTCCCTCCAAGTTCTGCACATTGATACCCAGAGATGTAAC |
| | ATAAGCTGGAAGGTCTCCCAGGTCTCTGACTTCATTGAACCATACTTGGAATTTGAGGCCCGTA |
| | GACGTCTTCTGGGCCACAGCTGGGAGGATGCATCCGTATTAAGCCTCAAGCAGAGACAGCAGT |
| | GGCTCTTCTTGGAGATGCTGATCCCTAGTACCTCATATGAGGTCCAGGTGAGGGTCAAAGCTCA |
| | ACGAAACAATACCGGGACCTGGAGTCCCTGGAGCCAGCCCCTGACCTTTCGGACAAGGCCAGC |
| | 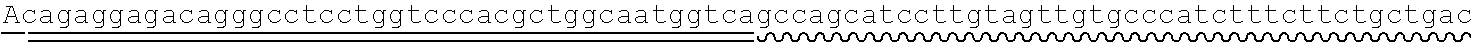 |
| | 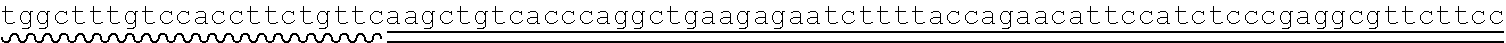 |
| | atcctctctacagtgtgtaccatggggacttccagagttggacaggggcccgcagagccggaccacaagcaagacagaatg |
| | gtgtcagtacttcatcagcaggctcagagtccagcatctgggaggccgtcgccacactcacctatagcccggcatgccctgtg |
| | cagtttgcctgcctgaagtgggaggccacagccccgggcttcccagggctcccaggctcagagcatgtgctgccggcagggt |
| | gtctggagttggaaggacagccatctgcctacctgnnncaggaggactgggccccactgggctctgccaggccccctcctcc |
| | agactcagacagcggcagcagcgactattgcatgttggactgctgtgaggaatgccacctctcagccttcccaggacacacc |
| | gagagtcctgagctcacgctagctcagcctgtggcccttcctgtgtccagcagggcctga |
| SEQ ID | MATIALPWSLSLYVFLLLLATPWASAAVKNCSHLECFYNSRANVSCMWSHEEALNVTTCHVHAKS |
| NO: 30 | NLRHWNKTCELTLVRQASWACNLILGSFPESQSLTSVDLLDINVVCWEEKGWRRVKTCDFHPFDN |
| | LRLVAPHSLQVLHIDTQRCNISWKVSQVSDFIEPYLEFEARRRLLGHSWEDASVLSLKQRQQWLFLE |
| | 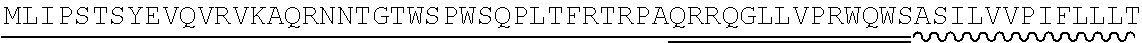 |
| | 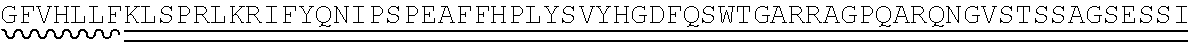 |
| | WEAVATLTYSPACPVQFACLKWEATAPGFPGLPGSEHVLPAGCLELEGQPSAYLXQEDWAPLGSA |
| | RPPPPDSDSGSSDYCMLDCCEECHLSAFPGHTESPELTLAQPVALPVSSRA* |
| | 16. mouse orthoIL2Rb-IL9R 1x pY (molL9R 1xpY) |
| SEQ ID | ATGGCTACCATAGCTCTTCCCTGGAGCCTGTCCCTCTACGTCTTCCTCCTGCTCCTGGCTACACCT |
| NO: 31 | TGGGCATCTGCAGCAGTGAAAAACTGTTCCCATCTTGAATGCTTCTACAACTCAAGAGCCAATG |
| | TCTCTTGCATGTGGAGCCATGAAGAGGCTCTGAATGTCACAACCTGCCACGTCCATGCCAAGTC |
| | GAACCTGCGACACTGGAACAAAACCTGTGAGCTAACTCTTGTGAGGCAGGCATCCTGGGCCTG |
| | CAACCTGATCCTCGGGTCGTTCCCAGAGTCCCAGTCACTGACCTCCGTGGACCTCCTTGACATAA |
| | ATGTGGTGTGCTGGGAAGAGAAGGGTTGGCGTAGGGTAAAGACCTGCGACTTCCATCCCTTTG |
| | ACAACCTTCGCCTGGTGGCCCCTCATTCCCTCCAAGTTCTGCACATTGATACCCAGAGATGTAAC |
| | ATAAGCTGGAAGGTCTCCCAGGTCTCTGACTTCATTGAACCATACTTGGAATTTGAGGCCCGTA |
| | GACGTCTTCTGGGCCACAGCTGGGAGGATGCATCCGTATTAAGCCTCAAGCAGAGACAGCAGT |
| | GGCTCTTCTTGGAGATGCTGATCCCTAGTACCTCATATGAGGTCCAGGTGAGGGTCAAAGCTCA |
| | ACGAAACAATACCGGGACCTGGAGTCCCTGGAGCCAGCCCCTGACCTTTCGGACAAGGCCAGC |
| | 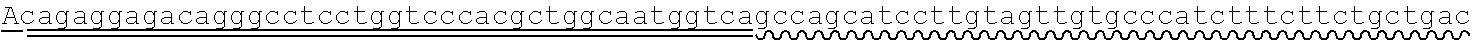 |
| | 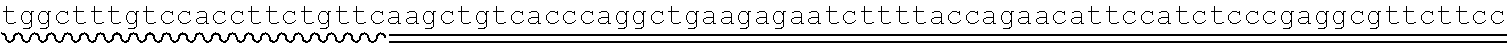 |
| | atcctctctacagtgtgtaccatggggacttccagagttggacaggggcccgcagagccggaccacaagcaagacagaatg |
| | gtgtcagtacttcatcagcaggctcagagtccagcatctgggaggccgtcgccacactcacctatagcccggcatgccctgtg |
| | cagtttgcctgcctgaagtgggaggccacagccccgggcttcccagggctcccaggctcagagcatgtgctgccggcagggt |
| | gtctggagttggaaggacagccatctgcctacctgccccaggaggactgggccccactgggctctgccaggccccctcctcca |
| | 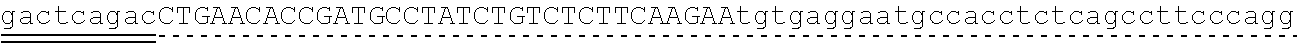 |
| | 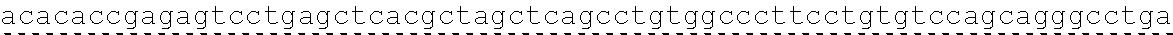 |
| SEQ ID | MATIALPWSLSLYVFLLLLATPWASAAVKNCSHLECFYNSRANVSCMWSHEEALNVTTCHVHAKS |
| NO: 32 | NLRHWNKTCELTLVRQASWACNLILGSFPESQSLTSVDLLDINVVCWEEKGWRRVKTCDFHPFDN |
| | LRLVAPHSLQVLHIDTQRCNISWKVSQVSDFIEPYLEFEARRRLLGHSWEDASVLSLKQRQQWLFLE |
| | 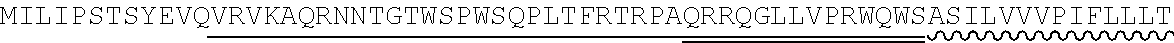 |
| | 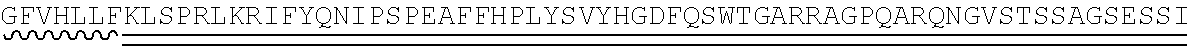 |
| | WEAVATLTYSPACPVQFACLKWEATAPGFPGLPGSEHVLPAGCLELEGQPSAYLPQEDWAPLGSA |
| | 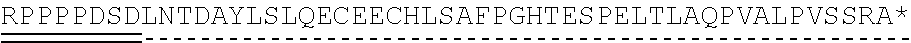 |
| | 17. mouse orthoIL2Rb-IL9R 2x pY (molL9R 2xpY) |
| SEQ ID | ATGGCTACCATAGCTCTTCCCTGGAGCCTGTCCCTCTACGTCTTCCTCCTGCTCCTGGCTACACCT |
| NO: 33 | TGGGCATCTGCAGCAGTGAAAAACTGTTCCCATCTTGAATGCTTCTACAACTCAAGAGCCAATG |
| | TCTCTTGCATGTGGAGCCATGAAGAGGCTCTGAATGTCACAACCTGCCACGTCCATGCCAAGTC |
| | GAACCTGCGACACTGGAACAAAACCTGTGAGCTAACTCTTGTGAGGCAGGCATCCTGGGCCTG |
| | CAACCTGATCCTCGGGTCGTTCCCAGAGTCCCAGTCACTGACCTCCGTGGACCTCCTTGACATAA |
| | ATGTGGTGTGCTGGGAAGAGAAGGGTTGGCGTAGGGTAAAGACCTGCGACTTCCATCCCTTTG |
| | ACAACCTTCGCCTGGTGGCCCCTCATTCCCTCCAAGTTCTGCACATTGATACCCAGAGATGTAAC |
| | ATAAGCTGGAAGGTCTCCCAGGTCTCTGACTTCATTGAACCATACTTGGAATTTGAGGCCCGTA |
| | GACGTCTTCTGGGCCACAGCTGGGAGGATGCATCCGTATTAAGCCTCAAGCAGAGACAGCAGT |
| | GGCTCTTCTTGGAGATGCTGATCCCTAGTACCTCATATGAGGTCCAGGTGAGGGTCAAAGCTCA |
| | ACGAAACAATACCGGGACCTGGAGTCCCTGGAGCCAGCCCCTGACCTTTCGGACAAGGCCAGC |
| | 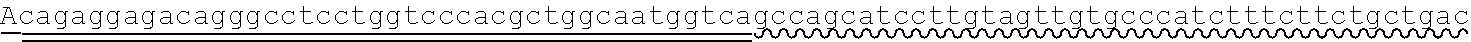 |
| | 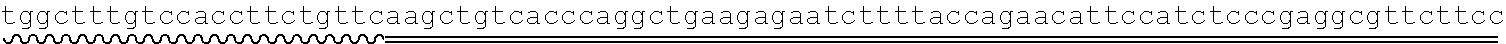 |
| | atcctctctacagtgtgtaccatggggacttccagagttggacaggggcccgcagagccggaccacaagcaagacagaatg |
| | gtgtcagtacttcatcagcaggctcagagtccagcatctgggaggccgtcgccacactcacctatagcccggcatgccctgtg |
| | cagtttgcctgcctgaagtgggaggccacagccccgggcttcccagggctcccaggctcagagcatgtgctgccggcagggt |
| | gtctggagttggaaggacagccatctgcctacctgccccaggaggactgggccccactgggctctgccaggccccctcctcca |
| | 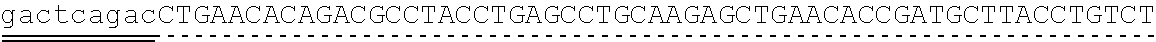 |
| | 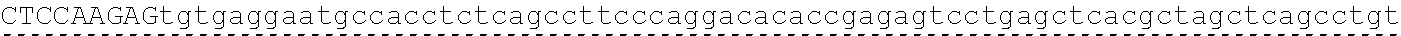 |
| | 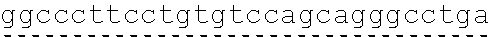 |
| SEQ ID | MATIALPWSLSLYVFLLLLATPWASAAVKNCSHLECFYNSRANVSCMWSHEEALNVTTCHVHAKS |
| NO: 34 | NLRHWNKTCELTLVRQASWACNLILGSFPESQSLTSVDLLDINVVCWEEKGWRRVKTCDFHPFDN |
| | LRLVAPHSLQVLHIDTQRCNISWKVSQVSDFIEPYLEFEARRRLLGHSWEDASVLSLKQRQQWLFLE |
| | 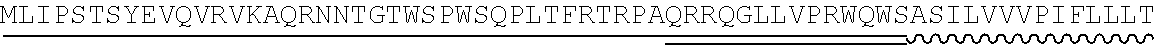 |
| | 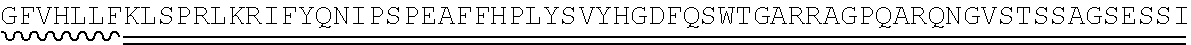 |
| | WEAVATLTYSPACPVQFACLKWEATAPGFPGLPGSEHVLPAGCLELEGQPSAYLPQEDWAPLGSA |
| | 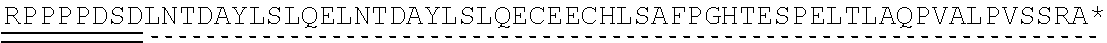 |
| | 18. mouse orthoIL2Rb-IL9R 3x pY (molL9R 3xpY) |
| SEQ ID | ATGGCTACCATAGCTCTTCCCTGGAGCCTGTCCCTCTACGTCTTCCTCCTGCTCCTGGCTACACCT |
| NO: 35 | TGGGCATCTGCAGCAGTGAAAAACTGTTCCCATCTTGAATGCTTCTACAACTCAAGAGCCAATG |
| | TCTCTTGCATGTGGAGCCATGAAGAGGCTCTGAATGTCACAACCTGCCACGTCCATGCCAAGTC |
| | GAACCTGCGACACTGGAACAAAACCTGTGAGCTAACTCTTGTGAGGCAGGCATCCTGGGCCTG |
| | CAACCTGATCCTCGGGTCGTTCCCAGAGTCCCAGTCACTGACCTCCGTGGACCTCCTTGACATAA |
| | ATGTGGTGTGCTGGGAAGAGAAGGGTTGGCGTAGGGTAAAGACCTGCGACTTCCATCCCTTTG |
| | ACAACCTTCGCCTGGTGGCCCCTCATTCCCTCCAAGTTCTGCACATTGATACCCAGAGATGTAAC |
| | ATAAGCTGGAAGGTCTCCCAGGTCTCTGACTTCATTGAACCATACTTGGAATTTGAGGCCCGTA |
| | GACGTCTTCTGGGCCACAGCTGGGAGGATGCATCCGTATTAAGCCTCAAGCAGAGACAGCAGT |
| | GGCTCTTCTTGGAGATGCTGATCCCTAGTACCTCATATGAGGTCCAGGTGAGGGTCAAAGCTCA |
| | ACGAAACAATACCGGGACCTGGAGTCCCTGGAGCCAGCCCCTGACCTTTCGGACAAGGCCAGC |
| | 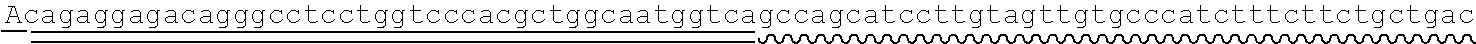 |
| | 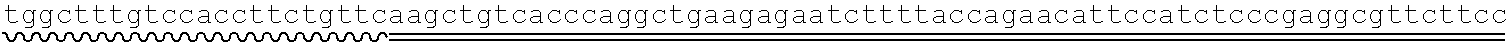 |
| | atcctctctacagtgtgtaccatggggacttccagagttggacaggggcccgcagagccggaccacaagcaagacagaatg |
| | gtgtcagtacttcatcagcaggctcagagtccagcatctgggaggccgtcgccacactcacctatagcccggcatgccctgtg |
| | cagtttgcctgcctgaagtgggaggccacagccccgggcttcccagggctcccaggctcagagcatgtgctgccggcagggt |
| | gtctggagttggaaggacagccatctgcctacctgccccaggaggactgggccccactgggctctgccaggccccctcctcca |
| | 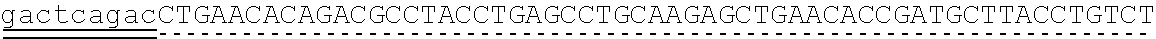 |
| | 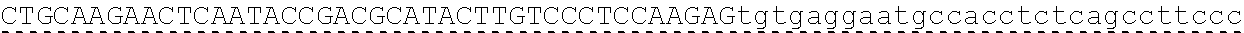 |
| | 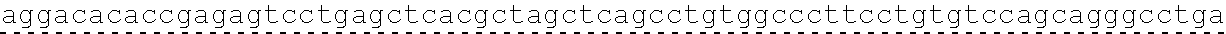 |
| SEQ ID | MATIALPWSLSLYVFLLLLATPWASAAVKNCSHLECFYNSRANVSCMWSHEEALNVTTCHVHAKS |
| NO: 36 | NLRHWNKTCELTLVRQASWACNLILGSFPESQSLTSVDLLDINVVCWEEKGWRRVKTCDFHPFDN |
| | LRLVAPHSLQVLHIDTQRCNISWKVSQVSDFIEPYLEFEARRRLLGHSWEDASVLSLKQRQQWLFLE |
| | 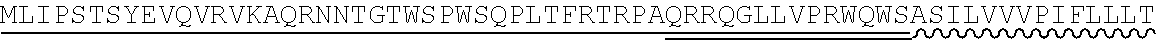 |
| | 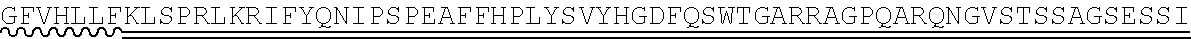 |
| | WEAVATLTYSPACPVQFACLKWEATAPGFPGLPGSEHVLPAGCLELEGQPSAYLPQEDWAPLGSA |
| | 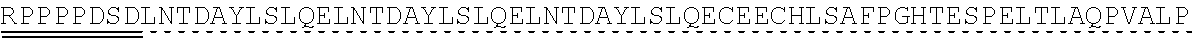 |
| | 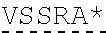 |
| | 19. mouse orthoIL2Rb-IL9R (YLXQ) 1-3x pY (moIL9R YLXQ 1-3xpY), X = any 20 AA |
| SEQ ID | ATGGCTACCATAGCTCTTCCCTGGAGCCTGTCCCTCTACGTCTTCCTCCTGCTCCTGGCTACACCT |
| NO: 37 | TGGGCATCTGCAGCAGTGAAAAACTGTTCCCATCTTGAATGCTTCTACAACTCAAGAGCCAATG |
| | TCTCTTGCATGTGGAGCCATGAAGAGGCTCTGAATGTCACAACCTGCCACGTCCATGCCAAGTC |
| | GAACCTGCGACACTGGAACAAAACCTGTGAGCTAACTCTTGTGAGGCAGGCATCCTGGGCCTG |
| | CAACCTGATCCTCGGGTCGTTCCCAGAGTCCCAGTCACTGACCTCCGTGGACCTCCTTGACATAA |
| | ATGTGGTGTGCTGGGAAGAGAAGGGTTGGCGTAGGGTAAAGACCTGCGACTTCCATCCCTTTG |
| | ACAACCTTCGCCTGGTGGCCCCTCATTCCCTCCAAGTTCTGCACATTGATACCCAGAGATGTAAC |
| | ATAAGCTGGAAGGTCTCCCAGGTCTCTGACTTCATTGAACCATACTTGGAATTTGAGGCCCGTA |
| | GACGTCTTCTGGGCCACAGCTGGGAGGATGCATCCGTATTAAGCCTCAAGCAGAGACAGCAGT |
| | GGCTCTTCTTGGAGATGCTGATCCCTAGTACCTCATATGAGGTCCAGGTGAGGGTCAAAGCTCA |
| | ACGAAACAATACCGGGACCTGGAGTCCCTGGAGCCAGCCCCTGACCTTTCGGACAAGGCCAGC |
| | 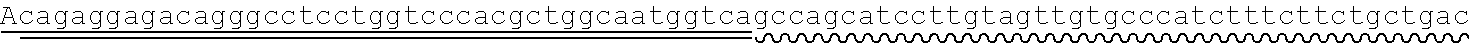 |
| | 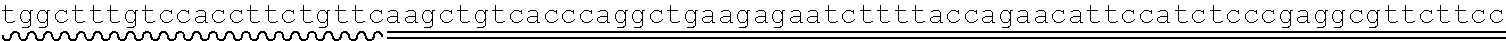 |
| | atcctctctacagtgtgtaccatggggacttccagagttggacaggggcccgcagagccggaccacaagcaagacagaatg |
| | gtgtcagtacttcatcagcaggctcagagtccagcatctgggaggccgtcgccacactcacctatagcccggcatgccctgtg |
| | cagtttgcctgcctgaagtgggaggccacagccccgggcttcccagggctcccaggctcagagcatgtgctgccggcagggt |
| | gtctggagttggaaggacagccatctgcctacctgnnncaggaggactgggccccactgggctctgccaggccccctcctcc |
| | 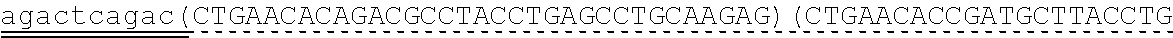 |
| | 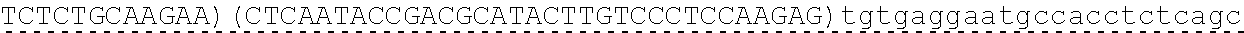 |
| | 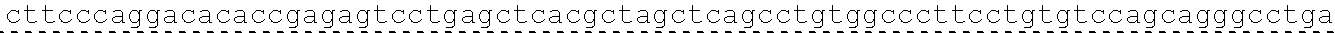 |
| SEQ ID | MATIALPWSLSLYVFLLLLATPWASAAVKNCSHLECFYNSRANVSCMWSHEEALNVTTCHVHAKS |
| NO: 38 | NLRHWNKTCELTLVRQASWACNLILGSFPESQSLTSVDLLDINVVCWEEKGWRRVKTCDFHPFDN |
| | LRLVAPHSLQVLHIDTQRCNISWKVSQVSDFIEPYLEFEARRRLLGHSWEDASVLSLKQRQQWLFLE |
| | 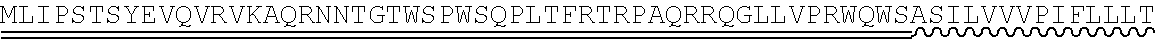 |
| | 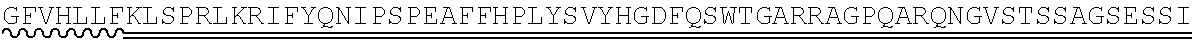 |
| | WEAVATLTYSPACPVQFACLKWEATAPGFPGLPGSEHVLPAGCLELEGQPSAYLXQEDWAPLGSA |
| | 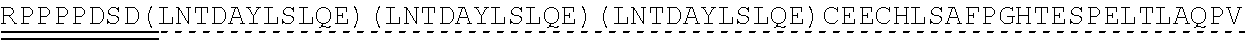 |
| | 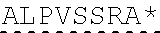 |
| | 20. mouse orthoIL2Rb-IL21R (mo21R) |
| SEQ ID | ATGGCTACCATAGCTCTTCCCTGGAGCCTGTCCCTCTACGTCTTCCTCCTGCTCCTGGCTACACCT |
| NO: 39 | TGGGCATCTGCAGCAGTGAAAAACTGTTCCCATCTTGAATGCTTCTACAACTCAAGAGCCAATG |
| | TCTCTTGCATGTGGAGCCATGAAGAGGCTCTGAATGTCACAACCTGCCACGTCCATGCCAAGTC |
| | GAACCTGCGACACTGGAACAAAACCTGTGAGCTAACTCTTGTGAGGCAGGCATCCTGGGCCTG |
| | CAACCTGATCCTCGGGTCGTTCCCAGAGTCCCAGTCACTGACCTCCGTGGACCTCCTTGACATAA |
| | ATGTGGTGTGCTGGGAAGAGAAGGGTTGGCGTAGGGTAAAGACCTGCGACTTCCATCCCTTTG |
| | ACAACCTTCGCCTGGTGGCCCCTCATTCCCTCCAAGTTCTGCACATTGATACCCAGAGATGTAAC |
| | ATAAGCTGGAAGGTCTCCCAGGTCTCTGACTTCATTGAACCATACTTGGAATTTGAGGCCCGTA |
| | GACGTCTTCTGGGCCACAGCTGGGAGGATGCATCCGTATTAAGCCTCAAGCAGAGACAGCAGT |
| | GGCTCTTCTTGGAGATGCTGATCCCTAGTACCTCATATGAGGTCCAGGTGAGGGTCAAAGCTCA |
| | ACGAAACAATACCGGGACCTGGAGTCCCTGGAGCCAGCCCCTGACCTTTCGGACAAGACCTGC |
| | 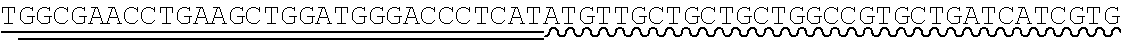 |
| | 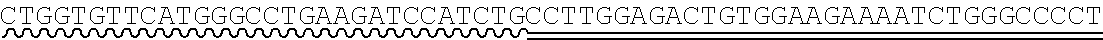 |
| | GTGCCTACTCCTGAGAGCTTCTTCCAGCCACTGTACAGAGAGCACAGCGGCAACTTCAAGAAAT |
| | GGGTCAACACCCCTTTCACCGCCAGCAGTATCGAGCTGGTGCCTCAGAGCAGCACCACAACATC |
| | TGCCCTGCACCTGTCTCTGTACCCCGCCAAAGAGAAGAAGTTCCCTGGCCTGCCTGGACTGGAA |
| | GAACAGCTGGAATGTGACGGCATGAGCGAGCCTGGCCACTGGTGTATCATTCCTCTGGCTGCT |
| | GGACAGGCCGTGTCCGCCTATAGCGAGGAAAGAGACAGACCCTACGGCCTGGTGTCCATCGAC |
| | ACAGTGACAGTGGGAGATGCCGAGGGCCTGTGTGTGTGGCCTTGTAGCTGTGAAGATGACGG |
| | CTACCCTGCCATGAACCTGGATGCCGGAAGAGAGAGCGGCCCTAACTCTGAGGATCTGCTGCT |
| | CGTGACCGATCCTGCCTTCCTGTCTTGCGGCTGTGTGTCTGGATCTGGCCTGAGACTCGGAGGC |
| | TCTCCTGGAAGCCTGCTGGATAGACTGAGACTGAGCTTCGCCAAAGAAGGCGACTGGACCGCC |
| | GATCCTACTTGGAGAACAGGATCTCCTGGCGGCGGAAGCGAATCTGAAGCAGGTTCTCCACCT |
| | GGCCTGGACATGGACACATTCGACTCTGGCTTCGCCGGCAGCGATTGTGGAAGCCCTGTGGAA |
| | ACAGACGAGGGCCCACCTAGAAGCTACCTGAGACAGTGGGTCGTGCGGACACCTCCTCCAGTT |
| | GATTCTGGCGCCCAGTCCTCTTGA |
| SEQ ID | MATIALPWSLSLYVFLLLLATPWASAAVKNCSHLECFYNSRANVSCMWSHEEALNVTTCHVHAKS |
| NO: 40 | NLRHWNKTCELTLVRQASWACNLILGSFPESQSLTSVDLLDINVVCWEEKGWRRVKTCDFHPFDN |
| | LRLVAPHSLQVLHIDTQRCNISWKVSQVSDFIEPYLEFEARRRLLGHSWEDASVLSLKQRQQWLFLE |
| | 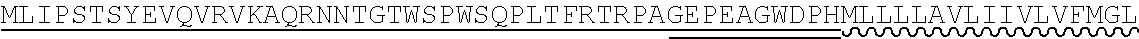 |
| | 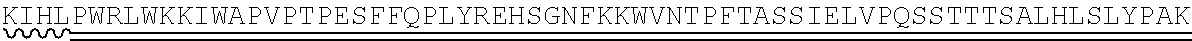 |
| | EKKFPGLPGLEEQLECDGMSEPGHWCIIPLAAGQAVSAYSEERDRPYGLVSIDTVTVGDAEGLCVW |
| | PCSCEDDGYPAMNLDAGRESGPNSEDLLLVTDPAFLSCGCVSGSGLRLGGSPGSLLDRLRLSFAKEG |
| | DWTADPTWRTGSPGGGSESEAGSPPGLDMDTFDSGFAGSDCGSPVETDEGPPRSYLRQWVVRT |
| | PPPVDSGAQSS* |
| | 21. mouse orthoIL2Rb-IL21R 1-3x pY (molL21R 1-3xpY) |
| SEQ ID | ATGGCTACCATAGCTCTTCCCTGGAGCCTGTCCCTCTACGTCTTCCTCCTGCTCCTGGCTACACCT |
| NO: 41 | TGGGCATCTGCAGCAGTGAAAAACTGTTCCCATCTTGAATGCTTCTACAACTCAAGAGCCAATG |
| | TCTCTTGCATGTGGAGCCATGAAGAGGCTCTGAATGTCACAACCTGCCACGTCCATGCCAAGTC |
| | GAACCTGCGACACTGGAACAAAACCTGTGAGCTAACTCTTGTGAGGCAGGCATCCTGGGCCTG |
| | CAACCTGATCCTCGGGTCGTTCCCAGAGTCCCAGTCACTGACCTCCGTGGACCTCCTTGACATAA |
| | ATGTGGTGTGCTGGGAAGAGAAGGGTTGGCGTAGGGTAAAGACCTGCGACTTCCATCCCTTTG |
| | ACAACCTTCGCCTGGTGGCCCCTCATTCCCTCCAAGTTCTGCACATTGATACCCAGAGATGTAAC |
| | ATAAGCTGGAAGGTCTCCCAGGTCTCTGACTTCATTGAACCATACTTGGAATTTGAGGCCCGTA |
| | GACGTCTTCTGGGCCACAGCTGGGAGGATGCATCCGTATTAAGCCTCAAGCAGAGACAGCAGT |
| | GGCTCTTCTTGGAGATGCTGATCCCTAGTACCTCATATGAGGTCCAGGTGAGGGTCAAAGCTCA |
| | ACGAAACAATACCGGGACCTGGAGTCCCTGGAGCCAGCCCCTGACCTTTCGGACAAGACCTGC |
| | 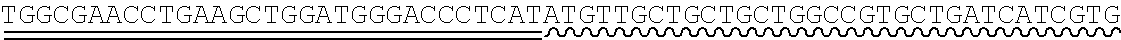 |
| | 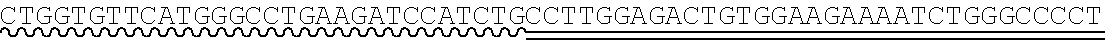 |
| | GTGCCTACTCCTGAGAGCTTCTTCCAGCCACTGTACAGAGAGCACAGCGGCAACTTCAAGAAAT |
| | GGGTCAACACCCCTTTCACCGCCAGCAGTATCGAGCTGGTGCCTCAGAGCAGCACCACAACATC |
| | TGCCCTGCACCTGTCTCTGTACCCCGCCAAAGAGAAGAAGTTCCCTGGCCTGCCTGGACTGGAA |
| | GAACAGCTGGAATGTGACGGCATGAGCGAGCCTGGCCACTGGTGTATCATTCCTCTGGCTGCT |
| | GGACAGGCCGTGTCCGCCTATAGCGAGGAAAGAGACAGACCCTACGGCCTGGTGTCCATCGAC |
| | ACAGTGACAGTGGGAGATGCCGAGGGCCTGTGTGTGTGGCCTTGTAGCTGTGAAGATGACGG |
| | CTACCCTGCCATGAACCTGGATGCCGGAAGAGAGAGCGGCCCTAACTCTGAGGATCTGCTGCT |
| | CGTGACCGATCCTGCCTTCCTGTCTTGCGGCTGTGTGTCTGGATCTGGCCTGAGACTCGGAGGC |
| | TCTCCTGGAAGCCTGCTGGATAGACTGAGACTGAGCTTCGCCAAAGAAGGCGACTGGACCGCC |
| | GATCCTACTTGGAGAACAGGATCTCCTGGCGGCGGAAGCGAATCTGAAGCAGGTTCTCCACCT |
| | GGCCTGGACATGGACACATTCGACTCTGGCTTCGCCGGCAGCGATTGTGGAAGCCCTGTGGAA |
| | ACAGACGAGGGCCCACCTAGAAGCTACCTGAGACAGTGGGTCGTGCGGACACCTCCTCCAGTT |
| | GATTCTGGCGCCCAGTCCTCT(CTGAACACAGACGCCTACCTGAGCCTGCAAGAG)(CTGAACAC |
| | CGATGCTTACCTGTCTCTGCAAGAA)(CTCAATACCGACGCATACTTGTCCCTCCAAGAG)CTGCA |
| | GGCCCAGGACTCTGTGCACCTGATTTGA |
| SEQ ID | MATIALPWSLSLYVFLLLLATPWASAAVKNCSHLECFYNSRANVSCMWSHEEALNVTTCHVHAKS |
| NO: 42 | NLRHWNKTCELTLVRQASWACNLILGSFPESQSLTSVDLLDINVVCWEEKGWRRVKTCDFHPFDN |
| | LRLVAPHSLQVLHIDTQRCNISWKVSQVSDFIEPYLEFEARRRLLGHSWEDASVLSLKQRQQWLFLE |
| | 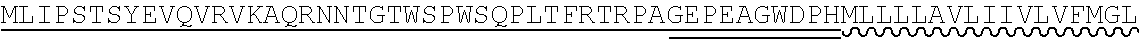 |
| | 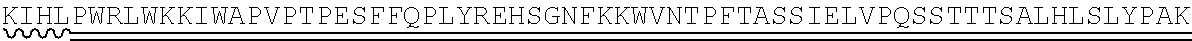 |
| | EKKFPGLPGLEEQLECDGMSEPGHWCIIPLAAGQAVSAYSEERDRPYGLVSIDTVTVGDAEGLCVW |
| | PCSCEDDGYPAMNLDAGRESGPNSEDLLLVTDPAFLSCGCVSGSGLRLGGSPGSLLDRLRLSFAKEG |
| | DWTADPTWRTGSPGGGSESEAGSPPGLDMDTFDSGFAGSDCGSPVETDEGPPRSYLRQWVVRT |
| | PPPVDSGAQSS(LNTDAYLSLQE)(LNTDAYLSLQE)(LNTDAYLSLQE)LQAQDSVHLI* |
| | 22. mouse orthoIL2Rb-IL21R SAY (mo21R SAY) |
| SEQ ID | ATGGCTACCATAGCTCTTCCCTGGAGCCTGTCCCTCTACGTCTTCCTCCTGCTCCTGGCTACACCT |
| NO: 43 | TGGGCATCTGCAGCAGTGAAAAACTGTTCCCATCTTGAATGCTTCTACAACTCAAGAGCCAATG |
| | TCTCTTGCATGTGGAGCCATGAAGAGGCTCTGAATGTCACAACCTGCCACGTCCATGCCAAGTC |
| | GAACCTGCGACACTGGAACAAAACCTGTGAGCTAACTCTTGTGAGGCAGGCATCCTGGGCCTG |
| | CAACCTGATCCTCGGGTCGTTCCCAGAGTCCCAGTCACTGACCTCCGTGGACCTCCTTGACATAA |
| | ATGTGGTGTGCTGGGAAGAGAAGGGTTGGCGTAGGGTAAAGACCTGCGACTTCCATCCCTTTG |
| | ACAACCTTCGCCTGGTGGCCCCTCATTCCCTCCAAGTTCTGCACATTGATACCCAGAGATGTAAC |
| | ATAAGCTGGAAGGTCTCCCAGGTCTCTGACTTCATTGAACCATACTTGGAATTTGAGGCCCGTA |
| | GACGTCTTCTGGGCCACAGCTGGGAGGATGCATCCGTATTAAGCCTCAAGCAGAGACAGCAGT |
| | GGCTCTTCTTGGAGATGCTGATCCCTAGTACCTCATATGAGGTCCAGGTGAGGGTCAAAGCTCA |
| | ACGAAACAATACCGGGACCTGGAGTCCCTGGAGCCAGCCCCTGACCTTTCGGACAAGACCTGC |
| | 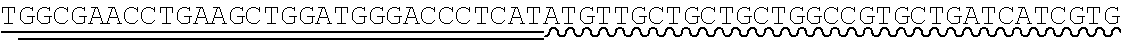 |
| | 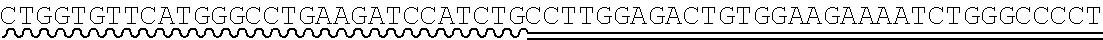 |
| | GTGCCTACTCCTGAGAGCTTCTTCCAGCCACTGTACAGAGAGCACAGCGGCAACTTCAAGAAAT |
| | GGGTCAACACCCCTTTCACCGCCAGCAGTATCGAGCTGGTGCCTCAGAGCAGCACCACAACATC |
| | TGCCCTGCACCTGTCTCTGTACCCCGCCAAAGAGAAGAAGTTCCCTGGCCTGCCTGGACTGGAA |
| | GAACAGCTGGAATGTGACGGCATGAGCGAGCCTGGCCACTGGTGTATCATTCCTCTGGCTGCT |
| | GGACAGGCCGTGTCCGCCTATAGCGAGGAAAGAGACAGACCCTACGGCCTGGTGTCCATCGAC |
| | ACAGTGACAGTGGGAGATGCCGAGGGCCTGTGTGTGTGGCCTTGTAGCTGTGAAGATGACGG |
| | CTACCCTGCCATGAACCTGGATGCCGGAAGAGAGAGCGGCCCTAACTCTGAGGATCTGCTGCT |
| | CGTGACCGATCCTGCCTTCCTGTCTTGCGGCTGTGTGTCTGGATCTGGCCTGAGACTCGGAGGC |
| | TCTCCTGGAAGCCTGCTGGATAGACTGAGACTGAGCTTCGCCAAAGAAGGCGACTGGACCGCC |
| | GATCCTACTTGGAGAACAGGATCTCCTGGCGGCGGAAGCGAATCTGAAGCAGGTTCTCCACCT |
| | GGCCTGGACATGGACACATTCGACTCTGGCTTCGCCGGCAGCGATTGTGGAAGCCCTGTGGAA |
| | ACAGACGAGGGCCCACCTagcgccTACCTGAGACAGTGGGTCGTGCGGACACCTCCTCCAGTTG |
| | ATTCTGGCGCCCAGTCCTCTTGA |
| SEQ ID | MATIALPWSLSLYVFLLLLATPWASAAVKNCSHLECFYNSRANVSCMWSHEEALNVTTCHVHAKS |
| NO: 44 | NLRHWNKTCELTLVRQASWACNLILGSFPESQSLTSVDLLDINVVCWEEKGWRRVKTCDFHPFDN |
| | LRLVAPHSLQVLHIDTQRCNISWKVSQVSDFIEPYLEFEARRRLLGHSWEDASVLSLKQRQQWLFLE |
| | 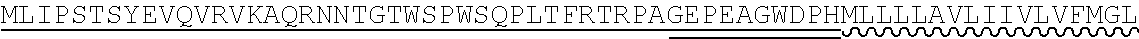 |
| | 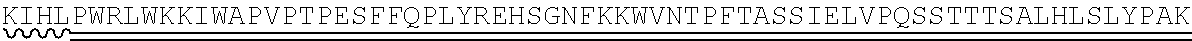 |
| | EKKFPGLPGLEEQLECDGMSEPGHWCIIPLAAGQAVSAYSEERDRPYGLVSIDTVTVGDAEGLCVW |
| | PCSCEDDGYPAMNLDAGRESGPNSEDLLLVTDPAFLSCGCVSGSGLRLGGSPGSLLDRLRLSFAKEG |
| | DWTADPTWRTGSPGGGSESEAGSPPGLDMDTFDSGFAGSDCGSPVETDEGPPSAYLRQWVVRT |
| | PPPVDSGAQSS* |
| | 23. mouse orthoIL2Rb-IL21R DAY (mo21R DAY) |
| SEQ ID | ATGGCTACCATAGCTCTTCCCTGGAGCCTGTCCCTCTACGTCTTCCTCCTGCTCCTGGCTACACCT |
| NO: 45 | TGGGCATCTGCAGCAGTGAAAAACTGTTCCCATCTTGAATGCTTCTACAACTCAAGAGCCAATG |
| | TCTCTTGCATGTGGAGCCATGAAGAGGCTCTGAATGTCACAACCTGCCACGTCCATGCCAAGTC |
| | GAACCTGCGACACTGGAACAAAACCTGTGAGCTAACTCTTGTGAGGCAGGCATCCTGGGCCTG |
| | CAACCTGATCCTCGGGTCGTTCCCAGAGTCCCAGTCACTGACCTCCGTGGACCTCCTTGACATAA |
| | ATGTGGTGTGCTGGGAAGAGAAGGGTTGGCGTAGGGTAAAGACCTGCGACTTCCATCCCTTTG |
| | ACAACCTTCGCCTGGTGGCCCCTCATTCCCTCCAAGTTCTGCACATTGATACCCAGAGATGTAAC |
| | ATAAGCTGGAAGGTCTCCCAGGTCTCTGACTTCATTGAACCATACTTGGAATTTGAGGCCCGTA |
| | GACGTCTTCTGGGCCACAGCTGGGAGGATGCATCCGTATTAAGCCTCAAGCAGAGACAGCAGT |
| | GGCTCTTCTTGGAGATGCTGATCCCTAGTACCTCATATGAGGTCCAGGTGAGGGTCAAAGCTCA |
| | ACGAAACAATACCGGGACCTGGAGTCCCTGGAGCCAGCCCCTGACCTTTCGGACAAGACCTGC |
| | 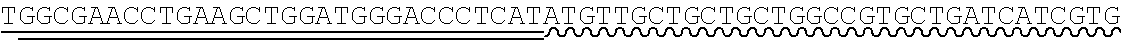 |
| | 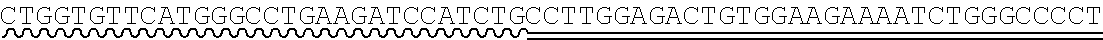 |
| | GTGCCTACTCCTGAGAGCTTCTTCCAGCCACTGTACAGAGAGCACAGCGGCAACTTCAAGAAAT |
| | GGGTCAACACCCCTTTCACCGCCAGCAGTATCGAGCTGGTGCCTCAGAGCAGCACCACAACATC |
| | TGCCCTGCACCTGTCTCTGTACCCCGCCAAAGAGAAGAAGTTCCCTGGCCTGCCTGGACTGGAA |
| | GAACAGCTGGAATGTGACGGCATGAGCGAGCCTGGCCACTGGTGTATCATTCCTCTGGCTGCT |
| | GGACAGGCCGTGTCCGCCTATAGCGAGGAAAGAGACAGACCCTACGGCCTGGTGTCCATCGAC |
| | ACAGTGACAGTGGGAGATGCCGAGGGCCTGTGTGTGTGGCCTTGTAGCTGTGAAGATGACGG |
| | CTACCCTGCCATGAACCTGGATGCCGGAAGAGAGAGCGGCCCTAACTCTGAGGATCTGCTGCT |
| | CGTGACCGATCCTGCCTTCCTGTCTTGCGGCTGTGTGTCTGGATCTGGCCTGAGACTCGGAGGC |
| | TCTCCTGGAAGCCTGCTGGATAGACTGAGACTGAGCTTCGCCAAAGAAGGCGACTGGACCGCC |
| | GATCCTACTTGGAGAACAGGATCTCCTGGCGGCGGAAGCGAATCTGAAGCAGGTTCTCCACCT |
| | GGCCTGGACATGGACACATTCGACTCTGGCTTCGCCGGCAGCGATTGTGGAAGCCCTGTGGAA |
| | ACAGACGAGGGCCCACCTgacgccTACCTGAGACAGTGGGTCGTGCGGACACCTCCTCCAGTTG |
| | ATTCTGGCGCCCAGTCCTCTTGA |
| SEQ ID | MATIALPWSLSLYVFLLLLATPWASAAVKNCSHLECFYNSRANVSCMWSHEEALNVTTCHVHAKS |
| NO: 46 | NLRHWNKTCELTLVRQASWACNLILGSFPESQSLTSVDLLDINVVCWEEKGWRRVKTCDFHPFDN |
| | LRLVAPHSLQVLHIDTQRCNISWKVSQVSDFIEPYLEFEARRRLLGHSWEDASVLSLKQRQQWLFLE |
| | 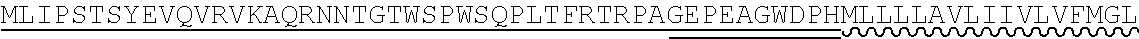 |
| | 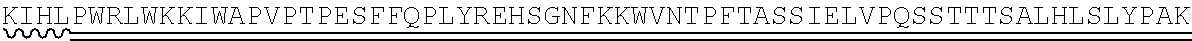 |
| | EKKFPGLPGLEEQLECDGMSEPGHWCIIPLAAGQAVSAYSEERDRPYGLVSIDTVTVGDAEGLCVW |
| | PCSCEDDGYPAMNLDAGRESGPNSEDLLLVTDPAFLSCGCVSGSGLRLGGSPGSLLDRLRLSFAKEG |
| | DWTADPTWRTGSPGGGSESEAGSPPGLDMDTFDSGFAGSDCGSPVETDEGPPDAYLRQWVVRT |
| | PPPVDSGAQSS* |
| | 24. mouse orthoIL2Rb-IL21R DAYLXQ (mo21R DAYLXQ) |
| SEQ ID | ATGGCTACCATAGCTCTTCCCTGGAGCCTGTCCCTCTACGTCTTCCTCCTGCTCCTGGCTACACCT |
| NO: 47 | TGGGCATCTGCAGCAGTGAAAAACTGTTCCCATCTTGAATGCTTCTACAACTCAAGAGCCAATG |
| | TCTCTTGCATGTGGAGCCATGAAGAGGCTCTGAATGTCACAACCTGCCACGTCCATGCCAAGTC |
| | GAACCTGCGACACTGGAACAAAACCTGTGAGCTAACTCTTGTGAGGCAGGCATCCTGGGCCTG |
| | CAACCTGATCCTCGGGTCGTTCCCAGAGTCCCAGTCACTGACCTCCGTGGACCTCCTTGACATAA |
| | ATGTGGTGTGCTGGGAAGAGAAGGGTTGGCGTAGGGTAAAGACCTGCGACTTCCATCCCTTTG |
| | ACAACCTTCGCCTGGTGGCCCCTCATTCCCTCCAAGTTCTGCACATTGATACCCAGAGATGTAAC |
| | ATAAGCTGGAAGGTCTCCCAGGTCTCTGACTTCATTGAACCATACTTGGAATTTGAGGCCCGTA |
| | GACGTCTTCTGGGCCACAGCTGGGAGGATGCATCCGTATTAAGCCTCAAGCAGAGACAGCAGT |
| | GGCTCTTCTTGGAGATGCTGATCCCTAGTACCTCATATGAGGTCCAGGTGAGGGTCAAAGCTCA |
| | ACGAAACAATACCGGGACCTGGAGTCCCTGGAGCCAGCCCCTGACCTTTCGGACAAGACCTGC |
| | 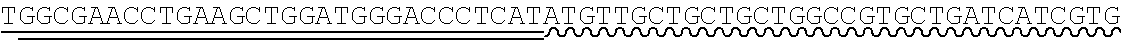 |
| | 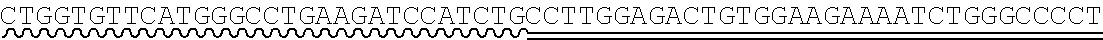 |
| | GTGCCTACTCCTGAGAGCTTCTTCCAGCCACTGTACAGAGAGCACAGCGGCAACTTCAAGAAAT |
| | GGGTCAACACCCCTTTCACCGCCAGCAGTATCGAGCTGGTGCCTCAGAGCAGCACCACAACATC |
| | TGCCCTGCACCTGTCTCTGTACCCCGCCAAAGAGAAGAAGTTCCCTGGCCTGCCTGGACTGGAA |
| | GAACAGCTGGAATGTGACGGCATGAGCGAGCCTGGCCACTGGTGTATCATTCCTCTGGCTGCT |
| | GGACAGGCCGTGTCCGCCTATAGCGAGGAAAGAGACAGACCCTACGGCCTGGTGTCCATCGAC |
| | ACAGTGACAGTGGGAGATGCCGAGGGCCTGTGTGTGTGGCCTTGTAGCTGTGAAGATGACGG |
| | CTACCCTGCCATGAACCTGGATGCCGGAAGAGAGAGCGGCCCTAACTCTGAGGATCTGCTGCT |
| | CGTGACCGATCCTGCCTTCCTGTCTTGCGGCTGTGTGTCTGGATCTGGCCTGAGACTCGGAGGC |
| | TCTCCTGGAAGCCTGCTGGATAGACTGAGACTGAGCTTCGCCAAAGAAGGCGACTGGACCGCC |
| | GATCCTACTTGGAGAACAGGATCTCCTGGCGGCGGAAGCGAATCTGAAGCAGGTTCTCCACCT |
| | GGCCTGGACATGGACACATTCGACTCTGGCTTCGCCGGCAGCGATTGTGGAAGCCCTGTGGAA |
| | ACAGACGAGGGCCCACCTgacgccTACCTGNNNCAGTGGGTCGTGCGGACACCTCCTCCAGTTG |
| | ATTCTGGCGCCCAGTCCTCTTGA |
| SEQ ID | MATIALPWSLSLYVFLLLLATPWASAAVKNCSHLECFYNSRANVSCMWSHEEALNVTTCHVHAKS |
| NO: 48 | NLRHWNKTCELTLVRQASWACNLILGSFPESQSLTSVDLLDINVVCWEEKGWRRVKTCDFHPFDN |
| | LRLVAPHSLQVLHIDTQRCNISWKVSQVSDFIEPYLEFEARRRLLGHSWEDASVLSLKQRQQWLFLE |
| | 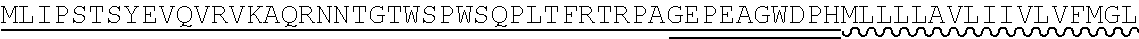 |
| | 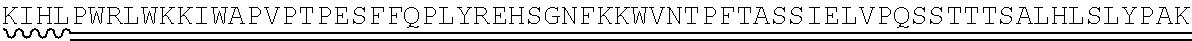 |
| | EKKFPGLPGLEEQLECDGMSEPGHWCIIPLAAGQAVSAYSEERDRPYGLVSIDTVTVGDAEGLCVW |
| | PCSCEDDGYPAMNLDAGRESGPNSEDLLLVTDPAFLSCGCVSGSGLRLGGSPGSLLDRLRLSFAKEG |
| | DWTADPTWRTGSPGGGSESEAGSPPGLDMDTFDSGFAGSDCGSPVETDEGPPDAYLXQWVVRT |
| | PPPVDSGAQSS* |
| | 25. human orthoIL2Rb-IFNAR2 (holFNAR2) |
| SEQ ID | ATGGCGGCCCCTGCTCTGTCCTGGCGTCTGCCCCTCCTCATCCTCCTCCTGCCCCTGGCTACCTCT |
| NO: 49 | TGGGCATCTGCAGCGGTGAATGGCACTTCCCAGTTCACATGCTTCTACAACTCGAGAGCCAACA |
| | TCTCCTGTGTCTGGAGCCAAGATGGGGCTCTGCAGGACACTTCCTGCCAAGTCCATGCCTGGCC |
| | GGACAGACGGCGGTGGAACCAAACCTGTGAGCTGCTCCCCGTGAGTCAAGCATCCTGGGCCTG |
| | CAACCTGATCCTCGGAGCCCCAGATTCTCAGAAACTGACCACAGTTGACATCGTCACCCTGAGG |
| | GTGCTGTGCCGTGAGGGGGTGCGATGGAGGGTGATGGCCATCCAGGACTTCAAGCCCTTTGA |
| | GAACCTTCGCCTGATGGCCCCCATCTCCCTCCAAGTTGTCCACGTGGAGACCCACAGATGCAAC |
| | ATAAGCTGGGAAATCTCCCAAGCCTCCgACTtCTTTGAAAGACACCTGGAGTTCGAGGCCCGGA |
| | CGCTGTCCCCAGGCCACACCTGGGAGGAGGCCCCCCTGCTGACTCTCAAGCAGAAGCAGGAAT |
| | GGATCTGCCTGGAGACGCTCACCCCAGACACCCAGTATGAGTTTCAGGTGCGGGTCAAGCCTC |
| | TGCAAGGCGAGTTCACGACCTGGAGCCCCTGGAGCCAGCCCCTGGCCTTCAGGACAAAGCCTG |
| | 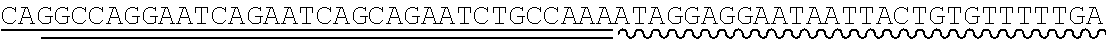 |
| | 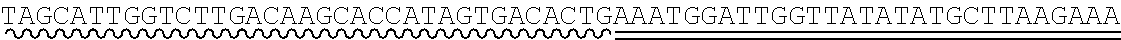 |
| | TAGCCTCCCCAAAGTCTTGAATTTTCATAACTTTTTAGCCTGGCCATTTCCTAACCTGCCACCGTT |
| | GGAAGCCATGGATATGGTGGAGGTCATTTACATCAACAGAAAGAAGAAAGTGTGGGATTATA |
| | ATTATGATGATGAAAGTGATAGCGATACTGAGGCAGCGCCCAGGACAAGTGGCGGTGGCTAT |
| | ACCATGCATGGACTGACTGTCAGGCCTCTGGGTCAGGCCTCTGCCACCTCTACAGAATCCCAGT |
| | TGATAGACCCGGAGTCCGAGGAGGAGCCTGACCTGCCTGAGGTTGATGTGGAGCTCCCCACGA |
| | TGCCAAAGGACAGCCCTCAGCAGTTGGAACTCTTGAGTGGGCCCTGTGAGAGGAGAAAGAGT |
| | CCACTCCAGGACCCTTTTCCCGAAGAGGACTACAGCTCCACGGAGGGGTCTGGGGGCAGAATT |
| | ACCTTCAATGTGGACTTAAACTCTGTGTTTTTGAGAGTTCTTGATGACGAGGACAGTGACGACT |
| | TAGAAGCCCCTCTGATGCTATCGTCTCATCTGGAAGAGATGGTTGACCCAGAGGATCCTGATAA |
| | TGTGCAATCAAACCATTTGCTGGCCAGCGGGGAAGGGACACAGCCAACCTTTCCCAGCCCCTCT |
| | TCAGAGGGCCTGTGGTCCGAAGATGCTCCATCTGATCAAAGTGACACTTCTGAGTCAGATGTTG |
| | ACCTTGGGGATGGTTATATAATGAGATGA |
| SEQ ID | MAAPALSWRLPLLILLLPLATSWASAAVNGTSQFTCFYNSRANISCVWSQDGALQDTSCQVHAWP |
| NO: 50 | DRRRWNQTCELLPVSQASWACNLILGAPDSQKLTTVDIVTLRVLCREGVRWRVMAIQDFKPFENL |
| | RLMAPISLQVVHVETHRCNISWEISQASDFFERHLEFEARTLSPGHTWEEAPLLTLKQKQEWICLETL |
| | 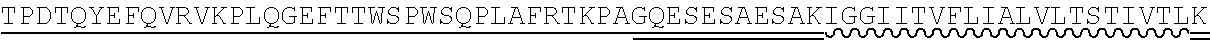 |
| | WIGYICLRNSLPKVLNFHNFLAWPFPNLPPLEAMDMVEVIYINRKKKVWDYNYDDESDSDTEAAPR |
| | TSGGGYTMHGLTVRPLGQASATSTESQLIDPESEEEPDLPEVDVELPTMPKDSPQQLELLSGPCERR |
| | KSPLQDPFPEEDYSSTEGSGGRITFNVDLNSVFLRVLDDEDSDDLEAPLMLSSHLEEMVDPEDPDNV |
| | QSNHLLASGEGTQPTFPSPSSEGLWSEDAPSDQSDTSESDVDLGDGYIMR* |
| | 26. human orthoIL2Rb-IFNGR1 (holFNGR1) |
| SEQ ID | ATGGCGGCCCCTGCTCTGTCCTGGCGTCTGCCCCTCCTCATCCTCCTCCTGCCCCTGGCTACCTCT |
| NO: 51 | TGGGCATCTGCAGCGGTGAATGGCACTTCCCAGTTCACATGCTTCTACAACTCGAGAGCCAACA |
| | TCTCCTGTGTCTGGAGCCAAGATGGGGCTCTGCAGGACACTTCCTGCCAAGTCCATGCCTGGCC |
| | GGACAGACGGCGGTGGAACCAAACCTGTGAGCTGCTCCCCGTGAGTCAAGCATCCTGGGCCTG |
| | CAACCTGATCCTCGGAGCCCCAGATTCTCAGAAACTGACCACAGTTGACATCGTCACCCTGAGG |
| | GTGCTGTGCCGTGAGGGGGTGCGATGGAGGGTGATGGCCATCCAGGACTTCAAGCCCTTTGA |
| | GAACCTTCGCCTGATGGCCCCCATCTCCCTCCAAGTTGTCCACGTGGAGACCCACAGATGCAAC |
| | ATAAGCTGGGAAATCTCCCAAGCCTCCgACTtCTTTGAAAGACACCTGGAGTTCGAGGCCCGGA |
| | CGCTGTCCCCAGGCCACACCTGGGAGGAGGCCCCCCTGCTGACTCTCAAGCAGAAGCAGGAAT |
| | GGATCTGCCTGGAGACGCTCACCCCAGACACCCAGTATGAGTTTCAGGTGCGGGTCAAGCCTC |
| | TGCAAGGCGAGTTCACGACCTGGAGCCCCTGGAGCCAGCCCCTGGCCTTCAGGACAAAGCCTG |
| | 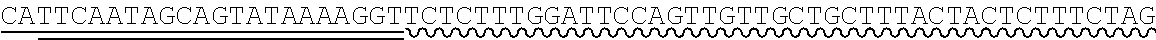 |
| | 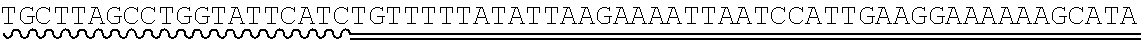 |
| | ATATTACCCAAGTCCTTGATCTCTGTGGTAAGAAGTGCTACTTTAGAGACAAAACCTGAATCAA |
| | AATATGTATCACTCATCACGTCATACCAGCCATTTTCCTTAGAAAAGGAGGTGGTCTGTGAAGA |
| | GCCGTTGTCTCCAGCAACAGTTCCAGGCATGCATACCGAAGACAATCCAGGAAAAGTGGAACA |
| | TACAGAAGAACTTTCTAGTATAACAGAAGTGGTGACTACTGAAGAAAATATTCCTGACGTGGTC |
| | CCGGGCAGCCATCTGACTCCAATAGAGAGAGAGAGTTCTTCACCTTTAAGTAGTAACCAGTCTG |
| | AACCTGGCAGCATCGCTTTAAACTCGTATCACTCCAGAAATTGTTCTGAGAGTGATCACTCCAG |
| | AAATGGTTTTGATACTGATTCCAGCTGTCTGGAATCACATAGCTCCTTATCTGACTCAGAATTTC |
| | CCCCAAATAATAAAGGTGAAATAAAAACAGAAGGACAAGAGCTCATAACCGTAATAAAAGCCC |
| | CCACCTCCTTTGGTTATGATAAACCACATGTGCTAGTGGATCTACTTGTGGATGATAGCGGTAA |
| | AGAGTCCTTGATTGGTTATAGACCAACAGAAGATTCCAAAGAATTTTCATGA |
| SEQ ID | MAAPALSWRLPLLILLLPLATSWASAAVNGTSQFTCFYNSRANISCVWSQDGALQDTSCQVHAWP |
| NO: 52 | DRRRWNQTCELLPVSQASWACNLILGAPDSQKLTTVDIVTLRVLCREGVRWRVMAIQDFKPFENL |
| | RLMAPISLQVVHVETHRCNISWEISQASDFFERHLEFEARTLSPGHTWEEAPLLTLKQKQEWICLETL |
| | 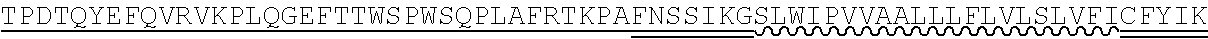 |
| | KINPLKEKSIILPKSLISVVRSATLETKPESKYVSLITSYQPFSLEKEVVCEEPLSPATVPGMHTEDNPGK |
| | VEHTEELSSITEVVTTEENIPDVVPGSHLTPIERESSSPLSSNQSEPGSIALNSYHSRNCSESDHSRNGE |
| | DTDSSCLESHSSLSDSEFPPNNKGEIKTEGQELITVIKAPTSFGYDKPHVLVDLLVDDSGKESLIGYRPT |
| | EDSKEFS* |
| | 27. human orthoIL2Rb-IFNLR1 (holFNLR1) |
| SEQ ID | ATGGCGGCCCCTGCTCTGTCCTGGCGTCTGCCCCTCCTCATCCTCCTCCTGCCCCTGGCTACCTCT |
| NO: 53 | TGGGCATCTGCAGCGGTGAATGGCACTTCCCAGTTCACATGCTTCTACAACTCGAGAGCCAACA |
| | TCTCCTGTGTCTGGAGCCAAGATGGGGCTCTGCAGGACACTTCCTGCCAAGTCCATGCCTGGCC |
| | GGACAGACGGCGGTGGAACCAAACCTGTGAGCTGCTCCCCGTGAGTCAAGCATCCTGGGCCTG |
| | CAACCTGATCCTCGGAGCCCCAGATTCTCAGAAACTGACCACAGTTGACATCGTCACCCTGAGG |
| | GTGCTGTGCCGTGAGGGGGTGCGATGGAGGGTGATGGCCATCCAGGACTTCAAGCCCTTTGA |
| | GAACCTTCGCCTGATGGCCCCCATCTCCCTCCAAGTTGTCCACGTGGAGACCCACAGATGCAAC |
| | ATAAGCTGGGAAATCTCCCAAGCCTCCgACTtCTTTGAAAGACACCTGGAGTTCGAGGCCCGGA |
| | CGCTGTCCCCAGGCCACACCTGGGAGGAGGCCCCCCTGCTGACTCTCAAGCAGAAGCAGGAAT |
| | GGATCTGCCTGGAGACGCTCACCCCAGACACCCAGTATGAGTTTCAGGTGCGGGTCAAGCCTC |
| | TGCAAGGCGAGTTCACGACCTGGAGCCCCTGGAGCCAGCCCCTGGCCTTCAGGACAAAGCCTG |
| | 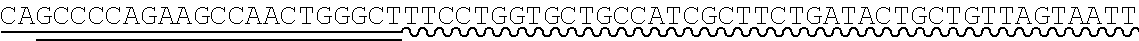 |
| | 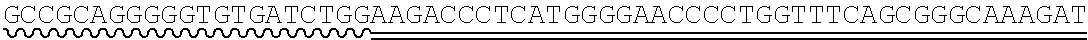 |
| | GCCACGGGCCCTGGACTTTTCTGGACACACACACCCTGTGGCAACCTTTCAGCCCAGCAGACCA |
| | GAGTCCGTGAATGACTTGTTCCTCTGTCCCCAAAAGGAACTGACCAGAGGGGTCAGGCCGACG |
| | CCTCGAGTCAGGGCCCCAGCCACCCAACAGACAAGATGGAAGAAGGACCTTGCAGAGGACGA |
| | AGAGGAGGAGGATGAGGAGGACACAGAAGATGGCGTCAGCTTCCAGCCCTACATTGAACCAC |
| | CTTCTTTCCTGGGGCAAGAGCACCAGGCTCCAGGGCACTCGGAGGCTGGTGGGGTGGACTCAG |
| | GGAGGCCCAGGGCTCCTCTGGTCCCAAGCGAAGGCTCCTCTGCTTGGGATTCTTCAGACAGAA |
| | GCTGGGCCAGCACTGTGGACTCCTCCTGGGACAGGGCTGGGTCCTCTGGCTATTTGGCTGAGA |
| | AGGGGCCAGGCCAAGGGCCGGGTGGGGATGGGCACCAAGAATCTCTCCCACCACCTGAATTC |
| | TCCAAGGACTCGGGTTTCCTGGAAGAGCTCCCAGAAGATAACCTCTCCTCCTGGGCCACCTGGG |
| | GCACCTTACCACCGGAGCCGAATCTGGTCCCTGGGGGACCCCCAGTTTCTCTTCAGACACTGAC |
| | CTTCTGCTGGGAAAGCAGCCCTGAGGAGGAAGAGGAGGCGAGGGAATCAGAAATTGAGGAC |
| | AGCGATGCGGGCAGCTGGGGGGCTGAGAGCACCCAGAGGACCGAGGACAGGGGCCGGACAT |
| | TGGGGCATTACATGGCCAGGTGA |
| SEQ ID | MAAPALSWRLPLLILLLPLATSWASAAVNGTSQFTCFYNSRANISCVWSQDGALQDTSCQVHAWP |
| NO: 54 | DRRRWNQTCELLPVSQASWACNLILGAPDSQKLTTVDIVTLRVLCREGVRWRVMAIQDFKPFENL |
| | RLMAPISLQVVHVETHRCNISWEISQASDFFERHLEFEARTLSPGHTWEEAPLLTLKQKQEWICLETL |
| | 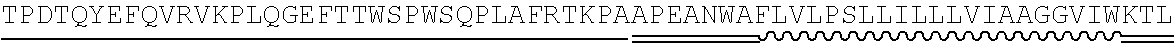 |
| | MGNPWFQRAKMPRALDFSGHTHPVATFQPSRPESVNDLFLCPQKELTRGVRPTPRVRAPATQQT |
| | RWKKDLAEDEEEEDEEDTEDGVSFQPYIEPPSFLGQEHQAPGHSEAGGVDSGRPRAPLVPSEGSSA |
| | WDSSDRSWASTVDSSWDRAGSSGYLAEKGPGQGPGGDGHQESLPPPEFSKDSGFLEELPEDNLSS |
| | WATWGTLPPEPNLVPGGPPVSLQTLTFCWESSPEEEEEARESEIEDSDAGSWGAESTQRTEDRGRT |
| | LGHYMAR* |
| | 28. human orthoIL2Rb-IL10Ra (hoIL10Ra) |
| SEQ ID | ATGGCGGCCCCTGCTCTGTCCTGGCGTCTGCCCCTCCTCATCCTCCTCCTGCCCCTGGCTACCTCT |
| NO: 55 | TGGGCATCTGCAGCGGTGAATGGCACTTCCCAGTTCACATGCTTCTACAACTCGAGAGCCAACA |
| | TCTCCTGTGTCTGGAGCCAAGATGGGGCTCTGCAGGACACTTCCTGCCAAGTCCATGCCTGGCC |
| | GGACAGACGGCGGTGGAACCAAACCTGTGAGCTGCTCCCCGTGAGTCAAGCATCCTGGGCCTG |
| | CAACCTGATCCTCGGAGCCCCAGATTCTCAGAAACTGACCACAGTTGACATCGTCACCCTGAGG |
| | GTGCTGTGCCGTGAGGGGGTGCGATGGAGGGTGATGGCCATCCAGGACTTCAAGCCCTTTGA |
| | GAACCTTCGCCTGATGGCCCCCATCTCCCTCCAAGTTGTCCACGTGGAGACCCACAGATGCAAC |
| | ATAAGCTGGGAAATCTCCCAAGCCTCCgACTtCTTTGAAAGACACCTGGAGTTCGAGGCCCGGA |
| | CGCTGTCCCCAGGCCACACCTGGGAGGAGGCCCCCCTGCTGACTCTCAAGCAGAAGCAGGAAT |
| | GGATCTGCCTGGAGACGCTCACCCCAGACACCCAGTATGAGTTTCAGGTGCGGGTCAAGCCTC |
| | TGCAAGGCGAGTTCACGACCTGGAGCCCCTGGAGCCAGCCCCTGGCCTTCAGGACAAAGCCTG |
| | 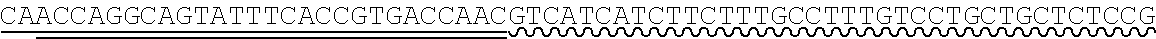 |
| | 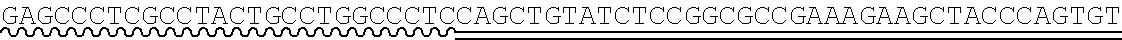 |
| | CCTGCTCTTCAAGAAGCCCAGCCCCTTCATCTTCATCAGCCAGCGTCCCTCCCCAGAGACCCAAG |
| | ACACCATCCACCCGCTTGATGAGGAGGCCTTTTTGAAGGTGTCCCCAGAGCTGAAGAACTTGG |
| | ACCTGCACGGCAGCACAGACAGTGGCTTTGGCAGCACCAAGCCATCCCTGCAGACTGAAGAGC |
| | CCCAGTTCCTCCTCCCTGACCCTCACCCCCAGGCTGACAGAACGCTGGGAAACAGGGAGCCCCC |
| | TGTGCTGGGGGACAGCTGCAGTAGTGGCAGCAGCAATAGCACAGACAGCGGGATCTGCCTGC |
| | AGGAGCCCAGCCTGAGCCCCAGCACAGGGCCCACCTGGGAGCAACAGGTGGGGAGCAACAGC |
| | AGGGGCCAGGATGACAGTGGCATTGACTTAGTTCAAAACTCTGAGGGCCGGGCTGGGGACAC |
| | ACAGGGTGGCTCGGCCTTGGGCCACCACAGTCCCCCGGAGCCTGAGGTGCCTGGGGAAGAAG |
| | ACCCAGCTGCTGTGGCATTCCAGGGTTACCTGAGGCAGACCAGATGTGCTGAAGAGAAGGCAA |
| | CCAAGACAGGCTGCCTGGAGGAAGAATCGCCCTTGACAGATGGCCTTGGCCCCAAATTCGGGA |
| | GATGCCTGGTTGATGAGGCAGGCTTGCATCCACCAGCCCTGGCCAAGGGCTATTTGAAACAGG |
| | ATCCTCTAGAAATGACTCTGGCTTCCTCAGGGGCCCCAACGGGACAGTGGAACCAGCCCACTG |
| | AGGAATGGTCACTCCTGGCCTTGAGCAGCTGCAGTGACCTGGGAATATCTGACTGGAGCTTTG |
| | CCCATGACCTTGCCCCTCTAGGCTGTGTGGCAGCCCCAGGTGGTCTCCTGGGCAGCTTTAACTC |
| | AGACCTGGTCACCCTGCCCCTCATCTCTAGCCTGCAGTCAAGTGAGTGA |
| SEQ ID | MAAPALSWRLPLLILLLPLATSWASAAVNGTSQFTCFYNSRANISCVWSQDGALQDTSCQVHAWP |
| NO: 56 | DRRRWNQTCELLPVSQASWACNLILGAPDSQKLTTVDIVTLRVLCREGVRWRVMAIQDFKPFENL |
| | RLMAPISLQVVHVETHRCNISWEISQASDFFERHLEFEARTLSPGHTWEEAPLLTLKQKQEWICLETL |
| | 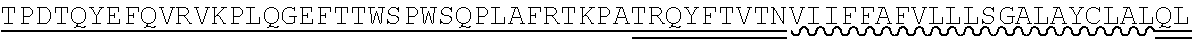 |
| | YVRRRKKLPSVLLFKKPSPFIFISQRPSPETQDTIHPLDEEAFLKVSPELKNLDLHGSTDSGFGSTKPSLQ |
| | TEEPQFLLPDPHPQADRTLGNREPPVLGDSCSSGSSNSTDSGICLQEPSLSPSTGPTWEQQVGSNSR |
| | GQDDSGIDLVQNSEGRAGDTQGGSALGHHSPPEPEVPGEEDPAAVAFQGYLRQTRCAEEKATKTG |
| | CLEEESPLTDGLGPKFGRCLVDEAGLHPPALAKGYLKQDPLEMTLASSGAPTGQWNQPTEEWSLLA |
| | LSSCSDLGISDWSFAHDLAPLGCVAAPGGLLGSFNSDLVTLPLISSLQSSE* |
| | 29. human orthoIL2Rb-IL20Ra (hoIL20Ra) |
| SEQ ID | ATGGCGGCCCCTGCTCTGTCCTGGCGTCTGCCCCTCCTCATCCTCCTCCTGCCCCTGGCTACCTCT |
| NO: 57 | TGGGCATCTGCAGCGGTGAATGGCACTTCCCAGTTCACATGCTTCTACAACTCGAGAGCCAACA |
| | TCTCCTGTGTCTGGAGCCAAGATGGGGCTCTGCAGGACACTTCCTGCCAAGTCCATGCCTGGCC |
| | GGACAGACGGCGGTGGAACCAAACCTGTGAGCTGCTCCCCGTGAGTCAAGCATCCTGGGCCTG |
| | CAACCTGATCCTCGGAGCCCCAGATTCTCAGAAACTGACCACAGTTGACATCGTCACCCTGAGG |
| | GTGCTGTGCCGTGAGGGGGTGCGATGGAGGGTGATGGCCATCCAGGACTTCAAGCCCTTTGA |
| | GAACCTTCGCCTGATGGCCCCCATCTCCCTCCAAGTTGTCCACGTGGAGACCCACAGATGCAAC |
| | ATAAGCTGGGAAATCTCCCAAGCCTCCgACTtCTTTGAAAGACACCTGGAGTTCGAGGCCCGGA |
| | CGCTGTCCCCAGGCCACACCTGGGAGGAGGCCCCCCTGCTGACTCTCAAGCAGAAGCAGGAAT |
| | GGATCTGCCTGGAGACGCTCACCCCAGACACCCAGTATGAGTTTCAGGTGCGGGTCAAGCCTC |
| | TGCAAGGCGAGTTCACGACCTGGAGCCCCTGGAGCCAGCCCCTGGCCTTCAGGACAAAGCCTG |
| | 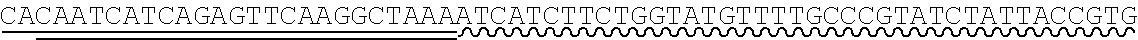 |
| | 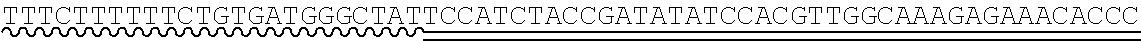 |
| | AGCAAATTTGATTTTGATTTATGGAAATGAATTTGACAAAAGATTCTTTGTGCCTGCTGAAAAA |
| | ATCGTGATTAACTTTATCACCCTCAATATCTCGGATGATTCTAAAATTTCTCATCAGGATATGAG |
| | TTTACTGGGAAAAAGCAGTGATGTATCCAGCCTTAATGATCCTCAGCCCAGCGGGAACCTGAG |
| | GCCCCCTCAGGAGGAAGAGGAGGTGAAACATTTAGGGTATGCTTCGCATTTGATGGAAATTTT |
| | TTGTGACTCTGAAGAAAACACGGAAGGTACTTCTCTCACCCAGCAAGAGTCCCTCAGCAGAACA |
| | ATACCCCCGGATAAAACAGTCATTGAATATGAATATGATGTCAGAACCACTGACATTTGTGCGG |
| | GGCCTGAAGAGCAGGAGCTCAGTTTGCAGGAGGAGGTGTCCACACAAGGAACATTATTGGAG |
| | TCGCAGGCAGCGTTGGCAGTCTTGGGCCCGCAAACGTTACAGTACTCATACACCCCTCAGCTCC |
| | AAGACTTAGACCCCCTGGCGCAGGAGCACACAGACTCGGAGGAGGGGCCGGAGGAAGAGCC |
| | ATCGACGACCCTGGTCGACTGGGATCCCCAAACTGGCAGGCTGTGTATTCCTTCGCTGTCCAGC |
| | TTCGACCAGGATTCAGAGGGCTGCGAGCCTTCTGAGGGGGATGGGCTCGGAGAGGAGGGTCT |
| | TCTATCTAGACTCTATGAGGAGCCGGCTCCAGACAGGCCACCAGGAGAAAATGAAACCTATCTC |
| | ATGCAATTCATGGAGGAATGGGGGTTATATGTGCAGATGGAAAACTGA |
| SEQ ID | MAAPALSWRLPLLILLLPLATSWASAAVNGTSQFTCFYNSRANISCVWSQDGALQDTSCQVHAWP |
| NO: 58 | DRRRWNQTCELLPVSQASWACNLILGAPDSQKLTTVDIVTLRVLCREGVRWRVMAIQDFKPFENL |
| | RLMAPISLQVVHVETHRCNISWEISQASDFFERHLEFEARTLSPGHTWEEAPLLTLKQKQEWICLETL |
| | 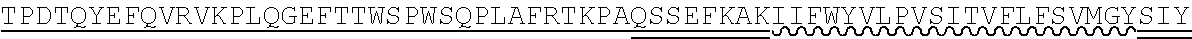 |
| | RYIHVGKEKHPANLILIYGNEFDKRFFVPAEKIVINFITLNISDDSKISHQDMSLLGKSSDVSSLNDPQP |
| | SGNLRPPQEEEEVKHLGYASHLMEIFCDSEENTEGTSLTQQESLSRTIPPDKTVIEYEYDVRTTDICAG |
| | PEEQELSLQEEVSTQGTLLESQAALAVLGPQTLQYSYTPQLQDLDPLAQEHTDSEEGPEEEPSTTLVD |
| | WDPQTGRLCIPSLSSFDQDSEGCEPSEGDGLGEEGLLSRLYEEPAPDRPPGENETYLMQFMEEWGL |
| | YVQMEN* |
| | 30. human orthoIL2Rb-IL22R (hoIL22R) |
| SEQ ID | ATGGCGGCCCCTGCTCTGTCCTGGCGTCTGCCCCTCCTCATCCTCCTCCTGCCCCTGGCTACCTCT |
| NO: 59 | TGGGCATCTGCAGCGGTGAATGGCACTTCCCAGTTCACATGCTTCTACAACTCGAGAGCCAACA |
| | TCTCCTGTGTCTGGAGCCAAGATGGGGCTCTGCAGGACACTTCCTGCCAAGTCCATGCCTGGCC |
| | GGACAGACGGCGGTGGAACCAAACCTGTGAGCTGCTCCCCGTGAGTCAAGCATCCTGGGCCTG |
| | CAACCTGATCCTCGGAGCCCCAGATTCTCAGAAACTGACCACAGTTGACATCGTCACCCTGAGG |
| | GTGCTGTGCCGTGAGGGGGTGCGATGGAGGGTGATGGCCATCCAGGACTTCAAGCCCTTTGA |
| | GAACCTTCGCCTGATGGCCCCCATCTCCCTCCAAGTTGTCCACGTGGAGACCCACAGATGCAAC |
| | ATAAGCTGGGAAATCTCCCAAGCCTCCgACTtCTTTGAAAGACACCTGGAGTTCGAGGCCCGGA |
| | CGCTGTCCCCAGGCCACACCTGGGAGGAGGCCCCCCTGCTGACTCTCAAGCAGAAGCAGGAAT |
| | GGATCTGCCTGGAGACGCTCACCCCAGACACCCAGTATGAGTTTCAGGTGCGGGTCAAGCCTC |
| | TGCAAGGCGAGTTCACGACCTGGAGCCCCTGGAGCCAGCCCCTGGCCTTCAGGACAAAGCCTG |
| | CACCAGACCGGACATGGACCTACTCCTTCTCCGGAGCCTTCCTGTTCTCCATGGGCTTCCTCGTC |
| | GCAGTACTCTGCTACCTGAGCTACAGATATGTCACCAAGCCGCCTGCACCTCCCAACTCCCTGA |
| | ACGTCCAGCGAGTCCTGACTTTCCAGCCGCTGCGCTTCATCCAGGAGCACGTCCTGATCCCTGT |
| | CTTTGACCTCAGCGGCCCCAGCAGTCTGGCCCAGCCTGTCCAGTACTCCCAGATCAGGGTGTCT |
| | GGACCCAGGGAGCCCGCAGGAGCTCCACAGCGGCATAGCCTGTCCGAGATCACCTACTTAGGG |
| | CAGCCAGACATCTCCATCCTCCAGCCCTCCAACGTGCCACCTCCCCAGATCCTCTCCCCACTGTC |
| | CTATGCCCCAAACGCTGCCCCTGAGGTCGGGCCCCCATCCTATGCACCTCAGGTGACCCCCGAA |
| | GCTCAATTCCCATTCTACGCCCCACAGGCCATCTCTAAGGTCCAGCCTTCCTCCTATGCCCCTCAA |
| | GCCACTCCGGACAGCTGGCCTCCCTCCTATGGGGTATGCATGGAAGGTTCTGGCAAAGACTCCC |
| | CCACTGGGACACTTTCTAGTCCTAAACACCTTAGGCCTAAAGGTCAGCTTCAGAAAGAGCCACC |
| | AGCTGGAAGCTGCATGTTAGGTGGCCTTTCTCTGCAGGAGGTGACCTCCTTGGCTATGGAGGA |
| | ATCCCAAGAAGCAAAATCATTGCACCAGCCCCTGGGGATTTGCACAGACAGAACATCTGACCCA |
| | AATGTGCTACACAGTGGGGAGGAAGGGACACCACAGTACCTAAAGGGCCAGCTCCCCCTCCTC |
| | TCCTCAGTCCAGATCGAGGGCCACCCCATGTCCCTCCCTTTGCAACCTCCTTCCCGTCCATGTTCC |
| | CCCTCGGACCAAGGTCCAAGTCCCTGGGGCCTGCTGGAGTCCCTTGTGTGTCCCAAGGATGAA |
| | GCCAAGAGCCCAGCCCCTGAGACCTCAGACCTGGAGCAGCCCACAGAACTGGATTCTCTTTTCA |
| | GAGGCCTGGCCCTGACTGTGCAGTGGGAGTCCTGA |
| SEQ ID | MAAPALSWRLPLLILLLPLATSWASAAVNGTSQFTCFYNSRANISCVWSQDGALQDTSCQVHAWP |
| NO: 60 | DRRRWNQTCELLPVSQASWACNLILGAPDSQKLTTVDIVTLRVLCREGVRWRVMAIQDFKPFENL |
| | RLMAPISLQVVHVETHRCNISWEISQASDFFERHLEFEARTLSPGHTWEEAPLLTLKQKQEWICLETL |
| | 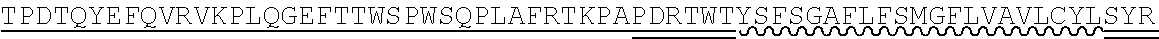 |
| | YVTKPPAPPNSLNVQRVLTFQPLRFIQEHVLIPVFDLSGPSSLAQPVQYSQIRVSGPREPAGAPQRHS |
| | LSEITYLGQPDISILQPSNVPPPQILSPLSYAPNAAPEVGPPSYAPQVTPEAQFPFYAPQAISKVQPSSY |
| | APQATPDSWPPSYGVCMEGSGKDSPTGTLSSPKHLRPKGQLQKEPPAGSCMLGGLSLQEVTSLAM |
| | EESQEAKSLHQPLGICTDRTSDPNVLHSGEEGTPQYLKGQLPLLSSVQIEGHPMSLPLQPPSRPCSPS |
| | DQGPSPWGLLESLVCPKDEAKSPAPETSDLEQPTELDSLFRGLALTVQWES* |
| | 31. human orthoIL2Rb-IL9R WT (YLPQ) (hoIL9R-YLPQ) |
| SEQ ID | ATGGCGGCCCCTGCTCTGTCCTGGCGTCTGCCCCTCCTCATCCTCCTCCTGCCCCTGGCTACCTCT |
| NO: 61 | TGGGCATCTGCAGCGGTGAATGGCACTTCCCAGTTCACATGCTTCTACAACTCGAGAGCCAACA |
| | TCTCCTGTGTCTGGAGCCAAGATGGGGCTCTGCAGGACACTTCCTGCCAAGTCCATGCCTGGCC |
| | GGACAGACGGCGGTGGAACCAAACCTGTGAGCTGCTCCCCGTGAGTCAAGCATCCTGGGCCTG |
| | CAACCTGATCCTCGGAGCCCCAGATTCTCAGAAACTGACCACAGTTGACATCGTCACCCTGAGG |
| | GTGCTGTGCCGTGAGGGGGTGCGATGGAGGGTGATGGCCATCCAGGACTTCAAGCCCTTTGA |
| | GAACCTTCGCCTGATGGCCCCCATCTCCCTCCAAGTTGTCCACGTGGAGACCCACAGATGCAAC |
| | ATAAGCTGGGAAATCTCCCAAGCCTCCgACTtCTTTGAAAGACACCTGGAGTTCGAGGCCCGGA |
| | CGCTGTCCCCAGGCCACACCTGGGAGGAGGCCCCCCTGCTGACTCTCAAGCAGAAGCAGGAAT |
| | GGATCTGCCTGGAGACGCTCACCCCAGACACCCAGTATGAGTTTCAGGTGCGGGTCAAGCCTC |
| | TGCAAGGCGAGTTCACGACCTGGAGCCCCTGGAGCCAGCCCCTGGCCTTCAGGACAAAGCCTG |
| | 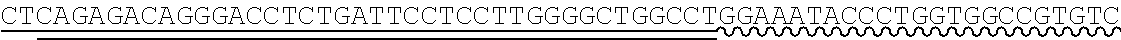 |
| | 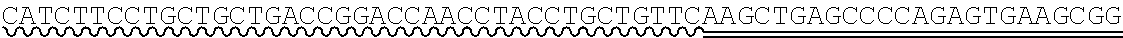 |
| | ATCTTCTACCAGAACGTGCCCTCTCCTGCCATGTTCTTCCAGCCTCTGTACAGCGTGCACAACGG |
| | CAACTTCCAGACATGGATGGGAGCACATGGTGCCGGCGTGCTGCTGTCTCAAGATTGTGCTGG |
| | AACACCCCAGGGCGCTCTGGAACCTTGTGTGCAAGAAGCCACCGCTCTGCTGACATGCGGACC |
| | TGCCAGACCTTGGAAAAGCGTGGCCCTGGAAGAGGAACAAGAAGGCCCTGGCACAAGACTGC |
| | CCGGCAATCTGTCTAGCGAGGATGTGCTGCCTGCCGGCTGTACTGAATGGCGAGTTCAGACCC |
| | TGGCCTACCTGCCTCAAGAAGATTGGGCCCCTACCAGCCTGACCAGACCTGCTCCTCCTGATAG |
| | CGAGGGCAGCAGAAGCAGCAGCAGCTCCAGCTCCTCCAACAACAACAATTACTGTGCCCTGGG |
| | CTGCTACGGCGGCTGGCATCTTTCTGCTCTGCCTGGCAACACACAGAGCAGCGGCCCTATTCCT |
| | GCTCTGGCCTGTGGCCTGAGCTGTGATCACCAGGGACTCGAGACACAGCAGGGCGTTGCATGG |
| | GTTCTCGCCGGCCATTGTCAAAGACCTGGCCTGCACGAAGATCTGCAGGGCATGCTGCTGCCTA |
| | GCGTGCTGTCTAAGGCCAGAAGCTGGACCTTCTGA |
| SEQ ID | MAAPALSWRLPLLILLLPLATSWASAAVNGTSQFTCFYNSRANISCVWSQDGALQDTSCQVHAWP |
| NO: 62 | DRRRWNQTCELLPVSQASWACNLILGAPDSQKLTTVDIVTLRVLCREGVRWRVMAIQDFKPFENL |
| | RLMAPISLQVVHVETHRCNISWEISQASDFFERHLEFEARTLSPGHTWEEAPLLTLKQKQEWICLETL |
| | 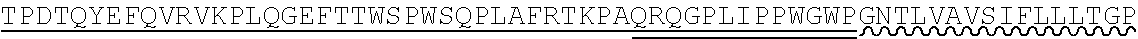 |
| | 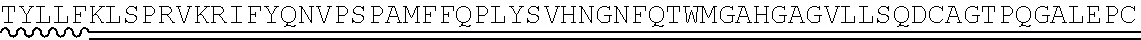 |
| | VQEATALLTCGPARPWKSVALEEEQEGPGTRLPGNLSSEDVLPAGCTEWRVQTLAYLPQEDWAPT |
| | SLTRPAPPDSEGSRSSSSSSSSNNNNYCALGCYGGWHLSALPGNTQSSGPIPALACGLSCDHQGLET |
| | QQGVAWVLAGHCQRPGLHEDLQGMLLPSVLSKARSWTF* |
| | 32. human orthoIL2Rb-IL9R mut1 (FLPQ) (holL9R-FLPQ) |
| SEQ ID | ATGGCGGCCCCTGCTCTGTCCTGGCGTCTGCCCCTCCTCATCCTCCTCCTGCCCCTGGCTACCTCT |
| NO: 63 | TGGGCATCTGCAGCGGTGAATGGCACTTCCCAGTTCACATGCTTCTACAACTCGAGAGCCAACA |
| | TCTCCTGTGTCTGGAGCCAAGATGGGGCTCTGCAGGACACTTCCTGCCAAGTCCATGCCTGGCC |
| | GGACAGACGGCGGTGGAACCAAACCTGTGAGCTGCTCCCCGTGAGTCAAGCATCCTGGGCCTG |
| | CAACCTGATCCTCGGAGCCCCAGATTCTCAGAAACTGACCACAGTTGACATCGTCACCCTGAGG |
| | GTGCTGTGCCGTGAGGGGGTGCGATGGAGGGTGATGGCCATCCAGGACTTCAAGCCCTTTGA |
| | GAACCTTCGCCTGATGGCCCCCATCTCCCTCCAAGTTGTCCACGTGGAGACCCACAGATGCAAC |
| | ATAAGCTGGGAAATCTCCCAAGCCTCCgACTtCTTTGAAAGACACCTGGAGTTCGAGGCCCGGA |
| | CGCTGTCCCCAGGCCACACCTGGGAGGAGGCCCCCCTGCTGACTCTCAAGCAGAAGCAGGAAT |
| | GGATCTGCCTGGAGACGCTCACCCCAGACACCCAGTATGAGTTTCAGGTGCGGGTCAAGCCTC |
| | TGCAAGGCGAGTTCACGACCTGGAGCCCCTGGAGCCAGCCCCTGGCCTTCAGGACAAAGCCTG |
| | 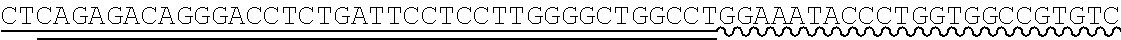 |
| | 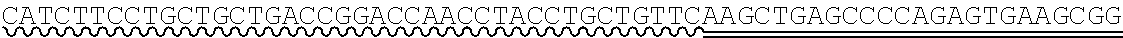 |
| | ATCTTCTACCAGAACGTGCCCTCTCCTGCCATGTTCTTCCAGCCTCTGTACAGCGTGCACAACGG |
| | CAACTTCCAGACATGGATGGGAGCACATGGTGCCGGCGTGCTGCTGTCTCAAGATTGTGCTGG |
| | AACACCCCAGGGCGCTCTGGAACCTTGTGTGCAAGAAGCCACCGCTCTGCTGACATGCGGACC |
| | TGCCAGACCTTGGAAAAGCGTGGCCCTGGAAGAGGAACAAGAAGGCCCTGGCACAAGACTGC |
| | CCGGCAATCTGTCTAGCGAGGATGTGCTGCCTGCCGGCTGTACTGAATGGCGAGTTCAGACCC |
| | TGGCCTTCCTGCCTCAAGAAGATTGGGCCCCTACCAGCCTGACCAGACCTGCTCCTCCTGATAG |
| | CGAGGGCAGCAGAAGCAGCAGCAGCTCCAGCTCCTCCAACAACAACAATTACTGTGCCCTGGG |
| | CTGCTACGGCGGCTGGCATCTTTCTGCTCTGCCTGGCAACACACAGAGCAGCGGCCCTATTCCT |
| | GCTCTGGCCTGTGGCCTGAGCTGTGATCACCAGGGACTCGAGACACAGCAGGGCGTTGCATGG |
| | GTTCTCGCCGGCCATTGTCAAAGACCTGGCCTGCACGAAGATCTGCAGGGCATGCTGCTGCCTA |
| | GCGTGCTGTCTAAGGCCAGAAGCTGGACCTTCTGA |
| SEQ ID | MAAPALSWRLPLLILLLPLATSWASAAVNGTSQFTCFYNSRANISCVWSQDGALQDTSCQVHAWP |
| NO: 64 | DRRRWNQTCELLPVSQASWACNLILGAPDSQKLTTVDIVTLRVLCREGVRWRVMAIQDFKPFENL |
| | RLMAPISLQVVHVETHRCNISWEISQASDFFERHLEFEARTLSPGHTWEEAPLLTLKQKQEWICLETL |
| | 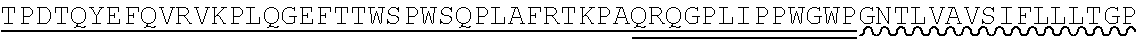 |
| | 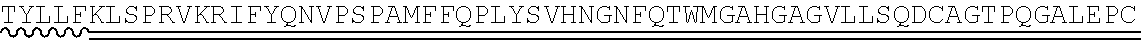 |
| | VQEATALLTCGPARPWKSVALEEEQEGPGTRLPGNLSSEDVLPAGCTEWRVQTLAFLPQEDWAPT |
| | SLTRPAPPDSEGSRSSSSSSSSNNNNYCALGCYGGWHLSALPGNTQSSGPIPALACGLSCDHQGLET |
| | QQGVAWVLAGHCQRPGLHEDLQGMLLPSVLSKARSWTF* |
| | 33. human orthoIL2Rb-IL9R mut6 (YRPQ) (hoIL9R-YRPQ) |
| SEQ ID | ATGGCGGCCCCTGCTCTGTCCTGGCGTCTGCCCCTCCTCATCCTCCTCCTGCCCCTGGCTACCTCT |
| NO: 65 | TGGGCATCTGCAGCGGTGAATGGCACTTCCCAGTTCACATGCTTCTACAACTCGAGAGCCAACA |
| | TCTCCTGTGTCTGGAGCCAAGATGGGGCTCTGCAGGACACTTCCTGCCAAGTCCATGCCTGGCC |
| | GGACAGACGGCGGTGGAACCAAACCTGTGAGCTGCTCCCCGTGAGTCAAGCATCCTGGGCCTG |
| | CAACCTGATCCTCGGAGCCCCAGATTCTCAGAAACTGACCACAGTTGACATCGTCACCCTGAGG |
| | GTGCTGTGCCGTGAGGGGGTGCGATGGAGGGTGATGGCCATCCAGGACTTCAAGCCCTTTGA |
| | GAACCTTCGCCTGATGGCCCCCATCTCCCTCCAAGTTGTCCACGTGGAGACCCACAGATGCAAC |
| | ATAAGCTGGGAAATCTCCCAAGCCTCCgACTtCTTTGAAAGACACCTGGAGTTCGAGGCCCGGA |
| | CGCTGTCCCCAGGCCACACCTGGGAGGAGGCCCCCCTGCTGACTCTCAAGCAGAAGCAGGAAT |
| | GGATCTGCCTGGAGACGCTCACCCCAGACACCCAGTATGAGTTTCAGGTGCGGGTCAAGCCTC |
| | TGCAAGGCGAGTTCACGACCTGGAGCCCCTGGAGCCAGCCCCTGGCCTTCAGGACAAAGCCTG |
| | 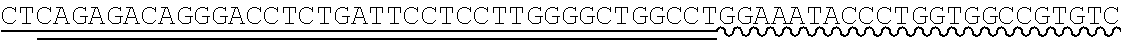 |
| | 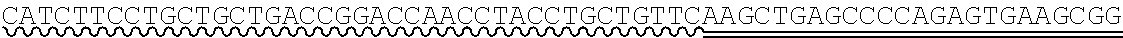 |
| | ATCTTCTACCAGAACGTGCCCTCTCCTGCCATGTTCTTCCAGCCTCTGTACAGCGTGCACAACGG |
| | CAACTTCCAGACATGGATGGGAGCACATGGTGCCGGCGTGCTGCTGTCTCAAGATTGTGCTGG |
| | AACACCCCAGGGCGCTCTGGAACCTTGTGTGCAAGAAGCCACCGCTCTGCTGACATGCGGACC |
| | TGCCAGACCTTGGAAAAGCGTGGCCCTGGAAGAGGAACAAGAAGGCCCTGGCACAAGACTGC |
| | CCGGCAATCTGTCTAGCGAGGATGTGCTGCCTGCCGGCTGTACTGAATGGCGAGTTCAGACCC |
| | TGGCCTACaggCCTCAAGAAGATTGGGCCCCTACCAGCCTGACCAGACCTGCTCCTCCTGATAGC |
| | GAGGGCAGCAGAAGCAGCAGCAGCTCCAGCTCCTCCAACAACAACAATTACTGTGCCCTGGGC |
| | TGCTACGGCGGCTGGCATCTTTCTGCTCTGCCTGGCAACACACAGAGCAGCGGCCCTATTCCTG |
| | CTCTGGCCTGTGGCCTGAGCTGTGATCACCAGGGACTCGAGACACAGCAGGGCGTTGCATGGG |
| | TTCTCGCCGGCCATTGTCAAAGACCTGGCCTGCACGAAGATCTGCAGGGCATGCTGCTGCCTAG |
| | CGTGCTGTCTAAGGCCAGAAGCTGGACCTTCTGA |
| SEQ ID | MAAPALSWRLPLLILLLPLATSWASAAVNGTSQFTCFYNSRANISCVWSQDGALQDTSCQVHAWP |
| NO: 66 | DRRRWNQTCELLPVSQASWACNLILGAPDSQKLTTVDIVTLRVLCREGVRWRVMAIQDFKPFENL |
| | RLMAPISLQVVHVETHRCNISWEISQASDFFERHLEFEARTLSPGHTWEEAPLLTLKQKQEWICLETL |
| | 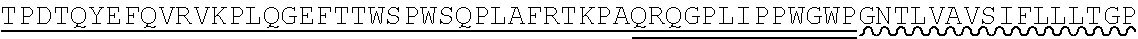 |
| | 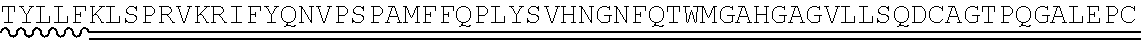 |
| | VQEATALLTCGPARPWKSVALEEEQEGPGTRLPGNLSSEDVLPAGCTEWRVQTLAYRPQEDWAPT |
| | SLTRPAPPDSEGSRSSSSSSSSNNNNYCALGCYGGWHLSALPGNTQSSGPIPALACGLSCDHQGLET |
| | QQGVAWVLAGHCQRPGLHEDLQGMLLPSVLSKARSWTF* |
| | 34. human orthoIL2Rb-IL9R mut7 (YLPL) (holL9R-YLPL) |
| SEQ ID | ATGGCGGCCCCTGCTCTGTCCTGGCGTCTGCCCCTCCTCATCCTCCTCCTGCCCCTGGCTACCTCT |
| NO: 67 | TGGGCATCTGCAGCGGTGAATGGCACTTCCCAGTTCACATGCTTCTACAACTCGAGAGCCAACA |
| | TCTCCTGTGTCTGGAGCCAAGATGGGGCTCTGCAGGACACTTCCTGCCAAGTCCATGCCTGGCC |
| | GGACAGACGGCGGTGGAACCAAACCTGTGAGCTGCTCCCCGTGAGTCAAGCATCCTGGGCCTG |
| | CAACCTGATCCTCGGAGCCCCAGATTCTCAGAAACTGACCACAGTTGACATCGTCACCCTGAGG |
| | GTGCTGTGCCGTGAGGGGGTGCGATGGAGGGTGATGGCCATCCAGGACTTCAAGCCCTTTGA |
| | GAACCTTCGCCTGATGGCCCCCATCTCCCTCCAAGTTGTCCACGTGGAGACCCACAGATGCAAC |
| | ATAAGCTGGGAAATCTCCCAAGCCTCCgACTtCTTTGAAAGACACCTGGAGTTCGAGGCCCGGA |
| | CGCTGTCCCCAGGCCACACCTGGGAGGAGGCCCCCCTGCTGACTCTCAAGCAGAAGCAGGAAT |
| | GGATCTGCCTGGAGACGCTCACCCCAGACACCCAGTATGAGTTTCAGGTGCGGGTCAAGCCTC |
| | TGCAAGGCGAGTTCACGACCTGGAGCCCCTGGAGCCAGCCCCTGGCCTTCAGGACAAAGCCTG |
| | 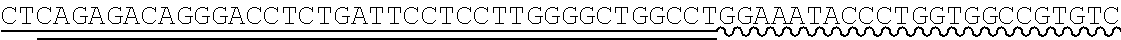 |
| | 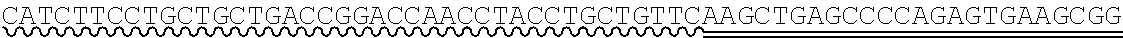 |
| | ATCTTCTACCAGAACGTGCCCTCTCCTGCCATGTTCTTCCAGCCTCTGTACAGCGTGCACAACGG |
| | CAACTTCCAGACATGGATGGGAGCACATGGTGCCGGCGTGCTGCTGTCTCAAGATTGTGCTGG |
| | AACACCCCAGGGCGCTCTGGAACCTTGTGTGCAAGAAGCCACCGCTCTGCTGACATGCGGACC |
| | TGCCAGACCTTGGAAAAGCGTGGCCCTGGAAGAGGAACAAGAAGGCCCTGGCACAAGACTGC |
| | CCGGCAATCTGTCTAGCGAGGATGTGCTGCCTGCCGGCTGTACTGAATGGCGAGTTCAGACCC |
| | TGGCCTACCTGCCTCTGGAAGATTGGGCCCCTACCAGCCTGACCAGACCTGCTCCTCCTGATAG |
| | CGAGGGCAGCAGAAGCAGCAGCAGCTCCAGCTCCTCCAACAACAACAATTACTGTGCCCTGGG |
| | CTGCTACGGCGGCTGGCATCTTTCTGCTCTGCCTGGCAACACACAGAGCAGCGGCCCTATTCCT |
| | GCTCTGGCCTGTGGCCTGAGCTGTGATCACCAGGGACTCGAGACACAGCAGGGCGTTGCATGG |
| | GTTCTCGCCGGCCATTGTCAAAGACCTGGCCTGCACGAAGATCTGCAGGGCATGCTGCTGCCTA |
| | GCGTGCTGTCTAAGGCCAGAAGCTGGACCTTCTGA |
| SEQ ID | MAAPALSWRLPLLILLLPLATSWASAAVNGTSQFTCFYNSRANISCVWSQDGALQDTSCQVHAWP |
| NO: 68 | DRRRWNQTCELLPVSQASWACNLILGAPDSQKLTTVDIVTLRVLCREGVRWRVMAIQDFKPFENL |
| | RLMAPISLQVVHVETHRCNISWEISQASDFFERHLEFEARTLSPGHTWEEAPLLTLKQKQEWICLETL |
| | 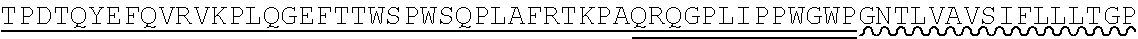 |
| | 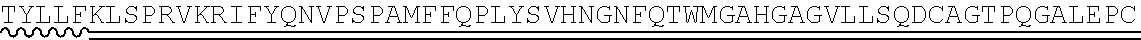 |
| | VQEATALLTCGPARPWKSVALEEEQEGPGTRLPGNLSSEDVLPAGCTEWRVQTLAYLPLEDWAPT |
| | SLTRPAPPDSEGSRSSSSSSSSNNNNYCALGCYGGWHLSALPGNTQSSGPIPALACGLSCDHQGLET |
| | QQGVAWVLAGHCQRPGLHEDLQGMLLPSVLSKARSWTF* |
| | 35. human orthoIL2Rb-IL9R mutX (YLXQ) (holL9R-YLXQ), X = any 20 AA |
| SEQ ID | ATGGCGGCCCCTGCTCTGTCCTGGCGTCTGCCCCTCCTCATCCTCCTCCTGCCCCTGGCTACCTCT |
| NO: 69 | TGGGCATCTGCAGCGGTGAATGGCACTTCCCAGTTCACATGCTTCTACAACTCGAGAGCCAACA |
| | TCTCCTGTGTCTGGAGCCAAGATGGGGCTCTGCAGGACACTTCCTGCCAAGTCCATGCCTGGCC |
| | GGACAGACGGCGGTGGAACCAAACCTGTGAGCTGCTCCCCGTGAGTCAAGCATCCTGGGCCTG |
| | CAACCTGATCCTCGGAGCCCCAGATTCTCAGAAACTGACCACAGTTGACATCGTCACCCTGAGG |
| | GTGCTGTGCCGTGAGGGGGTGCGATGGAGGGTGATGGCCATCCAGGACTTCAAGCCCTTTGA |
| | GAACCTTCGCCTGATGGCCCCCATCTCCCTCCAAGTTGTCCACGTGGAGACCCACAGATGCAAC |
| | ATAAGCTGGGAAATCTCCCAAGCCTCCgACTtCTTTGAAAGACACCTGGAGTTCGAGGCCCGGA |
| | CGCTGTCCCCAGGCCACACCTGGGAGGAGGCCCCCCTGCTGACTCTCAAGCAGAAGCAGGAAT |
| | GGATCTGCCTGGAGACGCTCACCCCAGACACCCAGTATGAGTTTCAGGTGCGGGTCAAGCCTC |
| | TGCAAGGCGAGTTCACGACCTGGAGCCCCTGGAGCCAGCCCCTGGCCTTCAGGACAAAGCCTG |
| | 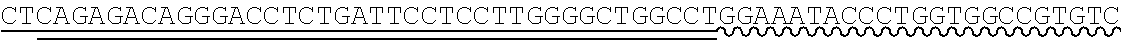 |
| | 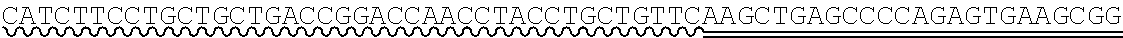 |
| | ATCTTCTACCAGAACGTGCCCTCTCCTGCCATGTTCTTCCAGCCTCTGTACAGCGTGCACAACGG |
| | CAACTTCCAGACATGGATGGGAGCACATGGTGCCGGCGTGCTGCTGTCTCAAGATTGTGCTGG |
| | AACACCCCAGGGCGCTCTGGAACCTTGTGTGCAAGAAGCCACCGCTCTGCTGACATGCGGACC |
| | TGCCAGACCTTGGAAAAGCGTGGCCCTGGAAGAGGAACAAGAAGGCCCTGGCACAAGACTGC |
| | CCGGCAATCTGTCTAGCGAGGATGTGCTGCCTGCCGGCTGTACTGAATGGCGAGTTCAGACCC |
| | TGGCCTACCTGnnnCAAGAAGATTGGGCCCCTACCAGCCTGACCAGACCTGCTCCTCCTGATAG |
| | CGAGGGCAGCAGAAGCAGCAGCAGCTCCAGCTCCTCCAACAACAACAATTACTGTGCCCTGGG |
| | CTGCTACGGCGGCTGGCATCTTTCTGCTCTGCCTGGCAACACACAGAGCAGCGGCCCTATTCCT |
| | GCTCTGGCCTGTGGCCTGAGCTGTGATCACCAGGGACTCGAGACACAGCAGGGCGTTGCATGG |
| | GTTCTCGCCGGCCATTGTCAAAGACCTGGCCTGCACGAAGATCTGCAGGGCATGCTGCTGCCTA |
| | GCGTGCTGTCTAAGGCCAGAAGCTGGACCTTCTGA |
| SEQ ID | MAAPALSWRLPLLILLLPLATSWASAAVNGTSQFTCFYNSRANISCVWSQDGALQDTSCQVHAWP |
| NO: 70 | DRRRWNQTCELLPVSQASWACNLILGAPDSQKLTTVDIVTLRVLCREGVRWRVMAIQDFKPFENL |
| | RLMAPISLQVVHVETHRCNISWEISQASDFFERHLEFEARTLSPGHTWEEAPLLTLKQKQEWICLETL |
| | 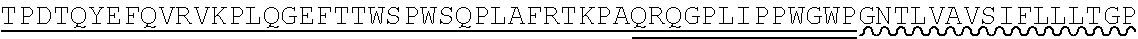 |
| | 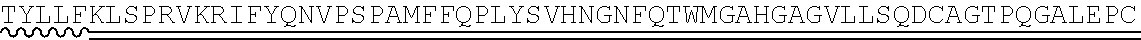 |
| | VQEATALLTCGPARPWKSVALEEEQEGPGTRLPGNLSSEDVLPAGCTEWRVQTLAYLXQEDWAPT |
| | SLTRPAPPDSEGSRSSSSSSSSNNNNYCALGCYGGWHLSALPGNTQSSGPIPALACGLSCDHQGLET |
| | QQGVAWVLAGHCQRPGLHEDLQGMLLPSVLSKARSWTF* |
| | 36. human orthoIL2Rb-IL9R 1-3x pY (holL9R-1-3xpY) |
| SEQ ID | ATGGCGGCCCCTGCTCTGTCCTGGCGTCTGCCCCTCCTCATCCTCCTCCTGCCCCTGGCTACCTCT |
| NO: 71 | TGGGCATCTGCAGCGGTGAATGGCACTTCCCAGTTCACATGCTTCTACAACTCGAGAGCCAACA |
| | TCTCCTGTGTCTGGAGCCAAGATGGGGCTCTGCAGGACACTTCCTGCCAAGTCCATGCCTGGCC |
| | GGACAGACGGCGGTGGAACCAAACCTGTGAGCTGCTCCCCGTGAGTCAAGCATCCTGGGCCTG |
| | CAACCTGATCCTCGGAGCCCCAGATTCTCAGAAACTGACCACAGTTGACATCGTCACCCTGAGG |
| | GTGCTGTGCCGTGAGGGGGTGCGATGGAGGGTGATGGCCATCCAGGACTTCAAGCCCTTTGA |
| | GAACCTTCGCCTGATGGCCCCCATCTCCCTCCAAGTTGTCCACGTGGAGACCCACAGATGCAAC |
| | ATAAGCTGGGAAATCTCCCAAGCCTCCgACTtCTTTGAAAGACACCTGGAGTTCGAGGCCCGGA |
| | CGCTGTCCCCAGGCCACACCTGGGAGGAGGCCCCCCTGCTGACTCTCAAGCAGAAGCAGGAAT |
| | GGATCTGCCTGGAGACGCTCACCCCAGACACCCAGTATGAGTTTCAGGTGCGGGTCAAGCCTC |
| | TGCAAGGCGAGTTCACGACCTGGAGCCCCTGGAGCCAGCCCCTGGCCTTCAGGACAAAGCCTG |
| | 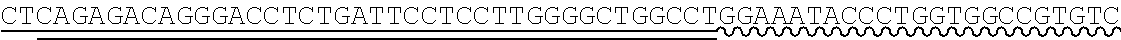 |
| | 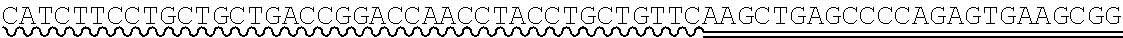 |
| | ATCTTCTACCAGAACGTGCCCTCTCCTGCCATGTTCTTCCAGCCTCTGTACAGCGTGCACAACGG |
| | CAACTTCCAGACATGGATGGGAGCACATGGTGCCGGCGTGCTGCTGTCTCAAGATTGTGCTGG |
| | AACACCCCAGGGCGCTCTGGAACCTTGTGTGCAAGAAGCCACCGCTCTGCTGACATGCGGACC |
| | TGCCAGACCTTGGAAAAGCGTGGCCCTGGAAGAGGAACAAGAAGGCCCTGGCACAAGACTGC |
| | CCGGCAATCTGTCTAGCGAGGATGTGCTGCCTGCCGGCTGTACTGAATGGCGAGTTCAGACCC |
| | TGGCCTACCTGCCTCAAGAAGATTGGGCCCCTACCAGCCTGACCAGACCTGCTCCTCCTGATAG |
| | CGAGGGCAGCAGAAGCAGCAGCAGCTCCAGCTCC(CTGAACACTGATGCCTACTTGTCCCTCCA |
| | AGAA)(CTGAACACTGATGCCTACTTGTCCCTCCAAGAA)(CTGAACACTGATGCCTACTTGTCCCT |
| | CCAAGAA)TACGGCGGCTGGCATCTTTCTGCTCTGCCTGGCAACACACAGAGCAGCGGCCCTAT |
| | TCCTGCTCTGGCCTGTGGCCTGAGCTGTGATCACCAGGGACTCGAGACACAGCAGGGCGTTGC |
| | ATGGGTTCTCGCCGGCCATTGTCAAAGACCTGGCCTGCACGAAGATCTGCAGGGCATGCTGCT |
| | GCCTAGCGTGCTGTCTAAGGCCAGAAGCTGGACCTTCTGA |
| SEQ ID | MAAPALSWRLPLLILLLPLATSWASAAVNGTSQFTCFYNSRANISCVWSQDGALQDTSCQVHAWP |
| NO: 72 | DRRRWNQTCELLPVSQASWACNLILGAPDSQKLTTVDIVTLRVLCREGVRWRVMAIQDFKPFENL |
| | RLMAPISLQVVHVETHRCNISWEISQASDFFERHLEFEARTLSPGHTWEEAPLLTLKQKQEWICLETL |
| | 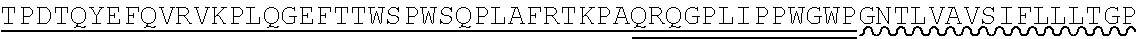 |
| | 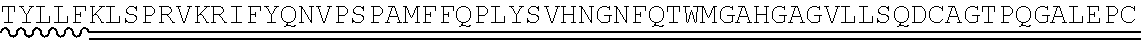 |
| | VQEATALLTCGPARPWKSVALEEEQEGPGTRLPGNLSSEDVLPAGCTEWRVQTLAYLPQEDWAPT |
| | SLTRPAPPDSEGSRSSSSSSS(LNTDAYLSLQE)(LNTDAYLSLQE)(LNTDAYLSLQE)YGGWHLSALPG |
| | NTQSSGPIPALACGLSCDHQGLETQQGVAWVLAGHCQRPGLHEDLQGMLLPSVLSKARSWTF* |
| | 37. human orthoIL2Rb-IL9R mutX 1-3x pY (holL9R mutX 1-3xpY), X = any 20 AA |
| SEQ ID | ATGGCGGCCCCTGCTCTGTCCTGGCGTCTGCCCCTCCTCATCCTCCTCCTGCCCCTGGCTACCTCT |
| NO: 73 | TGGGCATCTGCAGCGGTGAATGGCACTTCCCAGTTCACATGCTTCTACAACTCGAGAGCCAACA |
| | TCTCCTGTGTCTGGAGCCAAGATGGGGCTCTGCAGGACACTTCCTGCCAAGTCCATGCCTGGCC |
| | GGACAGACGGCGGTGGAACCAAACCTGTGAGCTGCTCCCCGTGAGTCAAGCATCCTGGGCCTG |
| | CAACCTGATCCTCGGAGCCCCAGATTCTCAGAAACTGACCACAGTTGACATCGTCACCCTGAGG |
| | GTGCTGTGCCGTGAGGGGGTGCGATGGAGGGTGATGGCCATCCAGGACTTCAAGCCCTTTGA |
| | GAACCTTCGCCTGATGGCCCCCATCTCCCTCCAAGTTGTCCACGTGGAGACCCACAGATGCAAC |
| | ATAAGCTGGGAAATCTCCCAAGCCTCCgACTtCTTTGAAAGACACCTGGAGTTCGAGGCCCGGA |
| | CGCTGTCCCCAGGCCACACCTGGGAGGAGGCCCCCCTGCTGACTCTCAAGCAGAAGCAGGAAT |
| | GGATCTGCCTGGAGACGCTCACCCCAGACACCCAGTATGAGTTTCAGGTGCGGGTCAAGCCTC |
| | TGCAAGGCGAGTTCACGACCTGGAGCCCCTGGAGCCAGCCCCTGGCCTTCAGGACAAAGCCTG |
| | 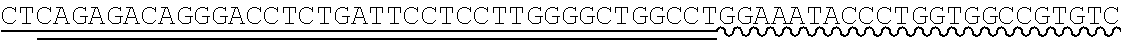 |
| | 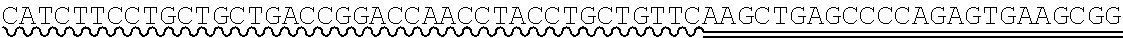 |
| | ATCTTCTACCAGAACGTGCCCTCTCCTGCCATGTTCTTCCAGCCTCTGTACAGCGTGCACAACGG |
| | CAACTTCCAGACATGGATGGGAGCACATGGTGCCGGCGTGCTGCTGTCTCAAGATTGTGCTGG |
| | AACACCCCAGGGCGCTCTGGAACCTTGTGTGCAAGAAGCCACCGCTCTGCTGACATGCGGACC |
| | TGCCAGACCTTGGAAAAGCGTGGCCCTGGAAGAGGAACAAGAAGGCCCTGGCACAAGACTGC |
| | CCGGCAATCTGTCTAGCGAGGATGTGCTGCCTGCCGGCTGTACTGAATGGCGAGTTCAGACCC |
| | TGGCCTACCTGnnnCAAGAAGATTGGGCCCCTACCAGCCTGACCAGACCTGCTCCTCCTGATAG |
| | CGAGGGCAGCAGAAGCAGCAGCAGCTCCAGCTCC(CTGAACACTGATGCCTACTTGTCCCTCCA |
| | AGAA)(CTGAACACTGATGCCTACTTGTCCCTCCAAGAA)(CTGAACACTGATGCCTACTTGTCCCT |
| | CCAAGAA)TACGGCGGCTGGCATCTTTCTGCTCTGCCTGGCAACACACAGAGCAGCGGCCCTAT |
| | TCCTGCTCTGGCCTGTGGCCTGAGCTGTGATCACCAGGGACTCGAGACACAGCAGGGCGTTGC |
| | ATGGGTTCTCGCCGGCCATTGTCAAAGACCTGGCCTGCACGAAGATCTGCAGGGCATGCTGCT |
| | GCCTAGCGTGCTGTCTAAGGCCAGAAGCTGGACCTTCTGA |
| SEQ ID | MAAPALSWRLPLLILLLPLATSWASAAVNGTSQFTCFYNSRANISCVWSQDGALQDTSCQVHAWP |
| NO: 74 | DRRRWNQTCELLPVSQASWACNLILGAPDSQKLTTVDIVTLRVLCREGVRWRVMAIQDFKPFENL |
| | RLMAPISLQVVHVETHRCNISWEISQASDFFERHLEFEARTLSPGHTWEEAPLLTLKQKQEWICLETL |
| | 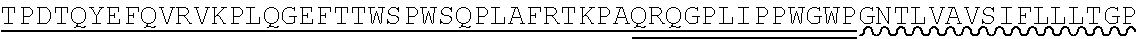 |
| | 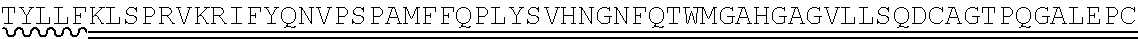 |
| | VQEATALLTCGPARPWKSVALEEEQEGPGTRLPGNLSSEDVLPAGCTEWRVQTLAYLXQEDWAPT |
| | SLTRPAPPDSEGSRSSSSSSS(LNTDAYLSLQE)(LNTDAYLSLQE)(LNTDAYLSLQE)YGGWHLSALPG |
| | NTQSSGPIPALACGLSCDHQGLETQQGVAWVLAGHCQRPGLHEDLQGMLLPSVLSKARSWTF* |
| | 38. human orthoIL2Rb-IL21R (hoIL21R) |
| SEQ ID | ATGGCGGCCCCTGCTCTGTCCTGGCGTCTGCCCCTCCTCATCCTCCTCCTGCCCCTGGCTACCTCT |
| NO: 75 | TGGGCATCTGCAGCGGTGAATGGCACTTCCCAGTTCACATGCTTCTACAACTCGAGAGCCAACA |
| | TCTCCTGTGTCTGGAGCCAAGATGGGGCTCTGCAGGACACTTCCTGCCAAGTCCATGCCTGGCC |
| | GGACAGACGGCGGTGGAACCAAACCTGTGAGCTGCTCCCCGTGAGTCAAGCATCCTGGGCCTG |
| | CAACCTGATCCTCGGAGCCCCAGATTCTCAGAAACTGACCACAGTTGACATCGTCACCCTGAGG |
| | GTGCTGTGCCGTGAGGGGGTGCGATGGAGGGTGATGGCCATCCAGGACTTCAAGCCCTTTGA |
| | GAACCTTCGCCTGATGGCCCCCATCTCCCTCCAAGTTGTCCACGTGGAGACCCACAGATGCAAC |
| | ATAAGCTGGGAAATCTCCCAAGCCTCCgACTtCTTTGAAAGACACCTGGAGTTCGAGGCCCGGA |
| | CGCTGTCCCCAGGCCACACCTGGGAGGAGGCCCCCCTGCTGACTCTCAAGCAGAAGCAGGAAT |
| | GGATCTGCCTGGAGACGCTCACCCCAGACACCCAGTATGAGTTTCAGGTGCGGGTCAAGCCTC |
| | TGCAAGGCGAGTTCACGACCTGGAGCCCCTGGAGCCAGCCCCTGGCCTTCAGGACAAAGCCTG |
| | 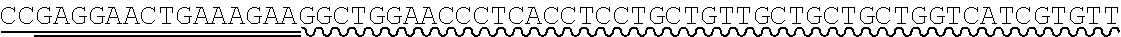 |
| | 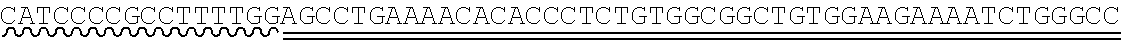 |
| | GTGCCATCTCCTGAGCGGTTCTTCATGCCTCTGTACAAGGGCTGCAGCGGCGACTTCAAGAAAT |
| | GGGTCGGAGCCCCTTTTACCGGCAGCTCTCTGGAACTTGGACCTTGGAGCCCTGAGGTGCCCA |
| | GCACACTGGAAGTGTACAGCTGTCACCCTCCTAGAAGCCCCGCCAAGAGACTGCAGCTGACAG |
| | AGCTGCAAGAACCCGCCGAGCTGGTGGAATCTGATGGCGTGCCAAAGCCTAGCTTCTGGCCCA |
| | CCGCTCAGAATAGCGGCGGCTCTGCCTACAGCGAGGAAAGAGATAGACCCTACGGCCTGGTGT |
| | CCATCGACACCGTGACAGTGCTGGATGCCGAGGGACCTTGTACCTGGCCTTGTAGCTGCGAGG |
| | ACGATGGCTACCCTGCTCTGGATCTGGACGCTGGCCTTGAGCCTTCTCCAGGACTGGAAGATCC |
| | TCTGCTGGACGCCGGAACAACCGTGCTGTCTTGTGGCTGTGTGTCTGCCGGATCTCCTGGACTT |
| | GGAGGCCCTCTGGGAAGCCTGCTGGATAGACTGAAACCTCCTCTGGCCGACGGCGAAGATTGG |
| | GCTGGTGGACTTCCTTGGGGCGGAAGATCTCCAGGCGGAGTGTCTGAGTCTGAAGCCGGTTCT |
| | CCACTGGCCGGCCTGGACATGGATACCTTCGATTCTGGCTTCGTGGGCAGCGACTGTAGCAGC |
| | CCTGTGGAATGCGACTTCACAAGCCCTGGCGACGAGGGCCCACCTAGAAGCTATCTGAGACAG |
| | TGGGTCGTGATCCCTCCACCTCTGTCTAGTCCTGGACCTCAGGCCTCTTGA |
| SEQ ID | MAAPALSWRLPLLILLLPLATSWASAAVNGTSQFTCFYNSRANISCVWSQDGALQDTSCQVHAWP |
| NO: 76 | DRRRWNQTCELLPVSQASWACNLILGAPDSQKLTTVDIVTLRVLCREGVRWRVMAIQDFKPFENL |
| | RLMAPISLQVVHVETHRCNISWEISQASDFFERHLEFEARTLSPGHTWEEAPLLTLKQKQEWICLETL |
| | 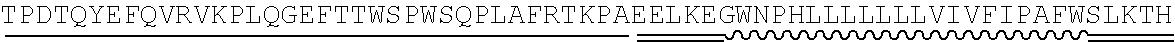 |
| | PLWRLWKKIWAVPSPERFFMPLYKGCSGDFKKWVGAPFTGSSLELGPWSPEVPSTLEVYSCHPPRS |
| | PAKRLQLTELQEPAELVESDGVPKPSFWPTAQNSGGSAYSEERDRPYGLVSIDTVTVLDAEGPCTW |
| | PCSCEDDGYPALDLDAGLEPSPGLEDPLLDAGTTVLSCGCVSAGSPGLGGPLGSLLDRLKPPLADGE |
| | DWAGGLPWGGRSPGGVSESEAGSPLAGLDMDTFDSGFVGSDCSSPVECDFTSPGDEGPPRSYLR |
| | QWVVIPPPLSSPGPQAS* |
| | 39. human orthoIL2Rb-IL21R 1-3 pY (hoIL21R 1-3xpY) |
| SEQ ID | ATGGCGGCCCCTGCTCTGTCCTGGCGTCTGCCCCTCCTCATCCTCCTCCTGCCCCTGGCTACCTCT |
| NO: 77 | TGGGCATCTGCAGCGGTGAATGGCACTTCCCAGTTCACATGCTTCTACAACTCGAGAGCCAACA |
| | TCTCCTGTGTCTGGAGCCAAGATGGGGCTCTGCAGGACACTTCCTGCCAAGTCCATGCCTGGCC |
| | GGACAGACGGCGGTGGAACCAAACCTGTGAGCTGCTCCCCGTGAGTCAAGCATCCTGGGCCTG |
| | CAACCTGATCCTCGGAGCCCCAGATTCTCAGAAACTGACCACAGTTGACATCGTCACCCTGAGG |
| | GTGCTGTGCCGTGAGGGGGTGCGATGGAGGGTGATGGCCATCCAGGACTTCAAGCCCTTTGA |
| | GAACCTTCGCCTGATGGCCCCCATCTCCCTCCAAGTTGTCCACGTGGAGACCCACAGATGCAAC |
| | ATAAGCTGGGAAATCTCCCAAGCCTCCgACTtCTTTGAAAGACACCTGGAGTTCGAGGCCCGGA |
| | CGCTGTCCCCAGGCCACACCTGGGAGGAGGCCCCCCTGCTGACTCTCAAGCAGAAGCAGGAAT |
| | GGATCTGCCTGGAGACGCTCACCCCAGACACCCAGTATGAGTTTCAGGTGCGGGTCAAGCCTC |
| | TGCAAGGCGAGTTCACGACCTGGAGCCCCTGGAGCCAGCCCCTGGCCTTCAGGACAAAGCCTG |
| | 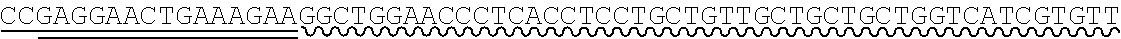 |
| | 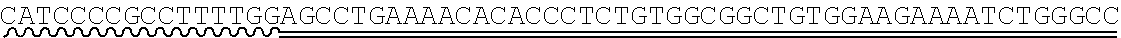 |
| | GTGCCATCTCCTGAGCGGTTCTTCATGCCTCTGTACAAGGGCTGCAGCGGCGACTTCAAGAAAT |
| | GGGTCGGAGCCCCTTTTACCGGCAGCTCTCTGGAACTTGGACCTTGGAGCCCTGAGGTGCCCA |
| | GCACACTGGAAGTGTACAGCTGTCACCCTCCTAGAAGCCCCGCCAAGAGACTGCAGCTGACAG |
| | AGCTGCAAGAACCCGCCGAGCTGGTGGAATCTGATGGCGTGCCAAAGCCTAGCTTCTGGCCCA |
| | CCGCTCAGAATAGCGGCGGCTCTGCCTACAGCGAGGAAAGAGATAGACCCTACGGCCTGGTGT |
| | CCATCGACACCGTGACAGTGCTGGATGCCGAGGGACCTTGTACCTGGCCTTGTAGCTGCGAGG |
| | ACGATGGCTACCCTGCTCTGGATCTGGACGCTGGCCTTGAGCCTTCTCCAGGACTGGAAGATCC |
| | TCTGCTGGACGCCGGAACAACCGTGCTGTCTTGTGGCTGTGTGTCTGCCGGATCTCCTGGACTT |
| | GGAGGCCCTCTGGGAAGCCTGCTGGATAGACTGAAACCTCCTCTGGCCGACGGCGAAGATTGG |
| | GCTGGTGGACTTCCTTGGGGCGGAAGATCTCCAGGCGGAGTGTCTGAGTCTGAAGCCGGTTCT |
| | CCACTGGCCGGCCTGGACATGGATACCTTCGATTCTGGCTTCGTGGGCAGCGACTGTAGCAGC |
| | CCTGTGGAATGCGACTTCACAAGCCCTGGCGACGAGGGCCCACCTAGAAGCTATCTGAGACAG |
| | TGGGTCGTGATCCCTCCACCTCTGTCTAGTCCTGGACCTCAGGCCTCT(CTGAACACGGACGCAT |
| | ATTTGTCATTGCAAGAA)(CTCAATACAGACGCCTATCTTAGCCTTCAGGAA)(TTGAATACAGAT |
| | GCTTACTTGTCTCTGCAAGAGTTGCAGGGGCAGGACCCGACCCACTTGGTTTAG) |
| SEQ ID | MAAPALSWRLPLLILLLPLATSWASAAVNGTSQFTCFYNSRANISCVWSQDGALQDTSCQVHAWP |
| NO: 78 | DRRRWNQTCELLPVSQASWACNLILGAPDSQKLTTVDIVTLRVLCREGVRWRVMAIQDFKPFENL |
| | RLMAPISLQVVHVETHRCNISWEISQASDFFERHLEFEARTLSPGHTWEEAPLLTLKQKQEWICLETL |
| | 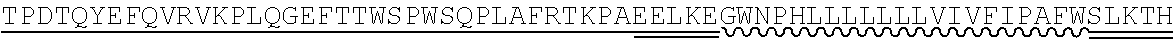 |
| | PLWRLWKKIWAVPSPERFFMPLYKGCSGDFKKWVGAPFTGSSLELGPWSPEVPSTLEVYSCHPPRS |
| | PAKRLQLTELQEPAELVESDGVPKPSFWPTAQNSGGSAYSEERDRPYGLVSIDTVTVLDAEGPCTW |
| | PCSCEDDGYPALDLDAGLEPSPGLEDPLLDAGTTVLSCGCVSAGSPGLGGPLGSLLDRLKPPLADGE |
| | DWAGGLPWGGRSPGGVSESEAGSPLAGLDMDTFDSGFVGSDCSSPVECDFTSPGDEGPPRSYLR |
| | QWVVIPPPLSSPGPQAS(LNTDAYLSLQE)(LNTDAYLSLQE)(LNTDAYLSLQELQGQDPTHLV)* |
| | 40. human orthoIL2Rb-IL21R (SAY) (hoIL21R SAY) |
| SEQ ID | ATGGCGGCCCCTGCTCTGTCCTGGCGTCTGCCCCTCCTCATCCTCCTCCTGCCCCTGGCTACCTCT |
| NO: 79 | TGGGCATCTGCAGCGGTGAATGGCACTTCCCAGTTCACATGCTTCTACAACTCGAGAGCCAACA |
| | TCTCCTGTGTCTGGAGCCAAGATGGGGCTCTGCAGGACACTTCCTGCCAAGTCCATGCCTGGCC |
| | GGACAGACGGCGGTGGAACCAAACCTGTGAGCTGCTCCCCGTGAGTCAAGCATCCTGGGCCTG |
| | CAACCTGATCCTCGGAGCCCCAGATTCTCAGAAACTGACCACAGTTGACATCGTCACCCTGAGG |
| | GTGCTGTGCCGTGAGGGGGTGCGATGGAGGGTGATGGCCATCCAGGACTTCAAGCCCTTTGA |
| | GAACCTTCGCCTGATGGCCCCCATCTCCCTCCAAGTTGTCCACGTGGAGACCCACAGATGCAAC |
| | ATAAGCTGGGAAATCTCCCAAGCCTCCgACTtCTTTGAAAGACACCTGGAGTTCGAGGCCCGGA |
| | CGCTGTCCCCAGGCCACACCTGGGAGGAGGCCCCCCTGCTGACTCTCAAGCAGAAGCAGGAAT |
| | GGATCTGCCTGGAGACGCTCACCCCAGACACCCAGTATGAGTTTCAGGTGCGGGTCAAGCCTC |
| | TGCAAGGCGAGTTCACGACCTGGAGCCCCTGGAGCCAGCCCCTGGCCTTCAGGACAAAGCCTG |
| | 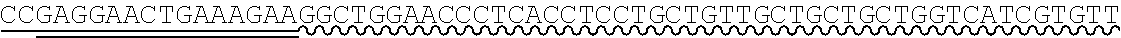 |
| | 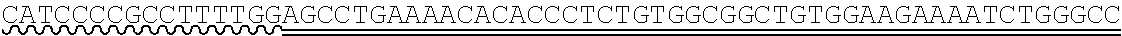 |
| | GTGCCATCTCCTGAGCGGTTCTTCATGCCTCTGTACAAGGGCTGCAGCGGCGACTTCAAGAAAT |
| | GGGTCGGAGCCCCTTTTACCGGCAGCTCTCTGGAACTTGGACCTTGGAGCCCTGAGGTGCCCA |
| | GCACACTGGAAGTGTACAGCTGTCACCCTCCTAGAAGCCCCGCCAAGAGACTGCAGCTGACAG |
| | AGCTGCAAGAACCCGCCGAGCTGGTGGAATCTGATGGCGTGCCAAAGCCTAGCTTCTGGCCCA |
| | CCGCTCAGAATAGCGGCGGCTCTGCCTACAGCGAGGAAAGAGATAGACCCTACGGCCTGGTGT |
| | CCATCGACACCGTGACAGTGCTGGATGCCGAGGGACCTTGTACCTGGCCTTGTAGCTGCGAGG |
| | ACGATGGCTACCCTGCTCTGGATCTGGACGCTGGCCTTGAGCCTTCTCCAGGACTGGAAGATCC |
| | TCTGCTGGACGCCGGAACAACCGTGCTGTCTTGTGGCTGTGTGTCTGCCGGATCTCCTGGACTT |
| | GGAGGCCCTCTGGGAAGCCTGCTGGATAGACTGAAACCTCCTCTGGCCGACGGCGAAGATTGG |
| | GCTGGTGGACTTCCTTGGGGCGGAAGATCTCCAGGCGGAGTGTCTGAGTCTGAAGCCGGTTCT |
| | CCACTGGCCGGCCTGGACATGGATACCTTCGATTCTGGCTTCGTGGGCAGCGACTGTAGCAGC |
| | CCTGTGGAATGCGACTTCACAAGCCCTGGCGACGAGGGCCCACCTAGcgcCTATCTGAGACAGT |
| | GGGTCGTGATCCCTCCACCTCTGTCTAGTCCTGGACCTCAGGCCTCTTGA |
| SEQ ID | MAAPALSWRLPLLILLLPLATSWASAAVNGTSQFTCFYNSRANISCVWSQDGALQDTSCQVHAWP |
| NO: 80 | DRRRWNQTCELLPVSQASWACNLILGAPDSQKLTTVDIVTLRVLCREGVRWRVMAIQDFKPFENL |
| | RLMAPISLQVVHVETHRCNISWEISQASDFFERHLEFEARTLSPGHTWEEAPLLTLKQKQEWICLETL |
| | 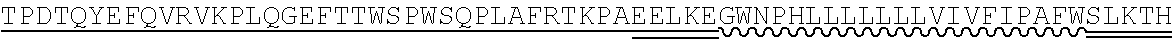 |
| | PLWRLWKKIWAVPSPERFFMPLYKGCSGDFKKWVGAPFTGSSLELGPWSPEVPSTLEVYSCHPPRS |
| | PAKRLQLTELQEPAELVESDGVPKPSFWPTAQNSGGSAYSEERDRPYGLVSIDTVTVLDAEGPCTW |
| | PCSCEDDGYPALDLDAGLEPSPGLEDPLLDAGTTVLSCGCVSAGSPGLGGPLGSLLDRLKPPLADGE |
| | DWAGGLPWGGRSPGGVSESEAGSPLAGLDMDTFDSGFVGSDCSSPVECDFTSPGDEGPPSAYLR |
| | QWVVIPPPLSSPGPQAS* |
| | 41. human orthoIL2Rb-IL21R (DAYLXQ) (holL21R DAYLXQ), X = any 20 AA |
| SEQ ID | ATGGCGGCCCCTGCTCTGTCCTGGCGTCTGCCCCTCCTCATCCTCCTCCTGCCCCTGGCTACCTCT |
| NO: 81 | TGGGCATCTGCAGCGGTGAATGGCACTTCCCAGTTCACATGCTTCTACAACTCGAGAGCCAACA |
| | TCTCCTGTGTCTGGAGCCAAGATGGGGCTCTGCAGGACACTTCCTGCCAAGTCCATGCCTGGCC |
| | GGACAGACGGCGGTGGAACCAAACCTGTGAGCTGCTCCCCGTGAGTCAAGCATCCTGGGCCTG |
| | CAACCTGATCCTCGGAGCCCCAGATTCTCAGAAACTGACCACAGTTGACATCGTCACCCTGAGG |
| | GTGCTGTGCCGTGAGGGGGTGCGATGGAGGGTGATGGCCATCCAGGACTTCAAGCCCTTTGA |
| | GAACCTTCGCCTGATGGCCCCCATCTCCCTCCAAGTTGTCCACGTGGAGACCCACAGATGCAAC |
| | ATAAGCTGGGAAATCTCCCAAGCCTCCgACTtCTTTGAAAGACACCTGGAGTTCGAGGCCCGGA |
| | CGCTGTCCCCAGGCCACACCTGGGAGGAGGCCCCCCTGCTGACTCTCAAGCAGAAGCAGGAAT |
| | GGATCTGCCTGGAGACGCTCACCCCAGACACCCAGTATGAGTTTCAGGTGCGGGTCAAGCCTC |
| | TGCAAGGCGAGTTCACGACCTGGAGCCCCTGGAGCCAGCCCCTGGCCTTCAGGACAAAGCCTG |
| | CCGAGGAACTGAAAGAAGGCTGGAACCCTCACCTCCTGCTGTTGCTGCTGCTGGTCATCGTGTT |
| | CATCCCCGCCTTTTGGAGCCTGAAAACACACCCTCTGTGGCGGCTGTGGAAGAAAATCTGGGCC |
| | GTGCCATCTCCTGAGCGGTTCTTCATGCCTCTGTACAAGGGCTGCAGCGGCGACTTCAAGAAAT |
| | GGGTCGGAGCCCCTTTTACCGGCAGCTCTCTGGAACTTGGACCTTGGAGCCCTGAGGTGCCCA |
| | GCACACTGGAAGTGTACAGCTGTCACCCTCCTAGAAGCCCCGCCAAGAGACTGCAGCTGACAG |
| | AGCTGCAAGAACCCGCCGAGCTGGTGGAATCTGATGGCGTGCCAAAGCCTAGCTTCTGGCCCA |
| | CCGCTCAGAATAGCGGCGGCTCTGCCTACAGCGAGGAAAGAGATAGACCCTACGGCCTGGTGT |
| | CCATCGACACCGTGACAGTGCTGGATGCCGAGGGACCTTGTACCTGGCCTTGTAGCTGCGAGG |
| | ACGATGGCTACCCTGCTCTGGATCTGGACGCTGGCCTTGAGCCTTCTCCAGGACTGGAAGATCC |
| | TCTGCTGGACGCCGGAACAACCGTGCTGTCTTGTGGCTGTGTGTCTGCCGGATCTCCTGGACTT |
| | GGAGGCCCTCTGGGAAGCCTGCTGGATAGACTGAAACCTCCTCTGGCCGACGGCGAAGATTGG |
| | GCTGGTGGACTTCCTTGGGGCGGAAGATCTCCAGGCGGAGTGTCTGAGTCTGAAGCCGGTTCT |
| | CCACTGGCCGGCCTGGACATGGATACCTTCGATTCTGGCTTCGTGGGCAGCGACTGTAGCAGC |
| | CCTGTGGAATGCGACTTCACAAGCCCTGGCGACGAGGGCCCACCTgacgcCTATCTGnnnCAGTG |
| | GGTCGTGATCCCTCCACCTCTGTCTAGTCCTGGACCTCAGGCCTCTTGA |
| SEQ ID | MAAPALSWRLPLLILLLPLATSWASAAVNGTSQFTCFYNSRANISCVWSQDGALQDTSCQVHAWP |
| NO: 82 | DRRRWNQTCELLPVSQASWACNLILGAPDSQKLTTVDIVTLRVLCREGVRWRVMAIQDFKPFENL |
| | RLMAPISLQVVHVETHRCNISWEISQASDFFERHLEFEARTLSPGHTWEEAPLLTLKQKQEWICLETL |
| | 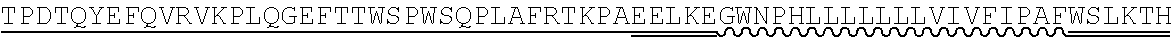 |
| | PLWRLWKKIWAVPSPERFFMPLYKGCSGDFKKWVGAPFTGSSLELGPWSPEVPSTLEVYSCHPPRS |
| | PAKRLQLTELQEPAELVESDGVPKPSFWPTAQNSGGSAYSEERDRPYGLVSIDTVTVLDAEGPCTW |
| | PCSCEDDGYPALDLDAGLEPSPGLEDPLLDAGTTVLSCGCVSAGSPGLGGPLGSLLDRLKPPLADGE |
| | DWAGGLPWGGRSPGGVSESEAGSPLAGLDMDTFDSGFVGSDCSSPVECDFTSPGDEGPPDAYLX |
| | QWVVIPPPLSSPGPQAS* |
| | IL2Rb Carboxy-terminus continaing 3x pSTAT5 binding sites |
| SEQ ID | 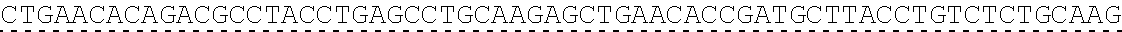 |
| NO: 83 | 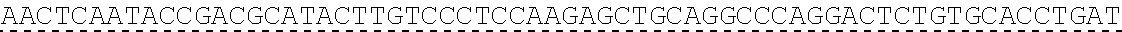 |
| | 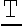 |
| | IL2Rb Carboxy-terminus containing 3x pSTAT5 binding sites |
| SEQ ID | (LNTDAYLSLQE)(LNTDAYLSLQE)(LNTDAYLSLQELQAQDSVHLI) |
| NO: 84 | |
| | mouse orthoIL2Rb-GCSFR_2Rb-TM (moGCSFR_2Rb) |
| SEQ ID | ATGGCTACCATAGCTCTTCCCTGGAGCCTGTCCCTCTACGTCTTCCTCCTGCTCCTGGCTACACCT |
| NO: 85 | TGGGCATCTGCAGCAGTGAAAAACTGTTCCCATCTTGAATGCTTCTACAACTCAAGAGCCAATG |
| | TCTCTTGCATGTGGAGCCATGAAGAGGCTCTGAATGTCACAACCTGCCACGTCCATGCCAAGTC |
| | GAACCTGCGACACTGGAACAAAACCTGTGAGCTAACTCTTGTGAGGCAGGCATCCTGGGCCTG |
| | CAACCTGATCCTCGGGTCGTTCCCAGAGTCCCAGTCACTGACCTCCGTGGACCTCCTTGACATAA |
| | ATGTGGTGTGCTGGGAAGAGAAGGGTTGGCGTAGGGTAAAGACCTGCGACTTCCATCCCTTTG |
| | ACAACCTTCGCCTGGTGGCCCCTCATTCCCTCCAAGTTCTGCACATTGATACCCAGAGATGTAAC |
| | ATAAGCTGGAAGGTCTCCCAGGTCTCTGACTTCATTGAACCATACTTGGAATTTGAGGCCCGTA |
| | GACGTCTTCTGGGCCACAGCTGGGAGGATGCATCCGTATTAAGCCTCAAGCAGAGACAGCAGT |
| | GGCTCTTCTTGGAGATGCTGATCCCTAGTACCTCATATGAGGTCCAGGTGAGGGTCAAAGCTCA |
| | ACGAAACAATACCGGGACCTGGAGTCCCTGGAGCCAGCCCCTGACCTTTCGGACAAGGCCAGC |
| | AGATCCCATGAAGGAGATCCTCCCCATGTCATGGCTCAGATACCTTCTGCTGGTCCTTGGTTGTT |
| | TTTCTGGCTTCTTCTCCTGCGTCTACATTTTGGTCAAACGCAGAGGAAAGACTTCCTTCTGGTCA |
| | GATGTGCCAGACCCAGCCCACAGTAGCCTGAGCTCCTGGTTGCCCACCATCATGACAGAGGAA |
| | ACCTTCCAGTTACCCAGCTTCTGGGACTCCAGCGTGCCATCAATCACCAAGATCACTGAACTGG |
| | AGGAAGACAAGAAACCGACCCACTGGGATTCCGAAAGCTCTGGGAATGGTAGCCTTCCAGCCC |
| | TGGTTCAGGCCTATGTGCTCCAAGGAGATCCAAGAGAAATTTCCAACCAGTCCCAGCCTCCCTC |
| | TCGCACTGGTGACCAGGTCCTCTATGGTCAGGTGCTTGAGAGCCCCACCAGCCCAGGAGTAAT |
| | GCAGTACATTCGCTCTGACTCCACTCAGCCCCTCTTGGGGGGCCCCACCCCTAGCCCTAAATCTT |
| | ATGAAAACATCTGGTTCCATTCAAGACCCCAGGAGACCTTTGTGCCCCAACCTCCAAACCAGGA |
| | AGATGACTGTGTCTTTGGGCCTCCATTTGATTTTCCCCTCTTTCAGGGGCTCCAGGTCCATGGAG |
| | TTGAAGAACAAGGGGGTTTCTGA |
| SEQ ID | MATIALPWSLSLYVFLLLLATPWASAAVKNCSHLECFYNSRANVSCMWSHEEALNVTTCHVHAKS |
| NO: 86 | NLRHWNKTCELTLVRQASWACNLILGSFPESQSLTSVDLLDINVVCWEEKGWRRVKTCDFHPFDN |
| | LRLVAPHSLQVLHIDTQRCNISWKVSQVSDFIEPYLEFEARRRLLGHSWEDASVLSLKQRQQWLFLE |
| | 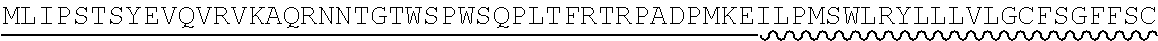 |
| | 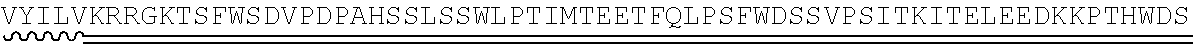 |
| | ESSGNGSLPALVQAYVLOGDPREISNQSQPPSRTGDQVLYGQVLESPTSPGVMQYIRSDSTOPLLG |
| | GPTPSPKSYENIWFHSRPQETFVPQPPNQEDDCVFGPPFDFPLFQGLOVHGVEEQGGF* |
| | mouse orthoIL2Rb-GCSFR_CSFR-TM (moGCSFR_CSFR) |
| SEQ ID | ATGGCTACCATAGCTCTTCCCTGGAGCCTGTCCCTCTACGTCTTCCTCCTGCTCCTGGCTACACCT |
| NO: 87 | TGGGCATCTGCAGCAGTGAAAAACTGTTCCCATCTTGAATGCTTCTACAACTCAAGAGCCAATG |
| | TCTCTTGCATGTGGAGCCATGAAGAGGCTCTGAATGTCACAACCTGCCACGTCCATGCCAAGTC |
| | GAACCTGCGACACTGGAACAAAACCTGTGAGCTAACTCTTGTGAGGCAGGCATCCTGGGCCTG |
| | CAACCTGATCCTCGGGTCGTTCCCAGAGTCCCAGTCACTGACCTCCGTGGACCTCCTTGACATAA |
| | ATGTGGTGTGCTGGGAAGAGAAGGGTTGGCGTAGGGTAAAGACCTGCGACTTCCATCCCTTTG |
| | ACAACCTTCGCCTGGTGGCCCCTCATTCCCTCCAAGTTCTGCACATTGATACCCAGAGATGTAAC |
| | ATAAGCTGGAAGGTCTCCCAGGTCTCTGACTTCATTGAACCATACTTGGAATTTGAGGCCCGTA |
| | GACGTCTTCTGGGCCACAGCTGGGAGGATGCATCCGTATTAAGCCTCAAGCAGAGACAGCAGT |
| | GGCTCTTCTTGGAGATGCTGATCCCTAGTACCTCATATGAGGTCCAGGTGAGGGTCAAAGCTCA |
| | ACGAAACAATACCGGGACCTGGAGTCCCTGGAGCCAGCCCCTGACCTTTCGGACAAGGCCAGC |
| | ATCTGACTTAAACATTTTCCTGGGCATACTTTGCTTAGTACTCTTGTCCACTACCTGTGTAGTGAC |
| | CTGGCTCTGCTGCAAACGCAGAGGAAAGACTTCCTTCTGGTCAGATGTGCCAGACCCAGCCCAC |
| | AGTAGCCTGAGCTCCTGGTTGCCCACCATCATGACAGAGGAAACCTTCCAGTTACCCAGCTTCT |
| | GGGACTCCAGCGTGCCATCAATCACCAAGATCACTGAACTGGAGGAAGACAAGAAACCGACCC |
| | ACTGGGATTCCGAAAGCTCTGGGAATGGTAGCCTTCCAGCCCTGGTTCAGGCCTATGTGCTCCA |
| | AGGAGATCCAAGAGAAATTTCCAACCAGTCCCAGCCTCCCTCTCGCACTGGTGACCAGGTCCTC |
| | TATGGTCAGGTGCTTGAGAGCCCCACCAGCCCAGGAGTAATGCAGTACATTCGCTCTGACTCCA |
| | CTCAGCCCCTCTTGGGGGGCCCCACCCCTAGCCCTAAATCTTATGAAAACATCTGGTTCCATTCA |
| | AGACCCCAGGAGACCTTTGTGCCCCAACCTCCAAACCAGGAAGATGACTGTGTCTTTGGGCCTC |
| | CATTTGATTTTCCCCTCTTTCAGGGGCTCCAGGTCCATGGAGTTGAAGAACAAGGGGGTTTCTG |
| | A |
| SEQ ID | MATIALPWSLSLYVFLLLLATPWASAAVKNCSHLECFYNSRANVSCMWSHEEALNVTTCHVHAKS |
| NO: 88 | NLRHWNKTCELTLVRQASWACNLILGSFPESQSLTSVDLLDINVVCWEEKGWRRVKTCDFHPFDN |
| | LRLVAPHSLQVLHIDTQRCNISWKVSQVSDFIEPYLEFEARRRLLGHSWEDASVLSLKQRQQWLFLE |
| | 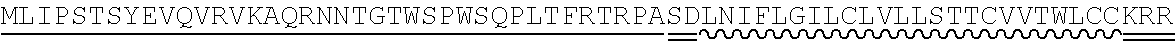 |
| | GKTSFWSDVPDPAHSSLSSWLPTIMTEETFQLPSFWDSSVPSITKITELEEDKKPTHWDSESSGNGSL |
| | PALVQAYVLQGDPREISNQSQPPSRTGDQVLYGQVLESPTSPGVMQYIRSDSTQPLLGGPTPSPKS |
| | YENIWFHSRPQETFVPQPPNQEDDCVFGPPFDFPLFQGLQVHGVEEQGGF* |
| | Human orthoIL2Rb-GCSFR_2Rb-TM (hoGCSFR_2Rb) |
| SEQ ID | ATGGCGGCCCCTGCTCTGTCCTGGCGTCTGCCCCTCCTCATCCTCCTCCTGCCCCTGGCTACCTCT |
| NO: 89 | TGGGCATCTGCAGCGGTGAATGGCACTTCCCAGTTCACATGCTTCTACAACTCGAGAGCCAACA |
| | TCTCCTGTGTCTGGAGCCAAGATGGGGCTCTGCAGGACACTTCCTGCCAAGTCCATGCCTGGCC |
| | GGACAGACGGCGGTGGAACCAAACCTGTGAGCTGCTCCCCGTGAGTCAAGCATCCTGGGCCTG |
| | CAACCTGATCCTCGGAGCCCCAGATTCTCAGAAACTGACCACAGTTGACATCGTCACCCTGAGG |
| | GTGCTGTGCCGTGAGGGGGTGCGATGGAGGGTGATGGCCATCCAGGACTTCAAGCCCTTTGA |
| | GAACCTTCGCCTGATGGCCCCCATCTCCCTCCAAGTTGTCCACGTGGAGACCCACAGATGCAAC |
| | ATAAGCTGGGAAATCTCCCAAGCCTCCGACTTCTTTGAAAGACACCTGGAGTTCGAGGCCCGG |
| | ACGCTGTCCCCAGGCCACACCTGGGAGGAGGCCCCCCTGCTGACTCTCAAGCAGAAGCAGGAA |
| | TGGATCTGCCTGGAGACGCTCACCCCAGACACCCAGTATGAGTTTCAGGTGCGGGTCAAGCCT |
| | CTGCAAGGCGAGTTCACGACCTGGAGCCCCTGGAGCCAGCCCCTGGCCTTCAGGACAAAGCCT |
| | GCAGCCCTTGGGAAGGACACCATTCCGTGGCTCGGCCACCTCCTCGTGGGTCTCAGCGGGGCT |
| | TTTGGCTTCATCATCTTAGTGTACTTGCTGATCAGCCCCAACAGGAAGAATCCCCTCTGGCCAAG |
| | TGTCCCAGACCCAGCTCACAGCAGCCTGGGCTCCTGGGTGCCCACAATCATGGAGGAGGATGC |
| | CTTCCAGCTGCCCGGCCTTGGCACGCCACCCATCACCAAGCTCACAGTGCTGGAGGAGGATGA |
| | AAAGAAGCCGGTGCCCTGGGAGTCCCATAACAGCTCAGAGACCTGTGGCCTCCCCACTCTGGT |
| | CCAGACCTATGTGCTCCAGGGGGACCCAAGAGCAGTTTCCACCCAGCCCCAATCCCAGTCTGGC |
| | ACCAGCGATCAGGTCCTTTATGGGCAGCTGCTGGGCAGCCCCACAAGCCCAGGGCCAGGGCAC |
| | TATCTCCGCTGTGACTCCACTCAGCCCCTCTTGGCGGGCCTCACCCCCAGCCCCAAGTCCTATGA |
| | GAACCTCTGGTTCCAGGCCAGCCCCTTGGGGACCCTGGTAACCCCAGCCCCAAGCCAGGAGGA |
| | CGACTGTGTCTTTGGGCCACTGCTCAACTTCCCCCTCCTGCAGGGGATCCGGGTCCATGGGATG |
| | GAGGCGCTGGGGAGCTTCTAG |
| SEQ ID | MAAPALSWRLPLLILLLPLATSWASAAVNGTSQFTCFYNSRANISCVWSQDGALQDTSCQVHAWP |
| NO:90 | DRRRWNQTCELLPVSQASWACNLILGAPDSQKLTTVDIVTLRVLCREGVRWRVMAIQDFKPFENL |
| | RLMAPISLOVVHVETHRCNISWEISQASDFFERHLEFEARTLSPGHTWEEAPLLTLKOKQEWICLETL |
| | 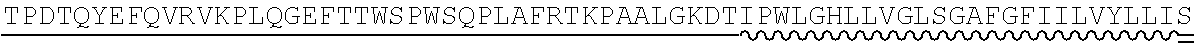 |
| | PNRKNPLWPSVPDPAHSSLGSWVPTIMEEDAFQLPGLGTPPITKLTVLEEDEKKPVPWESHNSSET |
| | CGLPTLVQTYVLOGDPRAVSTOPQSQSGTSDQVLYGOLLGSPTSPGPGHYLRCDSTOPLLAGLTPS |
| | PKSYENLWFQASPLGTLVTPAPSQEDDCVFGPLLNFPLLQGIRVHGMEALGSF* |
| | Human orthoIL2Rb-GCSFR_CSFR-TM (hoGCSFR_CSFR) |
| SEQ ID | ATGGCGGCCCCTGCTCTGTCCTGGCGTCTGCCCCTCCTCATCCTCCTCCTGCCCCTGGCTACCTCT |
| NO: 91 | TGGGCATCTGCAGCGGTGAATGGCACTTCCCAGTTCACATGCTTCTACAACTCGAGAGCCAACA |
| | TCTCCTGTGTCTGGAGCCAAGATGGGGCTCTGCAGGACACTTCCTGCCAAGTCCATGCCTGGCC |
| | GGACAGACGGCGGTGGAACCAAACCTGTGAGCTGCTCCCCGTGAGTCAAGCATCCTGGGCCTG |
| | CAACCTGATCCTCGGAGCCCCAGATTCTCAGAAACTGACCACAGTTGACATCGTCACCCTGAGG |
| | GTGCTGTGCCGTGAGGGGGTGCGATGGAGGGTGATGGCCATCCAGGACTTCAAGCCCTTTGA |
| | GAACCTTCGCCTGATGGCCCCCATCTCCCTCCAAGTTGTCCACGTGGAGACCCACAGATGCAAC |
| | ATAAGCTGGGAAATCTCCCAAGCCTCCGACTTCTTTGAAAGACACCTGGAGTTCGAGGCCCGG |
| | ACGCTGTCCCCAGGCCACACCTGGGAGGAGGCCCCCCTGCTGACTCTCAAGCAGAAGCAGGAA |
| | TGGATCTGCCTGGAGACGCTCACCCCAGACACCCAGTATGAGTTTCAGGTGCGGGTCAAGCCT |
| | CTGCAAGGCGAGTTCACGACCTGGAGCCCCTGGAGCCAGCCCCTGGCCTTCAGGACAAAGCCT |
| | TCGGAGCTACACATCATCCTGGGCCTGTTCGGCCTCCTGCTGTTGCTCACCTGCCTCTGTGGAAC |
| | TGCCTGGCTCTGTTGCAGCCCCAACAGGAAGAATCCCCTCTGGCCAAGTGTCCCAGACCCAGCT |
| | CACAGCAGCCTGGGCTCCTGGGTGCCCACAATCATGGAGGAGGATGCCTTCCAGCTGCCCGGC |
| | CTTGGCACGCCACCCATCACCAAGCTCACAGTGCTGGAGGAGGATGAAAAGAAGCCGGTGCCC |
| | TGGGAGTCCCATAACAGCTCAGAGACCTGTGGCCTCCCCACTCTGGTCCAGACCTATGTGCTCC |
| | AGGGGGACCCAAGAGCAGTTTCCACCCAGCCCCAATCCCAGTCTGGCACCAGCGATCAGGTCC |
| | TTTATGGGCAGCTGCTGGGCAGCCCCACAAGCCCAGGGCCAGGGCACTATCTCCGCTGTGACT |
| | CCACTCAGCCCCTCTTGGCGGGCCTCACCCCCAGCCCCAAGTCCTATGAGAACCTCTGGTTCCA |
| | GGCCAGCCCCTTGGGGACCCTGGTAACCCCAGCCCCAAGCCAGGAGGACGACTGTGTCTTTGG |
| | GCCACTGCTCAACTTCCCCCTCCTGCAGGGGATCCGGGTCCATGGGATGGAGGCGCTGGGGAG |
| | CTTCTAG |
| SEQ ID | MAAPALSWRLPLLILLLPLATSWASAAVNGTSQFTCFYNSRANISCVWSQDGALQDTSCQVHAWP |
| NO: 92 | DRRRWNQTCELLPVSQASWACNLILGAPDSQKLTTVDIVTLRVLCREGVRWRVMAIQDFKPFENL |
| | RLMAPISLQVVHVETHRCNISWEISQASDFFERHLEFEARTLSPGHTWEEAPLLTLKQKQEWICLETL |
| | 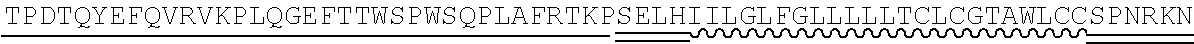 |
| | PLWPSVPDPAHSSLGSWVPTIMEEDAFQLPGLGTPPITKLTVLEEDEKKPVPWESHNSSETCGLPTL |
| | VQTYVLQGDPRAVSTQPQSQSGTSDQVLYGQLLGSPTSPGPGHYLRCDSTQPLLAGLTPSPKSYEN |
| | LWFQASPLGTLVTPAPSQEDDCVFGPLLNFPLLQGIRVHGMEALGSF* |
| SEQ ID | LNTDAYLSLQE |
| NO: 93 | |

### Results

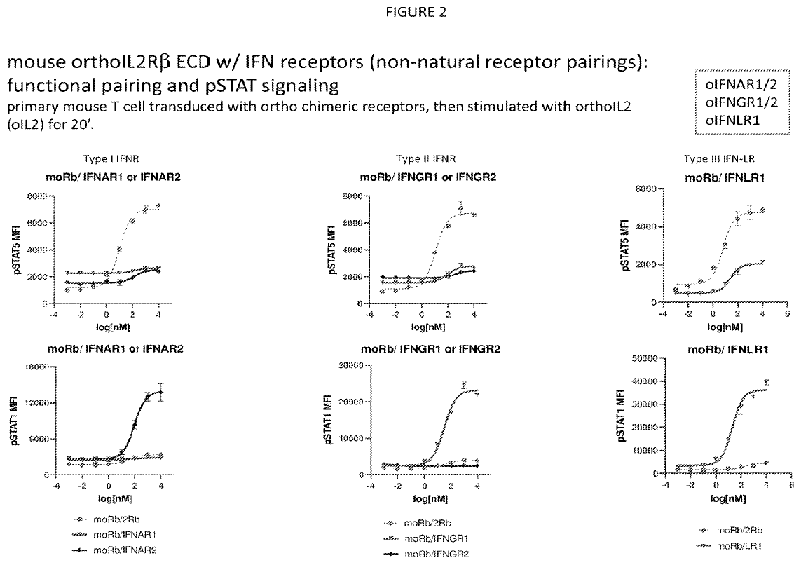

Mouse T cell blasts were virally transduced with either moRb/IFNAR1, moRb/IFNAR2 (Typel IFN receptors); moRb/IFNGR1, moRb/IFNGR2 (Type II IFN receptors); or moRb/IFNLR1 (Typelll IFN receptor). Transduced cells were stimulated with dose titration concentrations of mouse ortho-IL2 for 20′, then fixed in paraformaldehyde (PFA), methanol (MeOH) permeablilized and stained with anti-pSTAT5-A647 or anti-pSTAT1-A647. Samples were analyzed on a Cytoflex (Beckman Coulter), gating on YFP+ cells; data were plotted in Prism (GraphPad) n=3, SEM. MoRb/2Rb receptor was included as positive control for orthoIL2 signaling. It can be seen in FIG. 2 that the STAT signaling follows the specificity of the ICD, and that the tunable receptor was able to activate STAT1 from an IFNAR2 ICD and IFNGR1 ICD. The activation takes advantage of pairing with the γc chain, and is specific for the ECD ligand binding. Mouse ortho-IL-2 is as described in U.S. Pat. No. 10,869,887.

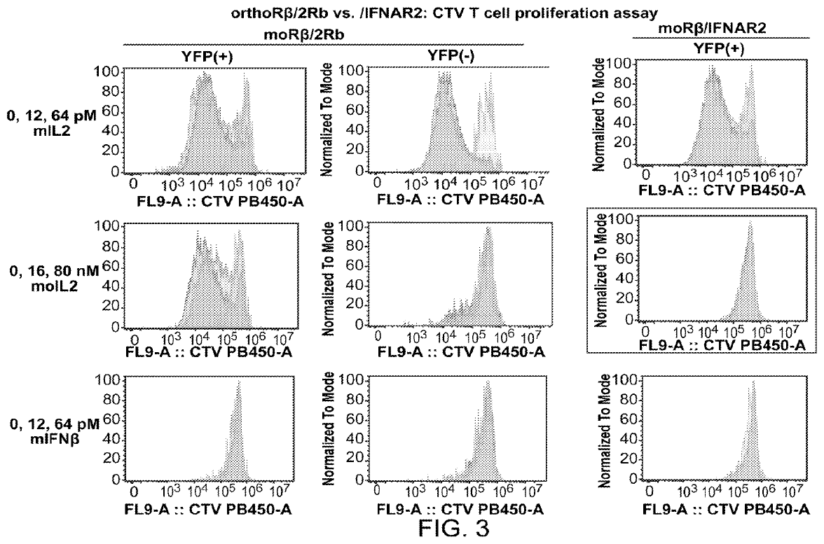

The moRb/IFNAR2 chimera phenocopies the native IFN anti-proliferative effect, shown in FIG. 3. moRb/IFNAR2 expressing cells proliferate in presence of IL2, but are anti-proliferative in presence of ortholl2. Mouse T cell blasts were transduced with moRb/IFNAR2-IRES-YFP retrovirus. Cells were labeled with CellTracer Violet (CTV) on day 0, and cultured with indicated concentration of mIL2, moIL2, or m1FNb. On day 4, samples were analyzed on Cytoflex, gating on live, YFP (+) or (−) cells.

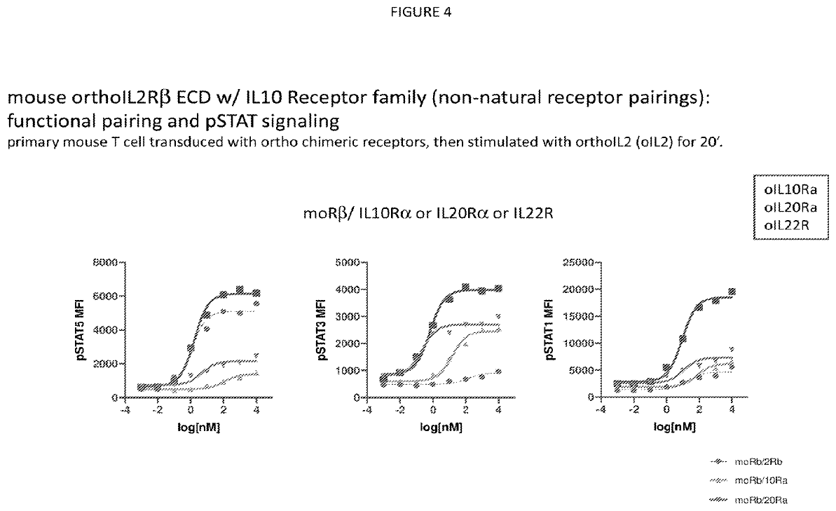

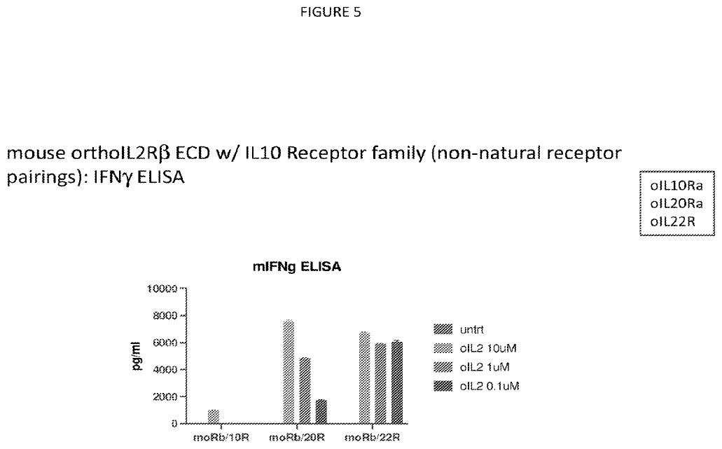

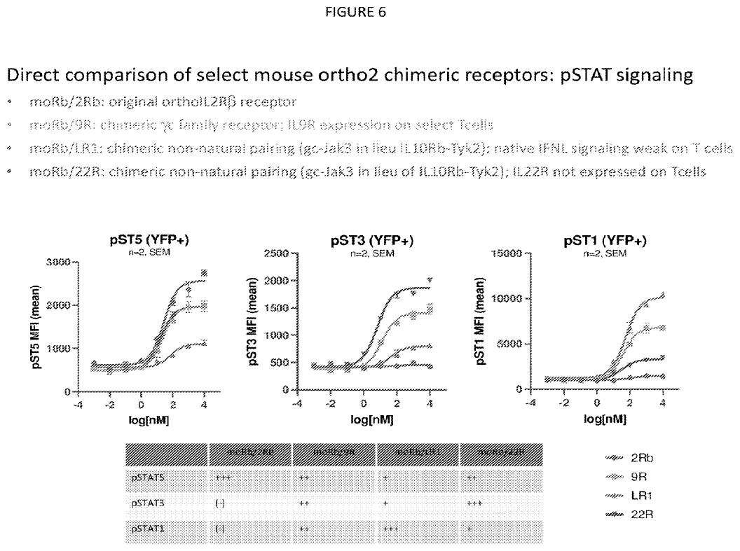

Constructs with the IL-10R ICD and the IL-22R ICD showed a strong STAT3 activation profile, and also a STAT1 response, as shown in FIG. 4. Mouse T cell blasts were virally transduced with either moRb/IL10Ra, moRb/IL20Ra, moRb/IL22R. Transduced cells were stimulated with dose titration concentrations of orthoIL2 for 20′, then fixed in paraformaldehyde (PFA), methanol (MeOH) permeabilized and stained with anti-pSTAT5-A647, anti-pSTAT3-A647 or anti-pSTAT1-A647. Samples were analyzed on a Cytoflex (Beckman Coulter), gating on YFP+ cells; data were plotted in Prism (GraphPad). MoRb/2Rb receptor was included as positive control for orthoIL2 signaling. There is a physiological effect from this signaling as well, shown in FIG. 6. Mouse T cell blasts were virally transduced with either moRb/IL10Ra, /IL20Ra, /IL22R, then cultured in indicated concentration of oIL2 for 3 days, then recall stimulated with anti-CD3 for 4H. Supernatant was harvested and level of IFNγ was determined by ELISA (Biolegend).

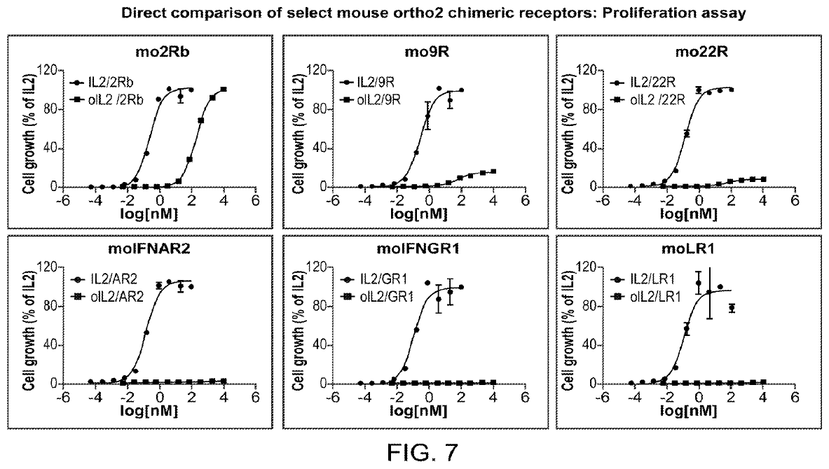

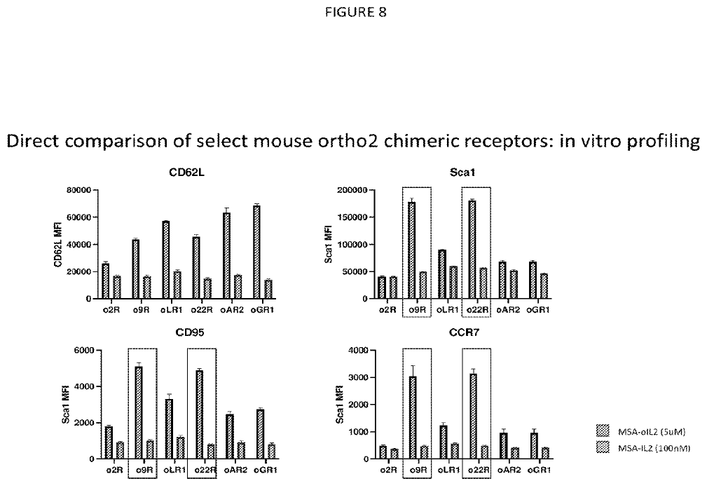

A direct comparison of moRb chimeric receptor on pSTAT signaling is shown in FIG. 6, on proliferation in FIG. 7, and on cell surface markers in FIG. 8. Mouse T cell blasts were virally transduced with either moRb/2Rb, moRb/9R, moRb/IFNLR1, or moRb/IL22R. Transduced cells were stimulated with orthoIL2 for 20′, then fixed in paraformaldehyde (PFA), methanol (MeOH) permeablilized and stained with anti-pSTAT5-A647, anti-pSTAT3-A647 or anti-pSTAT1-A647. Samples were analyzed on a Cytoflex (Beckman Coulter), gating on YFP+ cells; data were plotted in Prism (GraphPad). For proliferation, on day 4, samples were acquired on a Cytoflex (Beckman Coulter), gating on YFP+ cells; data were plotted in Prism (GraphPad). For cell surface markers, on day 4, samples were stained with antibodies to indicated cell surface markers and acquired on a Cytoflex (Beckman Coulter), gating on YFP+ cells; data were plotted in Prism (GraphPad).

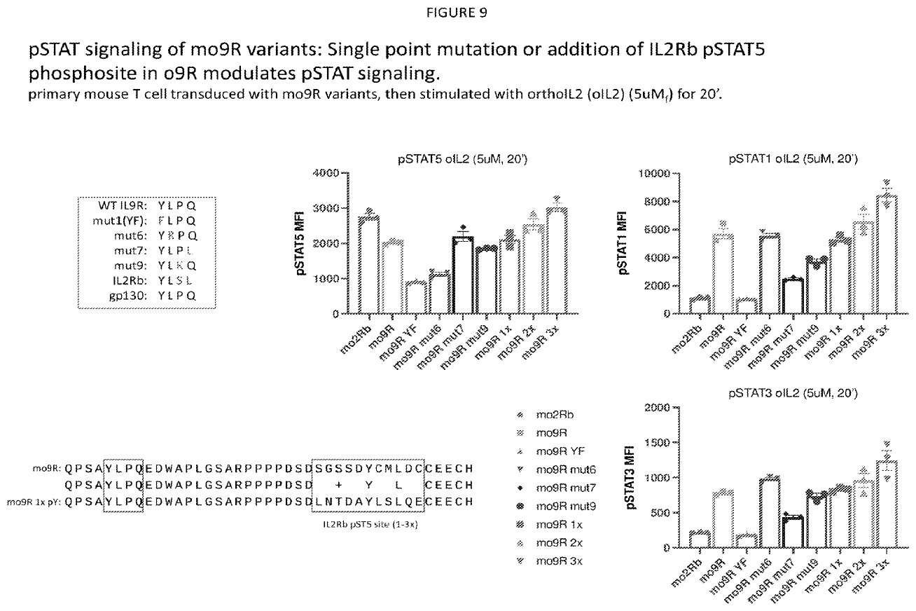

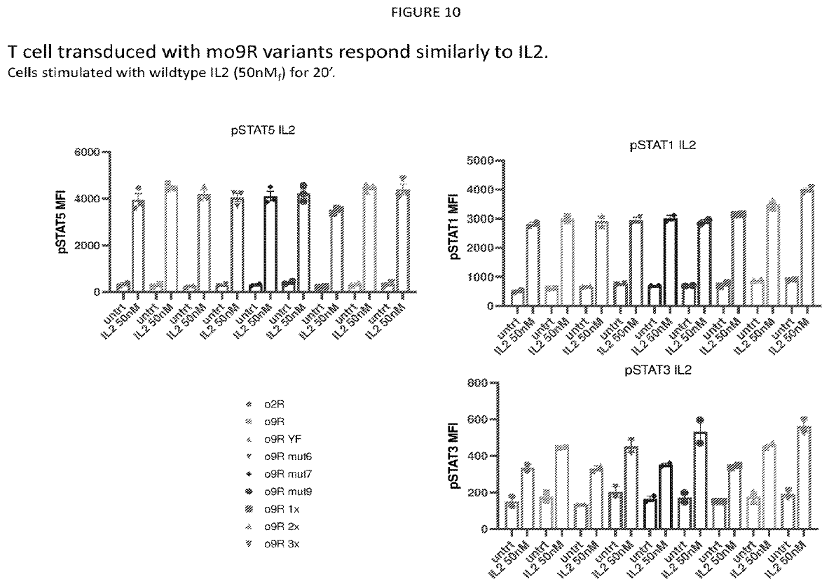

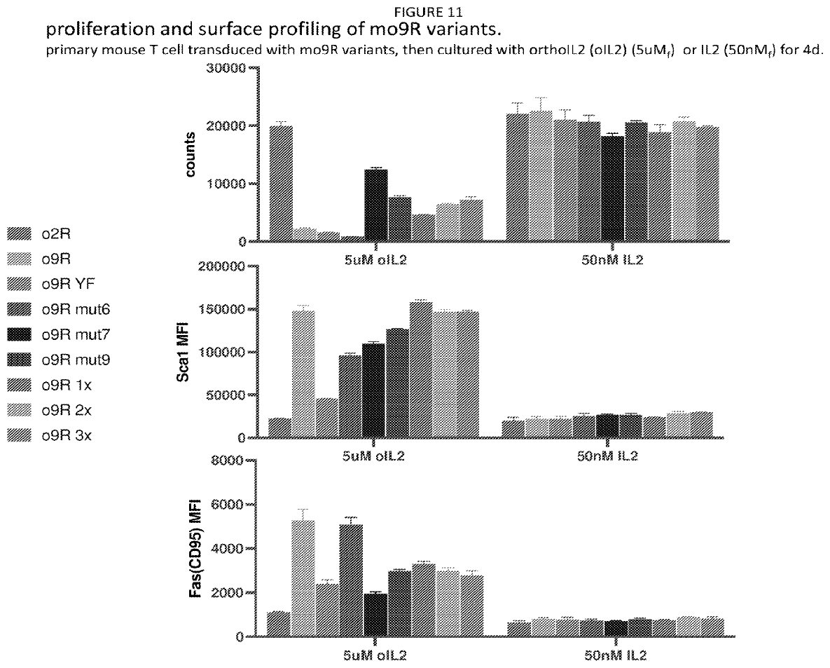

The STAT activation profile of IL-9R phosophosite variants was determined. Mouse T cell blasts were virally transduced with mo9R containing either a single point mutation in the STAT binding phosphosite or addition of the IL2Rb pSTAT5 phosphosite, in single, double, or triple tandem repeats, to the C-terminus of the o9R receptor. Transduced cells were stimulated with orthoIL2 for 20′, then fixed in paraformaldehyde (PFA), methanol (MeOH) permeablilized and stained with anti-pSTAT5-A647, anti-pSTAT3-A647 or anti-pSTAT1-A647. Samples were analyzed on a Cytoflex (Beckman Coulter), gating on YFP+ cells; data were plotted in Prism (GraphPad). Mo2Rb receptor was included as positive control for orthoIL2 signaling. Data is shown in FIG. 9. T cells transduced with mo9R phosphosite variants respond similarly to IL2, shown in FIG. 10. T cells tranduced with the mo9R phosphosite variants exhibit varying levels of proliferation and surface expression of stem-ness markers, shown in FIG. 11.

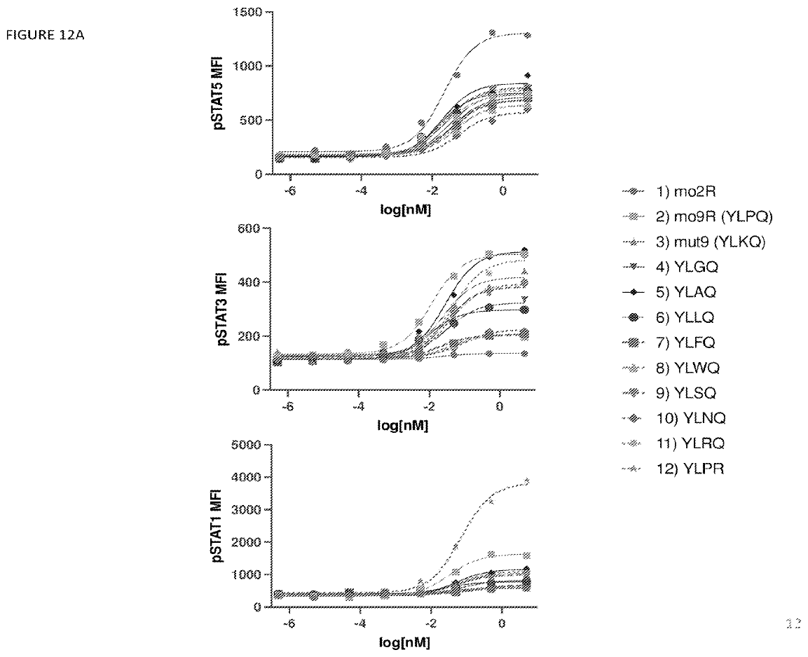

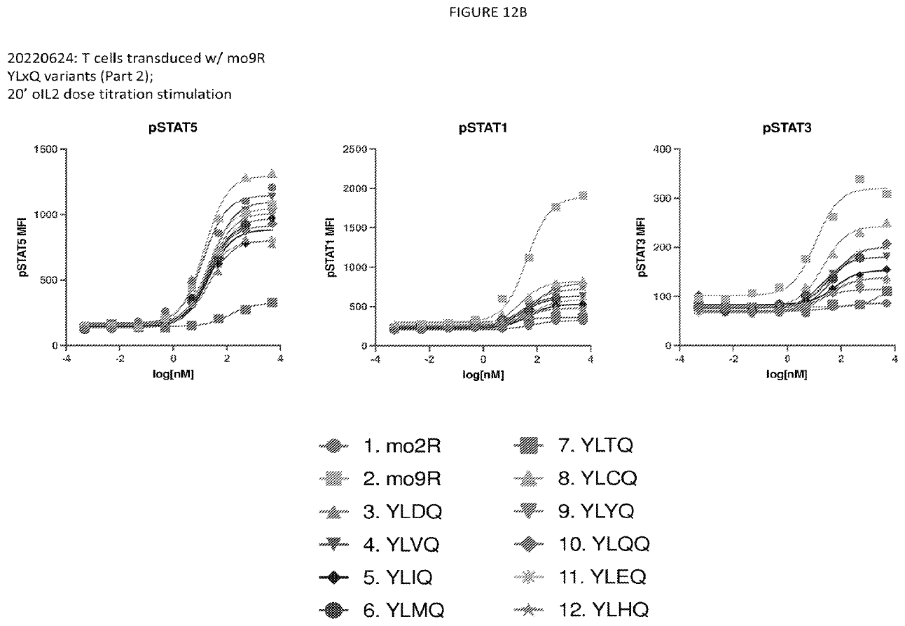

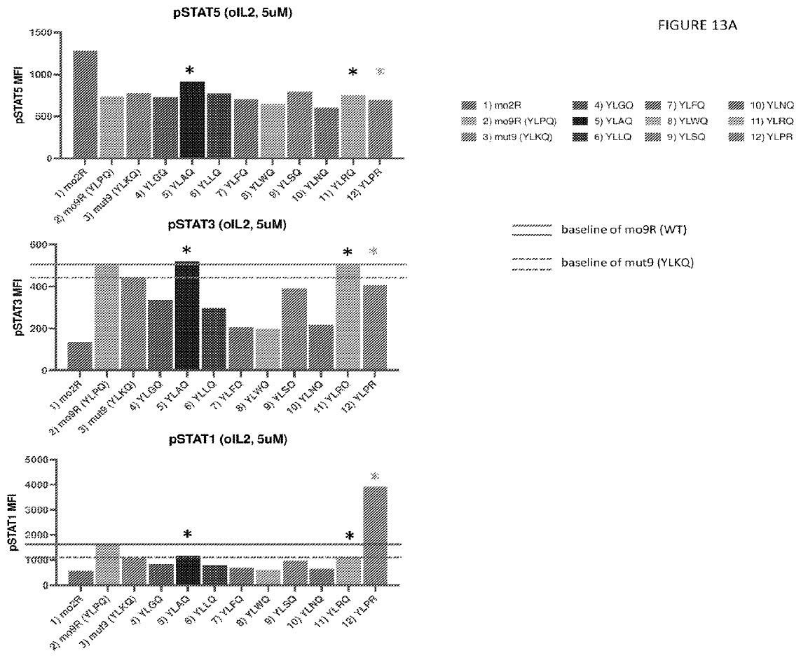

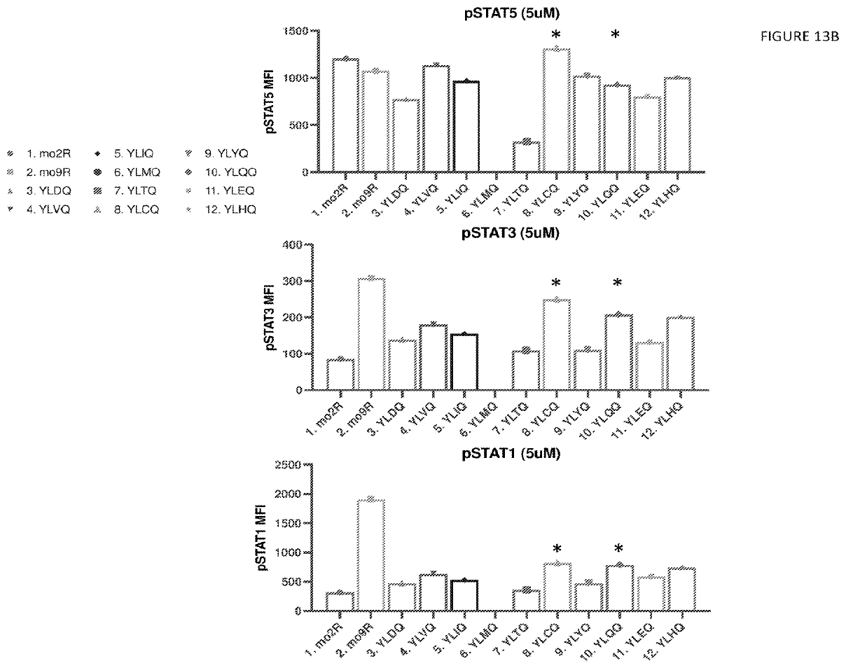

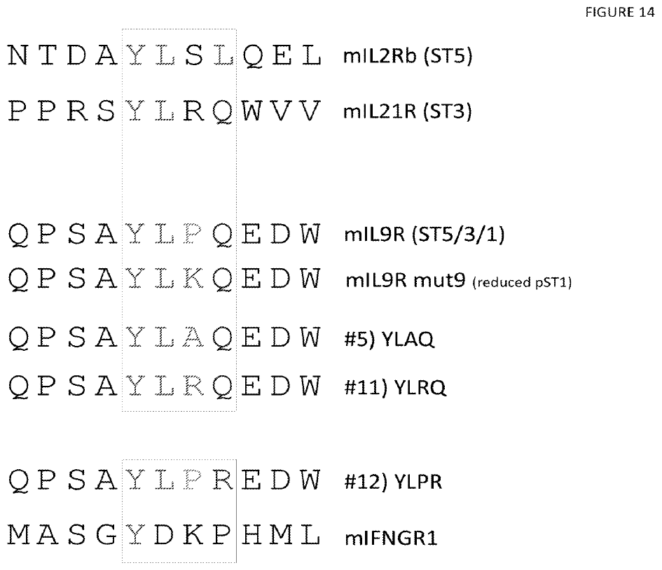

FIGS. 12A-12B and 13A-13B provide a STAT activation profile for a library of phosphosite variants. Mouse T cell blasts were virally transduced with mo9R containing a single point mutation in the STAT binding phosphosite.

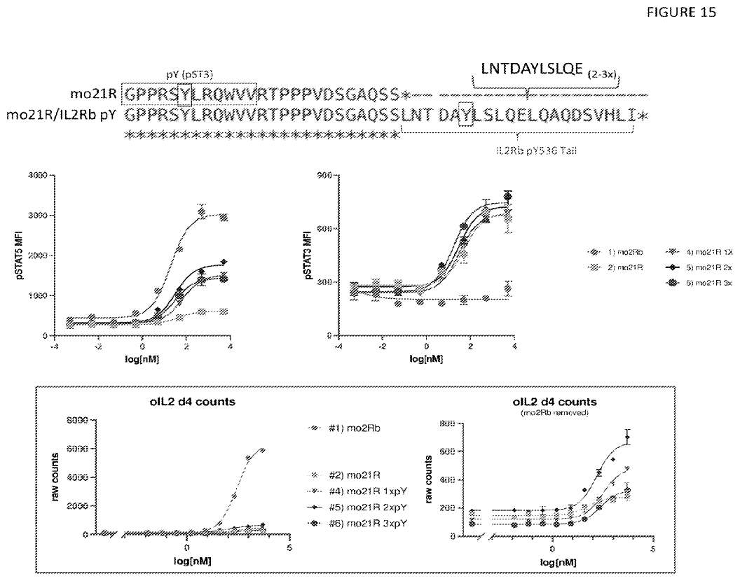

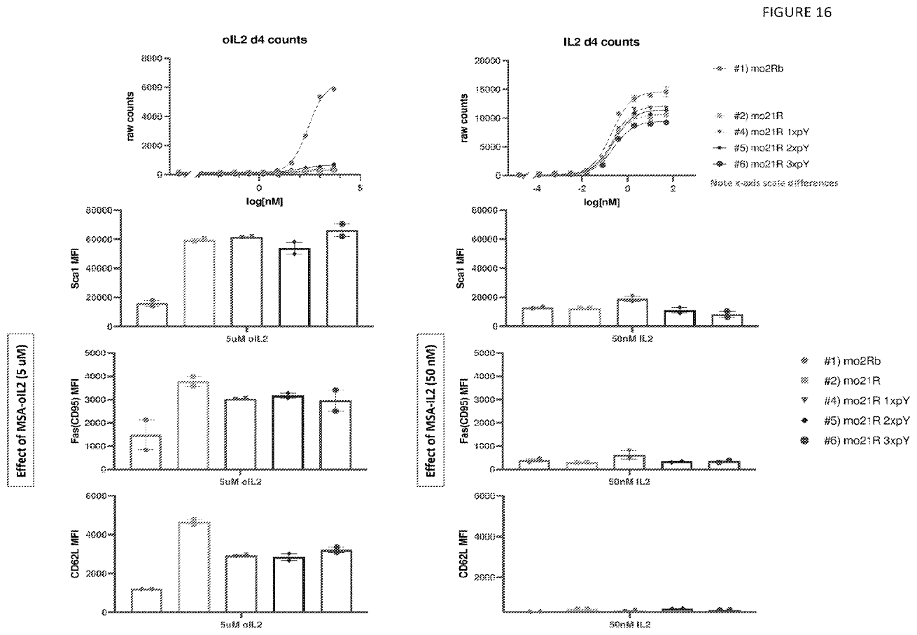

As shown in FIG. 15, STAT signaling and proliferation of mouse orthoIL2Rb/IL21Receptor (mo21R) variants, mouse T cell blasts were virally transduced with mo21R containing addition of the IL2Rb pSTAT5 phosphosite, in single, tandem or triple repeat, to the C-terminus of the o21R receptor. Transduced cells were stimulated with dose titration concentrations of orthoIL2 for 20′, then fixed in paraformaldehyde (PFA), methanol (MeOH) permeablilized and stained with anti-pSTAT5-A647, anti-pSTAT3-A647 or anti-pSTAT1-A647. Samples were analyzed on a Cytoflex (Beckman Coulter), gating on YFP+ cells; data were plotted in Prism (GraphPad). Mo2Rb receptor was included as positive control for orthoIL2 signaling. T cells transduced with mo21R phosphosite variants show slight increase in proliferation while retaining stem-ness markers, shown in FIG. 16.

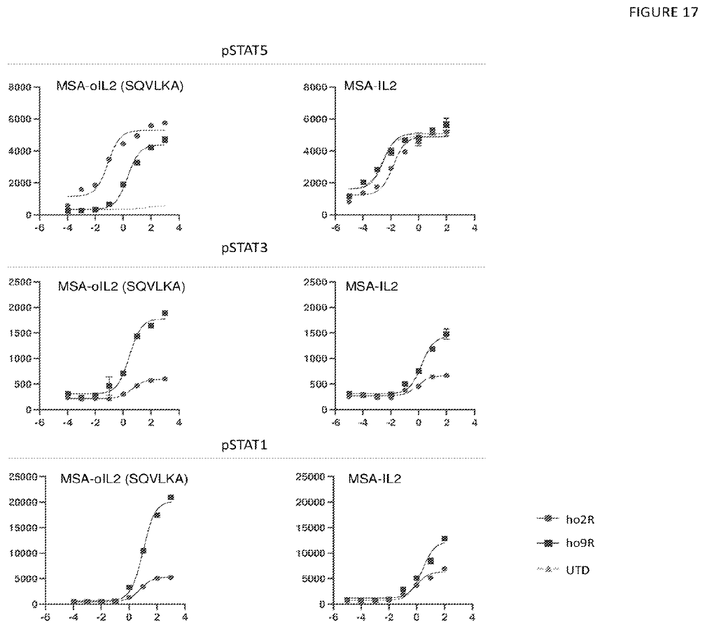

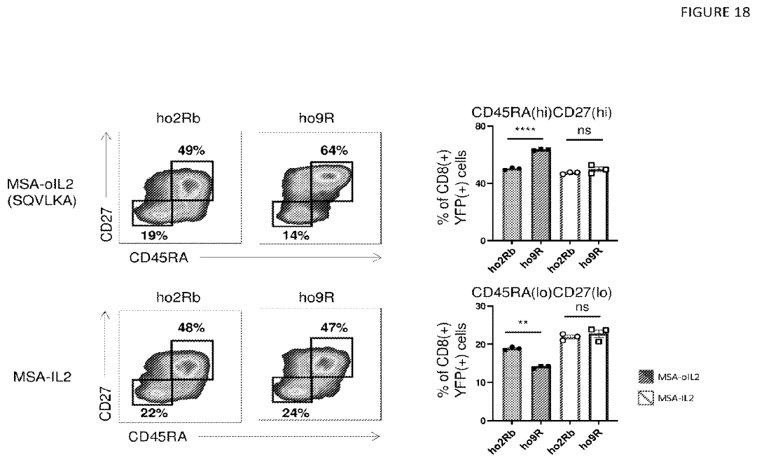

The STAT signaling of human orthoIL2Rb/IL9Receptor (ho9R) is shown in FIG. 17. Human PBMCs were virally transduced with ho2R or ho9R. Transduced cells were stimulated with human orthoIL2 or human IL2 for 20′, then fixed in paraformaldehyde (PFA), methanol (MeOH) permeablilized and stained with anti-pSTAT5-A647, anti-pSTAT3-A647 or anti-pSTAT1-A647. Samples were analyzed on a Cytoflex (Beckman Coulter), gating on YFP+ cells; data were plotted in Prism (GraphPad). UTD: untransduced control. The cell phenotypes were determined by staining the cells for CD27 and CD45RA, shown in FIG. 18. Human ortho-IL-2 is as

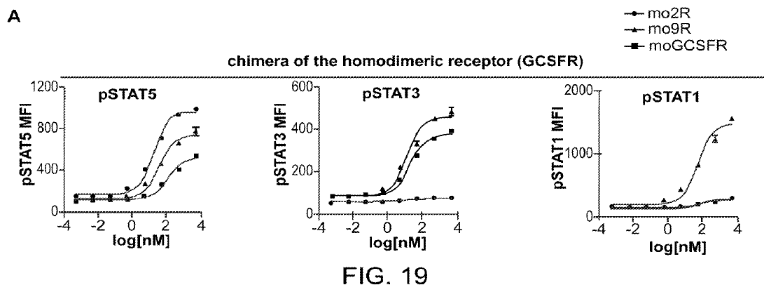

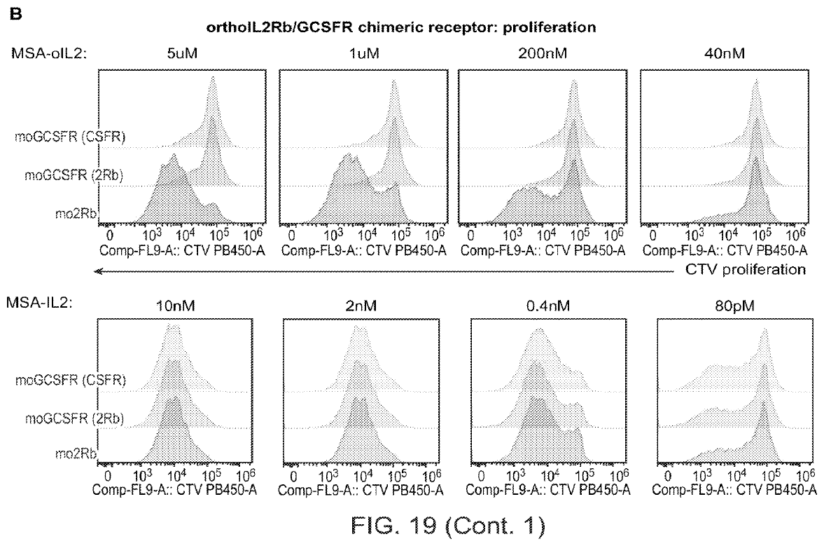

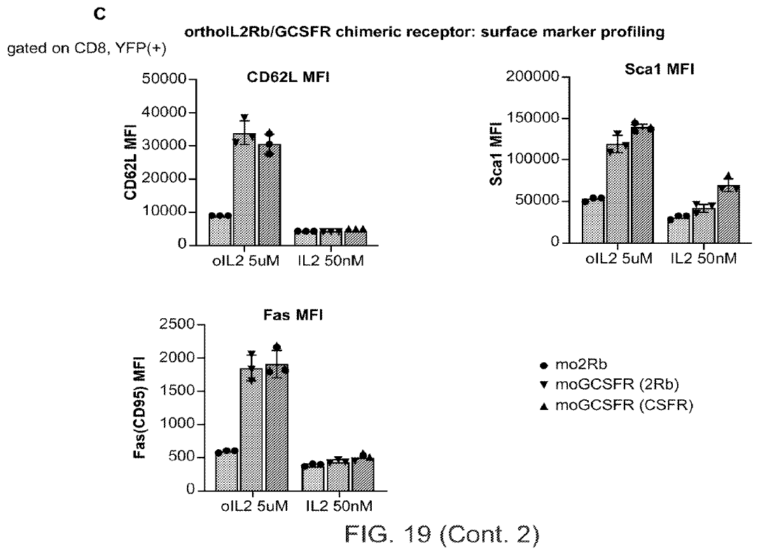

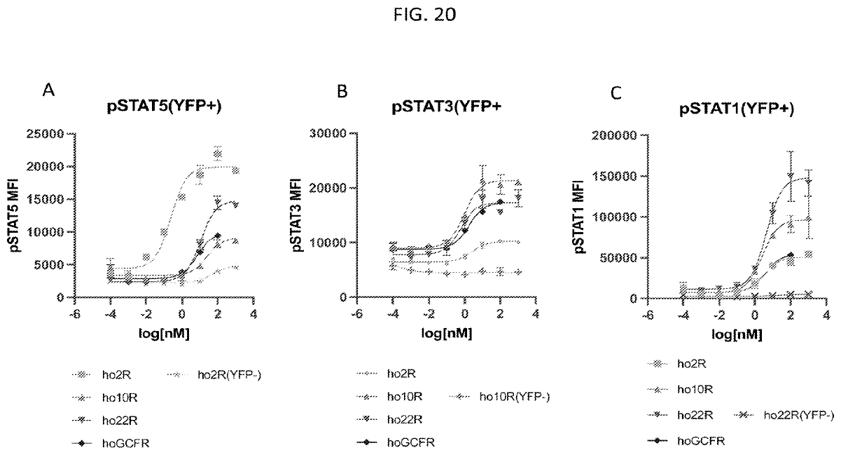

Mouse orthoIL2Rb chimeric receptors (mo2R, mo9R, or moGCSFR (SEQ ID NO:87) were transduced into mouse T cells. Cells were stimulated with dose titration of MSA-orthoIL2 for 20′, then fixed, permeabilized, stained for pSTAT and analyzed by flow cytometry. T cells were transduced with orthoIL2Rb/GCSFR chimeric receptor (SEQ ID NO:85). Cells were labeled w/CTV and cultured in either dose titration of MSA-orthoIL2 or MSA-IL2. 96h later, cells were analyzed by flow cytometry. T cells were transduced with orthoIL2Rb/GCSFR chimeric receptor (SEQ ID NO:85), then cultured in either MSA-orthoIL2 or MSA-IL2. 96h later, cells were stained for indicated surface markers, and analyzed by flow cytometry. Data is shown in FIG. 19

TABLE 3
Summary of the select mouse ortho2 chimeric receptors:

| | mo2R | mo9R | molFNLR1 | molL22R | molFNAR2 | molFGR1 |
|---|---|---|---|---|---|---|
| pSTAT5 | +++ | ++ | + | ++ | +/− | +/− |
| pSTAT3 | (−) | ++ | + | +++ | n.d. | n.d. |
| pSTAT1 | (−) | ++ | +++ | + | ++ | +++ |
| proliferation | +++ | + | (−) | + | (−) | (−) |
| Sca1 | + | +++ | ++ | +++ | + | + |
| CD95 | + | +++ | ++ | +++ | ++ | ++ |
| CCR7 | + | +++ | ++ | +++ | ++ | ++ |
| CD62L | + | ++ | +++ | ++ | +++ | +++ |

Each receptor construct was transduced in a T cell line and tested for activation activity with the mouse ortho-IL2 ligand. The receptor constructs were:

mo2R (SEQ ID NO:2), ortho IL2-RB receptor.

mo9R (SEQ ID NO:4), which is a chimera with the ECD of ortho-IL2Rβ and an ICD of mouse IL-9R, which ICD normally associates with γc. IL-9R is expressed on select T cells.

molFNLR1 (SEQ ID NO:44), which is a chimera with the ECD of ortho-IL2Rβ and an ICD of IFNLR1. This chimera provides for a non-natural pairing with γc-Jak3. Native IFNA signaling is weak on T cells.

moIL22R (SEQ ID NO:50), which is a chimera with the ECD of ortho-IL2Rβ and an ICD

of IL-22R. This chimera provides for a non-natural pairing with γc-Jak3. IL22R is not expressed on T cells.

molFNAR2 (SEQ ID NO:38), which is a chimera with the ECD of ortho-IL2Rβ and an ICD of IFNAR2. This chimera provides for a non-natural pairing with γc-Jak3.

molFNGR1 (SEQ ID NO:40), which is a chimera with the ECD of ortho-IL2Rβ and an ICD of IFNGR1. This chimera provides for a non-natural pairing with γc-Jak3.

What is claimed is:

1. A tunable chimeric receptor subunit polypeptide, comprising:
(a) an extracellular domain (ECD) that, when bound to its cognate ligand, multimerizes and activates intracellular signaling pathways;
(b) a transmembrane domain (TM) and
(c) an intracellular domain (ICD) having a sequence other than the naturally occurring ICD of the extracellular domain.

2. The tunable chimeric receptor subunit polypeptide of claim 1, wherein the ICD further comprises targeted amino acid modifications that alter the STAT protein profile activated by the receptor.

3. The tunable chimeric receptor subunit polypeptide of claim 1 or claim 2, wherein the ICD comprises the TM and ICD sequence of a receptor subunit selected from IL3Rα; IL4Rα; IL5Rα; IL6Rα; IL7Rα; IL9Rα; IL10Rα; IL10Rβ; IL12Rβ1; IL12Rβ2; IL12p40; IL13RA1; IL15Rα; IL20Rβ; IL21Rα; IL22R; IL23R; IL28R; IL31Rα; GMCSFRα; LIFRβ; CNTFR; CLF1; OSMR; GCSFR; EPOR; TPOR; GHR; PRLR; LEPR; IFNAR2; IFNAR1; IFNGR1; and IFNGR2.

4. The tunable chimeric receptor subunit polypeptide of any of claims 1-3, wherein the ICD comprises an ICD of SEQ ID NO:2; SEQ ID NO:4; SEQ ID NO:6; SEQ ID NO:8; SEQ ID NO: 10; SEQ ID NO: 12; SEQ ID NO: 14; SEQ ID NO: 16; SEQ ID NO: 18; SEQ ID NO:22; SEQ ID NO: 24; SEQ ID NO: 26; SEQ ID NO: 28; SEQ ID NO:30; SEQ ID NO:32; SEQ ID NO:34; SEQ ID NO: 36; SEQ ID NO:38; SEQ ID NO: 42; SEQ ID NO: 44; SEQ ID NO:46; SEQ ID NO:48; SEQ ID NO: 50; SEQ ID NO:52; SEQ ID NO:54; SEQ ID NO:56; SEQ ID NO:58; SEQ ID NO:60; SEQ ID NO: 64; SEQ ID NO: 66; SEQ ID NO: 68; SEQ ID NO:70; SEQ ID NO:72; SEQ ID NO:74; SEQ ID NO: 76; SEQ ID NO: 78; SEQ ID NO: 80; SEQ ID NO:82; SEQ ID NO:86, SEQ ID NO:88; SEQ ID NO: 90; or SEQ ID NO:92 or a variant thereof.

5. The tunable chimeric receptor subunit polypeptide of any of the preceding claims, wherein the ICD comprises a human or mouse interferon receptor subunit ICD selected from the ICD of IFNAR2; IFNAR1; IFNGR1; and IFNGR2.

6. The tunable chimeric receptor subunit polypeptide of any of the preceding claims, wherein the ICD comprises a human or mouse ICD selected from the ICD of IL-9R, IL-10R, IL-21R, IL-22R, and GCSFR ICD.

7. The tunable chimeric receptor subunit polypeptide of any of the preceding claims, wherein the ICD comprises modifications at a phosphosite that is phosphorylated by a JAK kinase.

8. The tunable chimeric receptor subunit polypeptide of any of the preceding claims, wherein the ICD Y(+2) site is mutagenized to generate a library of ICD sequences comprising each possible amino acid.

9. The tunable chimeric receptor subunit polypeptide of claim 8, wherein the ICD Y(+2) site comprises the sequence YLXQ where X is any amino acid or any amino acid other than the wild-type residue.

10. The tunable chimeric receptor subunit polypeptide of any of the preceding claims, wherein the ICD Y(−2), (−1), and (+2) sites are modified to comprise the motif SAYLXQ, or DAYLXQ, where X is any amino acid, or any amino acid other than the wild-type residue.

11. The tunable chimeric receptor subunit polypeptide of any of the preceding claims, wherein the ICD is modified to comprise an additional phosphosite.

12. The tunable chimeric receptor subunit polypeptide of claim 11, wherein the additional phosphosite comprises SEQ ID NO:93 (LNTDAYLSLQE)ₓ, where X is 1, 2, 3, or more.

13. The tunable chimeric receptor subunit polypeptide of claim 9 or claim 10, wherein the additional phosphosite is placed close to the C-terminus of the ICD.

14. The tunable chimeric receptor subunit polypeptide of any of the preceding claims, wherein the ECD is a ECD of a receptor subunit associated with the common gamma chain (CD132).

15. The tunable chimeric receptor subunit polypeptide of any of the preceding claims, wherein the ECD is a ECD of IL-2RB.

16. The tunable chimeric receptor subunit polypeptide of claim 13, wherein the ECD is an ECD of an orthogonal variant of IL-2RB.

17. The tunable chimeric receptor subunit polypeptide of any of the preceding claims, comprising an amino acid sequence with at least 95% sequence identity to the ICD of SEQ ID NO: 2; SEQ ID NO:4; SEQ ID NO:6; SEQ ID NO:8; SEQ ID NO:10; SEQ ID NO:12; SEQ ID NO: 14; SEQ ID NO: 16; SEQ ID NO: 18; SEQ ID NO:22; SEQ ID NO:24; SEQ ID NO:26; SEQ ID NO: 28; SEQ ID NO: 30; SEQ ID NO:32; SEQ ID NO:34; SEQ ID NO:36; SEQ ID NO:38; SEQ ID NO: 42; SEQ ID NO: 44; SEQ ID NO: 46; SEQ ID NO:48; SEQ ID NO:50; SEQ ID NO:52; SEQ ID NO: 54; SEQ ID NO:56; SEQ ID NO:58; SEQ ID NO:60; SEQ ID NO: 64; SEQ ID NO:66; SEQ ID NO: 68; SEQ ID NO: 70; SEQ ID NO: 72; SEQ ID NO:74; SEQ ID NO:76; SEQ ID NO:78; SEQ ID NO: 80; SEQ ID NO: 82; SEQ ID NO: 86, SEQ ID NO:88; SEQ ID NO:90; or SEQ ID NO:92; fused to a non-naturally occurring ECD.

18. The tunable chimeric receptor subunit polypeptide of any of the preceding claims, comprising an amino acid sequence with at least 95% sequence identity to the ICD sequence of any one of SEQ ID NO:18; SEQ ID NO:30; SEQ ID NO:32; SEQ ID NO:34; SEQ ID NO:36; SEQ ID NO: 38; SEQ ID NO: 42; SEQ ID NO:44; SEQ ID NO:46; SEQ ID NO:48; SEQ ID NO:64; SEQ ID NO: 66; SEQ ID NO:68; SEQ ID NO:70; SEQ ID NO:72; SEQ ID NO:74; SEQ ID NO:78; SEQ ID NO: 80; SEQ ID NO:82; SEQ ID NO:86, SEQ ID NO:88; SEQ ID NO:90; or SEQ ID NO:92; fused to a non-naturally occurring ECD.

19. The tunable chimeric receptor subunit polypeptide of any of the preceding claims, where the TM is the naturally-occurring TM of the ICD.

20. The tunable chimeric receptor subunit polypeptide of any of the preceding claims, where the TM is the naturally-occurring TM of the ECD.

21. A system for tunable cytokine activation of a cell, the system comprising:
(a) tunable cytokine receptor subunit polypeptide of any of the preceding claims; and
(b) a cognate ligand for the ECD.

22. The system of claim 21, wherein the cognate ligand is an orthogonal ligand.

23. The system of claim 21 or 22, wherein the cell is an immune cell or a stem cell.

24. The system of claim 16, wherein the immune cell is a T cell.

25. The system of claim 24, wherein the T cell is present in a tumor infiltrating lymphocyte population.

26. The system of claim 25, wherein the T cell is a CAR T cell.

27. A nucleic acid encoding the tunable cytokine receptor subunit polypeptide of any of claims 1-20.

28. An expression vector comprising the nucleic acid of claim 27.

29. A cell genetically engineered to comprise the vector of claim 28.

30. A method of treating an individual, the method comprising introducing an immune effector cell expressing a tunable cytokine receptor subunit polypeptide according to any of claim 1-20, and activating the cell by contacting with a cognate ligand for the ECD.

31. The method of claim 30, wherein the immune effector cell is a T cell.

32. The method of claim 31, wherein the T cell is obtained from a tumor infiltrating lymphocyte population.

33. The method of claim 32, wherein the T cell is a CAR T cell.

34. The method of any of claims 30-33, wherein the individual is treated for cancer.

35. The method of any of claims 30-33, wherein the individual is treated for autoimmune disease.

36. The method of any of claims 30-33, wherein the individual is treated for infection.

37. A kit comprising the system of claim 14.


SEQUENCE LISTING (93 sequences)

SEQ ID NO: 1 - DNA, 1203 bp, synthetic construct
  source 1..1203: mol_type = other DNA
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaaggccagcaagtttttccacgatctggattataactggattaggtgttgtgttcttctctgtcatggtcctttatgctttgaggagcgtctggaaatacctgtgtcatgtgtgcttcccaccactcaagcctccccgcagtattgatgagtttttctctgagccgccttcaaaaaaccttgtacttctgacggctgaggagcacacggaaagatgcttcatcattgagaatacagacacggtcgctgtagaagtaaagcacgcgcctgaggaggacctcaggaagtacagctcacagaccagccaggactcgggcaactattccaacgaagaggaggagagtgtgggcaccgagagcggccaagctgtgctctccaaagctccctgcggggggccatgcagcgtgcctagccctcctgggaccttggaagacgggacctgcttcctgggaaatgaaaaatatcttcagagcccagccctgaggacagagccagctcttctctgctga

SEQ ID NO: 2 - protein, 400 aa, synthetic construct
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPASFSTIWIITGLGVVFFSVMVLYALRSVWKYLCHVCFPPLKPPRSIDEFFSEPPSKNLVLLTAEEHTERCFIIENTDTVAVEVKHAPEEDLRKYSSQTSQDSGNYSNEEEESVGTESGQAVLSKAPCGGPCSVPSPPGTLEDGTCFLGNEKYLQSPALRTEPALLC

SEQ ID NO: 3 - DNA, 1551 bp, synthetic construct
  source 1..1551: mol_type = other DNA
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaaggccagcaggccaggaatcaggattatcagaatctgctatagtaggaataactacttcgtgtttggtagtgatggttttcgtgagcactatcgtaatgctgaaacggattggttatatatgcctaaaagacaatttgcccaatgtcttgaacttccgccactttttaacctggataatccctgaacggtcaccttcagaagccatcgatcggctggaaatcatccccacaaacaagaagaagagactgtggaattacgattatgaggatggcagtgacagtgacgaagaggtccccacagcaagtgtcactggctacaccatgcatggactgacgggcaagcctctgcaacaaacctctgacacctcagccagccccgaggatcccctgcatgaagaagattcaggcgctgaggaatctgatgaagctggagcaggggctggagctgagccagaactccccacagaggcgggggcggggccttcagaagaccccactggcccctatgagagaagaaagagtgtgctcgaggactcattccccagggaggacaacagctccatggatgagcctggggacaacattatcttcaacgtgaacttaaactctgtgtttctgagggttctccatgatgaagatgcctcagagacattatctctcgaagaagacaccatcctcctagacgaaggtccccagaggacagagtcagaccttcggatagctggtggggacaggacacagccgcccctccccagccttccttcccaggatctatggactgaagatgggtcatctgagaaatcagacacctcagattccgatgctgatgtgggggacggctacatcatgagatga

SEQ ID NO: 4 - protein, 516 aa, synthetic construct
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPAGQESGLSESAIVGITTSCLVVMVFVSTIVMLKRIGYICLKDNLPNVLNFRHFLTWIIPERSPSEAIDRLEIIPTNKKKRLWNYDYEDGSDSDEEVPTASVTGYTMHGLTGKPLQQTSDTSASPEDPLHEEDSGAEESDEAGAGAGAEPELPTEAGAGPSEDPTGPYERRKSVLEDSFPREDNSSMDEPGDNIIFNVNLNSVFLRVLHDEDASETLSLEEDTILLDEGPQRTESDLRIAGGDRTQPPLPSLPSQDLWTEDGSSEKSDTSDSDADVGDGYIMR

SEQ ID NO: 5 - DNA, 1398 bp, synthetic construct
  source 1..1398: mol_type = other DNA
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaaggccagcacatgatgacagaaaggattcaatttggattctggtggttgctcctcttaccgtctttacagtagttatcctggtatttgcgtattggtatactaagaagaattcattcaagagaaaaagcataatgttacctaagtccttgctctctgtggtaaaaagtgccacgttagagacaaaacctgaatcgaagtattcacttgtcacaccgcaccagccagctgtcctagagagtgagacggtgatctgtgaagagcccctgtccacagtgacagctccagacagccccgaagcagcagaacaggaagaactttcaaaagaaacaaaggctctggaggctggaggaagcacgtctgccatgaccccagacagccctccaactccgacacaaagacgcagcttttccctgttaagtagtaaccagtcaggcccttgtagcctcaccgcctatcactcccgaaacggctctgacagtggcctcgtgggatcgggcagctccatatcggacttggaatctctcccaaacaacaactcagaaacaaagatggcagagcacgaccctccacccgtgagaaaggcccccatggcctccggttatgacaaaccgcacatgttggtggacgtgcttgtggatgttggggggaaggagtctctcatggggtatagactcacaggagaggcccaggagctgtcctaa

SEQ ID NO: 6 - protein, 465 aa, synthetic construct
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPAHDDRKDSIWILVVAPLTVFTVVILVFAYWYTKKNSFKRKSIMLPKSLLSVVKSATLETKPESKYSLVTPHQPAVLESETVICEEPLSTVTAPDSPEAAEQEELSKETKALEAGGSTSAMTPDSPPTPTQRRSFSLLSSNQSGPCSLTAYHSRNGSDSGLVGSGSSISDLESLPNNNSETKMAEHDPPPVRKAPMASGYDKPHMLVDVLVDVGGKESLMGYRLTGEAQELS

SEQ ID NO: 7 - DNA, 1005 bp, synthetic construct
  source 1..1005: mol_type = other DNA
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaaggccagcagcaaatgcctccgccaggctgcagcaagtcatcctgattccgttgggcatcttcgcattgctgctcggcctgacgggcgcctgcttcaccctgttcctcaaataccaaagccgagtgaagtactggtttcaggctccgccaaacatcccggaacaaatcgaagagtatctaaaggacccagaccaattcatcttagaggtcttggacaaggacggttcaccgaaggaggactcctgggactccgtgtcaattatttcttctccagaaaaggagcgagatgatgtgctccaaacaccgtga

SEQ ID NO: 8 - protein, 334 aa, synthetic construct
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPAANASARLQQVILIPLGIFALLLGLTGACFTLFLKYQSRVKYWFQAPPNIPEQIEEYLKDPDQFILEVLDKDGSPKEDSWDSVSIISSPEKERDDVLQTP

SEQ ID NO: 9 - DNA, 1653 bp, synthetic construct
  source 1..1653: mol_type = other DNA
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaaggccagcagctccaggggacaaaagagctgtcctggcaatgccctcactcttgcttctactgatagcagccgttgcagcaggtgtggcatggaagataatgaaaggaaacccctggtttcagggggtgaagacgccccgggcactggacttttctgaatacagatacccagtggcaacctttcagcccagtggacctgagttctctgatgacttgatcctttgtccccagaaggaactgaccataaggaacaggccagcccctcaggtcagaaacccagccacgctacaggcaggaccagaaagagacagtactgaggatgaagacgaggacacagactacgatgacgacggtgacagcgtccagccctacctggaacggcccctcttcatcagcgagaagccccgggttatggaacactcggagacagacgagtctggggtggattcaggggggccttggacatccccagttgggagtgacggctcctctgcatgggactcttcagacaggagctggtccagcacaggggactcctcatataaggatgaagttgggtcttccagctgtttggaccgaaaggagccggaccaggcgccctgtggggattggctccaggaggctctcccatgcctggaattttctgaggacttgggcaccgtggaagagcctctgaaggatggcctctccgggtggaggatttctggttccttatcctcaaagagagatctggctcctgtggagcccccagtttctcttcagacactgactttctgctgggtcaacaatcctgagggggaggaggaacaggaggacgaggaggaagaggaggaggaggaggaggaggaagactgggaatcagaacctaagggcagcaatgccggctgttggggcacttcaagcgtgcagaggacggaggtcaggggccggatgctgggagattacttggtcaggtga

SEQ ID NO: 10 - protein, 550 aa, synthetic construct
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPAAPGDKRAVLAMPSLLLLLIAAVAAGVAWKIMKGNPWFQGVKTPRALDFSEYRYPVATFQPSGPEFSDDLILCPQKELTIRNRPAPQVRNPATLQAGPERDSTEDEDEDTDYDDDGDSVQPYLERPLFISEKPRVMEHSETDESGVDSGGPWTSPVGSDGSSAWDSSDRSWSSTGDSSYKDEVGSSSCLDRKEPDQAPCGDWLQEALPCLEFSEDLGTVEEPLKDGLSGWRISGSLSSKRDLAPVEPPVSLQTLTFCWVNNPEGEEEQEDEEEEEEEEEEEDWESEPKGSNAGCWGTSSVQRTEVRGRMLGDYLVR

SEQ ID NO: 11 - DNA, 1746 bp, synthetic construct
  source 1..1746: mol_type = other DNA
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaaggccagcaacggagcagtatttcactgtgaccaacctgagcatcttagtcatatctatgctgctattctgtggaatcctggtctgtctggttctccagtggtacatccggcacccggggaagttgcctacagtcctggtcttcaagaagcctcacgacttcttcccagccaaccctctctgcccagaaactcccgatgccattcacatcgtggacctggaggttttcccaaaggtgtcactagagctgagagactcagtcctgcatggcagcaccgacagtggctttggcagtggtaaaccatcacttcagactgaagagtcccaattcctcctccctggctcccacccccagatacaggggactctgggaaaagaagagtctccagggctacaggccacctgtggggacaacacggacagtgggatctgcctgcaggagcccggcttacactccagcatggggcccgcctggaagcagcagcttggatatacccatcaggaccaggatgacagtgacgttaacctagtccagaactctccagggcagcctaagtacacacaggatgcatctgccttgggccatgtctgtctcctagaacctaaagcccctgaggagaaagaccaagtcatggtgacattccagggctaccagaaacagaccagatggaaggcagaggcagcaggcccagcagaatgcttggacgaagagattcccttgacagatgcctttgatcctgaacttggggtacacctgcaggatgatttggcttggcctccaccagctctggccgcaggttatttgaaacaggagtctcaagggatggcttctgctccaccagggacaccaagtagacagtggaatcaactgaccgaagagtggtcactcctgggtgtggttagctgtgaagatctaagcatagaaagttggaggtttgcccataaacttgaccctctggactgtggggcagcccctggtggcctcctggatagccttggctctaacctggtcaccctgccgttgatctccagcctgcaggtagaagaatga

SEQ ID NO: 12 - protein, 581 aa, synthetic construct
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPATEQYFTVTNLSILVISMLLFCGILVCLVLQWYIRHPGKLPTVLVFKKPHDFFPANPLCPETPDAIHIVDLEVFPKVSLELRDSVLHGSTDSGFGSGKPSLQTEESQFLLPGSHPQIQGTLGKEESPGLQATCGDNTDSGICLQEPGLHSSMGPAWKQQLGYTHQDQDDSDVNLVQNSPGQPKYTQDASALGHVCLLEPKAPEEKDQVMVTFQGYQKQTRWKAEAAGPAECLDEEIPLTDAFDPELGVHLQDDLAWPPPALAAGYLKQESQGMASAPPGTPSRQWNQLTEEWSLLGVVSCEDLSIESWRFAHKLDPLDCGAAPGGLLDSLGSNLVTLPLISSLQVEE

SEQ ID NO: 13 - DNA, 1614 bp, synthetic construct
  source 1..1614: mol_type = other DNA
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaaggccagcagttcaaacatcagcatggaaggctaaagtcatcttctggtatgtcttcctcacatctgttatcgtgtttcttttctccgcaattggctacttggtttaccgttacatccatgttggcaaggaaaaacacccagcaaatttggtactgatttatagaaatgaaattggcacaagagtctttgaacctactgaaacaatcacacttaattttatcaccttcagtatgttggatgatactaaaatttctccaaaggatatgaatttactggacaaaagcagtgatgacatcagtgttaatgaccctgagcacaatgaggcctgggagccgcactgggaggaggtggaggggcaacatttaggatgctcttcgcatttgatggacgctgtctgtggtgctgagcaaagagacggagacacctccctaacccagcatgggtggcttaacagcaccatccccacaggagagacagacactgagcctcaatacaaagtcctaagtgacttctacggggagggtgaaatccaactgtcctgtgagccggaagaggcggccagaacagagaaaatatctgagccactggtgacttcagcaaacttggacccacagcttgaagacctacatcacctgggtcaggagcatactgtctccgaggatgggccagaggaagagacatctataacagtagtggattgggaccctcaaactggcaggctgtgtatcccttccttacctatctttggccgtgatcctgagaactatggtcattatgagagagaccagctcttagagggtggccttttgtctagactctatgagaaccaggcacctgacaagccagagaaagaaaatgaaaactgtctcacacggtttatggaggaatgggggttacatgtacaaatggaaagctag

SEQ ID NO: 14 - protein, 537 aa, synthetic construct
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPAVQTSAWKAKVIFWYVFLTSVIVFLFSAIGYLVYRYIHVGKEKHPANLVLIYRNEIGTRVFEPTETITLNFITFSMLDDTKISPKDMNLLDKSSDDISVNDPEHNEAWEPHWEEVEGQHLGCSSHLMDAVCGAEQRDGDTSLTQHGWLNSTIPTGETDTEPQYKVLSDFYGEGEIQLSCEPEEAARTEKISEPLVTSANLDPQLEDLHHLGQEHTVSEDGPEEETSITVVDWDPQTGRLCIPSLPIFGRDPENYGHYERDQLLEGGLLSRLYENQAPDKPEKENENCLTRFMEEWGLHVQMES

SEQ ID NO: 15 - DNA, 1785 bp, synthetic construct
  source 1..1785: mol_type = other DNA
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaaggccagcacctgacagaacctgggcctactctttctctggcgccgtgctgttctctatgggctttctcgtgggactgctgtgctacctgggctacaagtacatcaccaagcctccagtgcctccaaacagcctgaacgtgcagagagtgctgaccttccagcctctgagattcatccaagagcatgtgctgatccccgtgctggacctgtctggaccatcttctctgccccagcctatccagtacagccaggtggtggtttctggccccagagaacctcctggcgcagtttggagacagagcctgagcgacctgacatacgtgggccagagcgacgtgtcaatcctgcagcctacaaacgtgcccgctcagcagacactgagccctccatcttacgcccctaaggctgtgccagaagtgcagcctcctagctacgctcctcaggtggcctctgatgccaaggctctgttctacagccctcagcagggcatgaagaccagacctgccacatacgaccctcaggacatcctggactcttgccctgccagctacgctgtgtgcgtggaagatagcggcaaggacagcacacctggcatcctgagcacccctaagtacctcaagaccaagggccagctgcaagaggacacactcgtgcggtcttgtctgcctggcgatctgagcctgcagaaagtgacatctctcggcgagggcgagacacagaggcctaagtctctgccttctcctctgggcttctgcaccgacagaggacctgatctgcacaccctgagaagcgaggaacccgagacacccagatacctgaagggcgcactgtccctgctgtccagcgtgcagattgagggacaccctgtgtctctgccactgcatgtgcacagcgtgtcctgctctccatctgacgagggaccttctccatggggcctgctggattctctcgtgtgccctaaggatgagggccctgccgtggaaacagaggccatgtgtccatctgctgccgcctctgagctggaacagtctaccgaactggacagcctgttcaaaggcctggctctgacagtgcagtgggagagctga

SEQ ID NO: 16 - protein, 594 aa, synthetic construct
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPAPDRTWAYSFSGAVLFSMGFLVGLLCYLGYKYITKPPVPPNSLNVQRVLTFQPLRFIQEHVLIPVLDLSGPSSLPQPIQYSQVVVSGPREPPGAVWRQSLSDLTYVGQSDVSILQPTNVPAQQTLSPPSYAPKAVPEVQPPSYAPQVASDAKALFYSPQQGMKTRPATYDPQDILDSCPASYAVCVEDSGKDSTPGILSTPKYLKTKGQLQEDTLVRSCLPGDLSLQKVTSLGEGETQRPKSLPSPLGFCTDRGPDLHTLRSEEPETPRYLKGALSLLSSVQIEGHPVSLPLHVHSVSCSPSDEGPSPWGLLDSLVCPKDEGPAVETEAMCPSAAASELEQSTELDSLFKGLALTVQWES

SEQ ID NO: 17 - DNA, 1785 bp, synthetic construct
  source 1..1785: mol_type = other DNA
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaaggccagcacctgacagaacctgggcctactctttctctggcgccgtgctgttctctatgggctttctcgtgggactgctgtgctacctgggctacaagtacatcaccaagcctccagtgcctccaaacagcctgaacgtgcagagagtgctgaccttccagcctctgagattcatccaagagcatgtgctgatccccgtgctggacctgtctggaccatcttctctgccccagcctatccagtacagccaggtggtggtttctggccccagagaacctcctggcgcagtttggagacagagcctgagcgacctgacatacgtgggccagagcgacgtgtcaatcctgcagcctacaaacgtgcccgctcagcagacactgagccctccatcttacgcccctaaggctgtgccagaagtgcagcctcctagctacgctcctcaggtggcctctgatgccaaggctctgttctacagccctcagcagggcatgaagaccagacctgccacatacgaccctcaggacatcctggactcttgccctgccagctacgctgtgtgcgtggaagatagcggcaaggacagcacacctggcatcctgagcacccctaagttcctcaagaccaagggccagctgcaagaggacacactcgtgcggtcttgtctgcctggcgatctgagcctgcagaaagtgacatctctcggcgagggcgagacacagaggcctaagtctctgccttctcctctgggcttctgcaccgacagaggacctgatctgcacaccctgagaagcgaggaacccgagacacccagatacctgaagggcgcactgtccctgctgtccagcgtgcagattgagggacaccctgtgtctctgccactgcatgtgcacagcgtgtcctgctctccatctgacgagggaccttctccatggggcctgctggattctctcgtgtgccctaaggatgagggccctgccgtggaaacagaggccatgtgtccatctgctgccgcctctgagctggaacagtctaccgaactggacagcctgttcaaaggcctggctctgacagtgcagtgggagagctga

SEQ ID NO: 18 - protein, 594 aa, synthetic construct
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPAPDRTWAYSFSGAVLFSMGFLVGLLCYLGYKYITKPPVPPNSLNVQRVLTFQPLRFIQEHVLIPVLDLSGPSSLPQPIQYSQVVVSGPREPPGAVWRQSLSDLTYVGQSDVSILQPTNVPAQQTLSPPSYAPKAVPEVQPPSYAPQVASDAKALFYSPQQGMKTRPATYDPQDILDSCPASYAVCVEDSGKDSTPGILSTPKFLKTKGQLQEDTLVRSCLPGDLSLQKVTSLGEGETQRPKSLPSPLGFCTDRGPDLHTLRSEEPETPRYLKGALSLLSSVQIEGHPVSLPLHVHSVSCSPSDEGPSPWGLLDSLVCPKDEGPAVETEAMCPSAAASELEQSTELDSLFKGLALTVQWES

SEQ ID NO: 19 - DNA, 1344 bp, synthetic construct
  source 1..1344: mol_type = other DNA
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaaggccagcacagaggagacagggcctcctggtcccacgctggcaatggtcagccagcatccttgtagttgtgcccatctttcttctgctgactggctttgtccaccttctgttcaagctgtcacccaggctgaagagaatcttttaccagaacattccatctcccgaggcgttcttccatcctctctacagtgtgtaccatggggacttccagagttggacaggggcccgcagagccggaccacaagcaagacagaatggtgtcagtacttcatcagcaggctcagagtccagcatctgggaggccgtcgccacactcacctatagcccggcatgccctgtgcagtttgcctgcctgaagtgggaggccacagccccgggcttcccagggctcccaggctcagagcatgtgctgccggcagggtgtctggagttggaaggacagccatctgcctacctgccccaggaggactgggccccactgggctctgccaggccccctcctccagactcagacagcggcagcagcgactattgcatgttggactgctgtgaggaatgccacctctcagccttcccaggacacaccgagagtcctgagctcacgctagctcagcctgtggcccttcctgtgtccagcagggcctga

SEQ ID NO: 20 - protein, 447 aa, synthetic construct
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPAQRRQGLLVPRWQWSASILVVVPIFLLLTGFVHLLFKLSPRLKRIFYQNIPSPEAFFHPLYSVYHGDFQSWTGARRAGPQARQNGVSTSSAGSESSIWEAVATLTYSPACPVQFACLKWEATAPGFPGLPGSEHVLPAGCLELEGQPSAYLPQEDWAPLGSARPPPPDSDSGSSDYCMLDCCEECHLSAFPGHTESPELTLAQPVALPVSSRA

SEQ ID NO: 21 - DNA, 1344 bp, synthetic construct
  source 1..1344: mol_type = other DNA
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaaggccagcacagaggagacagggcctcctggtcccacgctggcaatggtcagccagcatccttgtagttgtgcccatctttcttctgctgactggctttgtccaccttctgttcaagctgtcacccaggctgaagagaatcttttaccagaacattccatctcccgaggcgttcttccatcctctctacagtgtgtaccatggggacttccagagttggacaggggcccgcagagccggaccacaagcaagacagaatggtgtcagtacttcatcagcaggctcagagtccagcatctgggaggccgtcgccacactcacctatagcccggcatgccctgtgcagtttgcctgcctgaagtgggaggccacagccccgggcttcccagggctcccaggctcagagcatgtgctgccggcagggtgtctggagttggaaggacagccatctgccttcctgccccaggaggactgggccccactgggctctgccaggccccctcctccagactcagacagcggcagcagcgactattgcatgttggactgctgtgaggaatgccacctctcagccttcccaggacacaccgagagtcctgagctcacgctagctcagcctgtggcccttcctgtgtccagcagggcctga

SEQ ID NO: 22 - protein, 447 aa, synthetic construct
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPAQRRQGLLVPRWQWSASILVVVPIFLLLTGFVHLLFKLSPRLKRIFYQNIPSPEAFFHPLYSVYHGDFQSWTGARRAGPQARQNGVSTSSAGSESSIWEAVATLTYSPACPVQFACLKWEATAPGFPGLPGSEHVLPAGCLELEGQPSAFLPQEDWAPLGSARPPPPDSDSGSSDYCMLDCCEECHLSAFPGHTESPELTLAQPVALPVSSRA

SEQ ID NO: 23 - DNA, 1344 bp, synthetic construct
  source 1..1344: mol_type = other DNA
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaaggccagcacagaggagacagggcctcctggtcccacgctggcaatggtcagccagcatccttgtagttgtgcccatctttcttctgctgactggctttgtccaccttctgttcaagctgtcacccaggctgaagagaatcttttaccagaacattccatctcccgaggcgttcttccatcctctctacagtgtgtaccatggggacttccagagttggacaggggcccgcagagccggaccacaagcaagacagaatggtgtcagtacttcatcagcaggctcagagtccagcatctgggaggccgtcgccacactcacctatagcccggcatgccctgtgcagtttgcctgcctgaagtgggaggccacagccccgggcttcccagggctcccaggctcagagcatgtgctgccggcagggtgtctggagttggaaggacagccatctgcctacaggccccaggaggactgggccccactgggctctgccaggccccctcctccagactcagacagcggcagcagcgactattgcatgttggactgctgtgaggaatgccacctctcagccttcccaggacacaccgagagtcctgagctcacgctagctcagcctgtggcccttcctgtgtccagcagggcctga

SEQ ID NO: 24 - protein, 447 aa, synthetic construct
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPAQRRQGLLVPRWQWSASILVVVPIFLLLTGFVHLLFKLSPRLKRIFYQNIPSPEAFFHPLYSVYHGDFQSWTGARRAGPQARQNGVSTSSAGSESSIWEAVATLTYSPACPVQFACLKWEATAPGFPGLPGSEHVLPAGCLELEGQPSAYRPQEDWAPLGSARPPPPDSDSGSSDYCMLDCCEECHLSAFPGHTESPELTLAQPVALPVSSRA

SEQ ID NO: 25 - DNA, 1344 bp, synthetic construct
  source 1..1344: mol_type = other DNA
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaaggccagcacagaggagacagggcctcctggtcccacgctggcaatggtcagccagcatccttgtagttgtgcccatctttcttctgctgactggctttgtccaccttctgttcaagctgtcacccaggctgaagagaatcttttaccagaacattccatctcccgaggcgttcttccatcctctctacagtgtgtaccatggggacttccagagttggacaggggcccgcagagccggaccacaagcaagacagaatggtgtcagtacttcatcagcaggctcagagtccagcatctgggaggccgtcgccacactcacctatagcccggcatgccctgtgcagtttgcctgcctgaagtgggaggccacagccccgggcttcccagggctcccaggctcagagcatgtgctgccggcagggtgtctggagttggaaggacagccatctgcctacctgcccctggaggactgggccccactgggctctgccaggccccctcctccagactcagacagcggcagcagcgactattgcatgttggactgctgtgaggaatgccacctctcagccttcccaggacacaccgagagtcctgagctcacgctagctcagcctgtggcccttcctgtgtccagcagggcctga

SEQ ID NO: 26 - protein, 447 aa, synthetic construct
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPAQRRQGLLVPRWQWSASILVVVPIFLLLTGFVHLLFKLSPRLKRIFYQNIPSPEAFFHPLYSVYHGDFQSWTGARRAGPQARQNGVSTSSAGSESSIWEAVATLTYSPACPVQFACLKWEATAPGFPGLPGSEHVLPAGCLELEGQPSAYLPLEDWAPLGSARPPPPDSDSGSSDYCMLDCCEECHLSAFPGHTESPELTLAQPVALPVSSRA

SEQ ID NO: 27 - DNA, 1344 bp, synthetic construct
  source 1..1344: mol_type = other DNA
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaaggccagcacagaggagacagggcctcctggtcccacgctggcaatggtcagccagcatccttgtagttgtgcccatctttcttctgctgactggctttgtccaccttctgttcaagctgtcacccaggctgaagagaatcttttaccagaacattccatctcccgaggcgttcttccatcctctctacagtgtgtaccatggggacttccagagttggacaggggcccgcagagccggaccacaagcaagacagaatggtgtcagtacttcatcagcaggctcagagtccagcatctgggaggccgtcgccacactcacctatagcccggcatgccctgtgcagtttgcctgcctgaagtgggaggccacagccccgggcttcccagggctcccaggctcagagcatgtgctgccggcagggtgtctggagttggaaggacagccatctgcctacctgaagcaggaggactgggccccactgggctctgccaggccccctcctccagactcagacagcggcagcagcgactattgcatgttggactgctgtgaggaatgccacctctcagccttcccaggacacaccgagagtcctgagctcacgctagctcagcctgtggcccttcctgtgtccagcagggcctga

SEQ ID NO: 28 - protein, 447 aa, synthetic construct
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPAQRRQGLLVPRWQWSASILVVVPIFLLLTGFVHLLFKLSPRLKRIFYQNIPSPEAFFHPLYSVYHGDFQSWTGARRAGPQARQNGVSTSSAGSESSIWEAVATLTYSPACPVQFACLKWEATAPGFPGLPGSEHVLPAGCLELEGQPSAYLKQEDWAPLGSARPPPPDSDSGSSDYCMLDCCEECHLSAFPGHTESPELTLAQPVALPVSSRA

SEQ ID NO: 29 - DNA, 1344 bp, synthetic construct
  source 1..1344: mol_type = other DNA
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaaggccagcacagaggagacagggcctcctggtcccacgctggcaatggtcagccagcatccttgtagttgtgcccatctttcttctgctgactggctttgtccaccttctgttcaagctgtcacccaggctgaagagaatcttttaccagaacattccatctcccgaggcgttcttccatcctctctacagtgtgtaccatggggacttccagagttggacaggggcccgcagagccggaccacaagcaagacagaatggtgtcagtacttcatcagcaggctcagagtccagcatctgggaggccgtcgccacactcacctatagcccggcatgccctgtgcagtttgcctgcctgaagtgggaggccacagccccgggcttcccagggctcccaggctcagagcatgtgctgccggcagggtgtctggagttggaaggacagccatctgcctacctgnnncaggaggactgggccccactgggctctgccaggccccctcctccagactcagacagcggcagcagcgactattgcatgttggactgctgtgaggaatgccacctctcagccttcccaggacacaccgagagtcctgagctcacgctagctcagcctgtggcccttcctgtgtccagcagggcctga

SEQ ID NO: 30 - protein, 447 aa, synthetic construct
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPAQRRQGLLVPRWQWSASILVVVPIFLLLTGFVHLLFKLSPRLKRIFYQNIPSPEAFFHPLYSVYHGDFQSWTGARRAGPQARQNGVSTSSAGSESSIWEAVATLTYSPACPVQFACLKWEATAPGFPGLPGSEHVLPAGCLELEGQPSAYLXQEDWAPLGSARPPPPDSDSGSSDYCMLDCCEECHLSAFPGHTESPELTLAQPVALPVSSRA

SEQ ID NO: 31 - DNA, 1344 bp, synthetic construct
  source 1..1344: mol_type = other DNA
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaaggccagcacagaggagacagggcctcctggtcccacgctggcaatggtcagccagcatccttgtagttgtgcccatctttcttctgctgactggctttgtccaccttctgttcaagctgtcacccaggctgaagagaatcttttaccagaacattccatctcccgaggcgttcttccatcctctctacagtgtgtaccatggggacttccagagttggacaggggcccgcagagccggaccacaagcaagacagaatggtgtcagtacttcatcagcaggctcagagtccagcatctgggaggccgtcgccacactcacctatagcccggcatgccctgtgcagtttgcctgcctgaagtgggaggccacagccccgggcttcccagggctcccaggctcagagcatgtgctgccggcagggtgtctggagttggaaggacagccatctgcctacctgccccaggaggactgggccccactgggctctgccaggccccctcctccagactcagacctgaacaccgatgcctatctgtctcttcaagaatgtgaggaatgccacctctcagccttcccaggacacaccgagagtcctgagctcacgctagctcagcctgtggcccttcctgtgtccagcagggcctga

SEQ ID NO: 32 - protein, 447 aa, synthetic construct
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPAQRRQGLLVPRWQWSASILVVVPIFLLLTGFVHLLFKLSPRLKRIFYQNIPSPEAFFHPLYSVYHGDFQSWTGARRAGPQARQNGVSTSSAGSESSIWEAVATLTYSPACPVQFACLKWEATAPGFPGLPGSEHVLPAGCLELEGQPSAYLPQEDWAPLGSARPPPPDSDLNTDAYLSLQECEECHLSAFPGHTESPELTLAQPVALPVSSRA

SEQ ID NO: 33 - DNA, 1377 bp, synthetic construct
  source 1..1377: mol_type = other DNA
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaaggccagcacagaggagacagggcctcctggtcccacgctggcaatggtcagccagcatccttgtagttgtgcccatctttcttctgctgactggctttgtccaccttctgttcaagctgtcacccaggctgaagagaatcttttaccagaacattccatctcccgaggcgttcttccatcctctctacagtgtgtaccatggggacttccagagttggacaggggcccgcagagccggaccacaagcaagacagaatggtgtcagtacttcatcagcaggctcagagtccagcatctgggaggccgtcgccacactcacctatagcccggcatgccctgtgcagtttgcctgcctgaagtgggaggccacagccccgggcttcccagggctcccaggctcagagcatgtgctgccggcagggtgtctggagttggaaggacagccatctgcctacctgccccaggaggactgggccccactgggctctgccaggccccctcctccagactcagacctgaacacagacgcctacctgagcctgcaagagctgaacaccgatgcttacctgtctctccaagagtgtgaggaatgccacctctcagccttcccaggacacaccgagagtcctgagctcacgctagctcagcctgtggcccttcctgtgtccagcagggcctga

SEQ ID NO: 34 - protein, 458 aa, synthetic construct
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPAQRRQGLLVPRWQWSASILVVVPIFLLLTGFVHLLFKLSPRLKRIFYQNIPSPEAFFHPLYSVYHGDFQSWTGARRAGPQARQNGVSTSSAGSESSIWEAVATLTYSPACPVQFACLKWEATAPGFPGLPGSEHVLPAGCLELEGQPSAYLPQEDWAPLGSARPPPPDSDLNTDAYLSLQELNTDAYLSLQECEECHLSAFPGHTESPELTLAQPVALPVSSRA

SEQ ID NO: 35 - DNA, 1410 bp, synthetic construct
  source 1..1410: mol_type = other DNA
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaaggccagcacagaggagacagggcctcctggtcccacgctggcaatggtcagccagcatccttgtagttgtgcccatctttcttctgctgactggctttgtccaccttctgttcaagctgtcacccaggctgaagagaatcttttaccagaacattccatctcccgaggcgttcttccatcctctctacagtgtgtaccatggggacttccagagttggacaggggcccgcagagccggaccacaagcaagacagaatggtgtcagtacttcatcagcaggctcagagtccagcatctgggaggccgtcgccacactcacctatagcccggcatgccctgtgcagtttgcctgcctgaagtgggaggccacagccccgggcttcccagggctcccaggctcagagcatgtgctgccggcagggtgtctggagttggaaggacagccatctgcctacctgccccaggaggactgggccccactgggctctgccaggccccctcctccagactcagacctgaacacagacgcctacctgagcctgcaagagctgaacaccgatgcttacctgtctctgcaagaactcaataccgacgcatacttgtccctccaagagtgtgaggaatgccacctctcagccttcccaggacacaccgagagtcctgagctcacgctagctcagcctgtggcccttcctgtgtccagcagggcctga

SEQ ID NO: 36 - protein, 469 aa, synthetic construct
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPAQRRQGLLVPRWQWSASILVVVPIFLLLTGFVHLLFKLSPRLKRIFYQNIPSPEAFFHPLYSVYHGDFQSWTGARRAGPQARQNGVSTSSAGSESSIWEAVATLTYSPACPVQFACLKWEATAPGFPGLPGSEHVLPAGCLELEGQPSAYLPQEDWAPLGSARPPPPDSDLNTDAYLSLQELNTDAYLSLQELNTDAYLSLQECEECHLSAFPGHTESPELTLAQPVALPVSSRA

SEQ ID NO: 37 - DNA, 1410 bp, synthetic construct
  source 1..1410: mol_type = other DNA
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaaggccagcacagaggagacagggcctcctggtcccacgctggcaatggtcagccagcatccttgtagttgtgcccatctttcttctgctgactggctttgtccaccttctgttcaagctgtcacccaggctgaagagaatcttttaccagaacattccatctcccgaggcgttcttccatcctctctacagtgtgtaccatggggacttccagagttggacaggggcccgcagagccggaccacaagcaagacagaatggtgtcagtacttcatcagcaggctcagagtccagcatctgggaggccgtcgccacactcacctatagcccggcatgccctgtgcagtttgcctgcctgaagtgggaggccacagccccgggcttcccagggctcccaggctcagagcatgtgctgccggcagggtgtctggagttggaaggacagccatctgcctacctgnnncaggaggactgggccccactgggctctgccaggccccctcctccagactcagacctgaacacagacgcctacctgagcctgcaagagctgaacaccgatgcttacctgtctctgcaagaactcaataccgacgcatacttgtccctccaagagtgtgaggaatgccacctctcagccttcccaggacacaccgagagtcctgagctcacgctagctcagcctgtggcccttcctgtgtccagcagggcctga

SEQ ID NO: 38 - protein, 469 aa, synthetic construct
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPAQRRQGLLVPRWQWSASILVVVPIFLLLTGFVHLLFKLSPRLKRIFYQNIPSPEAFFHPLYSVYHGDFQSWTGARRAGPQARQNGVSTSSAGSESSIWEAVATLTYSPACPVQFACLKWEATAPGFPGLPGSEHVLPAGCLELEGQPSAYLXQEDWAPLGSARPPPPDSDLNTDAYLSLQELNTDAYLSLQELNTDAYLSLQECEECHLSAFPGHTESPELTLAQPVALPVSSRA

SEQ ID NO: 39 - DNA, 1614 bp, synthetic construct
  source 1..1614: mol_type = other DNA
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaagacctgctggcgaacctgaagctggatgggaccctcatatgttgctgctgctggccgtgctgatcatcgtgctggtgttcatgggcctgaagatccatctgccttggagactgtggaagaaaatctgggcccctgtgcctactcctgagagcttcttccagccactgtacagagagcacagcggcaacttcaagaaatgggtcaacacccctttcaccgccagcagtatcgagctggtgcctcagagcagcaccacaacatctgccctgcacctgtctctgtaccccgccaaagagaagaagttccctggcctgcctggactggaagaacagctggaatgtgacggcatgagcgagcctggccactggtgtatcattcctctggctgctggacaggccgtgtccgcctatagcgaggaaagagacagaccctacggcctggtgtccatcgacacagtgacagtgggagatgccgagggcctgtgtgtgtggccttgtagctgtgaagatgacggctaccctgccatgaacctggatgccggaagagagagcggccctaactctgaggatctgctgctcgtgaccgatcctgccttcctgtcttgcggctgtgtgtctggatctggcctgagactcggaggctctcctggaagcctgctggatagactgagactgagcttcgccaaagaaggcgactggaccgccgatcctacttggagaacaggatctcctggcggcggaagcgaatctgaagcaggttctccacctggcctggacatggacacattcgactctggcttcgccggcagcgattgtggaagccctgtggaaacagacgagggcccacctagaagctacctgagacagtgggtcgtgcggacacctcctccagttgattctggcgcccagtcctcttga

SEQ ID NO: 40 - protein, 537 aa, synthetic construct
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPAGEPEAGWDPHMLLLLAVLIIVLVFMGLKIHLPWRLWKKIWAPVPTPESFFQPLYREHSGNFKKWVNTPFTASSIELVPQSSTTTSALHLSLYPAKEKKFPGLPGLEEQLECDGMSEPGHWCIIPLAAGQAVSAYSEERDRPYGLVSIDTVTVGDAEGLCVWPCSCEDDGYPAMNLDAGRESGPNSEDLLLVTDPAFLSCGCVSGSGLRLGGSPGSLLDRLRLSFAKEGDWTADPTWRTGSPGGGSESEAGSPPGLDMDTFDSGFAGSDCGSPVETDEGPPRSYLRQWVVRTPPPVDSGAQSS

SEQ ID NO: 41 - DNA, 1743 bp, synthetic construct
  source 1..1743: mol_type = other DNA
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaagacctgctggcgaacctgaagctggatgggaccctcatatgttgctgctgctggccgtgctgatcatcgtgctggtgttcatgggcctgaagatccatctgccttggagactgtggaagaaaatctgggcccctgtgcctactcctgagagcttcttccagccactgtacagagagcacagcggcaacttcaagaaatgggtcaacacccctttcaccgccagcagtatcgagctggtgcctcagagcagcaccacaacatctgccctgcacctgtctctgtaccccgccaaagagaagaagttccctggcctgcctggactggaagaacagctggaatgtgacggcatgagcgagcctggccactggtgtatcattcctctggctgctggacaggccgtgtccgcctatagcgaggaaagagacagaccctacggcctggtgtccatcgacacagtgacagtgggagatgccgagggcctgtgtgtgtggccttgtagctgtgaagatgacggctaccctgccatgaacctggatgccggaagagagagcggccctaactctgaggatctgctgctcgtgaccgatcctgccttcctgtcttgcggctgtgtgtctggatctggcctgagactcggaggctctcctggaagcctgctggatagactgagactgagcttcgccaaagaaggcgactggaccgccgatcctacttggagaacaggatctcctggcggcggaagcgaatctgaagcaggttctccacctggcctggacatggacacattcgactctggcttcgccggcagcgattgtggaagccctgtggaaacagacgagggcccacctagaagctacctgagacagtgggtcgtgcggacacctcctccagttgattctggcgcccagtcctctctgaacacagacgcctacctgagcctgcaagagctgaacaccgatgcttacctgtctctgcaagaactcaataccgacgcatacttgtccctccaagagctgcaggcccaggactctgtgcacctgatttga

SEQ ID NO: 42 - protein, 580 aa, synthetic construct
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPAGEPEAGWDPHMLLLLAVLIIVLVFMGLKIHLPWRLWKKIWAPVPTPESFFQPLYREHSGNFKKWVNTPFTASSIELVPQSSTTTSALHLSLYPAKEKKFPGLPGLEEQLECDGMSEPGHWCIIPLAAGQAVSAYSEERDRPYGLVSIDTVTVGDAEGLCVWPCSCEDDGYPAMNLDAGRESGPNSEDLLLVTDPAFLSCGCVSGSGLRLGGSPGSLLDRLRLSFAKEGDWTADPTWRTGSPGGGSESEAGSPPGLDMDTFDSGFAGSDCGSPVETDEGPPRSYLRQWVVRTPPPVDSGAQSSLNTDAYLSLQELNTDAYLSLQELNTDAYLSLQELQAQDSVHLI

SEQ ID NO: 43 - DNA, 1614 bp, synthetic construct
  source 1..1614: mol_type = other DNA
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaagacctgctggcgaacctgaagctggatgggaccctcatatgttgctgctgctggccgtgctgatcatcgtgctggtgttcatgggcctgaagatccatctgccttggagactgtggaagaaaatctgggcccctgtgcctactcctgagagcttcttccagccactgtacagagagcacagcggcaacttcaagaaatgggtcaacacccctttcaccgccagcagtatcgagctggtgcctcagagcagcaccacaacatctgccctgcacctgtctctgtaccccgccaaagagaagaagttccctggcctgcctggactggaagaacagctggaatgtgacggcatgagcgagcctggccactggtgtatcattcctctggctgctggacaggccgtgtccgcctatagcgaggaaagagacagaccctacggcctggtgtccatcgacacagtgacagtgggagatgccgagggcctgtgtgtgtggccttgtagctgtgaagatgacggctaccctgccatgaacctggatgccggaagagagagcggccctaactctgaggatctgctgctcgtgaccgatcctgccttcctgtcttgcggctgtgtgtctggatctggcctgagactcggaggctctcctggaagcctgctggatagactgagactgagcttcgccaaagaaggcgactggaccgccgatcctacttggagaacaggatctcctggcggcggaagcgaatctgaagcaggttctccacctggcctggacatggacacattcgactctggcttcgccggcagcgattgtggaagccctgtggaaacagacgagggcccacctagcgcctacctgagacagtgggtcgtgcggacacctcctccagttgattctggcgcccagtcctcttga

SEQ ID NO: 44 - protein, 537 aa, synthetic construct
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPAGEPEAGWDPHMLLLLAVLIIVLVFMGLKIHLPWRLWKKIWAPVPTPESFFQPLYREHSGNFKKWVNTPFTASSIELVPQSSTTTSALHLSLYPAKEKKFPGLPGLEEQLECDGMSEPGHWCIIPLAAGQAVSAYSEERDRPYGLVSIDTVTVGDAEGLCVWPCSCEDDGYPAMNLDAGRESGPNSEDLLLVTDPAFLSCGCVSGSGLRLGGSPGSLLDRLRLSFAKEGDWTADPTWRTGSPGGGSESEAGSPPGLDMDTFDSGFAGSDCGSPVETDEGPPSAYLRQWVVRTPPPVDSGAQSS

SEQ ID NO: 45 - DNA, 1614 bp, synthetic construct
  source 1..1614: mol_type = other DNA
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaagacctgctggcgaacctgaagctggatgggaccctcatatgttgctgctgctggccgtgctgatcatcgtgctggtgttcatgggcctgaagatccatctgccttggagactgtggaagaaaatctgggcccctgtgcctactcctgagagcttcttccagccactgtacagagagcacagcggcaacttcaagaaatgggtcaacacccctttcaccgccagcagtatcgagctggtgcctcagagcagcaccacaacatctgccctgcacctgtctctgtaccccgccaaagagaagaagttccctggcctgcctggactggaagaacagctggaatgtgacggcatgagcgagcctggccactggtgtatcattcctctggctgctggacaggccgtgtccgcctatagcgaggaaagagacagaccctacggcctggtgtccatcgacacagtgacagtgggagatgccgagggcctgtgtgtgtggccttgtagctgtgaagatgacggctaccctgccatgaacctggatgccggaagagagagcggccctaactctgaggatctgctgctcgtgaccgatcctgccttcctgtcttgcggctgtgtgtctggatctggcctgagactcggaggctctcctggaagcctgctggatagactgagactgagcttcgccaaagaaggcgactggaccgccgatcctacttggagaacaggatctcctggcggcggaagcgaatctgaagcaggttctccacctggcctggacatggacacattcgactctggcttcgccggcagcgattgtggaagccctgtggaaacagacgagggcccacctgacgcctacctgagacagtgggtcgtgcggacacctcctccagttgattctggcgcccagtcctcttga

SEQ ID NO: 46 - protein, 537 aa, synthetic construct
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPAGEPEAGWDPHMLLLLAVLIIVLVFMGLKIHLPWRLWKKIWAPVPTPESFFQPLYREHSGNFKKWVNTPFTASSIELVPQSSTTTSALHLSLYPAKEKKFPGLPGLEEQLECDGMSEPGHWCIIPLAAGQAVSAYSEERDRPYGLVSIDTVTVGDAEGLCVWPCSCEDDGYPAMNLDAGRESGPNSEDLLLVTDPAFLSCGCVSGSGLRLGGSPGSLLDRLRLSFAKEGDWTADPTWRTGSPGGGSESEAGSPPGLDMDTFDSGFAGSDCGSPVETDEGPPDAYLRQWVVRTPPPVDSGAQSS

SEQ ID NO: 47 - DNA, 1614 bp, synthetic construct
  source 1..1614: mol_type = other DNA
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaagacctgctggcgaacctgaagctggatgggaccctcatatgttgctgctgctggccgtgctgatcatcgtgctggtgttcatgggcctgaagatccatctgccttggagactgtggaagaaaatctgggcccctgtgcctactcctgagagcttcttccagccactgtacagagagcacagcggcaacttcaagaaatgggtcaacacccctttcaccgccagcagtatcgagctggtgcctcagagcagcaccacaacatctgccctgcacctgtctctgtaccccgccaaagagaagaagttccctggcctgcctggactggaagaacagctggaatgtgacggcatgagcgagcctggccactggtgtatcattcctctggctgctggacaggccgtgtccgcctatagcgaggaaagagacagaccctacggcctggtgtccatcgacacagtgacagtgggagatgccgagggcctgtgtgtgtggccttgtagctgtgaagatgacggctaccctgccatgaacctggatgccggaagagagagcggccctaactctgaggatctgctgctcgtgaccgatcctgccttcctgtcttgcggctgtgtgtctggatctggcctgagactcggaggctctcctggaagcctgctggatagactgagactgagcttcgccaaagaaggcgactggaccgccgatcctacttggagaacaggatctcctggcggcggaagcgaatctgaagcaggttctccacctggcctggacatggacacattcgactctggcttcgccggcagcgattgtggaagccctgtggaaacagacgagggcccacctgacgcctacctgnnncagtgggtcgtgcggacacctcctccagttgattctggcgcccagtcctcttga

SEQ ID NO: 48 - protein, 537 aa, synthetic construct
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPAGEPEAGWDPHMLLLLAVLIIVLVFMGLKIHLPWRLWKKIWAPVPTPESFFQPLYREHSGNFKKWVNTPFTASSIELVPQSSTTTSALHLSLYPAKEKKFPGLPGLEEQLECDGMSEPGHWCIIPLAAGQAVSAYSEERDRPYGLVSIDTVTVGDAEGLCVWPCSCEDDGYPAMNLDAGRESGPNSEDLLLVTDPAFLSCGCVSGSGLRLGGSPGSLLDRLRLSFAKEGDWTADPTWRTGSPGGGSESEAGSPPGLDMDTFDSGFAGSDCGSPVETDEGPPDAYLXQWVVRTPPPVDSGAQSS

SEQ ID NO: 49 - DNA, 1554 bp, synthetic construct
  source 1..1554: mol_type = other DNA
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcaggccaggaatcagaatcagcagaatctgccaaaataggaggaataattactgtgtttttgatagcattggtcttgacaagcaccatagtgacactgaaatggattggttatatatgcttaagaaatagcctccccaaagtcttgaattttcataactttttagcctggccatttcctaacctgccaccgttggaagccatggatatggtggaggtcatttacatcaacagaaagaagaaagtgtgggattataattatgatgatgaaagtgatagcgatactgaggcagcgcccaggacaagtggcggtggctataccatgcatggactgactgtcaggcctctgggtcaggcctctgccacctctacagaatcccagttgatagacccggagtccgaggaggagcctgacctgcctgaggttgatgtggagctccccacgatgccaaaggacagccctcagcagttggaactcttgagtgggccctgtgagaggagaaagagtccactccaggacccttttcccgaagaggactacagctccacggaggggtctgggggcagaattaccttcaatgtggacttaaactctgtgtttttgagagttcttgatgacgaggacagtgacgacttagaagcccctctgatgctatcgtctcatctggaagagatggttgacccagaggatcctgataatgtgcaatcaaaccatttgctggccagcggggaagggacacagccaacctttcccagcccctcttcagagggcctgtggtccgaagatgctccatctgatcaaagtgacacttctgagtcagatgttgaccttggggatggttatataatgagatga

SEQ ID NO: 50 - protein, 517 aa, synthetic construct
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAGQESESAESAKIGGIITVFLIALVLTSTIVTLKWIGYICLRNSLPKVLNFHNFLAWPFPNLPPLEAMDMVEVIYINRKKKVWDYNYDDESDSDTEAAPRTSGGGYTMHGLTVRPLGQASATSTESQLIDPESEEEPDLPEVDVELPTMPKDSPQQLELLSGPCERRKSPLQDPFPEEDYSSTEGSGGRITFNVDLNSVFLRVLDDEDSDDLEAPLMLSSHLEEMVDPEDPDNVQSNHLLASGEGTQPTFPSPSSEGLWSEDAPSDQSDTSESDVDLGDGYIMR

SEQ ID NO: 51 - DNA, 1458 bp, synthetic construct
  source 1..1458: mol_type = other DNA
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcattcaatagcagtataaaaggttctctttggattccagttgttgctgctttactactctttctagtgcttagcctggtattcatctgtttttatattaagaaaattaatccattgaaggaaaaaagcataatattacccaagtccttgatctctgtggtaagaagtgctactttagagacaaaacctgaatcaaaatatgtatcactcatcacgtcataccagccattttccttagaaaaggaggtggtctgtgaagagccgttgtctccagcaacagttccaggcatgcataccgaagacaatccaggaaaagtggaacatacagaagaactttctagtataacagaagtggtgactactgaagaaaatattcctgacgtggtcccgggcagccatctgactccaatagagagagagagttcttcacctttaagtagtaaccagtctgaacctggcagcatcgctttaaactcgtatcactccagaaattgttctgagagtgatcactccagaaatggttttgatactgattccagctgtctggaatcacatagctccttatctgactcagaatttcccccaaataataaaggtgaaataaaaacagaaggacaagagctcataaccgtaataaaagcccccacctcctttggttatgataaaccacatgtgctagtggatctacttgtggatgatagcggtaaagagtccttgattggttatagaccaacagaagattccaaagaattttcatga

SEQ ID NO: 52 - protein, 485 aa, synthetic construct
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAFNSSIKGSLWIPVVAALLLFLVLSLVFICFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFS

SEQ ID NO: 53 - DNA, 1602 bp, synthetic construct
  source 1..1602: mol_type = other DNA
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcagccccagaagccaactgggctttcctggtgctgccatcgcttctgatactgctgttagtaattgccgcagggggtgtgatctggaagaccctcatggggaacccctggtttcagcgggcaaagatgccacgggccctggacttttctggacacacacaccctgtggcaacctttcagcccagcagaccagagtccgtgaatgacttgttcctctgtccccaaaaggaactgaccagaggggtcaggccgacgcctcgagtcagggccccagccacccaacagacaagatggaagaaggaccttgcagaggacgaagaggaggaggatgaggaggacacagaagatggcgtcagcttccagccctacattgaaccaccttctttcctggggcaagagcaccaggctccagggcactcggaggctggtggggtggactcagggaggcccagggctcctctggtcccaagcgaaggctcctctgcttgggattcttcagacagaagctgggccagcactgtggactcctcctgggacagggctgggtcctctggctatttggctgagaaggggccaggccaagggccgggtggggatgggcaccaagaatctctcccaccacctgaattctccaaggactcgggtttcctggaagagctcccagaagataacctctcctcctgggccacctggggcaccttaccaccggagccgaatctggtccctgggggacccccagtttctcttcagacactgaccttctgctgggaaagcagccctgaggaggaagaggaggcgagggaatcagaaattgaggacagcgatgcgggcagctggggggctgagagcacccagaggaccgaggacaggggccggacattggggcattacatggccaggtga

SEQ ID NO: 54 - protein, 533 aa, synthetic construct
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAAPEANWAFLVLPSLLILLLVIAAGGVIWKTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 55 - DNA, 1761 bp, synthetic construct
  source 1..1761: mol_type = other DNA
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcaaccaggcagtatttcaccgtgaccaacgtcatcatcttctttgcctttgtcctgctgctctccggagccctcgcctactgcctggccctccagctgtatgtgcggcgccgaaagaagctacccagtgtcctgctcttcaagaagcccagccccttcatcttcatcagccagcgtccctccccagagacccaagacaccatccacccgcttgatgaggaggcctttttgaaggtgtccccagagctgaagaacttggacctgcacggcagcacagacagtggctttggcagcaccaagccatccctgcagactgaagagccccagttcctcctccctgaccctcacccccaggctgacagaacgctgggaaacagggagccccctgtgctgggggacagctgcagtagtggcagcagcaatagcacagacagcgggatctgcctgcaggagcccagcctgagccccagcacagggcccacctgggagcaacaggtggggagcaacagcaggggccaggatgacagtggcattgacttagttcaaaactctgagggccgggctggggacacacagggtggctcggccttgggccaccacagtcccccggagcctgaggtgcctggggaagaagacccagctgctgtggcattccagggttacctgaggcagaccagatgtgctgaagagaaggcaaccaagacaggctgcctggaggaagaatcgcccttgacagatggccttggccccaaattcgggagatgcctggttgatgaggcaggcttgcatccaccagccctggccaagggctatttgaaacaggatcctctagaaatgactctggcttcctcaggggccccaacgggacagtggaaccagcccactgaggaatggtcactcctggccttgagcagctgcagtgacctgggaatatctgactggagctttgcccatgaccttgcccctctaggctgtgtggcagccccaggtggtctcctgggcagctttaactcagacctggtcaccctgcccctcatctctagcctgcagtcaagtgagtga

SEQ ID NO: 56 - protein, 586 aa, synthetic construct
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPATRQYFTVTNVIIFFAFVLLLSGALAYCLALQLYVRRRKKLPSVLLFKKPSPFIFISQRPSPETQDTIHPLDEEAFLKVSPELKNLDLHGSTDSGFGSTKPSLQTEEPQFLLPDPHPQADRTLGNREPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSE

SEQ ID NO: 57 - DNA, 1638 bp, synthetic construct
  source 1..1638: mol_type = other DNA
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcacaatcatcagagttcaaggctaaaatcatcttctggtatgttttgcccgtatctattaccgtgtttcttttttctgtgatgggctattccatctaccgatatatccacgttggcaaagagaaacacccagcaaatttgattttgatttatggaaatgaatttgacaaaagattctttgtgcctgctgaaaaaatcgtgattaactttatcaccctcaatatctcggatgattctaaaatttctcatcaggatatgagtttactgggaaaaagcagtgatgtatccagccttaatgatcctcagcccagcgggaacctgaggccccctcaggaggaagaggaggtgaaacatttagggtatgcttcgcatttgatggaaattttttgtgactctgaagaaaacacggaaggtacttctctcacccagcaagagtccctcagcagaacaatacccccggataaaacagtcattgaatatgaatatgatgtcagaaccactgacatttgtgcggggcctgaagagcaggagctcagtttgcaggaggaggtgtccacacaaggaacattattggagtcgcaggcagcgttggcagtcttgggcccgcaaacgttacagtactcatacacccctcagctccaagacttagaccccctggcgcaggagcacacagactcggaggaggggccggaggaagagccatcgacgaccctggtcgactgggatccccaaactggcaggctgtgtattccttcgctgtccagcttcgaccaggattcagagggctgcgagccttctgagggggatgggctcggagaggagggtcttctatctagactctatgaggagccggctccagacaggccaccaggagaaaatgaaacctatctcatgcaattcatggaggaatgggggttatatgtgcagatggaaaactga

SEQ ID NO: 58 - protein, 545 aa, synthetic construct
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAQSSEFKAKIIFWYVLPVSITVFLFSVMGYSIYRYIHVGKEKHPANLILIYGNEFDKRFFVPAEKIVINFITLNISDDSKISHQDMSLLGKSSDVSSLNDPQPSGNLRPPQEEEEVKHLGYASHLMEIFCDSEENTEGTSLTQQESLSRTIPPDKTVIEYEYDVRTTDICAGPEEQELSLQEEVSTQGTLLESQAALAVLGPQTLQYSYTPQLQDLDPLAQEHTDSEEGPEEEPSTTLVDWDPQTGRLCIPSLSSFDQDSEGCEPSEGDGLGEEGLLSRLYEEPAPDRPPGENETYLMQFMEEWGLYVQMEN

SEQ ID NO: 59 - DNA, 1761 bp, synthetic construct
  source 1..1761: mol_type = other DNA
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcaccagaccggacatggacctactccttctccggagccttcctgttctccatgggcttcctcgtcgcagtactctgctacctgagctacagatatgtcaccaagccgcctgcacctcccaactccctgaacgtccagcgagtcctgactttccagccgctgcgcttcatccaggagcacgtcctgatccctgtctttgacctcagcggccccagcagtctggcccagcctgtccagtactcccagatcagggtgtctggacccagggagcccgcaggagctccacagcggcatagcctgtccgagatcacctacttagggcagccagacatctccatcctccagccctccaacgtgccacctccccagatcctctccccactgtcctatgccccaaacgctgcccctgaggtcgggcccccatcctatgcacctcaggtgacccccgaagctcaattcccattctacgccccacaggccatctctaaggtccagccttcctcctatgcccctcaagccactccggacagctggcctccctcctatggggtatgcatggaaggttctggcaaagactcccccactgggacactttctagtcctaaacaccttaggcctaaaggtcagcttcagaaagagccaccagctggaagctgcatgttaggtggcctttctctgcaggaggtgacctccttggctatggaggaatcccaagaagcaaaatcattgcaccagcccctggggatttgcacagacagaacatctgacccaaatgtgctacacagtggggaggaagggacaccacagtacctaaagggccagctccccctcctctcctcagtccagatcgagggccaccccatgtccctccctttgcaacctccttcccgtccatgttccccctcggaccaaggtccaagtccctggggcctgctggagtcccttgtgtgtcccaaggatgaagccaagagcccagcccctgagacctcagacctggagcagcccacagaactggattctcttttcagaggcctggccctgactgtgcagtgggagtcctga

SEQ ID NO: 60 - protein, 586 aa, synthetic construct
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAPDRTWTYSFSGAFLFSMGFLVAVLCYLSYRYVTKPPAPPNSLNVQRVLTFQPLRFIQEHVLIPVFDLSGPSSLAQPVQYSQIRVSGPREPAGAPQRHSLSEITYLGQPDISILQPSNVPPPQILSPLSYAPNAAPEVGPPSYAPQVTPEAQFPFYAPQAISKVQPSSYAPQATPDSWPPSYGVCMEGSGKDSPTGTLSSPKHLRPKGQLQKEPPAGSCMLGGLSLQEVTSLAMEESQEAKSLHQPLGICTDRTSDPNVLHSGEEGTPQYLKGQLPLLSSVQIEGHPMSLPLQPPSRPCSPSDQGPSPWGLLESLVCPKDEAKSPAPETSDLEQPTELDSLFRGLALTVQWES

SEQ ID NO: 61 - DNA, 1497 bp, synthetic construct
  source 1..1497: mol_type = other DNA
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgctcagagacagggacctctgattcctccttggggctggcctggaaataccctggtggccgtgtccatcttcctgctgctgaccggaccaacctacctgctgttcaagctgagccccagagtgaagcggatcttctaccagaacgtgccctctcctgccatgttcttccagcctctgtacagcgtgcacaacggcaacttccagacatggatgggagcacatggtgccggcgtgctgctgtctcaagattgtgctggaacaccccagggcgctctggaaccttgtgtgcaagaagccaccgctctgctgacatgcggacctgccagaccttggaaaagcgtggccctggaagaggaacaagaaggccctggcacaagactgcccggcaatctgtctagcgaggatgtgctgcctgccggctgtactgaatggcgagttcagaccctggcctacctgcctcaagaagattgggcccctaccagcctgaccagacctgctcctcctgatagcgagggcagcagaagcagcagcagctccagctcctccaacaacaacaattactgtgccctgggctgctacggcggctggcatctttctgctctgcctggcaacacacagagcagcggccctattcctgctctggcctgtggcctgagctgtgatcaccagggactcgagacacagcagggcgttgcatgggttctcgccggccattgtcaaagacctggcctgcacgaagatctgcagggcatgctgctgcctagcgtgctgtctaaggccagaagctggaccttctga

SEQ ID NO: 62 - protein, 498 aa, synthetic construct
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAQRQGPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 63 - DNA, 1497 bp, synthetic construct
  source 1..1497: mol_type = other DNA
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgctcagagacagggacctctgattcctccttggggctggcctggaaataccctggtggccgtgtccatcttcctgctgctgaccggaccaacctacctgctgttcaagctgagccccagagtgaagcggatcttctaccagaacgtgccctctcctgccatgttcttccagcctctgtacagcgtgcacaacggcaacttccagacatggatgggagcacatggtgccggcgtgctgctgtctcaagattgtgctggaacaccccagggcgctctggaaccttgtgtgcaagaagccaccgctctgctgacatgcggacctgccagaccttggaaaagcgtggccctggaagaggaacaagaaggccctggcacaagactgcccggcaatctgtctagcgaggatgtgctgcctgccggctgtactgaatggcgagttcagaccctggccttcctgcctcaagaagattgggcccctaccagcctgaccagacctgctcctcctgatagcgagggcagcagaagcagcagcagctccagctcctccaacaacaacaattactgtgccctgggctgctacggcggctggcatctttctgctctgcctggcaacacacagagcagcggccctattcctgctctggcctgtggcctgagctgtgatcaccagggactcgagacacagcagggcgttgcatgggttctcgccggccattgtcaaagacctggcctgcacgaagatctgcagggcatgctgctgcctagcgtgctgtctaaggccagaagctggaccttctga

SEQ ID NO: 64 - protein, 498 aa, synthetic construct
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAQRQGPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAFLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 65 - DNA, 1497 bp, synthetic construct
  source 1..1497: mol_type = other DNA
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgctcagagacagggacctctgattcctccttggggctggcctggaaataccctggtggccgtgtccatcttcctgctgctgaccggaccaacctacctgctgttcaagctgagccccagagtgaagcggatcttctaccagaacgtgccctctcctgccatgttcttccagcctctgtacagcgtgcacaacggcaacttccagacatggatgggagcacatggtgccggcgtgctgctgtctcaagattgtgctggaacaccccagggcgctctggaaccttgtgtgcaagaagccaccgctctgctgacatgcggacctgccagaccttggaaaagcgtggccctggaagaggaacaagaaggccctggcacaagactgcccggcaatctgtctagcgaggatgtgctgcctgccggctgtactgaatggcgagttcagaccctggcctacaggcctcaagaagattgggcccctaccagcctgaccagacctgctcctcctgatagcgagggcagcagaagcagcagcagctccagctcctccaacaacaacaattactgtgccctgggctgctacggcggctggcatctttctgctctgcctggcaacacacagagcagcggccctattcctgctctggcctgtggcctgagctgtgatcaccagggactcgagacacagcagggcgttgcatgggttctcgccggccattgtcaaagacctggcctgcacgaagatctgcagggcatgctgctgcctagcgtgctgtctaaggccagaagctggaccttctga

SEQ ID NO: 66 - protein, 498 aa, synthetic construct
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAQRQGPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYRPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 67 - DNA, 1497 bp, synthetic construct
  source 1..1497: mol_type = other DNA
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgctcagagacagggacctctgattcctccttggggctggcctggaaataccctggtggccgtgtccatcttcctgctgctgaccggaccaacctacctgctgttcaagctgagccccagagtgaagcggatcttctaccagaacgtgccctctcctgccatgttcttccagcctctgtacagcgtgcacaacggcaacttccagacatggatgggagcacatggtgccggcgtgctgctgtctcaagattgtgctggaacaccccagggcgctctggaaccttgtgtgcaagaagccaccgctctgctgacatgcggacctgccagaccttggaaaagcgtggccctggaagaggaacaagaaggccctggcacaagactgcccggcaatctgtctagcgaggatgtgctgcctgccggctgtactgaatggcgagttcagaccctggcctacctgcctctggaagattgggcccctaccagcctgaccagacctgctcctcctgatagcgagggcagcagaagcagcagcagctccagctcctccaacaacaacaattactgtgccctgggctgctacggcggctggcatctttctgctctgcctggcaacacacagagcagcggccctattcctgctctggcctgtggcctgagctgtgatcaccagggactcgagacacagcagggcgttgcatgggttctcgccggccattgtcaaagacctggcctgcacgaagatctgcagggcatgctgctgcctagcgtgctgtctaaggccagaagctggaccttctga

SEQ ID NO: 68 - protein, 498 aa, synthetic construct
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAQRQGPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPLEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 69 - DNA, 1497 bp, synthetic construct
  source 1..1497: mol_type = other DNA
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgctcagagacagggacctctgattcctccttggggctggcctggaaataccctggtggccgtgtccatcttcctgctgctgaccggaccaacctacctgctgttcaagctgagccccagagtgaagcggatcttctaccagaacgtgccctctcctgccatgttcttccagcctctgtacagcgtgcacaacggcaacttccagacatggatgggagcacatggtgccggcgtgctgctgtctcaagattgtgctggaacaccccagggcgctctggaaccttgtgtgcaagaagccaccgctctgctgacatgcggacctgccagaccttggaaaagcgtggccctggaagaggaacaagaaggccctggcacaagactgcccggcaatctgtctagcgaggatgtgctgcctgccggctgtactgaatggcgagttcagaccctggcctacctgnnncaagaagattgggcccctaccagcctgaccagacctgctcctcctgatagcgagggcagcagaagcagcagcagctccagctcctccaacaacaacaattactgtgccctgggctgctacggcggctggcatctttctgctctgcctggcaacacacagagcagcggccctattcctgctctggcctgtggcctgagctgtgatcaccagggactcgagacacagcagggcgttgcatgggttctcgccggccattgtcaaagacctggcctgcacgaagatctgcagggcatgctgctgcctagcgtgctgtctaaggccagaagctggaccttctga

SEQ ID NO: 70 - protein, 498 aa, synthetic construct
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAQRQGPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLXQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 71 - DNA, 1563 bp, synthetic construct
  source 1..1563: mol_type = other DNA
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgctcagagacagggacctctgattcctccttggggctggcctggaaataccctggtggccgtgtccatcttcctgctgctgaccggaccaacctacctgctgttcaagctgagccccagagtgaagcggatcttctaccagaacgtgccctctcctgccatgttcttccagcctctgtacagcgtgcacaacggcaacttccagacatggatgggagcacatggtgccggcgtgctgctgtctcaagattgtgctggaacaccccagggcgctctggaaccttgtgtgcaagaagccaccgctctgctgacatgcggacctgccagaccttggaaaagcgtggccctggaagaggaacaagaaggccctggcacaagactgcccggcaatctgtctagcgaggatgtgctgcctgccggctgtactgaatggcgagttcagaccctggcctacctgcctcaagaagattgggcccctaccagcctgaccagacctgctcctcctgatagcgagggcagcagaagcagcagcagctccagctccctgaacactgatgcctacttgtccctccaagaactgaacactgatgcctacttgtccctccaagaactgaacactgatgcctacttgtccctccaagaatacggcggctggcatctttctgctctgcctggcaacacacagagcagcggccctattcctgctctggcctgtggcctgagctgtgatcaccagggactcgagacacagcagggcgttgcatgggttctcgccggccattgtcaaagacctggcctgcacgaagatctgcagggcatgctgctgcctagcgtgctgtctaaggccagaagctggaccttctga

SEQ ID NO: 72 - protein, 520 aa, synthetic construct
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAQRQGPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSLNTDAYLSLQELNTDAYLSLQELNTDAYLSLQEYGGWHLSALPGNTQSSGPIPALACGLSCDHQGLETQQGVAWVLAGHCQRPGLHEDLQGMLLPSVLSKARSWTF

SEQ ID NO: 73 - DNA, 1563 bp, synthetic construct
  source 1..1563: mol_type = other DNA
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgctcagagacagggacctctgattcctccttggggctggcctggaaataccctggtggccgtgtccatcttcctgctgctgaccggaccaacctacctgctgttcaagctgagccccagagtgaagcggatcttctaccagaacgtgccctctcctgccatgttcttccagcctctgtacagcgtgcacaacggcaacttccagacatggatgggagcacatggtgccggcgtgctgctgtctcaagattgtgctggaacaccccagggcgctctggaaccttgtgtgcaagaagccaccgctctgctgacatgcggacctgccagaccttggaaaagcgtggccctggaagaggaacaagaaggccctggcacaagactgcccggcaatctgtctagcgaggatgtgctgcctgccggctgtactgaatggcgagttcagaccctggcctacctgnnncaagaagattgggcccctaccagcctgaccagacctgctcctcctgatagcgagggcagcagaagcagcagcagctccagctccctgaacactgatgcctacttgtccctccaagaactgaacactgatgcctacttgtccctccaagaactgaacactgatgcctacttgtccctccaagaatacggcggctggcatctttctgctctgcctggcaacacacagagcagcggccctattcctgctctggcctgtggcctgagctgtgatcaccagggactcgagacacagcagggcgttgcatgggttctcgccggccattgtcaaagacctggcctgcacgaagatctgcagggcatgctgctgcctagcgtgctgtctaaggccagaagctggaccttctga

SEQ ID NO: 74 - protein, 520 aa, synthetic construct
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAQRQGPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLXQEDWAPTSLTRPAPPDSEGSRSSSSSSSLNTDAYLSLQELNTDAYLSLQELNTDAYLSLQEYGGWHLSALPGNTQSSGPIPALACGLSCDHQGLETQQGVAWVLAGHCQRPGLHEDLQGMLLPSVLSKARSWTF

SEQ ID NO: 75 - DNA, 1638 bp, synthetic construct
  source 1..1638: mol_type = other DNA
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgccgaggaactgaaagaaggctggaaccctcacctcctgctgttgctgctgctggtcatcgtgttcatccccgccttttggagcctgaaaacacaccctctgtggcggctgtggaagaaaatctgggccgtgccatctcctgagcggttcttcatgcctctgtacaagggctgcagcggcgacttcaagaaatgggtcggagccccttttaccggcagctctctggaacttggaccttggagccctgaggtgcccagcacactggaagtgtacagctgtcaccctcctagaagccccgccaagagactgcagctgacagagctgcaagaacccgccgagctggtggaatctgatggcgtgccaaagcctagcttctggcccaccgctcagaatagcggcggctctgcctacagcgaggaaagagatagaccctacggcctggtgtccatcgacaccgtgacagtgctggatgccgagggaccttgtacctggccttgtagctgcgaggacgatggctaccctgctctggatctggacgctggccttgagccttctccaggactggaagatcctctgctggacgccggaacaaccgtgctgtcttgtggctgtgtgtctgccggatctcctggacttggaggccctctgggaagcctgctggatagactgaaacctcctctggccgacggcgaagattgggctggtggacttccttggggcggaagatctccaggcggagtgtctgagtctgaagccggttctccactggccggcctggacatggataccttcgattctggcttcgtgggcagcgactgtagcagccctgtggaatgcgacttcacaagccctggcgacgagggcccacctagaagctatctgagacagtgggtcgtgatccctccacctctgtctagtcctggacctcaggcctcttga

SEQ ID NO: 76 - protein, 545 aa, synthetic construct
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 77 - DNA, 1767 bp, synthetic construct
  source 1..1767: mol_type = other DNA
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgccgaggaactgaaagaaggctggaaccctcacctcctgctgttgctgctgctggtcatcgtgttcatccccgccttttggagcctgaaaacacaccctctgtggcggctgtggaagaaaatctgggccgtgccatctcctgagcggttcttcatgcctctgtacaagggctgcagcggcgacttcaagaaatgggtcggagccccttttaccggcagctctctggaacttggaccttggagccctgaggtgcccagcacactggaagtgtacagctgtcaccctcctagaagccccgccaagagactgcagctgacagagctgcaagaacccgccgagctggtggaatctgatggcgtgccaaagcctagcttctggcccaccgctcagaatagcggcggctctgcctacagcgaggaaagagatagaccctacggcctggtgtccatcgacaccgtgacagtgctggatgccgagggaccttgtacctggccttgtagctgcgaggacgatggctaccctgctctggatctggacgctggccttgagccttctccaggactggaagatcctctgctggacgccggaacaaccgtgctgtcttgtggctgtgtgtctgccggatctcctggacttggaggccctctgggaagcctgctggatagactgaaacctcctctggccgacggcgaagattgggctggtggacttccttggggcggaagatctccaggcggagtgtctgagtctgaagccggttctccactggccggcctggacatggataccttcgattctggcttcgtgggcagcgactgtagcagccctgtggaatgcgacttcacaagccctggcgacgagggcccacctagaagctatctgagacagtgggtcgtgatccctccacctctgtctagtcctggacctcaggcctctctgaacacggacgcatatttgtcattgcaagaactcaatacagacgcctatcttagccttcaggaattgaatacagatgcttacttgtctctgcaagagttgcaggggcaggacccgacccacttggtttag

SEQ ID NO: 78 - protein, 588 aa, synthetic construct
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQASLNTDAYLSLQELNTDAYLSLQELNTDAYLSLQELQGQDPTHLV

SEQ ID NO: 79 - DNA, 1638 bp, synthetic construct
  source 1..1638: mol_type = other DNA
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgccgaggaactgaaagaaggctggaaccctcacctcctgctgttgctgctgctggtcatcgtgttcatccccgccttttggagcctgaaaacacaccctctgtggcggctgtggaagaaaatctgggccgtgccatctcctgagcggttcttcatgcctctgtacaagggctgcagcggcgacttcaagaaatgggtcggagccccttttaccggcagctctctggaacttggaccttggagccctgaggtgcccagcacactggaagtgtacagctgtcaccctcctagaagccccgccaagagactgcagctgacagagctgcaagaacccgccgagctggtggaatctgatggcgtgccaaagcctagcttctggcccaccgctcagaatagcggcggctctgcctacagcgaggaaagagatagaccctacggcctggtgtccatcgacaccgtgacagtgctggatgccgagggaccttgtacctggccttgtagctgcgaggacgatggctaccctgctctggatctggacgctggccttgagccttctccaggactggaagatcctctgctggacgccggaacaaccgtgctgtcttgtggctgtgtgtctgccggatctcctggacttggaggccctctgggaagcctgctggatagactgaaacctcctctggccgacggcgaagattgggctggtggacttccttggggcggaagatctccaggcggagtgtctgagtctgaagccggttctccactggccggcctggacatggataccttcgattctggcttcgtgggcagcgactgtagcagccctgtggaatgcgacttcacaagccctggcgacgagggcccacctagcgcctatctgagacagtgggtcgtgatccctccacctctgtctagtcctggacctcaggcctcttga

SEQ ID NO: 80 - protein, 545 aa, synthetic construct
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPSAYLRQWVVIPPPLSSPGPQAS

SEQ ID NO: 81 - DNA, 1638 bp, synthetic construct
  source 1..1638: mol_type = other DNA
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgccgaggaactgaaagaaggctggaaccctcacctcctgctgttgctgctgctggtcatcgtgttcatccccgccttttggagcctgaaaacacaccctctgtggcggctgtggaagaaaatctgggccgtgccatctcctgagcggttcttcatgcctctgtacaagggctgcagcggcgacttcaagaaatgggtcggagccccttttaccggcagctctctggaacttggaccttggagccctgaggtgcccagcacactggaagtgtacagctgtcaccctcctagaagccccgccaagagactgcagctgacagagctgcaagaacccgccgagctggtggaatctgatggcgtgccaaagcctagcttctggcccaccgctcagaatagcggcggctctgcctacagcgaggaaagagatagaccctacggcctggtgtccatcgacaccgtgacagtgctggatgccgagggaccttgtacctggccttgtagctgcgaggacgatggctaccctgctctggatctggacgctggccttgagccttctccaggactggaagatcctctgctggacgccggaacaaccgtgctgtcttgtggctgtgtgtctgccggatctcctggacttggaggccctctgggaagcctgctggatagactgaaacctcctctggccgacggcgaagattgggctggtggacttccttggggcggaagatctccaggcggagtgtctgagtctgaagccggttctccactggccggcctggacatggataccttcgattctggcttcgtgggcagcgactgtagcagccctgtggaatgcgacttcacaagccctggcgacgagggcccacctgacgcctatctgnnncagtgggtcgtgatccctccacctctgtctagtcctggacctcaggcctcttga

SEQ ID NO: 82 - protein, 545 aa, synthetic construct
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPDAYLXQWVVIPPPLSSPGPQAS

SEQ ID NO: 83 - DNA, 129 bp, synthetic construct
  source 1..129: mol_type = other DNA
ctgaacacagacgcctacctgagcctgcaagagctgaacaccgatgcttacctgtctctgcaagaactcaataccgacgcatacttgtccctccaagagctgcaggcccaggactctgtgcacctgatt

SEQ ID NO: 84 - protein, 43 aa, synthetic construct
LNTDAYLSLQELNTDAYLSLQELNTDAYLSLQELQAQDSVHLI

SEQ ID NO: 85 - DNA, 1368 bp, synthetic construct
  source 1..1368: mol_type = other DNA
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaaggccagcagatcccatgaaggagatcctccccatgtcatggctcagataccttctgctggtccttggttgtttttctggcttcttctcctgcgtctacattttggtcaaacgcagaggaaagacttccttctggtcagatgtgccagacccagcccacagtagcctgagctcctggttgcccaccatcatgacagaggaaaccttccagttacccagcttctgggactccagcgtgccatcaatcaccaagatcactgaactggaggaagacaagaaaccgacccactgggattccgaaagctctgggaatggtagccttccagccctggttcaggcctatgtgctccaaggagatccaagagaaatttccaaccagtcccagcctccctctcgcactggtgaccaggtcctctatggtcaggtgcttgagagccccaccagcccaggagtaatgcagtacattcgctctgactccactcagcccctcttggggggccccacccctagccctaaatcttatgaaaacatctggttccattcaagaccccaggagacctttgtgccccaacctccaaaccaggaagatgactgtgtctttgggcctccatttgattttcccctctttcaggggctccaggtccatggagttgaagaacaagggggtttctga

SEQ ID NO: 86 - protein, 455 aa, synthetic construct
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPADPMKEILPMSWLRYLLLVLGCFSGFFSCVYILVKRRGKTSFWSDVPDPAHSSLSSWLPTIMTEETFQLPSFWDSSVPSITKITELEEDKKPTHWDSESSGNGSLPALVQAYVLQGDPREISNQSQPPSRTGDQVLYGQVLESPTSPGVMQYIRSDSTQPLLGGPTPSPKSYENIWFHSRPQETFVPQPPNQEDDCVFGPPFDFPLFQGLQVHGVEEQGGF

SEQ ID NO: 87 - DNA, 1347 bp, synthetic construct
  source 1..1347: mol_type = other DNA
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaaggccagcatctgacttaaacattttcctgggcatactttgcttagtactcttgtccactacctgtgtagtgacctggctctgctgcaaacgcagaggaaagacttccttctggtcagatgtgccagacccagcccacagtagcctgagctcctggttgcccaccatcatgacagaggaaaccttccagttacccagcttctgggactccagcgtgccatcaatcaccaagatcactgaactggaggaagacaagaaaccgacccactgggattccgaaagctctgggaatggtagccttccagccctggttcaggcctatgtgctccaaggagatccaagagaaatttccaaccagtcccagcctccctctcgcactggtgaccaggtcctctatggtcaggtgcttgagagccccaccagcccaggagtaatgcagtacattcgctctgactccactcagcccctcttggggggccccacccctagccctaaatcttatgaaaacatctggttccattcaagaccccaggagacctttgtgccccaacctccaaaccaggaagatgactgtgtctttgggcctccatttgattttcccctctttcaggggctccaggtccatggagttgaagaacaagggggtttctga

SEQ ID NO: 88 - protein, 448 aa, synthetic construct
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPASDLNIFLGILCLVLLSTTCVVTWLCCKRRGKTSFWSDVPDPAHSSLSSWLPTIMTEETFQLPSFWDSSVPSITKITELEEDKKPTHWDSESSGNGSLPALVQAYVLQGDPREISNQSQPPSRTGDQVLYGQVLESPTSPGVMQYIRSDSTQPLLGGPTPSPKSYENIWFHSRPQETFVPQPPNQEDDCVFGPPFDFPLFQGLQVHGVEEQGGF

SEQ ID NO: 89 - DNA, 1356 bp, synthetic construct
  source 1..1356: mol_type = other DNA
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcagcccttgggaaggacaccattccgtggctcggccacctcctcgtgggtctcagcggggcttttggcttcatcatcttagtgtacttgctgatcagccccaacaggaagaatcccctctggccaagtgtcccagacccagctcacagcagcctgggctcctgggtgcccacaatcatggaggaggatgccttccagctgcccggccttggcacgccacccatcaccaagctcacagtgctggaggaggatgaaaagaagccggtgccctgggagtcccataacagctcagagacctgtggcctccccactctggtccagacctatgtgctccagggggacccaagagcagtttccacccagccccaatcccagtctggcaccagcgatcaggtcctttatgggcagctgctgggcagccccacaagcccagggccagggcactatctccgctgtgactccactcagcccctcttggcgggcctcacccccagccccaagtcctatgagaacctctggttccaggccagccccttggggaccctggtaaccccagccccaagccaggaggacgactgtgtctttgggccactgctcaacttccccctcctgcaggggatccgggtccatgggatggaggcgctggggagcttctag

SEQ ID NO: 90 - protein, 451 aa, synthetic construct
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLISPNRKNPLWPSVPDPAHSSLGSWVPTIMEEDAFQLPGLGTPPITKLTVLEEDEKKPVPWESHNSSETCGLPTLVQTYVLQGDPRAVSTQPQSQSGTSDQVLYGQLLGSPTSPGPGHYLRCDSTQPLLAGLTPSPKSYENLWFQASPLGTLVTPAPSQEDDCVFGPLLNFPLLQGIRVHGMEALGSF

SEQ ID NO: 91 - DNA, 1341 bp, synthetic construct
  source 1..1341: mol_type = other DNA
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagccttcggagctacacatcatcctgggcctgttcggcctcctgctgttgctcacctgcctctgtggaactgcctggctctgttgcagccccaacaggaagaatcccctctggccaagtgtcccagacccagctcacagcagcctgggctcctgggtgcccacaatcatggaggaggatgccttccagctgcccggccttggcacgccacccatcaccaagctcacagtgctggaggaggatgaaaagaagccggtgccctgggagtcccataacagctcagagacctgtggcctccccactctggtccagacctatgtgctccagggggacccaagagcagtttccacccagccccaatcccagtctggcaccagcgatcaggtcctttatgggcagctgctgggcagccccacaagcccagggccagggcactatctccgctgtgactccactcagcccctcttggcgggcctcacccccagccccaagtcctatgagaacctctggttccaggccagccccttggggaccctggtaaccccagccccaagccaggaggacgactgtgtctttgggccactgctcaacttccccctcctgcaggggatccgggtccatgggatggaggcgctggggagcttctag

SEQ ID NO: 92 - protein, 446 aa, synthetic construct
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPSELHIILGLFGLLLLLTCLCGTAWLCCSPNRKNPLWPSVPDPAHSSLGSWVPTIMEEDAFQLPGLGTPPITKLTVLEEDEKKPVPWESHNSSETCGLPTLVQTYVLQGDPRAVSTQPQSQSGTSDQVLYGQLLGSPTSPGPGHYLRCDSTQPLLAGLTPSPKSYENLWFQASPLGTLVTPAPSQEDDCVFGPLLNFPLLQGIRVHGMEALGSF

SEQ ID NO: 93 - protein, 11 aa, Mus musculus
LNTDAYLSLQE